(19) United States
(12) Patent Application Publication
(10) Pub. No.: US 2025/0154483 A1
(43) Pub. Date: May 15, 2025

(54) NOVEL CRISPR DNA AND RNA TARGETING ENZYMES AND SYSTEMS

(71) Applicant: Arbor Biotechnologies, Inc., Cambridge, MA (US)

(72) Inventors: Elise Keston-Smith, Cambridge, MA (US); David A. Scott, San Francisco, CA (US); David R. Cheng, Boston, MA (US); Winston X. Yan, Boston, MA (US); Pratyusha Hunnewell, Needham, MA (US); Jason Carte, Boston, MA (US)

(21) Appl. No.: 18/954,137

(22) Filed: Nov. 20, 2024

Related U.S. Application Data
(63) Continuation of application No. 16/980,246, filed on Jan. 28, 2021, now Pat. No. 12,195,770.
(60) Provisional application No. 62/775,874, filed on Dec. 5, 2018.
(60) Provisional application No. 62/772,043, filed on Nov. 27, 2018.
(60) Provisional application No. 62/746,539, filed on Oct. 16, 2018.
(60) Provisional application No. 62/740,867, filed on Oct. 3, 2018.
(60) Provisional application No. 62/729,393, filed on Sep. 10, 2018.
(60) Provisional application No. 62/699,498, filed on Jul. 17, 2018.
(60) Provisional application No. 62/672,489, filed on May 16, 2018.
(60) Provisional application No. 62/666,397, filed on May 3, 2018.
(60) Provisional application No. 62/642,919, filed on Mar. 14, 2018.

(51) Int. Cl. C12N 9/22; C12N 15/10

(52) U.S. Cl. CPC C12N 9/22; C12N 15/102; C12N 2310/20

(57) ABSTRACT

The disclosure describes novel systems, methods, and compositions for the manipulation of nucleic acids in a targeted fashion. The disclosure describes non-naturally occurring, engineered CRISPR systems, components, and methods for targeted modification of nucleic acids such as DNA. Each system includes one or more protein components and one or more nucleic acid components that together target nucleic acids.

### CROSS REFERENCE TO RELATED APPLICATIONS

This application is a continuation of U.S. application Ser. No. 16/980,246, filed Jan. 28, 2021, now issued as U.S. Pat. No. 12,195,770 on Jan. 14, 2025, which is a U.S. National Phase Application under 35 U.S.C. § 371 of International Application No. PCT/US2019/022376, filed Mar. 14, 2019, which claims the benefit of priority of U.S. Application No. 62/642,919, filed Mar. 14, 2018; U.S. Application No. 62/666,397, filed May 3, 2018; U.S. Application No. 62/672,489, filed May 16, 2018; U.S. Application No. 62/699,498, filed Jul. 17, 2018; U.S. Application No. 62/729,393, filed Sep. 10, 2018; U.S. Application No. 62/740,867, filed Oct. 3, 2018; U.S. Application No. 62/746,539, filed Oct. 16, 2018; U.S. Application No. 62/772,043, filed Nov. 27, 2018; and U.S. Application No. 62/775,874, filed Dec. 5, 2018. The content of each of the foregoing applications is hereby incorporated by reference in its entirety.

### SEQUENCE LISTING

The instant application contains a Sequence Listing which has been submitted electronically in XML format and is hereby incorporated by reference in its entirety. Said XML copy, created on Jan. 22, 2025, is named A2186-701320_0013US1_C1_SL.xml and is 348,699 bytes in size.

### FIELD OF THE INVENTION

The present disclosure relates to systems, methods, and compositions used for the control of gene expression involving sequence targeting and nucleic acid editing, which uses vector systems related to Clustered Regularly Interspaced Short Palindromic Repeats (CRISPR) and components thereof.

### BACKGROUND

Recent application of advances in genome sequencing technologies and analysis have yielded significant insights into the genetic underpinning of biological activities in many diverse areas of nature, ranging from prokaryotic biosynthetic pathways to human pathologies. To fully understand and evaluate the vast quantities of information produced by genetic sequencing technologies, equivalent increases in the scale, efficacy, and ease of technologies for genome and epigenome manipulation are needed. These novel genome and epigenome engineering technologies will accelerate the development of novel applications in numerous areas, including biotechnology, agriculture, and human therapeutics.

Clustered Regularly Interspaced Short Palindromic Repeats (CRISPR) and the CRISPR-associated (Cas) genes, collectively known as the CRISPR-Cas or CRISPR/Cas systems, are currently understood to provide immunity to bacteria and archaea against phage infection. The CRISPR-Cas systems of prokaryotic adaptive immunity are an extremely diverse group of proteins effectors, non-coding elements, as well as loci architectures, some examples of which have been engineered and adapted to produce important biotechnologies.

The components of the system involved in host defense include one or more effector proteins capable of modifying DNA or RNA and an RNA guide element that is responsible to targeting these protein activities to a specific sequence on the phage DNA or RNA. The RNA guide is composed of a CRISPR RNA (crRNA) and may require an additional trans-activating RNA (tracrRNA) to enable targeted nucleic acid manipulation by the effector protein(s). The crRNA consists of a direct repeat responsible for protein binding to the crRNA and a spacer sequence that is complementary to the desired nucleic acid target sequence. CRISPR systems can be reprogrammed to target alternative DNA or RNA targets by modifying the spacer sequence of the crRNA.

CRISPR-Cas systems can be broadly classified into two classes: Class 1 systems, which are composed of multiple effector proteins that together form a complex around a crRNA, and Class 2 systems, which consist of a single effector protein that complexes with the RNA guide to target DNA or RNA substrates. The single-subunit effector composition of the Class 2 systems provides a simpler component set for engineering and application translation, and have thus far been an important source of programmable effectors. Thus, the discovery, engineering, and optimization of novel Class 2 systems may lead to widespread and powerful programmable technologies for genome engineering and beyond.

The characterization and engineering of Class 2 CRISPR-Cas systems, exemplified by CRISPR-Cas9, have paved the way for a diverse array of biotechnology applications in genome editing and beyond. For example, the effector proteins Cas12a (Cpf1) and Cas13a (C2c2) possess non-specific “collateral” single-stranded-nuclease cleavage activities, which may be harnessed to create novel diagnostics, methods, and other applications. Nevertheless, there remains a need for additional programmable effectors and systems for modifying nucleic acids and polynucleotides (i.e., DNA, RNA, or any hybrid, derivative, or modification) beyond the current CRISPR-Cas systems that enable novel applications through their unique properties.

Citation or identification of any document in this application is not an admission that such document is available as prior art to the present invention.

### SUMMARY

This disclosure provides non-naturally-occurring, engineered systems and compositions for new single-effector Class 2 CRISPR-Cas systems, together with methods for computational identification from genomic databases, development of the natural loci into an engineered system, and experimental validation and application translation. These new effectors are divergent in sequence to orthologs and homologs of existing Class 2 CRISPR effectors, and also have unique domain organizations. They provide additional features that include, but are not limited to, 1) novel DNA/RNA editing properties and control mechanisms, 2) smaller size for greater versatility in delivery strategies, 3) genotype triggered cellular processes such as cell death, and 4) programmable RNA-guided DNA insertion, excision, and mobilization. Adding the novel DNA-targeting systems described herein to the toolbox of techniques for genome and epigenome manipulation enables broad applications for specific, programmed perturbations.

In general, this disclosure relates to new CRISPR-Cas systems including newly discovered enzymes and other components used to create minimal systems that can be used in non-natural environments, e.g., in bacteria other than those in which the system was initially discovered or mammalian cells.

In one aspect, the disclosure provides engineered, non-naturally occurring CRISPR-Cas systems that include: i) an RNA guide including or consisting of a direct repeat sequence and a spacer sequence capable of hybridizing to a target nucleic acid; and ii) a CLUST.019143 (Type V-G) CRISPR-Cas effector protein, wherein the effector protein includes or consists of an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence provided in Table 4 (e.g., SEQ ID NOs: 1-8, and 15-24); wherein the effector protein is capable of binding to the RNA guide and of targeting the target nucleic acid sequence complementary to the spacer sequence. In some embodiments, the CRISPR-associated protein has a RuvC domain.

As used herein, the Type V-G (CLUST.019143) CRISPR-Cas effector proteins are also referred to as Cas12g effector proteins, and these two terms are used interchangeably in this disclosure.

In some embodiments of any of the systems described herein, the Type V-G CRISPR-Cas effector protein includes the RuvC I domain XₐX_{b}DX_{b}Xₐ (SEQ ID NO: 210), wherein Xₐ is a small residue (A or G or S or T), and X_{b} is a hydrophobic residue (C or For I or L or M or P or V or W or Y). In some embodiments, the Type V-G CRISPR-Cas effector protein includes the RuvC II domain X_{b}X_{b}X_{b}X_{b}E (SEQ ID NO: 211), wherein X_{b} is a hydrophobic residue (C or F or I or L or M or P or V or W or Y). In some embodiments, the Type V-G CRISPR-Cas effector protein includes the RuvC III domain DXNAA (SEQ ID NO: 212), wherein X is any amino acid residue. In some embodiments, the Type V-G CRISPR-Cas effector protein includes more than one of the motifs in the set SEQ ID NO: 210, SEQ ID NO: 211, SEQ ID NO: 212.

In some embodiments of any of the systems described herein, the Type V-G CRISPR-Cas effector protein is the CLUST.019143 FLYL01000025 effector (Cas12g1) (SEQ ID NO: 6).

In some embodiments of any of the systems described herein, the Type V-G CRISPR-Cas effector protein includes or consists of at least one RuvC domain, e.g., a RuvC I, RuvC II, or RuvC III domain. In some embodiments, none, one, or more of the RuvC domains are catalytically inactivated.

In some embodiments of any of the systems described herein, the Type V-G CRISPR-Cas effector protein includes or consists of one or more amino acid substitutions within at least one of the RuvC domains. In some embodiments, the one or more amino acid substitutions include, e.g., an alanine substitution at an amino residue corresponding to D513 of SEQ ID NO: 6.

In some embodiments, the one or more amino acid substitutions result in a reduction of the nuclease activity of the Type V-G CRISPR-Cas effector protein as compared to the nuclease activity of the Type V-G CRISPR-Cas effector protein without the one or more amino acid substitutions.

In some embodiments of any of the systems described herein, the RNA guide includes a direct repeat sequence including or consisting of a nucleotide sequence or subsequence thereof provided in Table 5A (e.g., SEQ ID NOs: 9-13, 25-34). In some embodiments, the direct repeat sequence includes 5′-X₁X₂X₃GNX₆TX₈X₉GACACC-3′ (SEQ ID NO: 200) proximal to its 3′ end and adjacent to the spacer sequence, wherein X₁ is A or G, X₂ is A or C or G, X₃ is C or G, X₆ is A or C or Tor U, N is any nucleic acid, X₈ is C or G or T or U, and X₉ is C or G or Tor U. In some embodiments, the direct repeat sequence includes 5′-X₁GX₃GGTX₇X₈TTACAX₁₄C-3′ (SEQ ID NO: 201) proximal to its 3′ end and adjacent to the spacer sequence, wherein X₁ is C or G, X₃ is G or Tor U, X₇ is A or T or U, X₈ is C or G, and X₁₄ is A or C.

In some embodiments, the Type V-G RNA guide includes or consists of a nucleotide sequence or subsequence thereof provided in Table 5B (e.g., SEQ ID Nos: 150-167), wherein the spacer sequence is denoted by contiguous Ns. In some embodiments, the Type V-G RNA guide includes or consists of a nucleotide sequence constructed by the concatenation of a direct repeat, spacer, direct repeat sequence wherein the direct repeat sequence is provided in Table 5A and the length of the spacer is provided in the Spacer Lens column in Table 5B.

In some embodiments, the direct repeat of the RNA guide is truncated at the 5′ end by between 1 to about 25 nucleotides (e.g., 16, 17, 18, 19, 20, 21, 22, 23, or 24 nucleotides). In some embodiments, the direct repeat of the RNA guide is truncated at the 5′ end by 18 nucleotides. In some embodiments, the full length or truncated direct repeat of the RNA guide is linked at the 5′ end to a full length or truncated tracrRNA.

In some embodiments of any of the systems described herein, the spacer sequence of the RNA guide includes or consists of between about 20 to about 38 nucleotides (e.g., 16, 17, 18, 19, 20, 21, 22, 23, or 24 nucleotides).

In some embodiments of any of the systems described herein, the RNA guide includes a tracrRNA sequence including a nucleotide sequence provided in Table 6 (e.g., SEQ ID NOs: 100-116). In some embodiments of any of the systems described herein, the RNA guide includes a tracrRNA encoded by a sequence (or fragment thereof) listed in Table 7 (e.g., SEQ ID NOs: 117-134). In some embodiments, the RNA guide includes a tracrRNA sequence that includes the sequence

(SEQ ID NO: 250)
GAUGCUUACUUAGUCAUCUGGUUGGCAAACCUCCGCGGACCUUCGGGACC
AAUGGAGAGGAACCCAGCCGAGAAGCAUCGAGCCGGUAAAUGCCGGAAA.

In some embodiments of any of the systems provided herein, the target nucleic acid is a DNA. In some embodiments of any of the systems provided herein, the target nucleic acid is a single-stranded DNA. In some embodiments of any of the systems provided herein, the target nucleic acid is an RNA. In some embodiments of any of the systems provided herein, the target nucleic acid is a single-stranded RNA.

In certain embodiments of any of the systems provided herein, the targeting of the target nucleic acid by the CRISPR-associated protein and RNA guide results in a modification (e.g., a single-stranded or a double-stranded cleavage event) in the target nucleic acid. In some embodiments, the modification is a deletion event. In some embodiments, the modification is an insertion event. In some embodiments, the modification results in cell toxicity and/or cell death.

In some embodiments, the CRISPR associated protein has non-specific (i.e., “collateral”) nuclease (e.g., DNase or RNase) activity. In certain embodiments of any of the systems provided herein, the system further includes a donor template nucleic acid (e.g., a DNA or a RNA).

In some embodiments of any of the systems provided herein, the system is within a cell (e.g., a eukaryotic cell (e.g., a mammalian cell) or a prokaryotic cell (e.g., a bacterial cell).

In some embodiments of any of the systems provided herein, the RNA guide includes a tracrRNA.

In another aspect, the disclosure provides methods of targeting and editing a target nucleic acid, wherein the methods include contacting the target nucleic acid with any of the systems described herein.

In another aspect, the disclosure provides methods of targeting the insertion of a payload nucleic acid at a site of a target nucleic acid, wherein the methods include contacting the target nucleic acid with any of the systems described herein.

In yet another aspect, the disclosure provides methods of targeting the excision of a payload nucleic acid from a site at a target nucleic acid, wherein the methods include contacting the target nucleic acid with any of the systems described herein.

In some embodiments of any of the methods described herein, the target nucleic acid is present at a transcriptionally-active site.

In another aspect, the disclosure provides methods of cleaving or non-specifically degrading single-stranded DNA upon recognition of a DNA or RNA target nucleic acid, wherein the methods include contacting the target nucleic acid with any of the systems described herein.

In another aspect, the disclosure provides methods of cleaving or non-specifically degrading single-stranded RNA upon recognition of a DNA or RNA target nucleic acid, wherein the methods include contacting the target nucleic acid with any of the systems described herein.

In some embodiments of the methods, the target nucleic acid does not have a protospacer adjacent motif (PAM) or protospacer flanking sequence (PFS).

In some embodiments of the system, the spacer sequence comprises 18-23 nucleotides.

In some embodiments, the methods occur at a temperature between about 42° C. to 60° C.

In some embodiments, the methods occur at a temperature of 42° C., 50° C., or 60° C.

In another aspect, the disclosure provides methods of cleaving and/or degrading single-stranded DNA, single-stranded RNA, or double-stranded RNA upon recognition of a RNA target nucleic acid, the method comprising contacting the target nucleic acid with any of the systems disclosed herein.

In another aspect the disclosure provides methods of inducing dormancy or death of a cell which include contacting the cell with a system described herein (and compositions for use in such methods), wherein the spacer sequence is complementary to at least 15 nucleotides of the target nucleic acid, wherein the Type V-G CRISPR effector protein associates with the RNA guide to form a complex, wherein the complex binds to a target nucleic acid sequence that is complementary to the at least 15 nucleotides of the spacer sequence, and wherein upon binding of the complex to the target nucleic acid sequence the Type V-G CRISPR-Cas effector protein cleaves a non-target nucleic acid within the cell, thereby inducing dormancy or death of the cell. In some embodiments, the death is via apoptosis, necrosis, necroptosis, or a combination thereof.

In some embodiments of any of the methods described herein (and compositions for use in such methods), the target nucleic acid is an RNA selected from the group consisting of an mRNA, a tRNA, a ribosomal RNA, a non-coding RNA, a lncRNA, or a nuclear RNA. In some embodiments of any of the methods described herein, the target nucleic acid is a DNA selected from the group consisting of chromosomal DNA, mitochondrial DNA, single-stranded DNA, or plasmid DNA.

In some embodiments of any of the methods described herein (and compositions for use in such methods), upon binding of the complex to the target nucleic acid, the Type V-G CRISPR-Cas effector protein exhibits collateral RNase activity.

In some embodiments of any of the methods described herein (and compositions for use in such methods), the cell is a eukaryotic cell. In some embodiments, the cell is an animal cell. In some embodiments, the cell is a cancer cell (e.g., a tumor cell). In some embodiments, the cell is an infectious agent cell or a cell infected with an infectious agent. In some embodiments, the cell is a bacterial cell, a cell infected with a virus, a cell infected with a prion, a fungal cell, a protozoan, or a parasite cell.

In another aspect, the disclosure provides methods of treating a condition or disease in a subject in need thereof and compositions for use in such methods. The methods include administering to the subject a system described herein, wherein the spacer sequence is complementary to at least 15 nucleotides of a target nucleic acid associated with the condition or disease, wherein the Type V-G CRISPR-Cas effector protein associates with the RNA guide to form a complex, wherein the complex binds to a target nucleic acid sequence that is complementary to the at least 15 nucleotides of the spacer sequence, and wherein upon binding of the complex to the target nucleic acid sequence the Type V-G CRISPR-Cas effector protein cleaves or silences the target nucleic acid, thereby treating the condition or disease in the subject.

In some embodiments, the methods described here are used to treat a subject, e.g., a mammal, such as a human patient. The mammalian subject can also be a domesticated mammal, such as a dog, cat, horse, monkey, rabbit, rat, mouse, cow, goat, or sheep.

In some embodiments of the methods described herein (and compositions for use in such methods), the condition or disease is a cancer or an infectious disease. In some embodiments, the condition or disease is cancer, and wherein the cancer is selected from the group consisting of Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, and urinary bladder cancer.

In another aspect, the disclosure provides the use of a system described herein in a method selected from the group consisting of RNA sequence specific interference; RNA sequence-specific gene regulation; screening of RNA, RNA products, lncRNA, non-coding RNA, nuclear RNA, or mRNA; mutagenesis; inhibition of RNA splicing; fluorescence in situ hybridization; breeding; induction of cell dormancy; induction of cell cycle arrest; reduction of cell growth and/or cell proliferation; induction of cell anergy; induction of cell apoptosis; induction of cell necrosis; induction of cell death; or induction of programmed cell death.

In some embodiments of any of the systems described herein, the Type V-G CRISPR-Cas effector protein is fused to a base-editing domain, an RNA methyltransferase, an RNA demethylase, a splicing modifier, a localization factor, or a translation modification factor. In some embodiments of any of the systems described herein, the Type V-G CRISPR-Cas effector protein is fused to a base-editing domain (e.g., Adenosine Deaminase Acting on RNA (ADAR) 1 (ADAR1), ADAR2, apolipoprotein B mRNA editing enzyme, catalytic polypeptide-like (APOBEC)), and activation-induced cytidine deaminase (AID)), an RNA methyltransferase, an RNA demethylase, a splicing modifier, a localization factor, or a translation modification factor.

In some embodiments, the Type V-G CRISPR-Cas effector protein further includes a linker sequence. In some embodiments, the Type V-G CRISPR-Cas effector protein includes one or more mutations or amino acid substitutions that render the Type V-G CRISPR-Cas effector protein unable to cleave RNA.

In some embodiments, the systems described herein also include an RNA-binding fusion polypeptide that includes an RNA-binding domain (e.g. MS2) and a base-editing domain (e.g., ADAR1, ADAR2, APOBEC, and AID). In some embodiments, the RNA-binding domain is MS2, PP7, or Qbeta.

In another aspect, the disclosure provides method of modifying an RNA molecule, comprising contacting the RNA molecule with a system described herein. These methods can be carried out ex vivo or in vitro. In some embodiments, the methods described herein do not modify the germ line genetic identity of a human being.

In some embodiments, the Type V-G CRISPR-Cas effector protein includes or consist of at least one (e.g., two, three, four, five, six, or more) nuclear localization signal (NLS). In some embodiments, the Type V-G CRISPR-Cas effector protein includes or consist of at least one (e.g., two, three, four, five, six, or more) nuclear export signal (NES). In some embodiments, the Type V-G CRISPR-Cas effector protein includes at least one (e.g., two, three, four, five, six, or more) NLS and at least one (e.g., two, three, four, five, six, or more) NES.

In some embodiments, the systems described herein include a nucleic acid encoding one or more RNA guides. In some embodiments, the nucleic acid encoding the one or more RNA guides is operably linked to a promoter (e.g., a constitutive promoter or an inducible promoter).

In some embodiments, the systems described herein include a nucleic acid encoding a target nucleic acid (e.g., a target RNA). In some embodiments, the nucleic acid encoding the target nucleic acid is operably linked to a promoter (e.g., a constitutive promoter or an inducible promoter).

In some embodiments, the systems described herein include a nucleic acid encoding a Type V-G CRISPR-Cas effector protein in a vector. In some embodiments, the system further includes one or more nucleic acids encoding an RNA guide present in the vector.

In some embodiments, the vectors included in the systems are viral vectors (e.g., retroviral vectors, lentiviral vectors, adenoviral vectors, adeno-associated vectors, and herpes simplex vectors. In some embodiments, the vectors included in the system are phage vectors.

In some embodiments, the systems provided herein are in a delivery system. In some embodiments, the delivery system is a nanoparticle, a liposome, an exosome, a microvesicle, and a gene-gun.

The disclosure also provides a cell (e.g., a eukaryotic cell or a prokaryotic cell (e.g., a bacterial cell)) comprising a system described herein. In some embodiments, the eukaryotic cell is a mammalian cell (e.g., a human cell) or a plant cell. The disclosure also provides animal models (e.g., rodent, rabbit, dog, monkey, or ape models) and plant model that include the cells.

In yet another aspect, the disclosure provides methods of detecting a target nucleic acid (e.g., DNA or RNA) in a sample, the method including: (a) contacting the sample with any of the systems described herein and a labeled reporter nucleic acid, wherein hybridization of the effector: RNA guide to the target nucleic acid causes cleavage of the labeled reporter nucleic acid; and

(b) measuring a detectable signal produced by cleavage of the labeled reporter nucleic acid, thereby detecting the presence of the target nucleic acid in the sample.

In some embodiments, the methods of detecting a target nucleic acid can also include comparing a level of the detectable signal with a reference signal level, and determining an amount of target nucleic acid in the sample based on the level of the detectable signal.

In some embodiments, the methods of detecting a target nucleic acid can also include the usage of an RNA reporter nucleic acid and a DNA reporter nucleic acid on a different channel (e.g. fluorescent color), and determining the level of a detectable signal by measuring the signal level of both RNA and DNA reporters, and determining an amount of target nucleic acid in the sample based on combining (e.g. using the minimum or multiplicative product) the levels of the detectable signals.

In some embodiments, the measuring is performed using gold nanoparticle detection, fluorescence polarization, colloid phase transition/dispersion, electrochemical detection, or semiconductor based-sensing.

In some embodiments, the labeled reporter nucleic acid can include a fluorescence-emitting dye pair, a fluorescence resonance energy transfer (FRET) pair, or a quencher/fluorophore pair, wherein cleavage of the labeled reporter nucleic acid by the effector protein results in an increase or a decrease of the amount of signal produced by the labeled reporter nucleic acid.

Turning to another aspect of this disclosure, methods of modifying a target RNA are provided that generally include contacting the target RNA with a complex comprising a CRISPR-Cas Type V-G (i.e., Cas12g) effector protein and an engineered Type V-G RNA guide (e.g., a crRNA, guide RNA, RNA guide, or like structure, optionally comprising one or more nucleotide, nucleobase or backbone modifications). The Type V-G RNA guide is designed to hybridize with (e.g., is at least 70%, 75%, 80%, 85%, 90%, 91%, 92%, 93%, 94%, 95%, 96%, 97%, 98%, 99%, or 100% complementary to) a target sequence of the target RNA, and the system is distinguished by the Cas12g effector protein, Type V-G RNA guide, and Type V-G tracrRNA (either fused to expressed separately from the RNA guide) form a complex that associates with the target RNA, thereby modifying the target RNA.

In certain embodiments, modifying the target RNA includes cleaving at least one strand of the target RNA (e.g., creating a single-stranded break or, if the target RNA is an RNA duplex, creating a “nick,” or creating a double-stranded break). In certain embodiments, contacting the target RNA activates the Type V-G complex to cleave at least one strand of a non-target RNA (e.g., creating a single-strand break or, if the target RNA is an RNA duplex, creating a “nick,” or creating a double strand break). In certain embodiments, contacting the target RNA activates the Type V-G complex to cleave at least one strand of a non-target DNA (e.g., creating a single-strand break or, creating a “nick,” or creating a double strand break). Alternatively, or additionally, modification of the target RNA comprises either (i) binding to the target RNA, thereby preventing the target RNA from associating with another biomolecule or complex. The Cas12g effector protein is, in certain embodiments, a Cas12g1 effector protein.

Continuing with this aspect of the disclosure, in certain embodiments the contacting of the target RNA with the complex occurs in a diagnostic assay, for instance by contacting a target RNA with the complex, which complex is formed in vitro.

In another aspect, this disclosure relates to methods of altering a non-target RNA or non-target DNA, including contacting the target RNA with a complex including a Type V-G CRISPR-Cas (i.e., Cas12g) effector protein and a Type V-G RNA guide (e.g., a crRNA, guide RNA, or like structure, optionally comprising one or more nucleotide, nucleobase or backbone modifications) comprising a 27-39 nucleotide spacer sequence having at least 70%, 75%, 80%, 85%, 90%, or 95%, e.g., 96%, 97%, 98%, 99%, or 100%, complementarity to a sequence in the target RNA, and a Type V-G tracrRNA (either fused to expressed separately from the RNA guide) resulting in activation of the complex and alteration of the non-target RNA or non-target DNA.

In various embodiments, the non-target RNA or non-target DNA contain fluorophore quencher pairs at the sequence termini, and alteration by the activated Type V-G complex results in a fluorescent signal. In various embodiments, alteration the non-target RNA or non-target DNA by the activated Type V-G complex provides a diagnostic reporter indicating the presence of the target RNA. In various embodiments, the Cas12g protein comprises an amino acid sequence having at least 95%, e.g., at least 96%, 97%, 98%, 99%, or 100%, sequence identity to SEQ ID NO: 6, the Type V-G RNA guide comprises a direct repeat sequence with at least 95%, e.g., at least 96%, 97%, 98%, 99%, or 100%, sequence identity to one of SEQ ID NO: 13, and the Type V-G tracrRNA comprises a direct repeat sequence with at least 95%, e.g., at least 96%, 97%, 98%, 99%, or 100%, sequence identity to one of SEQ ID NO: 250. The target RNA is optionally a cellular RNA, a viral RNA, or a synthetic RNA.

Continuing with this aspect of the disclosure, in certain embodiments the contacting of the target RNA with the complex can occur in a cell, for instance by (a) contacting the cell with the complex, which complex is formed in vitro, or (b) contacting the cell with one or more nucleic acids encoding the Cas12g effector protein, the Type V-G RNA guide, and the Type V-G tracrRNA (either fused to expressed separately from the Type V-G RNA guide) which are then expressed by the cell and which form the complex within the cell. In some cases, the cell is a prokaryotic cell; in other cases, it is a eukaryotic cell.

In another aspect, this disclosure relates to method of altering a target RNA, comprising the step of contacting the target RNA within the cell with a genome editing system comprising a Cas12g protein and a Type V-G RNA guide (e.g., a crRNA, guide RNA or like structure, optionally including one or more nucleotide, nucleobase, or backbone modifications) comprising a 27-39 nucleotide spacer sequence having at least 70%, 75%, 80%, 85%, 90%, 91%, 92%, 93%, 94%, 95%, 96%, 97%, 98%, 99%, or 100% complementarity to a sequence in the target RNA, and a Type V-G tracrRNA (either fused to or expressed separately from the RNA guide). In various embodiments, the Cas12g protein comprises an amino acid sequence having at least 95%, e.g., 96%, 87%, 98%, 99%, or 100%, sequence identity to SEQ ID NO: 6, the Type V-G RNA guide comprises a direct repeat sequence with at least 95%, e.g., 96%, 87%, 98%, 99%, or 100%, sequence identity to one of SEQ ID NO: 13, and the Type V-G tracrRNA comprises a direct repeat sequence with at least 95%, e.g., 96%, 87%, 98%, 99%, or 100%, sequence identity to one of SEQ ID NO: 250. The target RNA is optionally a cellular RNA, and the contacting optionally occurs within a cell such as a prokaryotic cell or a eukaryotic cell (e.g., a mammalian cell, a plant cell, a human cell, etc.).

In yet another aspect, this disclosure relates to an engineered, non-naturally occurring Clustered Regularly Interspaced Short Palindromic Repeat (CRISPR)-associated (Cas) system comprising one or more Type V-G RNA guides or one or more nucleic acids encoding the one or more Type V-G RNA guides, wherein the Type V-G RNA guide includes a direct repeat sequence and a spacer sequence capable of hybridizing to a target nucleic acid; and a Type V-G CRISPR-Cas effector protein or a nucleic acid encoding the Type V-G CRISPR-Cas effector protein, wherein the Type V-G CRISPR-Cas effector protein is capable of binding to the RNA guide and of targeting the target nucleic acid sequence complementary to the spacer sequence, and wherein the target nucleic acid is a RNA.

In some embodiments, the Type V-G CRISPR-Cas effector protein comprises an amino acid sequence having at least 90%, or at least 95%, sequence identity to one of SEQ ID NOs: 1-24. According to certain embodiments, the Type V-G CRISPR-Cas effector protein comprises an amino acid sequence given by SEQ ID NO: 6. The total length of the CRISPR-Cas effector protein according to certain embodiments is less than 850 amino acids, excluding any amino acid signal sequence or peptide tag fused thereto. In some cases, the CRISPR-Cas effector protein comprises an amino acid substitution, for instance a substitution at an amino acid residue corresponding to D513, E655, or D745 of SEQ ID NO: 6. The substitution is optionally an alanine.

In yet another aspect, this disclosure relates to an engineered, non-naturally occurring CRISPR-Cas system, including a Cas12g effector protein, and an engineered Type V-G RNA guide (e.g., a crRNA, guide RNA, RNA guide or like structure, optionally comprising one or more nucleotide, nucleobase or backbone modifications) having a 27-39 nucleotide spacer sequence that is at least 80%, e.g., 85%, 90%, 91%, 92%, 93%, 94%, 95%, 96%, 97%, 98%, 99%, or 100%, complementary to a target sequence. The system also contains a Type V-G tracrRNA (either fused or expressed separately from the RNA guide). The Cas12g effector protein, the Type V-G RNA guide, and the Type V-G tracrRNA (either fused or expressed separately from the RNA guide) form a complex that associates with the target sequence.

In some instances, the complex of the Cas12g effector protein, Type V-G RNA guide, and Type V-G tracrRNA causes cleavage of at least one strand of a RNA comprising the target sequence. In certain embodiments, the Cas12g effector protein comprises an amino acid sequence having at least 95% sequence identity to SEQ ID NO: 6 and the direct repeat sequence has at least 95% sequence identity to SEQ ID NO: 13 and the tracr sequence has at least 95% sequence identity to SEQ ID NO: 250. Alternatively, or additionally, the Cas12g effector protein comprises an amino acid substitution (optionally, an alanine substitution) selected from the group consisting of (a) a substitution at an amino acid residue corresponding to D513, E655, or D745 of SEQ ID NO: 6.

In still another aspect, this disclosure relates to a composition including one or more nucleic acids encoding a CRISPR-Cas system (or a genome editing system) according to one of the aspects of the disclosure. In another aspect, the disclosure relates to a viral vector encoding a CRISPR-Cas system (or a genome editing system) according to one of the aspects of the disclosure.

In still another aspect, this disclosure relates to a composition including one or more nucleic acids encoding a CRISPR-Cas system (or a genome editing system) according to one of the aspects of the disclosure. In another aspect, the disclosure relates to a viral vector encoding a CRISPR-Cas system (or a genome editing system) according to one of the aspects of the disclosure.

The disclosure also includes methods of cleaving a single-stranded DNA substrate, a single-stranded RNA substrate, or a double-stranded RNA substrate with the CRISPR-Cas systems described herein. The methods include contacting the single-stranded DNA substrate, single-stranded RNA substrate, or double-stranded RNA substrate with the CRISPR-Cas system, optionally wherein the single-stranded DNA substrate, single-stranded RNA substrate, or double-stranded RNA substrate lacks a protospacer adjacent motif (PAM) sequence protospacer flanking sequence (PFS) with a target nucleic acid. In these methods, (a) when the method occurs at a temperature of about 37° C., the target nucleic acid substrate that is cleaved is a single-stranded RNA substrate is cleaved; or (b) when the method occurs at a temperature of about 50° C., a collateral nucleic acid is non-specifically cleaved, and the collateral nucleic acid is a single-stranded RNA or a single-stranded DNA, or both a single-stranded RNA and a single-stranded DNA.

In another aspect, this disclosure relates to methods of non-specifically cleaving a collateral nucleic acid, the method including contacting the collateral nucleic acid with a activated CRISPR complex disclosed herein, where the collateral nucleic is a single-stranded DNA, single-stranded RNA, or double-stranded RNA, and where the collateral nucleic acid comprises a nucleic acid sequence that does not have sequence similarity to the target nucleic acid.

In some embodiments, when the method occurs at a temperature of about 37° C., the collateral nucleic acid that is non-specifically cleaved can be a single-stranded RNA. In other embodiments, when the method occurs at a temperature of about 50° C., the collateral nucleic acid that is non-specifically cleaved can be a single-stranded RNA or a single-stranded DNA, or both a single-stranded RNA and a single-stranded DNA.

In another aspect, this disclosure relates to methods of detecting a target RNA in a sample, the method including contacting the sample with a CRISPR-Cas system disclosed herein and a labeled detector RNA, where hybridization of the RNA guide to the target RNA causes cleavage of the labeled detector RNA; and measuring a detectable signal produced by cleavage of the labeled detector RNA, thereby detecting the target RNA in the sample.

In some embodiments, the methods described herein can include comparing a level of the detectable signal with a reference signal level, and determining an amount of target RNA in the sample based on the level of the detectable signal.

In some embodiments, the measuring is performed using gold nanoparticle detection, fluorescence polarization, colloid phase transition/dispersion, electrochemical detection, or semiconductor based-sensing.

In some embodiments, the labeled detector RNA can include a fluorescence-emitting dye pair, a fluorescence resonance energy transfer (FRET) pair, or a quencher/fluor pair, wherein cleavage of the labeled detector RNA by the Type V-G CRISPR-Cas effector protein results in an increase or a decrease of an amount of signal produced by the labeled detector RNA.

In another aspect, this disclosure relates to methods of inducing genotype-specific or transcriptional-state-specific cell death or dormancy in a cell, the method including contacting a cell with a CRISPR-Cas system, e.g., the Type V-G CRISPR-Cas system, or a complex disclosed herein, where hybridization of the RNA guide to the target RNA causes a collateral RNAse activity-mediated cell death or dormancy.

In some embodiments, the cell is a prokaryotic cell or a eukaryotic cell. Further, in some embodiments the cell is a mammalian cell. In some embodiments, the cell is a cancer cell.

In some embodiments, the cell is an infectious cell or a cell infected with an infectious agent. In some embodiments, the cell is a cell infected with a virus, a cell infected with a prion, a fungal cell, a protozoan, or a parasite cell.

In some embodiments, the target nucleic acid does not have a protospacer adjacent motif (PAM) or protospacer flanking sequence (PFS).

In another aspect, this disclosure relates to methods of detecting a target nucleic acid in a sample, the method including contacting the sample with the CRISPR-Cas system, e.g., the TYPE V-G CRISPR-Cas system disclosed herein, and a labeled reporter nucleic acid, where hybridization of the crRNA to the target nucleic acid causes cleavage of the labeled reporter nucleic acid; and measuring a detectable signal produced by cleavage of the labeled reporter nucleic acid, thereby detecting a presence of the target nucleic acid in the sample.

In some embodiments, the methods described herein can include comparing a level of the detectable signal with a reference signal level, and determining an amount of target nucleic acid in the sample based on the level of the detectable signal.

In some embodiments, the measuring is performed using gold nanoparticle detection, fluorescence polarization, colloid phase transition/dispersion, electrochemical detection, or semiconductor based-sensing.

In some embodiments, the labeled reporter nucleic acid can include a fluorescence-emitting dye pair, a fluorescence resonance energy transfer (FRET) pair, or a quencher/fluor pair, wherein cleavage of the labeled reporter nucleic acid by the effector protein results in an increase or a decrease of the amount of signal produced by the labeled reporter nucleic acid.

In another aspect, this disclosure relates to methods of editing a double-stranded DNA, the method including contacting, under sufficient conditions and for a sufficient amount of time, a Cas12g enzyme and a RNA guide that directs the Cas12g enzyme to nascently transcribed RNA at a transcriptional fork; cleave one or both ssDNA strand of the double-stranded DNA at the transcriptional fork; where the method results in specific editing of a double-stranded nucleic acid with reduced off-target modification due to the cleavage prerequisite of active transcription and target recognition.

In another aspect, this disclosure relates to methods of editing a single-stranded nucleic acid, the method including contacting, under sufficient conditions and for a sufficient amount of time, a fusion protein including a Cas12g enzyme, a protein domain with RNA modifying activity, and an RNA guide targeting the double-stranded nucleic acid; and the single-stranded nucleic acid, where the contact yields a modification to the base identity of the single-stranded nucleic acid.

In another aspect, this disclosure relates to methods of treating a condition or disease in a subject in need thereof, the method comprising administering to the subject a CRISPR-Cas system, e.g., a Type V-G CRISPR-Cas system or a complex described herein, where the spacer sequence is complementary to at least 15 nucleotides of a target nucleic acid associated with the condition or disease; where the effector protein, e.g., Type V-G CRISPR-Cas effector protein, associates with the RNA guide to form a complex; where the complex binds to a target nucleic acid sequence that is complementary to the at least 15 nucleotides of the spacer sequence; and where upon binding of the complex to the target nucleic acid sequence, the effector protein, e.g., Type V-G CRISPR-Cas effector protein described herein, cleaves the target nucleic acid, thereby treating the condition or disease in the subject.

In some embodiments, the condition or disease is selected from the group consisting of Myotonic dystrophy, Huntington's disease, Familial hypobetalipoproteinemia, hereditary transthyretin-mediated amyloidosis, Familial hypercholesterolemia, Prader Willi syndrome, Spinal muscular atrophy, Dyskeratosis congenita, primary age-related tauopathy, neurofibrillary tangle predominant senile dementia, Dementia pugilistica, Duchenne Muscular Dystrophy, Becker Muscular Dystrophy, Frontotemporal dementia, Hereditary Parkinsonism Cystic fibrosis, Friedreich's ataxia, and chronic myelogenous leukemia.

In some embodiments, the condition or disease is a cancer or an infectious disease.

In some embodiments, the cancer can be Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, or urinary bladder cancer.

In some embodiments, the condition or disease is infectious in nature, and where the infectious agent is selected from the group consisting of human immunodeficiency virus (HIV), respiratory syncytial virus (RSV), hepatitis C virus (HCV), hepatitis B virus (HBV), herpes simplex virus-1 (HSV1), and herpes simplex virus-2 (HSV2).

In some embodiments, the system, activated CRISPR complex, or cells described herein is use as a medicament or for treatment or prevention of a cancer or an infectious disease, or as an antimicrobial or antiviral.

In some embodiments, the system, activated CRISPR complex, or cells described herein is used in an in vitro or ex vivo method of: targeting and editing a target nucleic acid; cleaving a single-stranded DNA substrate, a single-stranded RNA substrate, or a double-stranded RNA substrate; non-specifically cleaving and/or degrading a collateral nucleic acid; detecting a target RNA in a sample; specifically editing a double-stranded nucleic acid; base editing a double-stranded nucleic acid, specifically editing a single-stranded nucleic acid; base editing a single-stranded nucleic acid, inducing genotype-specific or transcriptional-state-specific cell death or dormancy in a cell.

In some embodiments, the system, activated CRISPR complex, or cells described herein is used in targeting and editing a target nucleic acid; cleaving a single-stranded DNA substrate, a single-stranded RNA substrate, or a double-stranded RNA substrate; non-specifically cleaving and/or degrading a collateral nucleic acid; detecting a target RNA in a sample; specifically editing a double-stranded nucleic acid; base editing a double-stranded nucleic acid, specifically editing a single-stranded nucleic acid; base editing a single-stranded nucleic acid, inducing genotype-specific or transcriptional-state-specific cell death or dormancy in a cell, where the use does not comprise a process for modifying the germ line genetic identity of a human being and does not comprise a method of treatment of the human or animal body.

Yet another aspect, method of cleaving a first and a second nucleic acid substrate, comprising contacting the first and second nucleic acid substrates with the system disclosed herein where the first nucleic acid substrate is a single-stranded (ss)DNA or an ssRNA comprising a target sequence that is at least partially complementary to the spacer sequence, the second nucleic acid substrate is an ssDNA or an ssRNA that does not comprise the target sequence.

In some embodiments, the first nucleic acid substrate is a first strand of a DNA and the second nucleic acid substrate is an RNA transcript of the first strand or a second strand of the DNA that is at least partially complementary to the first strand.

In some embodiments, the CRISPR-Cas effector protein lacks both an HNH domain and an HEPN domain.

The term “cleavage event,” as used herein, refers to: (1) a DNA or RNA break in a target nucleic acid created by a nuclease of a CRISPR system described herein; or (2) a DNA or RNA break in a collateral (i.e., non-specific or non-target) nucleic acid substrate. In some embodiments, the cleavage event is a double-stranded DNA break. In some embodiments, the cleavage event is a single-stranded DNA break. In some embodiments, the cleavage event is a single-stranded RNA break. In some embodiments, the cleavage event is a double-stranded RNA break.

The terms “CRISPR-Cas system,” “Type V-G CRISPR-Cas system,” “Type V-G system,” or “Cas12g effector protein,” as used herein refer to a Type V-G CRISPR-Cas effector protein and a Type V-G RNA guide, and/or nucleic acids encoding the Type V-G CRISPR-Cas effector protein or Type V-G RNA guide, where the Type V-G RNA guide includes a tracrRNA and a crRNA, and optionally promoters operably linked to the expression of the CRISPR effector or the RNA guide or to both.

The term “CRISPR array” as used herein refers to the nucleic acid (e.g., DNA) segment that includes CRISPR repeats and spacers, starting with the first nucleotide of the first CRISPR repeat and ending with the last nucleotide of the last (terminal) CRISPR repeat. Typically, each spacer in a CRISPR array is located between two repeats. The terms “CRISPR repeat,” or “CRISPR direct repeat,” or “direct repeat,” as used herein, refer to multiple short direct repeating sequences, which show very little or no sequence variation within a CRISPR array. Suitably, a Type V-G direct repeat may form a stem-loop structure and/or may form a stem-loop structure in combination with a trans-activating CRISPR RNA.

A “stem-loop structure” refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures are well known in the art and these terms are used consistently with their known meanings in the art. As is known in the art, a stem-loop structure does not require exact base-pairing. Thus, the stem may include one or more base mismatches. Alternatively, the base-pairing may be exact, i.e. not include any mismatches. The predicted stem-loop structures of some Type V-G direct repeats are illustrated in FIG. 3, and the predicted stem-loop structure of one Type V-G repeat when hybridized to a tracrRNA is shown in FIG. 13.

The terms “CRISPR RNA” or “crRNA” as used herein refer to an RNA molecule comprising a guide sequence used by a CRISPR effector to specifically target a nucleic acid sequence. Typically, crRNAs contain a spacer sequence that mediates target recognition, and a direct repeat sequence (referred to herein as a direct repeat or “DR” sequence) that forms a duplex with a tracrRNA. The crRNA:tracrRNA duplex binds to a CRISPR-Cas effector protein.

The term “donor template nucleic acid,” as used herein refers to a nucleic acid molecule that can be used by one or more cellular proteins to alter the structure of a target nucleic acid after a CRISPR enzyme described herein has altered a target nucleic acid. In some embodiments, the donor template nucleic acid is a double-stranded nucleic acid. In some embodiments, the donor template nucleic acid is a single-stranded nucleic acid. In some embodiments, the donor template nucleic acid is linear. In some embodiments, the donor template nucleic acid is circular (e.g., a plasmid). In some embodiments, the donor template nucleic acid is an exogenous nucleic acid molecule. In some embodiments, the donor template nucleic acid is an endogenous nucleic acid molecule (e.g., a chromosome).

The terms “CRISPR-Cas effector,” “CRISPR effector,” “effector,” “CRISPR-associated protein,” or “CRISPR enzyme,” “Type V-G CRISPR-Cas effector protein,” “Type V-G CRISPR-Cas effector,” “Type V-G effector,” or Cas12g effector protein,” as used herein refer to a protein that carries out an enzymatic activity or that binds to a target site on a nucleic acid specified by an RNA guide. In addition, a CRISPR-Cas effector protein associated with Type V-G CRISPR-Cas systems may be referred to herein as Cas12g or a Cas12g enzyme. In some embodiments, a Type V-G CRISPR-Cas effector protein has RNA nuclease activity and/or DNA nuclease activity.

The terms “CRISPR effector complex,” “effector complex,” or “surveillance complex” as used herein refer to a complex containing a CRISPR effector and an RNA guide.

The term “RNA guide” as used herein refers to any RNA molecule that facilitates the targeting of a protein described herein to a target nucleic acid. Exemplary “RNA guides” include, but are not limited to, a crRNA, as well as a crRNA hybridized to or fused to a tracrRNA. In some embodiments, an RNA guide includes a crRNA in the form of a pre-crRNA (e.g. DR-spacer-DR). In some embodiments, an RNA guide includes a crRNA in the form of a mature crRNA (e.g. mature_DR-spacer or mature_DR-spacer-mature_DR). In some embodiments, an RNA guide includes both a crRNA and a tracrRNA, either fused into a single RNA molecule or as separate RNA molecules.

As used herein, the term “targeting” refers to the ability of a complex including a CRISPR-associated protein and a RNA guide, such as a crRNA, to preferentially or specifically bind to, e.g., hybridize to, a specific target nucleic acid and compared to other nucleic acids that do not have the same sequence as the target nucleic acid.

As used herein, the term “target nucleic acid” refers to a specific nucleic acid substrate that contains a nucleotide sequence complementary to the entirety or a part of the spacer in the RNA guide. In some embodiments, the target nucleic acid comprises a gene or a sequence within a gene. In some embodiments, the target nucleic acid comprises a non-coding region (e.g., a promoter). In some embodiments, the target nucleic acid is single-stranded. In some embodiments, the target nucleic acid is double-stranded.

The terms “trans-activating crRNA” or “tracrRNA” as used herein refer to an RNA including an anti-repeat region complimentary to all or part of the direct repeat sequence of a CRISPR RNA (crRNA). The CRISPR effector bound to the crRNA and tracrRNA (RNA guide) form a functional complex capable of binding to a target nucleic acid.

A “transcriptionally-active site” as used herein refers to a site in a nucleic acid sequence that includes one or more promoter regions at which transcription is initiated and actively occurs.

The terms “activated CRISPR complex” or “activated complex” as used herein refer to a CRISPR effector complex after it has bound to or has modified a target nucleic acid.

The terms “collateral RNA” or “collateral DNA” as used herein refer to a nucleic acid substrate that is cleaved non-specifically by an activated CRISPR complex. Suitably, a “collateral RNA” or “collateral DNA” may have no sequence similarity to a target nucleic acid or spacer sequence.

The term “collateral RNase activity,” as used herein in reference to a CRISPR enzyme, refers to non-specific RNase activity of an activated CRISPR complex.

The term “collateral DNase activity,” as used herein in reference to a CRISPR enzyme, refers to non-specific DNase activity of an activated CRISPR complex.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF FIGURE DESCRIPTION

The figures are a series of schematics and nucleic acid and amino acid sequences that represent the results of locus analysis of various protein clusters.

FIGS. 1A and 1B together depict a classification tree of Type V effectors (Cas12 proteins). The corresponding CRISPR-Cas loci organization is shown for each branch, with the need for a tracrRNA depicted by a white rectangle adjacent to a CRISPR array. CLUST.019143 (Type V-G) systems are depicted as Cas12g.

FIG. 2A is a schematic representation of the functional domains of the CLUST.019143 (Type V-G) effector, designated Cas12g. The solid grey shading indicates the location of the C-terminal RuvC domain, with the catalytic residues in the three conserved sequence motifs (RuvC-I, RuvC-II and RuvC-III) indicated and shown to scale. The bridge helix (h) and Zn-finger domain (z, CxxC . . . CxxC) are shown only approximately to scale.

FIG. 2B is a schematic representation of a multiple sequence alignment of Cas12g effector proteins, with the relative locations of the conserved catalytic residues of the RuvC domain denoted by RuvC I/II/III.

FIG. 3 is a group of schematic diagrams that show predicted secondary structure of the RNA transcript of examples of CLUST.019143 (Type V-G) direct repeat sequences. From L to R, the sequences are (SEQ ID NO: 9), (SEQ ID NO: 10), (SEQ ID NO: 4), (SEQ ID NO: 30), (SEQ ID NO: 13), (SEQ ID NO: 28).

FIG. 4A is a schematic representation of the design of in vivo screen Effector and Non-coding Plasmids. CRISPR array libraries were designed including non-repetitive spacers uniformly sampled from both strands of pACYC184 or E. coli essential genes flanked by two DRs and expressed by J23119.

FIG. 4B is a schematic representation of the negative selection screening workflow; 1) CRISPR array libraries were cloned into the Effector Plasmid, 2) the Effector Plasmid and, when present, the Non-coding Plasmid were transformed into E. coli followed by outgrowth for negative selection of CRISPR arrays conferring interference against DNA or RNA transcripts from pACYC184 or E. coli essential genes, 3) Targeted sequencing of the Effector Plasmid was used to identify depleted CRISPR arrays and small RNA sequencing was used to identify mature crRNAs and tracrRNAs.

FIG. 5A is a graphic representation of the distribution of targets for Cas12g1 strongly depleted CRISPR arrays over the pACYC184 plasmid. Targets on the top strand and bottom strand are shown separately, and in relation to the orientation of the annotated genes.

FIG. 5B is a graphic representation of the distribution of targets for Cas12g1 strongly depleted CRISPR arrays over E. coli essential genes. Targets on the top strand and bottom strand are shown separately, and in relation to the orientation of the annotated genes.

FIG. 6A shows sequence logos the left flank and right flank of targets corresponding to strongly depleted Cas12g1 CRISPR arrays from in vivo screening.

FIG. 6B shows the bit scores for all nucleotide permutations target and flanking nucleotides up to length 3 for strongly depleted Cas12g1 CRISPR arrays from in vivo screening.

FIG. 7 is a heatmap that summarizes the number of strongly depleted CRISPR arrays detected for different Cas12g1 system compositions enumerated along the x-axis as follows; 1) Cas12g1 Effector Plasmid library with Non-coding Plasmid (minimal active system), 2) dCas12g1-D513A Effector Plasmid library with Non-coding Plasmid, and 3) Cas12g1-A513D Effector Plasmid library with Non-coding Plasmid (mutant clone restored to WT). Screen systems are additionally separated by CRISPR array expression direction. Along the y-axis, strongly depleted spacers for each system are separated by target substrate (pACYC184 or E. coli essential genes, EG) and DNA strand (S, sense; AS, antisense). CRISPR arrays strongly depleted in negative controls without Cas12g1 are subtracted from the analysis.

FIG. 8 is a scatter plot that shows the in vivo screening comparison of Cas12g1 WT versus the dCas12g1 D513A mutant (where the RuvC-I catalytic residue aspartate is mutated to alanine). Each point represents a spacer, and the value indicates the x-fold depletion under the condition specified for the axis (wild type vs mutant). Higher values indicate stronger depletion (i.e. fewer surviving colonies). Red lines indicate threshold for “strongly depleted” (>3 fold depletion).

FIG. 9 is a scatter plot that shows the in vivo screening comparison of Cas12g1 WT versus the A513D rescue mutant cloned from dCas12g1 D513A. Each point represents a spacer, and the value indicates the x-fold depletion under the condition specified for the axis. Higher values indicate stronger depletion (i.e. fewer surviving colonies). Red lines indicate threshold for “strongly depleted” (>3 fold depletion).

FIG. 10 is a scatter plot that shows the in vivo screening comparison of Cas12g1 WT with and without the Non-coding Plasmid. Each point represents a spacer, and the value indicates the x-fold depletion under the condition specified for the axis. Higher values indicate stronger depletion (i.e. fewer surviving colonies). Red lines indicate threshold for “strongly depleted” (>3 fold depletion).

FIG. 11 depicts the read mapping of small RNA sequencing of in vivo screening samples of the minimal Cas12g1 system, revealing the mature crRNA for Cas12g1 (SEQ ID NO: 202).

FIG. 12 depicts the read mapping of small RNA sequencing of in vivo screening samples of the minimal Cas12g1 system mapped to the Non-Coding Plasmid, revealing the tracrRNA for Cas12g1 (SEQ ID NO. 250).

FIG. 13 depicts a co-fold of the mature crRNA (SEQ ID NO: 202) and tracrRNA (SEQ ID NO. 250) with sequences determined by small RNAseq of E. coli expressing the Cas12g1 system from the in vivo screen.

FIG. 14 depicts a stain free 4-15% SDS-Page (BioRad) gel of effector proteins post His-tag purification, fraction pooling, and concentration.

FIGS. 15A-B depict denaturing gels showing lack of in vitro pre-crRNA processing by Cas12g1 effector protein at (FIG. 15A) 37° C. and (FIG. 15B) 50° C. Pre-crRNA with and without tracrRNA was incubated with Cas12g1 for 1 hour at 37° C. and analyzed on a 15% TBE-Urea gel.

FIG. 16A is a melting curve graph of Cas12g1 displaying the thermostability of apo Cas12g1 and increased thermostability of binary (Cas12g1+crRNA) and ternary (Cas12g1+crRNA+tracrRNA) complex of Cas12g1. Melting curve was obtained using differential scanning fluorimetry (DSF).

FIG. 16B depicts the first derivative of the thermal melts from showing the melting temperatures of apo, binary (Cas12g1+crRNA), and ternary (Cas12g1+crRNA+tracrRNA) complexes of Cas12g1. The first derivative traces of the Cas12g1 binary and ternary complexes are nearly overlapping.

FIGS. 17A-D depict denaturing gels demonstrating the absence of activity for Cas12g1 surveillance complex (Cas12g1+crRNA+tracrRNA) against ssDNA targets. Cas12g1 surveillance complex was tested for nuclease activity against DNA substrates at 37° C. (FIG. 17A), 42° C. (FIG. 17B), 50° C. (FIG. 17C) and 60° C. (FIG. 17D). The ssDNA target is 5′ end labeled with IR800 dye. For all reactions, the Cas12g1 surveillance complex was formed by pre-incubating Cas12g1, mature crRNA and tracrRNA for 10 minutes at 37° C. prior to adding the substrates and incubating for 1 hour at the specified temperature.

FIGS. 18A-D depict denaturing gels demonstrating the absence of activity for Cas12g1 surveillance complex (Cas12g1+crRNA+tracrRNA) against dsDNA targets. Cas12g1 surveillance complex was tested for nuclease activity against DNA substrates at 37° C. (FIG. 18A), 42° C. (FIG. 18B), 50° C. (FIG. 18C) and 60° C. (FIG. 18D). The dsDNA target is labeled with IR800 dye on the 5′ end of the non-spacer complementary strand. For all reactions, the Cas12g1 surveillance complex was formed by pre-incubating Cas12g1, mature crRNA and tracrRNA for 10 minutes at 37° C. prior to adding the substrates and incubating for 1 hour at the specified temperature.

FIG. 19 depicts a denaturing gel showing cleavage of sense(S) ssDNA (SEQ ID NO: 422) in the presence of sense(S) ssRNA transcripts (SEQ ID NO: 421) (with both ssDNA and ssRNA substrates having complementarity to the crRNA spacer (SEQ ID NO: 412)) by Cas12g1 apo (Cas12g only), binary (Cas12g+m-crRNA), and ternary (Cas12g+m-crRNA+tracrRNA) complexes at 50° C. Cas12g1 ternary complex was formed by pre-incubating Cas12g1 and mature crRNA and tracrRNA for 10 minutes at 37° C. prior to adding the substrates and incubating for 1 hour at the specified temperature. In the reactions, ssDNA and ssRNA substrates were hybridized prior to adding the Cas12g1 ternary complex, yielding a DNA-RNA hybrid for the sense-antisense pairings of ssDNA and ssRNA. Samples were RNase treated and analyzed on a 15% TBE-urea denaturing gel using SYBR Gold nucleic acid staining.

FIG. 20 depicts a denaturing gel showing the absence of cleavage of sense(S) ssDNA (SEQ ID NO: 422) in the presence of antisense (AS) ssRNA transcripts (SEQ ID NO: 423) by Cas12g1 apo (Cas12g only), binary (Cas12g+m-crRNA (SEQ ID NO: 412)), and ternary (Cas12g+m-crRNA+tracrRNA) complexes at 50° C. Note that only the sense ssDNA sequence has complementarity to the crRNA spacer. In the reactions, ssDNA and ssRNA substrates were hybridized prior to adding the Cas12g1 ternary complex, yielding a DNA-RNA hybrid for the sense-antisense pairings of ssDNA and ssRNA. Cas12g1 ternary complex was formed by pre-incubating Cas12g1 and mature crRNA and tracrRNA for 10 minutes at 37° C. prior to adding the substrates and incubating for 1 hour at the specified temperature. Samples were RNase treated and analyzed on a 15% TBE-urea denaturing gel using SYBR Gold nucleic acid staining.

FIG. 21 depicts a denaturing gel showing the absence of cleavage of antisense (AS) SSDNA (SEQ ID NO: 424) in the presence of sense(S) ssRNA transcripts (SEQ ID NO: 421) by Cas12g1 apo (Cas12g only), binary (Cas12g+m-crRNA (SEQ ID NO: 412)), and ternary (Cas12g+m-crRNA+tracrRNA) complexes at 50° C. The sense ssRNA substrate sequence has complementarity to the crRNA spacer. In the reactions, ssDNA and ssRNA substrates were hybridized prior to adding the Cas12g1 ternary complex, yielding a DNA-RNA hybrid for the sense-antisense pairings of ssDNA and ssRNA. Cas12g1 ternary complex was formed by pre-incubating Cas12g1 and mature crRNA and tracrRNA for 10 minutes at 37° C. prior to adding the substrates and incubating for 1 hour at the specified temperature. Samples were RNase treated and analyzed on a 15% TBE-urea denaturing gel using SYBR Gold nucleic acid staining.

FIG. 22 depicts a denaturing gel showing the absence of cleavage of antisense (AS) SSDNA (SEQ ID NO: 424) in the presence of antisense (AS) ssRNA transcripts (SEQ ID NO: 423) by Cas12g1 apo (Cas12g only), binary (Cas12g+m-crRNA (SEQ ID NO: 412)), and ternary (Cas12g+m-crRNA+tracrRNA) complexes at 50° C. Neither ssDNA nor ssRNA substrates have sequence complementarity to the crRNA spacer. In the reactions, ssDNA and ssRNA substrates were hybridized prior to adding the Cas12g1 ternary complex. Cas12g1 ternary complex was formed by pre-incubating Cas12g1 and mature crRNA and tracrRNA for 10 minutes at 37° C. prior to adding the substrates and incubating for 1 hour at the specified temperature. Samples were RNase treated and analyzed on a 15% TBE-urea denaturing gel using SYBR Gold nucleic acid staining.

FIGS. 23A-B depict denaturing gels displaying non-specific (i.e., collateral) nuclease activity of Cas12g1 surveillance complex (Cas12g1+crRNA+tracrRNA) against collateral ssDNA substrate upon recognition of the target ssRNA. IR800 dye-labeled collateral ssDNA substrate is cleaved at 50° C. by Cas12g1 surveillance complex only in the presence of unlabeled target ssRNA (FIG. 23A) and not in the presence of unlabeled non-target ssRNA (FIG. 23B). Cas12g1 surveillance complex was formed by pre-incubating Cas12g1 and mature crRNA and tracrRNA for 10 minutes at 37° C. prior to adding the substrates and incubating for 1 hour at 37° C.

FIG. 24 depicts a denaturing gel showing cleavage of sense(S) ssDNA (SEQ ID NO: 422) in the presence of sense(S) ssRNA transcripts (SEQ ID NO: 421) (with both ssDNA and ssRNA substrates having complementarity to the crRNA spacer (SEQ ID NO: 412)) by Cas12g1 apo (Cas12g only), binary (Cas12g+m-crRNA), and ternary (Cas12g+m-crRNA+tracrRNA) complexes at 37° C. Cas12g1 ternary complex was formed by pre-incubating Cas12g1 and mature crRNA and tracrRNA for 10 minutes at 37° C. prior to adding the substrates and incubating for 1 hour at the specified temperature. In the reactions, ssDNA and ssRNA substrates were hybridized prior to adding the Cas12g1 ternary complex, yielding a DNA-RNA hybrid for the sense-antisense pairings of ssDNA and ssRNA. Samples were RNase treated and analyzed on a 15% TBE-urea denaturing gel using SYBR Gold nucleic acid staining.

FIG. 25 depicts a denaturing gel showing the absence of cleavage of sense(S) ssDNA (SEQ ID NO: 422) in the presence of antisense (AS) ssRNA transcripts (SEQ ID NO: 423) by Cas12g1 apo (Cas12g only), binary (Cas12g+m-crRNA (SEQ ID NO: 412)), and ternary (Cas12g+m-crRNA+tracrRNA) complexes at 37° C. Note that only the sense ssDNA sequence has complementarity to the crRNA spacer. In the reactions, ssDNA and ssRNA substrates were hybridized prior to adding the Cas12g1 ternary complex, yielding a DNA-RNA hybrid for the sense-antisense pairings of ssDNA and ssRNA. Cas12g1 ternary complex was formed by pre-incubating Cas12g1 and mature crRNA and tracrRNA for 10 minutes at 37° C. prior to adding the substrates and incubating for 1 hour at the specified temperature. Samples were RNase treated and analyzed on a 15% TBE-urea denaturing gel using SYBR Gold nucleic acid staining.

FIG. 26 depicts a denaturing gel showing the absence of cleavage of antisense (AS) SSDNA (SEQ ID NO: 424) in the presence of sense(S) ssRNA transcripts (SEQ ID NO: 421) by Cas12g1 apo (Cas12g only), binary (Cas12g+m-crRNA (SEQ ID NO: 412)), and ternary (Cas12g+m-crRNA+tracrRNA) complexes at 37° C. The sense ssRNA substrate sequence has complementarity to the crRNA spacer. In the reactions, ssDNA and ssRNA substrates were hybridized prior to adding the Cas12g1 ternary complex, yielding a DNA-RNA hybrid for the sense-antisense pairings of ssDNA and ssRNA. Cas12g1 ternary complex was formed by pre-incubating Cas12g1 and mature crRNA and tracrRNA for 10 minutes at 37° C. prior to adding the substrates and incubating for 1 hour at the specified temperature. Samples were RNase treated and analyzed on a 15% TBE-urea denaturing gel using SYBR Gold nucleic acid staining.

FIG. 27 depicts a denaturing gel showing the absence of cleavage of antisense (AS) SSDNA (SEQ ID NO: 424) in the presence of antisense (AS) ssRNA transcripts (SEQ ID NO: 423) by Cas12g1 apo (Cas12g only), binary (Cas12g+m-crRNA (SEQ ID NO: 412)), and ternary (Cas12g+m-crRNA+tracrRNA) complexes at 37° C. Neither ssDNA nor ssRNA substrates have sequence complementarity to the crRNA spacer. In the reactions, ssDNA and ssRNA substrates were hybridized prior to adding the Cas12g1 ternary complex. Cas12g1 ternary complex was formed by pre-incubating Cas12g1 and mature crRNA and tracrRNA for 10 minutes at 37° C. prior to adding the substrates and incubating for 1 hour at the specified temperature. Samples were RNase treated and analyzed on a 15% TBE-urea denaturing gel using SYBR Gold nucleic acid staining.

FIGS. 28A-B depict denaturing gels displaying target and non-target ssRNA cleavage activity of the Cas12g1 apo (Cas12g only), binary (Cas12g+m-crRNA), and ternary (Cas12g+m-crRNA+tracrRNA) complexes at 37° C. FIG. 28A depicts manipulation of IR 800-labeled target ssRNA substrates, while FIG. 28B depicts the lack of cleavage of IR 800-labeled non-target ssRNA substrates.

FIGS. 29A-B depict denaturing gels displaying target and non-target ssRNA cleavage activity of the Cas12g1 apo (Cas12g only), binary (Cas12g+m-crRNA), and ternary (Cas12g+m-crRNA+tracrRNA) complexes at 50° C. FIG. 29A depicts cleavage of IR 800-labeled target ssRNA substrates, while FIG. 29B depicts the lack of cleavage of non-target ssRNA substrates.

FIG. 30A depicts a denaturing gel showing that Cas12g1 surveillance complex completely cleaves target ssRNA down to nucleotides at complex concentrations above 32 nM, and shows detectable RNase activity on target ssRNA at complex concentrations as low as 125 pM when assayed at 50° C.,

FIG. 30B depicts a denaturing gel showing that Cas12g1 surveillance complex displays no RNase activity with a non-target ssRNA at complex concentrations as high as 250 nM. All ssRNA substrates were labeled at the 5′ end with IR800 dye. Cas12g1 surveillance complex was formed by pre-incubating Cas12g1 and mature crRNA and tracrRNA for 10 minutes at 37° C. prior to adding the substrates and incubating for 1 hour at 50° C.

FIG. 31A depicts a denaturing gel showing robust cleavage of collateral ssRNA by Cas12g1 surveillance complex in the presence of increasing concentrations of unlabeled target SSRNA.

FIG. 31B depicts a denaturing gel showing that Cas12g1 surveillance complex does not cleave collateral ssRNA in the presence of non-target ssRNA. All collateral ssRNA substrates were labeled at the 5′ end with IR800 dye. Cas12g1 surveillance complex was formed by pre-incubating Cas12g1 with mature crRNA and tracrRNA for 10 minutes at 37° C. prior to adding the substrates and incubating for 1 hour at 50° C.

FIG. 32 depicts a denaturing gel showing nuclease activity of Cas12g1 surveillance complex on target ssRNA substrates inhibited by EDTA at 50° C.

FIG. 33 depicts a denaturing gel showing target ssRNA activated collateral ssDNA cleavage by Cas12g1 surveillance complex at 37° C. and 50° C. Samples were analyzed by 15% TBE-urea denaturing gel electrophoresis.

FIG. 34 depicts a denaturing gel showing target ssRNA activated target and collateral ssRNA cleavage by Cas12g1 surveillance complex at 37° C. Samples were analyzed by 15% TBE-urea denaturing gel electrophoresis.

FIG. 35 depicts a denaturing gel showing the absence of target ssRNA activated collateral ssDNA cleavage by dCas12g1 D513A surveillance complex at 37° C. and 50° C. Samples were analyzed by 15% TBE-urea denaturing gel electrophoresis.

FIG. 36 depicts a denaturing gel showing the absence of target ssRNA activated target and collateral ssRNA cleavage by dCas12g1 D513A surveillance complex at 37° C. Samples were analyzed by 15% TBE-urea denaturing gel electrophoresis.

FIGS. 37A-C are a series of schematics showing system components and interference mechanisms of Cas12g.

### DETAILED DESCRIPTION

The broad natural diversity of CRISPR-Cas defense systems contains a wide range of activity mechanisms and functional elements that can be harnessed for programmable biotechnologies. In a natural system, these mechanisms and parameters enable efficient defense against foreign DNA and viruses while providing self vs. non-self-discrimination to avoid self-targeting. In an engineered system, the same mechanisms and parameters also provide a diverse toolbox of molecular technologies and define the boundaries of the targeting space. For instance, systems Cas9 and Cas13a have canonical DNA and RNA endonuclease activity and their targeting spaces are defined by the protospacer adjacent motif (PAM) on targeted DNA and protospacer flanking sites (PFS) on targeted RNA, respectively.

The methods described herein have been used to discover additional mechanisms and parameters within single subunit Class 2 effector systems that can be more effectively harnessed for programmable biotechnologies.

In one aspect, the disclosure relates to the use of computational methods and algorithms to search for and identify novel protein families that exhibit a strong co-occurrence pattern with certain other features within naturally occurring genome sequences. In certain embodiments, these computational methods are directed to identifying protein families that co-occur in close proximity to CRISPR arrays. However, the methods disclosed herein are useful in identifying proteins that naturally occur within close proximity to other features, both non-coding and protein-coding (e.g., fragments of phage sequences in non-coding areas of bacterial loci; or CRISPR Cas1 proteins). It should be understood that the methods and calculations described herein may be performed on one or more computing devices.

In some embodiments, a set of genomic sequences may be obtained from genomic or metagenomic databases. The databases comprise short reads, or contig level data, or assembled scaffolds, or complete genomic sequences of organisms. Likewise, the database may comprise genomic sequence data from prokaryotic organisms, or eukaryotic organisms, or may include data from metagenomic environmental samples. Examples of database repositories include National Center for Biotechnology Information (NCBI) RefSeq, NCBI GenBank, NCBI Whole Genome Shotgun (WGS), and the Joint Genome JGI Integrated Microbial Genomes (IMG).

In some embodiments, a minimum size requirement is imposed to select genome sequence data of a specified minimum length. In certain exemplary embodiments, the minimum contig length may be 100 nucleotides, 500 nt, 1 kb, 1.5 kb, 2 kb, 3 kb, 4 kb, 5 kb, 10 kb, 20 kb, 40 kb, or 50 kb.

In some embodiments, known or predicted proteins are extracted from the complete or a selected set of genome sequence data. In some embodiments, known or predicted proteins are taken from extracting coding sequence (CDS) annotations provided by the source database. In some embodiments, predicted proteins are determined by applying a computational method to identify proteins from nucleotide sequences. In some embodiments, the GeneMark Suite is used to predict proteins from genome sequences. In some embodiments, Prodigal is used to predict proteins from genome sequences. In some embodiments, multiple protein prediction algorithms may be used over the same set of sequence data with the resulting set of proteins de-duplicated.

In some embodiments, CRISPR arrays are identified from the genome sequence data. In some embodiments, PILER-CR is used to identify CRISPR arrays. In some embodiments, CRISPR Recognition Tool (CRT) is used to identify CRISPR arrays. In some embodiments, CRISPR arrays are identified by a heuristic that identifies nucleotide motifs repeated a minimum number of times (e.g. 2, 3, or 4 times), where the spacing between consecutive occurrences of a repeated motif does not exceed a specified length (e.g. 50, 100, or 150 nucleotides). In some embodiments, multiple CRISPR array identification tools may be used over the same set of sequence data with the resulting set of CRISPR arrays de-duplicated.

In some embodiments, proteins in close proximity to CRISPR arrays are identified. In some embodiments, proximity is defined as a nucleotide distance, and may be within 20 kb, 15 kb, or 5 kb. In some embodiments, proximity is defined as the number of open reading frames (ORFs) between a protein and a CRISPR array, and certain exemplary distances may be 10, 5, 4, 3, 2, 1, or 0 ORFs. The proteins identified as being within close proximity to a CRISPR array are then grouped into clusters of homologous proteins. In some embodiments, blastclust is used to form protein clusters. In certain other embodiments, mmseqs2 is used to form protein clusters.

To establish a pattern of strong co-occurrence between the members of a protein cluster with CRISPR arrays, a BLAST search of each member of the protein family may be performed over the complete set of known and predicted proteins previously compiled. In some embodiments, UBLAST or mmseqs2 may be used to search for similar proteins. In some embodiments, a search may be performed only for a representative subset of proteins in the family.

In some embodiments, the clusters of proteins within close proximity to CRISPR arrays are ranked or filtered by a metric to determine co-occurrence. One example of a metric is the ratio of the number of elements in a protein cluster against the number of BLAST matches up to a certain E value threshold. In some embodiments, a constant E value threshold may be used. In other embodiments, the E value threshold may be determined by the most distant members of the protein cluster. In some embodiments, the global set of proteins is clustered and the co-occurrence metric is the ratio of the number of elements of the CRISPR associated cluster against the number of elements of the containing global cluster(s).

In some embodiments, a manual review process is used to evaluate the potential functionality and the minimal set of components of an engineered system based on the naturally occurring locus structure of the proteins in the cluster. In some embodiments, a graphical representation of the protein cluster may assist in the manual review, and may contain information including pairwise sequence similarity, phylogenetic tree, source organisms/environments, predicted functional domains, and a graphical depiction of locus structures. In some embodiments, the graphical depiction of locus structures may filter for nearby protein families that have a high representation. In some embodiments, representation may be calculated by the ratio of the number of related nearby proteins against the size(s) of the containing global cluster(s). In certain exemplary embodiments, the graphical representation of the protein cluster may contain a depiction of the CRISPR array structures of the naturally occurring loci. In some embodiments, the graphical representation of the protein cluster may contain a depiction of the number of conserved direct repeats versus the length of the putative CRISPR array, or the number of unique spacer sequences versus the length of the putative CRISPR array. In some embodiments, the graphical representation of the protein cluster may contain a depiction of various metrics of co-occurrence of the putative effector with CRISPR arrays predict new CRISPR-Cas systems and identify their components.

### Pooled-Screening

To efficiently validate the activity of the engineered novel CRISPR-Cas systems and simultaneously evaluate in an unbiased manner different activity mechanisms and functional parameters, a new pooled-screening approach is used in E. coli. First, from the computational identification of the conserved protein and noncoding elements of the novel CRISPR-Cas system, DNA synthesis and molecular cloning is used to assemble the separate components into a single artificial expression vector, which in one embodiment is based on a pET-28a+ backbone. In a second embodiment, the effectors and noncoding elements are transcribed on a single mRNA transcript, and different ribosomal binding sites are used to translate individual effectors.

Second, the natural crRNA and targeting spacers are replaced with a library of unprocessed crRNAs containing non-natural spacers targeting a second plasmid, pACYC184. This crRNA library is cloned into the vector backbone containing the protein effectors and noncoding elements (e.g. pET-28a+), and then subsequently transformed the library into E. coli along with the pACYC184 plasmid target. Consequently, each resulting E. coli cell contains no more than one targeting spacer. In an alternate embodiment, the library of unprocessed crRNAs containing non-natural spacers additionally target E. coli essential genes drawn from resources such as those described in Baba et al. (2006) Mol. Syst. Biol. 2:2006.0008; and Gerdes et al. (2003) J. Bacteriol. 185(19): 5673-84, the entire contents of each of which are incorporated herein by reference. In this embodiment, positive, targeted activity of the novel CRISPR-Cas systems that disrupts essential gene function results in cell death or growth arrest. In some embodiments, the essential gene targeting spacers can be combined with the pACYC184 targets to add another dimension to the assay.

Third, the E. coli are grown under antibiotic selection. In one embodiment, triple antibiotic selection is used: kanamycin for ensuring successful transformation of the pET-28a+ vector containing the engineered CRISPR-Cas effector system, and chloramphenicol and tetracycline for ensuring successful co-transformation of the pACYC184 target vector. Since pACYC184 normally confers resistance to chloramphenicol and tetracycline, under antibiotic selection, positive activity of the novel CRISPR-Cas system targeting the plasmid will eliminate cells that actively express the effectors, noncoding elements, and specific active elements of the crRNA library. Examining the population of surviving cells at a later time point compared to an earlier time point typically provides a depleted signal compared to the inactive crRNAs. In some embodiments, double antibiotic selection is used. For example, withdrawal of either chloramphenicol or tetracycline to remove selective pressure can provide novel information about the targeting substrate, sequence specificity, and potency.

In some embodiments, only kanamycin is used to ensure successful transformation of the pET-28a+ vector containing the engineered CRISPR-Cas effector system. This embodiment is suitable for libraries containing spacers targeting E. coli essential genes, as no additional selection beyond kanamycin is needed to observe growth alterations. In this embodiment, chloramphenicol and tetracycline dependence is removed, and their targets (if any) in the library provides an additional source of negative or positive information about the targeting substrate, sequence specificity, and potency.

Since the pACYC184 plasmid contains a diverse set of features and sequences that may affect the activity of a CRISPR-Cas system, mapping the active crRNAs from the pooled screen onto pACYC184 provides patterns of activity that can be suggestive of different activity mechanisms and functional parameters in a broad, hypothesis-agnostic manner. In this way, the features required for reconstituting the novel CRISPR-Cas system in a heterologous prokaryotic species can be more comprehensively tested and studied.

Certain important advantages of the in vivo pooled-screen described herein include:
(1) Versatility-plasmid design allows multiple effectors and/or noncoding elements to be expressed; library cloning strategy enables both transcriptional directions of the computationally predicted crRNA to be expressed;
(2) Comprehensive tests of activity mechanisms and functional parameters can be used to evaluate diverse interference mechanisms, including DNA or RNA cleavage; to examine co-occurrence of features such as transcription, plasmid DNA replication; and to examine flanking sequences for a crRNA library to reliably determine PAMs, wherein any combination of up to 4 bases (representing a PAM of 4N complexity) are present at least 3-10 times in the targeting library;
(3) Sensitivity-pACYC184 is a low copy plasmid, enabling high sensitivity for CRISPR-Cas activity, because even modest interference rates can eliminate the antibiotic resistance encoded by the plasmid; and
(4) Efficiency—the pooled-screen includes optimized molecular biology steps that enable greater speed and throughput for RNA-sequencing, and the protein expression samples can be directly harvested from the surviving cells in the screen.

As discussed in more detail in the Examples below, the novel CRISPR-Cas families described herein were evaluated using this in vivo pooled-screen to evaluate their operational elements, mechanisms, and parameters, as well as their ability to be active and reprogrammed in an engineered system outside of their natural cellular environment.

### Class 2 CRISPR-Cas Effectors Having a RuvC Domain

In one aspect, the disclosure provides Class 2 CRISPR-Cas systems referred to herein as CLUST.019143 (Type V-G). These Class 2 CRISPR-Cas systems contain an isolated CRISPR-Cas effector protein having a RuvC domain and an isolated RNA guide, comprising a tracrRNA and a crRNA, wherein the crRNA includes a spacer sequence that is complementary to a target nucleic acid sequence such as an RNA sequence.

In some embodiments, an CRISPR-Cas effector protein having a RuvC domain may include one or more of the motifs from the set of the RuvC I motif (SEQ ID NO: 210), the RuvC II motif (SEQ ID NO: 211), and the RuvC III motif (SEQ ID NO: 212).

In some implementations, a Type V-G CRISPR-Cas system includes a CRISPR-Cas effector having a RuvC domain, a Type V-G crRNA, and a Type V-G tracrRNA. Suitably, a Type V-G crRNA may include SEQ ID NO: 200 proximal to its 3′ end and adjacent to the spacer sequence. Suitably, a Type V-G crRNA may include SEQ ID NO: 201 proximal to its 3′ end and adjacent to the spacer sequence. Suitably, a Type V-G crRNA may form a stem-loop structure with a Type V-G tracrRNA.

In some embodiments, Type V-G systems can be used to bind to sequence-specific target RNA. Suitably, Type V-G systems can be used to degrade sequence-specific target RNA upon recognition and binding. Suitably, Type V-G systems can be used to degrade “collateral” RNA upon binding to sequence-specific target RNA. Suitably, Type V-G systems can be used at higher temperatures (e.g. 50° C.) to degrade “collateral” RNA upon binding to sequence-specific target RNA with greater efficiency compared to lower temperatures (e.g. 37° C.). Suitably, Type V-G systems can be used to degrade “collateral” single-stranded DNA upon binding to sequence-specific target RNA. Suitably, Type V-G systems can be used at higher temperatures (e.g. 50° C.) to degrade “collateral” ssDNA upon binding to sequence-specific target RNA with greater efficiency compared to lower temperatures (e.g. 37° C.).

Suitably, a Type V-G CRISPR-Cas effector has an amino acid sequence of less than approximately 860 amino acids. Suitably, a Type V-G CRISPR-Cas effector has an amino acid sequence between 650 and 860 amino acids or between 700 and 850 amino acids.

In some embodiments, the Type V-G CRISPR-associated effector protein and the RNA guide form a surveillance complex that can include other components. The surveillance complex is activated upon binding to a nucleic acid substrate that is complementary to a spacer sequence in the RNA guide (i.e., a sequence-specific substrate, also referred to herein as a target nucleic acid). In some embodiments, the target nucleic acid is a double-stranded DNA. In some embodiments, the target nucleic acid is a single-stranded DNA. In some embodiments, the target nucleic acid is a single-stranded RNA. In some embodiments, the target nucleic acid is a double-stranded RNA. In some embodiments, the sequence-specificity requires a complete match of the spacer sequence in the RNA guide to the target substrate. In other embodiments, the sequence specificity requires a partial (contiguous or non-contiguous) match of the spacer sequence in the RNA guide to the target substrate.

In some embodiments, the surveillance complex becomes activated upon binding to the target substrate. In some embodiments, the target substrate is a DNA (e.g., a single-stranded or a double-stranded DNA). In some embodiments, the target substrate is a RNA (e.g., a single-stranded or a double-stranded RNA). In some embodiments, the activated complex exhibits “multiple turnover” activity, whereby upon acting on (e.g., cleaving) the target substrate, the activated complex remains in an activated state. In some embodiments, the activated complex exhibits “single turnover” activity, whereby upon acting on the target substrate, the surveillance complex reverts to an inactive state. In some embodiments, the activated complex exhibits non-specific (i.e., “collateral”) cleavage activity, whereby the activated complex cleaves nucleic acids with no sequence similarity to the target (“collateral nucleic acids”). In some embodiments, the collateral nucleic acid substrate is a DNA (e.g., a single-stranded or a double-stranded DNA). In some embodiments, the collateral nucleic acid substrate is an RNA (e.g., a single-stranded or a double-stranded RNA).

### CRISPR Class 2 RNA-Guided RNases

In another aspect, this disclosure describes a novel family of CRISPR Class 2 effectors having RNA-targeting capabilities. The underlying one or more RNase domains confer the ability to bind to and cleave any target RNA molecule. The target RNA may be any form of RNA, including, but not limited to, mRNA, tRNA, ribosomal RNA, non-coding RNA, lincRNA, and nuclear RNA. For example, in some embodiments, the CRISPR-associated protein recognizes and cleaves targets located on the coding strand of open reading frames (ORFs).

In one embodiment, the disclosure provides a family of CRISPR Class 2 effectors, referred to herein generally as CLUST.019143 (Type V-G) CRISPR-Cas effector proteins. This newly-identified family of CRISPR Class 2 effectors can be used in a variety of applications, and the new effector proteins are particularly suitable for therapeutic applications, because they are significantly smaller than other RNA-targeting CRISPR effectors (e.g., CRISPR Cas13a, Cas13b, or Cas13c effectors), which allows for the packaging of the effectors and/or nucleic acids encoding the effectors into delivery systems having size limitations.

### Collateral RNase Activity

In some embodiments, a surveillance complex comprised of (but not necessarily limited to) a Cas12g CRISPR-associated protein and an RNA guide is activated upon binding to a target nucleic acid (e.g., a target RNA). Activation induces a conformational change, which results in the activated complex acting as a non-specific RNase, cleaving and/or degrading nearby RNA molecules (e.g., ssRNA or dsRNA molecules) (i.e., “collateral” effects).

### Collateral DNase Activity

In some embodiments, a surveillance complex comprised of (but not necessarily limited to) a Cas12g effector and an RNA guide is activated upon binding to a target nucleic acid (e.g., a target RNA). Activation induces a conformational change, which results in the complex acting as a non-specific DNase, cleaving and/or degrading nearby DNA molecules (e.g., ssDNA or dsDNA molecules) (i.e., “collateral” effects).

### Collateral-Free Target Recognition

In other embodiments, a surveillance complex comprised of (but not necessarily limited to) a Cas12g effector and an RNA guide does not exhibit collateral nuclease activity subsequent to target recognition. This “collateral-free” embodiment may comprise wild-type or engineered effector proteins.

### PAM/PFS-Independent Targeting

In some embodiments, a surveillance complex comprised of (but not necessarily limited to) a Cas12g effector and an RNA guide recognizes and cleaves the target nucleic acid without any additional requirements adjacent to or flanking the protospacer (i.e., protospacer adjacent motif “PAM” or protospacer flanking sequence “PFS” requirements).

### CRISPR Enzyme Modifications

### Nuclease-Deficient CRISPR Enzymes

Where the CRISPR enzymes described herein have nuclease activity, the CRISPR enzymes can be modified to have diminished nuclease activity, e.g., nuclease inactivation of at least 50%, at least 60%, at least 70%, at least 80%, at least 90%, at least 95%, at least 97%, or 100% as compared with the wild type CRISPR enzymes. The nuclease activity can be diminished by, for example, introducing mutations (such as amino acid insertions, deletions, or substitutions) into the nuclease domains of the CRISPR enzymes. In some embodiments, catalytic residues for the nuclease activities are identified, and these amino acid residues can be substituted by different amino acid residues (e.g., glycine or alanine) to diminish the nuclease activity. An example of such a mutation for Cas12g1 includes D513A.

The inactivated CRISPR enzymes can comprise (e.g., via fusion protein, linker peptides, Gly4Ser (GS) peptide linkers, etc.) or be associated (e.g., via co-expression of multiple proteins) with one or more functional domains. These functional domains can have various activities, e.g., methylase activity, demethylase activity, transcription activation activity, transcription repression activity, transcription release factor activity, histone modification activity, RNA cleavage activity, DNA cleavage activity, nucleic acid binding activity, and switch activity (e.g., light inducible). In some embodiments, the functional domains are Krüppel associated box (KRAB), VP64, VP16, Fok1, P65, HSF1, MyoD1, and biotin-APEX.

The positioning of the one or more functional domains on the inactivated CRISPR enzymes allows for correct spatial orientation for the functional domain to affect the target with the attributed functional effect. For example, if the functional domain is a transcription activator (e.g., VP16, VP64, or p65), the transcription activator is placed in a spatial orientation that allows it to affect the transcription of the target. Likewise, a transcription repressor (e.g., KRAB) is positioned to affect the transcription of the target, and a nuclease (e.g., Fok1) is positioned to cleave or partially cleave the target. In some embodiments, the functional domain is positioned at the N-terminus of the CRISPR enzyme. In some embodiments, the functional domain is positioned at the C-terminus of the CRISPR enzyme. In some embodiments, the inactivated CRISPR enzyme is modified to comprise a first functional domain at the N-terminus and a second functional domain at the C-terminus.

### Split Enzymes

The present disclosure also provides a split version of the CRISPR enzymes described herein. The split version of the CRISPR enzymes may be advantageous for delivery. In some embodiments, the CRISPR enzymes are split into two parts of the enzymes, which together substantially comprises a functioning CRISPR enzyme.

The split can be done in a way that the catalytic domain(s) are unaffected. The CRISPR enzymes may function as a nuclease or may be inactivated enzymes, which are essentially RNA-binding proteins with very little or no catalytic activity (e.g., due to mutation(s) in its catalytic domains).

In some embodiments, the nuclease lobe and α-helical lobe are expressed as separate polypeptides. Although the lobes do not interact on their own, the RNA guide recruits them into a surveillance complex that recapitulates the activity of full-length CRISPR enzymes and catalyzes site-specific DNA cleavage. The use of a modified RNA guide abrogates split-enzyme activity by preventing dimerization, allowing for the development of an inducible dimerization system. The split enzyme is described, e.g., in Wright, Addison V., et al. “Rational design of a split-Cas9 enzyme complex,” Proc. Nat'l. Acad. Sci., 112.10 (2015): 2984-2989, which is incorporated herein by reference in its entirety.

In some embodiments, the split enzyme can be fused to a dimerization partner, e.g., by employing rapamycin sensitive dimerization domains. This allows the generation of a chemically inducible CRISPR enzyme for temporal control of CRISPR enzyme activity. The CRISPR enzymes can thus be rendered chemically inducible by being split into two fragments and rapamycin-sensitive dimerization domains can be used for controlled reassembly of the CRISPR enzymes.

The split point is typically designed in silico and cloned into the constructs. During this process, mutations can be introduced to the split enzyme and non-functional domains can be removed. In some embodiments, the two parts or fragments of the split CRISPR enzyme (i.e., the N-terminal and C-terminal fragments), can form a full CRISPR enzyme, comprising, e.g., at least 70%, at least 80%, at least 90%, at least 95%, or at least 99% of the sequence of the wild-type CRISPR enzyme.

### Self-Activating or Inactivating Enzymes

The CRISPR enzymes described herein can be designed to be self-activating or self-inactivating. In some embodiments, the CRISPR enzymes are self-inactivating. For example, the target sequence can be introduced into the CRISPR enzyme coding constructs. Thus, the CRISPR enzymes can cleave the target sequence, as well as the construct encoding the enzyme thereby self-inactivating their expression. Methods of constructing a self-inactivating CRISPR system is described, e.g., in Epstein, Benjamin E., and David V. Schaffer. “Engineering a Self-Inactivating CRISPR System for AAV Vectors,” Mol. Ther., 24 (2016): S50, which is incorporated herein by reference in its entirety.

In some other embodiments, an additional RNA guide, expressed under the control of a weak promoter (e.g., 7SK promoter), can target the nucleic acid sequence encoding the CRISPR enzyme to prevent and/or block its expression (e.g., by preventing the transcription and/or translation of the nucleic acid). The transfection of cells with vectors expressing the CRISPR enzyme and RNA guide(s) that target the nucleic acid encoding the CRISPR enzyme can lead to efficient disruption of the nucleic acid encoding the CRISPR enzyme and decrease the levels of CRISPR enzyme, thereby limiting the genome editing activity.

In some embodiments, the genome editing activity of the CRISPR enzymes can be modulated through endogenous RNA signatures (e.g., miRNA) in mammalian cells. The CRISPR enzyme switch can be made by using a miRNA-complementary sequence in the 5′-UTR of mRNA encoding the CRISPR enzyme. The switches selectively and efficiently respond to miRNA in the target cells. Thus, the switches can differentially control the genome editing by sensing endogenous miRNA activities within a heterogeneous cell population. Therefore, the switch systems can provide a framework for cell-type selective genome editing and cell engineering based on intracellular miRNA information (Hirosawa, Moe et al. “Cell-type-specific genome editing with a microRNA-responsive CRISPR-Cas9 switch,” Nucl. Acids Res., 2017 Jul. 27; 45(13): e118).

### Inducible CRISPR Enzymes

The CRISPR enzymes can be inducible, e.g., light inducible or chemically inducible. This mechanism allows for activation of the functional domain in the CRISPR enzymes with a known trigger. Light inducibility can be achieved by various methods known in the art, e.g., by designing a fusion complex wherein CRY2 PHR/CIBN pairing is used in split CRISPR Enzymes (see, e.g., Konermann et al. “Optical control of mammalian endogenous transcription and epigenetic states,” Nature, 500.7463 (2013): 472). Chemical inducibility can be achieved, e.g., by designing a fusion complex wherein FKBP/FRB (FK506 binding protein/FKBP rapamycin binding domain) pairing is used in split CRISPR Enzymes. Rapamycin is required for forming the fusion complex, thereby activating the CRISPR enzymes (see, e.g., Zetsche, Volz, and Zhang, “A split-Cas9 architecture for inducible genome editing and transcription modulation,” Nature Biotech., 33.2 (2015): 139-142).

Furthermore, expression of the CRISPR enzymes can be modulated by inducible promoters, e.g., tetracycline or doxycycline controlled transcriptional activation (Tet-On and Tet-Off expression system), hormone inducible gene expression system (e.g., an ecdysone inducible gene expression system), and an arabinose-inducible gene expression system. When delivered as RNA, expression of the RNA targeting effector protein can be modulated via a riboswitch, which can sense a small molecule like tetracycline (see, e.g., Goldfless, Stephen J. et al. “Direct and specific chemical control of eukaryotic translation with a synthetic RNA-protein interaction,” Nucl. Acids Res., 40.9 (2012): e64-e64).

Various embodiments of inducible CRISPR enzymes and inducible CRISPR systems are described, e.g., in U.S. Pat. No. 8,871,445, US20160208243, and WO2016205764, each of which is incorporated herein by reference in its entirety.

### Functional Mutations

In some embodiments, the CRISPR-associated proteins include at least one (e.g., 1, 2, 3, 4, 5, 6, 7, 8, 9, or 10) Nuclear Localization Signal (NLS) attached to the N-terminal or C-terminal of the protein. Non-limiting examples of NLSs include an NLS sequence derived from: the NLS of the SV40 virus large T-antigen, having the amino acid sequence PKKKRKV (SEQ ID NO: 300); the NLS from nucleoplasmin (e.g., the nucleoplasmin bipartite NLS with the sequence KRPAATKKAGQAKKKK (SEQ ID NO: 301)); the c-myc NLS having the amino acid sequence PAAKRVKLD (SEQ ID NO: 302) or RQRRNELKRSP (SEQ ID NO: 303); the hRNPA1 M9 NLS having the sequence NQSSNFGPMKGGNFGGRSSGPYGGGGQYFAKPRNQGGY (SEQ ID NO: 304); the sequence RMRIZFKNKGKDTAELRRRRVEVSVELRKAKKDEQILKRRNV (SEQ ID NO: 305) of the IBB domain from importin-alpha; the sequences VSRKRPRP (SEQ ID NO: 306) and PPKKARED (SEQ ID NO: 307) of the myoma T protein; the sequence PQPKKKPL (SEQ ID NO: 308) of human p53; the sequence SALIKKKKKMAP (SEQ ID NO: 309) of mouse c-abl IV; the sequences DRLRR (SEQ ID NO: 310) and PKQKKRK (SEQ ID NO: 311) of the influenza virus NS1; the sequence RKLKKKIKKL (SEQ ID NO: 312) of the Hepatitis virus delta antigen; the sequence REKKKFLKRR (SEQ ID NO: 313) of the mouse Mx1 protein; the sequence KRKGDEVDGVDEVAKKKSKK (SEQ ID NO: 314) of the human poly(ADP-ribose) polymerase; and the sequence RKCLQAGMNLEARKTKK (SEQ ID NO: 315) of the human glucocorticoid receptor. In some embodiments, the CRISPR-associated protein includes at least one (e.g., 1, 2, 3, 4, 5, 6, 7, 8, 9, or 10) Nuclear Export Signal (NES) attached the N-terminal or C-terminal of the protein. In a preferred embodiment a C-terminal and/or N-terminal NLS or NES is attached for optimal expression and nuclear targeting in eukaryotic cells, e.g., human cells.

In some embodiments, the CRISPR enzymes described herein are mutated at one or more amino acid residues to alter one or more functional activities. For example, in some embodiments, the CRISPR enzyme is mutated at one or more amino acid residues to alter its helicase activity. In some embodiments, the CRISPR enzyme is mutated at one or more amino acid residues to alter its nuclease activity (e.g., endonuclease activity or exonuclease activity). In some embodiments, the CRISPR enzyme is mutated at one or more amino acid residues to alter its ability to functionally associate with a RNA guide. In some embodiments, the CRISPR enzyme is mutated at one or more amino acid residues to alter its ability to functionally associate with a target nucleic acid.

In some embodiments, the CRISPR enzymes described herein are capable of cleaving a target nucleic acid molecule. In some embodiments, the CRISPR enzyme cleaves both strands of the target nucleic acid molecule. However, in some embodiments, the CRISPR enzyme is mutated at one or more amino acid residues to alter its cleaving activity. For example, in some embodiments, the CRISPR enzyme may comprise one or more mutations that render the enzyme incapable of cleaving a target nucleic acid. In other embodiments, the CRISPR enzyme may comprise one or more mutations such that the enzyme is capable of cleaving a single strand of the target nucleic acid (i.e., nickase activity). In some embodiments, the CRISPR enzyme is capable of cleaving the strand of the target nucleic acid that is complementary to the strand to which the RNA guide hybridizes. In some embodiments, the CRISPR enzyme is capable of cleaving the strand of the target nucleic acid to which the RNA guide hybridizes.

In some embodiments, a CRISPR enzyme described herein can be engineered to include a deletion in one or more amino acid residues to reduce the size of the enzyme while retaining one or more desired functional activities (e.g., nuclease activity and the ability to interact functionally with a RNA guide). The truncated CRISPR enzyme may be advantageously used in combination with delivery systems having load limitations.

Nucleic acids encoding the proteins (e.g., a CRISPR-associated protein) and RNA guides (e.g., a crRNA) described herein are also provided. In some embodiments, the nucleic acid is a synthetic nucleic acid. In some embodiments, the nucleic acid is a DNA molecule. In some embodiments, the nucleic acid is an RNA molecule (e.g., an mRNA molecule). In some embodiments, the mRNA is capped, polyadenylated, substituted with 5-methylcytidine, substituted with pseudouridine, or a combination thereof. In some embodiments, the nucleic acid (e.g., DNA) is operably-linked to a regulatory element (e.g., a promoter) to control the expression of the nucleic acid. In some embodiments, the promoter is a constitutive promoter. In some embodiments, the promoter is an inducible promoter. In some embodiments, the promoter is a cell-specific promoter, such as Syn and CamKIIa for neuronal cell types, or thyroxine binding globulin (TBG) for hepatocyte expression. In some embodiments, the promoter is an organism-specific promoter. Suitable promoters are known in the art and include, for example, a pol I promoter, a pol II promoter, a pol III promoter. In some embodiments, short RNAs such as the RNA guide are effectively expressed using a pol III promoter, which includes a U6 promoter, a H1 promoter, a 7SK promoter. In some embodiments, the promoter is prokaryotic, such as a T7 promoter. In some embodiments, the promoters are eukaryotic and include retroviral Rous sarcoma virus LTR promoter, a cytomegalovirus (CMV) promoter, a SV40 promoter, a dihydrofolate reductase promoter, a β-actin promoter, elongation factor 1 alpha promoter, elongation factor 1 alpha short promoter, SV40 promoter, and the synthetic CAG promoter. In some embodiments, the termination signals for induction of mRNA polyadenylation include, but are not limited to, SV40, hGH, and bGH.

In some embodiments, the nucleic acid(s) are present in a vector (e.g., a viral vector or a phage). The vectors can include one or more regulatory elements that allow for the propagation of the vector in a cell of interest (e.g., a bacterial cell or a mammalian cell). In some embodiments, the vector includes a nucleic acid encoding a single component of a CRISPR-associated (Cas) system described herein. In some embodiments, the vector includes multiple nucleic acids, each encoding a component of a CRISPR-associated (Cas) system described herein.

In one aspect, the present disclosure provides nucleic acid sequences that are at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the nucleic sequences described herein. In another aspect, the present disclosure also provides amino acid sequences that are at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequences described herein.

In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are the same as the sequences described herein. In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from the sequences described herein.

In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as the sequences described herein. In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from the sequences described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In general, the length of a reference sequence aligned for comparison purposes should be at least 80% of the length of the reference sequence, and in some embodiments is at least 90%, 95%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

In some embodiments, the CRISPR-associated proteins and accessory proteins described herein can be fused to one or more peptide tags, including a His-tag, GST-tag, FLAG-tag, or myc-tag. In some embodiments, the CRISPR-associated proteins or accessory proteins described herein can be fused to a detectable moiety such as a fluorescent protein (e.g., green fluorescent protein or yellow fluorescent protein). In those embodiments where a tag is fused to a CRISPR-associated protein, such tag may facilitate affinity-based and/or charge-based purification of the CRISPR-associated protein, e.g., by liquid chromatography or bead separation utilizing an immobilized affinity or ion-exchange reagent. As a non-limiting example, a recombinant CRISPR-associated protein of this disclosure (such as a Cas12g) comprises a polyhistidine (His) tag, and for purification is loaded onto a chromatography column comprising an immobilized metal ion (e.g. a Zn²⁺, Ni²⁺, Cu²⁺ ion chelated by a chelating ligand immobilized on the resin, which resin may be an individually prepared resin or a commercially available resin). or ready to use column such as the HisTrap FF column commercialized by GE Healthcare Life Sciences, Marlborough, Massachusetts). Following the loading step, the column is optionally rinsed, e.g., using one or more suitable buffer solutions, and the His-tagged protein is then eluted using a suitable elution buffer. Alternatively, or additionally, if the recombinant CRISPR-associated protein of this disclosure utilizes a FLAG-tag, such protein may be purified using immunoprecipitation methods known in the industry. Other suitable purification methods for tagged CRISPR-associated proteins or accessory proteins of this disclosure will be evident to those of skill in the art.

The proteins described herein (e.g., CRISPR-associated proteins or accessory proteins) can be delivered or used as either nucleic acid molecules or polypeptides. When nucleic acid molecules are used, the nucleic acid molecule encoding the CRISPR-associated proteins can be codon-optimized, as described in further detail below. The nucleic acid can be codon optimized for use in any organism of interest, in particular human cells or bacteria. For example, the nucleic acid can be codon-optimized for any non-human eukaryote including mice, rats, rabbits, dogs, livestock, or non-human primates. Codon usage tables are readily available, for example, at the “Codon Usage Database” available online with a search for kazusa.or.jp/codon/and these tables can be adapted in a number of ways. See Nakamura et al. Nucl. Acids Res. 28:292 (2000), which is incorporated herein by reference in its entirety. Computer algorithms for codon optimizing a particular sequence for expression in a particular host cell are also available, such as Gene Forge (Aptagen; Jacobus, PA).

In some instances, nucleic acids of this disclosure which encode CRISPR-associated proteins or accessory proteins for expression in eukaryotic (e.g., human, mammalian, etc.) cells include one or more introns, i.e., one or more non-coding sequences comprising, at a first end (e.g., a 5′ end), a splice-donor sequence and, at second end (e.g., the 3′ end) a splice acceptor sequence. Any suitable splice donor/splice acceptor can be used in the various embodiments of this disclosure, including without limitation simian virus 40 (SV40) intron, beta-globin intron, and synthetic introns. Alternatively, or additionally, nucleic acids of this disclosure encoding CRISPR-associated proteins or accessory proteins may include, at a 3′ end of a DNA coding sequence, a transcription stop signal such as a polyadenylation (polyA) signal. In some instances, the polyA signal is located in close proximity to, or adjacent to, an intron such as the SV40 intron.

### RNA Guides

In some embodiments, the CRISPR systems described herein include at least one RNA guide. The architecture of multiple RNA guides is known in the art (see, e.g., International Publication Nos. WO 2014/093622 and WO 2015/070083, the entire contents of each of which are incorporated herein by reference). In some embodiments, the CRISPR systems described herein include multiple RNA guides (e.g., two, three, four, five, six, seven, eight, or more RNA guides). In some embodiments, the RNA guide includes a crRNA and a tracrRNA. In some embodiments, the RNA guide is an engineered construct that comprises a tracrRNA and a crRNA (in a single RNA guide). Sequences for RNA guides from multiple CRISPR systems are known in the art and can be searched using public databases (see, e.g., Grissa et al. (2007) Nucleic Acids Res. 35 (web server issue): W52-7; Grissa et al. (2007) BMC Bioinformatics 8:172; Grissa et al. (2008) Nucleic Acids Res. 36 (web server issue): W145-8; and Moller and Liang (2017) PeerJ 5: e3788; see also the CRISPR database available at: crispr.i2bc.paris-saclay.fr/crispr/BLAST/CRISPRsBlast.php; and MetaCRAST available at: github.com/molleraj/MetaCRAST).

In some embodiments, the CRISPR systems described herein include at least one RNA guide or a nucleic acid encoding at least one RNA guide. In some embodiments, the RNA guide includes a crRNA. Generally, the crRNAs described herein include a direct repeat sequence and a spacer sequence. In certain embodiments, the crRNA includes, consists essentially of, or consists of a direct repeat sequence linked to a guide sequence or spacer sequence. In some embodiments, the crRNA includes a direct repeat sequence, a spacer sequence, and a direct repeat sequence (DR-spacer-DR), which is typical of precursor crRNA (pre-crRNA) configurations in other CRISPR systems. In some embodiments, the crRNA includes a truncated direct repeat sequence and a spacer sequence, which is typical of processed or mature crRNA. In some embodiments, the crRNA hybridizes with an anti-repeat region of a tracrRNA complementary to the crRNA direct repeat region. In some embodiments, the CRISPR-associated protein forms a complex with the crRNA, and the spacer sequence directs the complex to a sequence-specific binding with the target nucleic acid that is complementary to the spacer sequence. In some embodiments, the CRISPR-associated protein forms a complex with the crRNA and tracrRNA, and the spacer sequence directs the complex to a sequence-specific binding with the target nucleic acid that is complementary to the spacer sequence.

In some embodiments, the CRISPR systems described herein include at least one RNA guide or a nucleic acid encoding at least one RNA guide. In some embodiments, the RNA guide includes a mature crRNA. In some embodiments, the CRISPR systems described herein include a mature crRNA and a tracrRNA. In some embodiments, the CRISPR systems described herein include a pre-crRNA. In some embodiments, the CRISPR systems described herein include a pre-crRNA and a tracrRNA.

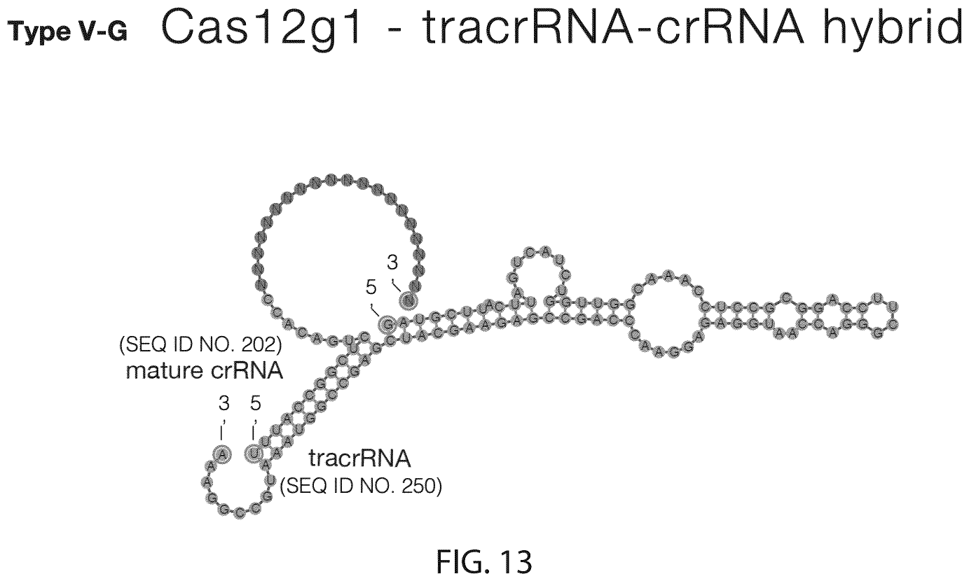

Suitably, the Type V-G RNA guide may form a secondary structure such as a stem-loop structure. Suitably, the Type V-G RNA guide may include both a Type V-G crRNA and a Type V-G tracrRNA, either fused into a single RNA molecule or as separate RNA molecules. In some embodiments, a Type V-G crRNA can hybridize with a Type V-G tracrRNA to form a stem-loop structure. An example stem-loop structure of one Type V-G mature crRNA:tracrRNA is shown in FIG. 13. The complementary sections of the crRNA and tracrRNA form the stem. For example, the stem may include at least 8 or at least 9 or at least 10 or at about 11 base pairs.

In some examples, the direct repeat may comprise at least 12 or at least 14 or at least 16 or about 18 nucleotides. The direct repeat can include the nucleic acid sequence X₁ACACC (SEQ ID NO:203) proximal to the spacer, wherein X₁ denotes G or T.

In some embodiments, the CRISPR systems described herein include a plurality of RNA guides (e.g., 2, 3, 4, 5, 10, 15, or more) or a plurality of nucleic acids encoding a plurality of RNA guides.

In some embodiments, the CRISPR system described herein includes an RNA guide or a nucleic acid encoding the RNA guide. In some embodiments, the RNA guide comprises or consists of a direct repeat sequence and a spacer sequence capable of hybridizing (e.g., hybridizes under appropriate conditions) to a target nucleic acid, wherein the direct repeat sequence includes SEQ ID NO: 200 proximal to its 3′ end and adjacent to the spacer sequence. In some embodiments, the RNA guide comprises or consists of a direct repeat sequence and a spacer sequence capable of hybridizing (e.g., hybridizes under appropriate conditions) to a target nucleic acid, wherein the direct repeat sequence includes SEQ ID NO: 201 proximal to its 3′ end and adjacent to the spacer sequence.

Examples of RNA guide direct repeat sequences and effector protein pairs are provided in Table 5A. In some embodiments, the direct repeat sequence comprises or consists of a nucleic acid sequence listed in Table 5A (e.g., SEQ ID NOs: 9-13, 25-34). In some embodiments, the direct repeat sequence comprises or consists of a nucleic acid having a nucleic acid sequence listed in Table 5A with a truncation of the initial three 5′ nucleotides. In some embodiments, the direct repeat sequence comprises or consists of a nucleic acid having a nucleic acid sequence listed in Table 5A with a truncation of the initial four 5′ nucleotides. In some embodiments, the direct repeat sequence comprises or consists of a nucleic acid having a nucleic acid sequence listed in Table 5A with a truncation of the initial five 5′ nucleotides. In some embodiments, the direct repeat sequence comprises or consists of a nucleic acid having a nucleic acid sequence listed in Table 5A with a truncation of the initial six 5′ nucleotides. In some embodiments, the direct repeat sequence comprises or consists of a nucleic acid having a nucleic acid sequence listed in Table 5A with a truncation of the initial seven 5′ nucleotides. In some embodiments, the direct repeat sequence comprises or consists of a nucleic acid having a nucleic acid sequence listed in Table 5A with a truncation of the initial eight 5′ nucleotides.

Examples of pairs of effector protein and tracrRNA-encoding sequences are provided in Table 7 (e.g., SEQ ID Nos: 117-134). Table 7 contains sequences with complementarity to the DRs of different Cas12g systems. This set of putative tracrRNA-containing loci includes a locus that contains the Cas12g1 tracrRNA.

In some embodiments, the Type V-G CRISPR-Cas system includes the effector protein that comprises the amino acid sequence of SEQ ID NO: 6, and a RNA guide that includes a tracrRNA sequence that contains the sequence GAUGCUUACUUAGUCAUCUGGUUGGCAAACCUCCGCGGACCUUCGGGACCAAUGG AGAGGAACCCAGCCGAGAAGCAUCGAGCCGGUAAAUGCCGGAAA (SEQ ID NO: 250) and a crRNA sequence that contains the sequence UUUACCGGCUCUGACACC (SEQ ID NO: 202).

### Multiplexing RNA Guides

Type V-G (CLUST.019143) CRISPR-Cas effector proteins have been demonstrated to employ more than one RNA guide, thus enabling the ability of these effectors, and systems and complexes that include them, to target multiple different nucleic acid targets. In some embodiments, the CRISPR systems described herein include multiple RNA guides (e.g., two, three, four, five, six, seven, eight, nine, ten, fifteen, twenty, thirty, forty, or more RNA guides). In some embodiments, the CRISPR systems described herein include a single RNA strand or a nucleic acid encoding a single RNA strand, wherein the RNA guides are arranged in tandem. The single RNA strand can include multiple copies of the same RNA guide, multiple copies of distinct RNA guides, or combinations thereof.

In some embodiments, the Type V-G CRISPR-Cas effector proteins are delivered complexed with multiple RNA guides directed to different target nucleic acids. In some embodiments, the Type V-G CRISPR-Cas effector proteins can be co-delivered with multiple RNA guides, each specific for a different target nucleic acid. Methods of multiplexing using CRISPR-associated proteins are described, for example, in U.S. Pat. No. 9,790,490, and EP 3009511, the entire contents of each of which are expressly incorporated herein by reference.

RNA guide Modifications

### Spacer Lengths

The spacer length of RNA guides can range from about 15 to 50 nucleotides. In some embodiments, the spacer length of a RNA guide is at least 16 nucleotides, at least 17 nucleotides, at least 18 nucleotides, at least 19 nucleotides, at least 20 nucleotides, at least 21 nucleotides, or at least 22 nucleotides. In some embodiments, the spacer length is from 15 to 17 nucleotides, from 15 to 23 nucleotides, from 15 to 30 nucleotides, from 16 to 22 nucleotides, from 17 to 20 nucleotides, from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 45 nucleotides (e.g., 30, 31, 32, 33, 34, 35, 40, or 45 nucleotides), from 30 or 35 to 40 nucleotides, from 41 to 45 nucleotides, from 45 to 50 nucleotides, or longer. In some embodiments, the spacer length of the RNA guide is at least 16 nucleotides, or is from 16 to 20 nucleotides (e.g., 16, 17, 18, 19, or 20 nucleotides). In some embodiments, the spacer length of the RNA guide is 19 nucleotides.

Exemplary RNA guide direct repeat sequences and effector protein pairs are provided in Table 5A. In some embodiments, the direct repeat sequence comprises or consists of a nucleic acid sequence listed in Table 5A (e.g., SEQ ID NOs: 9-14).

The RNA guide sequences can be modified in a manner that allows for formation of the CRISPR complex and successful binding to the target, while at the same time not allowing for successful nuclease activity (i.e., without nuclease activity/without causing indels). These modified guide sequences are referred to as “dead guides” or “dead guide sequences.” These dead guides or dead guide sequences may be catalytically inactive or conformationally inactive with regard to nuclease activity. Dead guide sequences are typically shorter than respective guide sequences that result in active RNA cleavage. In some embodiments, dead guides are 5%, 10%, 20%, 30%, 40%, or 50%, shorter than respective RNA guides that have nuclease activity. Dead guide sequences of RNA guides can be from 13 to 15 nucleotides in length (e.g., 13, 14, or 15 nucleotides in length), from 15 to 19 nucleotides in length, or from 17 to 18 nucleotides in length (e.g., 17 nucleotides in length).

Thus, in one aspect, the disclosure provides non-naturally occurring or engineered CRISPR systems including a functional CRISPR enzyme as described herein, and a RNA guide wherein the RNA guide includes a dead guide sequence whereby the RNA guide is capable of hybridizing to a target sequence such that the CRISPR system is directed to a genomic locus of interest in a cell without detectable cleavage activity.

A detailed description of dead guides is described, e.g., in WO 2016094872, which is incorporated herein by reference in its entirety.

### Inducible Guides

RNA guides can be generated as components of inducible systems. The inducible nature of the systems allows for spatiotemporal control of gene editing or gene expression. In some embodiments, the stimuli for the inducible systems include, e.g., electromagnetic radiation, sound energy, chemical energy, and/or thermal energy.

In some embodiments, the transcription of RNA guides can be modulated by inducible promoters, e.g., tetracycline or doxycycline controlled transcriptional activation (Tet-On and Tet-Off expression systems), hormone inducible gene expression systems (e.g., ecdysone inducible gene expression systems), and arabinose-inducible gene expression systems. Other examples of inducible systems include, e.g., small molecule two-hybrid transcription activations systems (FKBP, ABA, etc.), light inducible systems (Phytochrome, LOV domains, or cryptochrome), or Light Inducible Transcriptional Effector (LITE). These inducible systems are described, e.g., in WO 2016205764 and U.S. Pat. No. 8,795,965, both of which are incorporated herein by reference in their entirety.

### Chemical Modifications

Chemical modifications can be applied to the RNA guide's phosphate backbone, sugar, and/or base. Backbone modifications such as phosphorothioates modify the charge on the phosphate backbone and aid in the delivery and nuclease resistance of the oligonucleotide (see, e.g., Eckstein, “Phosphorothioates, essential components of therapeutic oligonucleotides,” Nucl. Acid Ther., 24 (2014), pp. 374-387); modifications of sugars, such as 2′-O-methyl (2′-OMe), 2′-F, and locked nucleic acid (LNA), enhance both base pairing and nuclease resistance (see, e.g., Allerson et al. “Fully 2′-modified oligonucleotide duplexes with improved in vitro potency and stability compared to unmodified small interfering RNA,” J. Med. Chem., 48.4 (2005): 901-904). Chemically modified bases such as 2-thiouridine or N6-methyladenosine, among others, can allow for either stronger or weaker base pairing (see, e.g., Bramsen et al., “Development of therapeutic-grade small interfering RNAs by chemical engineering,” Front. Genet. 2012 Aug. 20; 3:154). Additionally, RNA is amenable to both 5′ and 3′ end conjugations with a variety of functional moieties including fluorescent dyes, polyethylene glycol, or proteins.

A wide variety of modifications can be applied to chemically synthesized RNA guide molecules. For example, modifying an oligonucleotide with a 2′-OMe to improve nuclease resistance can change the binding energy of Watson-Crick base pairing. Furthermore, a 2′-OMe modification can affect how the oligonucleotide interacts with transfection reagents, proteins or any other molecules in the cell. The effects of these modifications can be determined by empirical testing.

In some embodiments, the RNA guide includes one or more phosphorothioate modifications. In some embodiments, the RNA guide includes one or more locked nucleic acids for the purpose of enhancing base pairing and/or increasing nuclease resistance.

A summary of these chemical modifications can be found, e.g., in Kelley et al., “Versatility of chemically synthesized guide RNAs for CRISPR-Cas9 genome editing,” J. Biotechnol. 2016 Sep. 10; 233:74-83; WO 2016205764; and U.S. Pat. No. 8,795,965 B2; each which is incorporated by reference in its entirety.

### Sequence Modifications

The sequences and the lengths of the RNA guides (e.g., tracrRNAs and crRNAs) described herein can be optimized. In some embodiments, the optimized length of RNA guides can be determined by identifying the processed form of tracrRNA and/or crRNA, or by empirical length studies for RNA guides, tracrRNAs, crRNAs, and the tracrRNA tetraloops.

The RNA guides can also include one or more aptamer sequences. Aptamers are oligonucleotide or peptide molecules that can bind to a specific target molecule. The aptamers can be specific to gene effectors, gene activators, or gene repressors. In some embodiments, the aptamers can be specific to a protein, which in turn is specific to and recruits/binds to specific gene effectors, gene activators, or gene repressors. The effectors, activators, or repressors can be present in the form of fusion proteins. In some embodiments, the RNA guide has two or more aptamer sequences that are specific to the same adaptor proteins. In some embodiments, the two or more aptamer sequences are specific to different adaptor proteins. The adaptor proteins can include, e.g., MS2, PP7, Qβ, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s, and PRR1. Accordingly, in some embodiments, the aptamer is selected from binding proteins specifically binding any one of the adaptor proteins as described herein. In some embodiments, the aptamer sequence is a MS2 binding loop, QBeta binding loop, or PP7 binding loop. A detailed description of aptamers can be found, e.g., in Nowak et al., “Guide RNA engineering for versatile Cas9 functionality,” Nucl. Acid. Res., 2016 Nov. 16; 44(20):9555-9564; and WO 2016205764, which are incorporated herein by reference in their entirety.

### Guide: Target Sequence Matching Requirements

In classic CRISPR systems, the degree of complementarity between a guide sequence and its corresponding target sequence can be about 50%, 60%, 75%, 80%, 85%, 90%, 95%, 97.5%, 99%, or 100%. In some embodiments, the degree of complementarity is 100%. The RNA guides can be about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length.

To reduce off-target interactions, e.g., to reduce the guide interacting with a target sequence having low complementarity, mutations can be introduced to the CRISPR systems so that the CRISPR systems can distinguish between target and off-target sequences that have greater than 80%, 85%, 90%, or 95% complementarity. In some embodiments, the degree of complementarity is from 80% to 95%, e.g., about 83%, 84%, 85%, 86%, 87%, 88%, 89%, 90%, 91%, 92%, 93%, 94%, or 95% (for example, distinguishing between a target having 18 nucleotides from an off-target of 18 nucleotides having 1, 2, or 3 mismatches). Accordingly, in some embodiments, the degree of complementarity between a guide sequence and its corresponding target sequence is greater than 94.5%, 95%, 95.5%, 96%, 96.5%, 97%, 97.5%, 98%, 98.5%, 99%, 99.5%, or 99.9%. In some embodiments, the degree of complementarity is 100%.

For all embodiments of the systems and methods described herein, it is known in the field that complete complementarity is not required for hybridization or binding as described herein, provided that there is sufficient complementarity to be functional. Modulations of cleavage efficiency can be exploited by introducing mismatches, e.g., one or more mismatches, such as 1 or 2 mismatches between a spacer sequence and a target sequence, including the position of the mismatch along the spacer/target. The more central (i.e., not at the 3′ or 5′ ends) a mismatch, e.g., a double mismatch, is located; the more cleavage efficiency is affected. Accordingly, by choosing mismatch positions along the spacer sequence, cleavage efficiency can be modulated. For example, if less than 100% cleavage of targets is desired (e.g., in a cell population), 1 or 2 mismatches between spacer and target sequence can be introduced in the spacer sequences.

### Optimization of CRISPR Systems for Use in Select Organisms

### Codon-Optimization

The invention contemplates all possible variations of nucleic acids, such as cDNA, that could be made by selecting combinations based on possible codon choices. These combinations are made in accordance with the standard triplet genetic code as applied to the polynucleotide encoding naturally occurring variant, and all such variations are to be considered as being specifically disclosed. Nucleotide sequences encoding type V-G CRISPR-Cas-associated effector protein variants that have been codon-optimized for expression in bacteria (e.g., E. coli) and in human cells are disclosed herein. For example, the codon-optimized sequences for human cells can be generated by substituting codons in the nucleotide sequence that occur at lower frequency in human cells for codons that occur at higher frequency in human cells. The frequency of occurrence for codons can be computationally determined by methods known in the art. An exemplary calculation of these codon frequencies for various host cells (e.g., E. coli, yeast, insect, C. elegans, D. melanogaster, human, mouse, rat, pig, P. pastoris, A. thalian, maize, and tobacco) have been published or made available by sources such as the GenScript® Codon Usage Frequence Table Tool (example codon usage tables for E. coli and Humans are included below.

TABLE 1
E. coli Codon Usage Table

| | Amino | Frac- | | | Amino | Frac- | |
|---|---|---|---|---|---|---|---|
| Triplet | acid | tion | Number | Triplet | acid | tion | Number |
| TTT | F | 0.58 | 80995 | TCT | S | 0.17 | 38027 |
| TTC | F | 0.42 | 58774 | TCC | S | 0.15 | 33430 |
| TTA | L | 0.14 | 52382 | TCA | S | 0.14 | 32715 |
| TTG | L | 0.13 | 47500 | TCG | S | 0.14 | 31146 |
| TAT | Y | 0.59 | 63937 | TGT | C | 0.46 | 19138 |
| TAC | Y | 0.41 | 44631 | TGC | C | 0.54 | 22188 |
| TAA | * | 0.61 | 7356 | TGA | * | 0.3 | 3623 |
| TAG | * | 0.09 | 989 | TGG | W | 1 | 50991 |
| CTT | L | 0.12 | 43449 | CCT | P | 0.18 | 27340 |
| CTC | L | 0.1 | 37347 | CCC | P | 0.13 | 19666 |
| CTA | L | 0.04 | 15409 | CCA | P | 0.2 | 31534 |
| CTG | L | 0.47 | 177210 | CCG | P | 0.49 | 76644 |
| CAT | H | 0.57 | 45879 | CGT | R | 0.36 | 73197 |
| CAC | H | 0.43 | 34078 | CGC | R | 0.36 | 72212 |
| CAA | Q | 0.34 | 53394 | CGA | R | 0.07 | 13844 |
| CAG | Q | 0.66 | 104171 | CGG | R | 0.11 | 21552 |
| ATT | I | 0.49 | 109072 | ACT | T | 0.19 | 37842 |
| ATC | I | 0.39 | 86796 | ACC | T | 0.4 | 80547 |
| ATA | I | 0.11 | 24984 | ACA | T | 0.17 | 33910 |
| ATG | M | 1 | 96695 | ACG | T | 0.25 | 50269 |
| AAT | N | 0.49 | 75436 | AGT | S | 0.16 | 36097 |
| AAC | N | 0.51 | 78443 | AGC | S | 0.25 | 55551 |
| AAA | K | 0.74 | 129137 | AGA | R | 0.07 | 13152 |
| AAG | K | 0.26 | 45459 | AGG | R | 0.04 | 7607 |
| GTT | V | 0.28 | 72584 | GCT | A | 0.18 | 62479 |
| GTC | V | 0.2 | 52439 | GCC | A | 0.26 | 88721 |
| GTA | V | 0.17 | 42420 | GCA | A | 0.23 | 77547 |
| GTG | V | 0.35 | 89265 | GCG | A | 0.33 | 110308 |
| GAT | D | 0.63 | 119939 | GGT | G | 0.35 | 93325 |
| GAC | D | 0.37 | 70394 | GGC | G | 0.37 | 99390 |
| GAA | E | 0.68 | 143353 | GGA | G | 0.13 | 34799 |
| GAG | E | 0.32 | 68609 | GGG | G | 0.15 | 41277 |

TABLE 2
Human Codon Usage Table

| | Amino | Frac- | | | Amino | Frac- | |
|---|---|---|---|---|---|---|---|
| Triplet | acid | tion | Number | Triplet | acid | tion | Number |
| TTT | F | 0.45 | 336562 | TCT | S | 0.18 | 291040 |
| TTC | F | 0.55 | 406571 | TCC | S | 0.22 | 346943 |
| TTA | L | 0.07 | 143715 | TCA | S | 0.15 | 233110 |
| TTG | L | 0.13 | 249879 | TCG | S | 0.06 | 89429 |
| TAT | Y | 0.43 | 239268 | TGT | C | 0.45 | 197293 |
| TAC | Y | 0.57 | 310695 | TGC | C | 0.55 | 243685 |
| TAA | * | 0.28 | 14322 | TGA | * | 0.52 | 25383 |
| TAG | * | 0.2 | 10915 | TGG | W | 1 | 255512 |
| CTT | L | 0.13 | 253795 | CCT | P | 0.28 | 343793 |
| CTC | L | 0.2 | 386182 | CCC | P | 0.33 | 397790 |
| CTA | L | 0.07 | 138154 | CCA | P | 0.27 | 331944 |
| CTG | L | 0.41 | 800774 | CCG | P | 0.11 | 139414 |
| CAT | H | 0.41 | 207826 | CGT | R | 0.08 | 93458 |
| CAC | H | 0.59 | 297048 | CGC | R | 0.19 | 217130 |
| CAA | Q | 0.25 | 234785 | CGA | R | 0.11 | 126113 |
| CAG | Q | 0.75 | 688316 | CGG | R | 0.21 | 235938 |
| ATT | I | 0.36 | 313225 | ACT | T | 0.24 | 255582 |
| ATC | I | 0.48 | 426570 | ACC | T | 0.36 | 382050 |
| ATA | I | 0.16 | 140652 | ACA | T | 0.28 | 294223 |
| ATG | M | 1 | 443795 | ACG | T | 0.12 | 123533 |
| AAT | N | 0.46 | 331714 | AGT | S | 0.15 | 237404 |
| AAC | N | 0.54 | 387148 | AGC | S | 0.24 | 385113 |
| AAA | K | 0.42 | 476554 | AGA | R | 0.2 | 228151 |
| AAG | K | 0.58 | 654280 | AGG | R | 0.2 | 227281 |

### Methods of Using CRISPR Systems

The CRISPR systems described herein have a wide variety of utilities including modifying (e.g., deleting, inserting, translocating, inactivating, or activating) a target polynucleotide in a multiplicity of cell types. The CRISPR systems have a broad spectrum of applications in, e.g., DNA/RNA detection (e.g., specific high sensitivity enzymatic reporter unlocking (SHERLOCK)), tracking and labeling of nucleic acids, enrichment assays (extracting desired sequence from background), detecting circulating tumor DNA, preparing next generation library, drug screening, disease diagnosis and prognosis, and treating various genetic disorders. Without wishing to be bound by any particular theory, CRISPR systems including a Cas12g protein may exhibit increased activity or may be preferentially active when targeting in certain classes of loci, such as transcriptionally-active genomic regions. Therefore, CRISPR systems including a Cas12g protein may be advantageously used in applications targeting transcriptionally-active loci.

### DNA/RNA Detection

In one aspect, the CRISPR-Cas systems described herein can be used in DNA/RNA detection by RNA sensing. Single effector RNA-guided nucleases can be reprogrammed with RNA guides to provide a platform for specific RNA sensing. Upon recognition of its RNA target, activated Type V-G single effector RNA-guided nucleases engage in “collateral” cleavage of nearby ssDNA and RNA with no sequence similarity to the target. This RNA guide-programmed collateral cleavage activity allows the CRISPR systems to detect the presence of a specific RNA by nonspecific degradation of labeled ssDNA and RNA.

The collateral nuclease activity of the CRISPR-Cas system described herein can be combined with a reporter in RNA detection applications such as described by methods called SHERLOCK (Specific High Sensitivity Enzymatic Reporter UnLOCKing) or the DNA Endonuclease-Targeted CRISPR trans reporter (DETECTR), which achieve attomolar sensitivity for nucleic acid detection. Methods of using CRISPR-Cas systems in SHERLOCK are described in detail, e.g., in Gootenberg, et al. “Nucleic acid detection with CRISPR-Cas13a/C2c2,” Science, 356 (6336): 438-442 (2017), which is incorporated herein by reference in its entirety. Methods of using CRISPR-Cas systems in DETECTR are described in detail, e.g., Chen et al., Science, 360 (6387): 436-439, 2018, which is incorporated herein by reference in its entirety.

The CRISPR-associated proteins can further be used in Northern blot assays, which use electrophoresis to separate RNA samples by size. The CRISPR-associated proteins can be used to specifically bind and detect the target RNA sequence. The CRISPR-associated proteins can also be fused to a fluorescent protein (e.g., GFP) and used to track RNA localization in living cells. More particularly, the CRISPR-associated proteins can be inactivated in that they no longer cleave RNAs as described above. Thus, CRISPR-associated proteins can be used to determine the localization of the RNA or specific splice variants, the level of mRNA transcripts, up- or down-regulation of transcripts and disease-specific diagnosis. The CRISPR-associated proteins can be used for visualization of RNA in (living) cells using, for example, fluorescent microscopy or flow cytometry, such as fluorescence-activated cell sorting (FACS), which allows for high-throughput screening of cells and recovery of living cells following cell sorting. A detailed description regarding how to detect DNA and RNA can be found, e.g., in International Publication No. WO 2017/070605, which is incorporated herein by reference in its entirety.

In some embodiments, the CRISPR systems described herein can be used in multiplexed error-robust fluorescence in situ hybridization (MERFISH). These methods are described in, e.g., Chen et al., “Spatially resolved, highly multiplexed RNA profiling in single cells,” Science, 2015 Apr. 24; 348(6233):aaa6090, which is incorporated herein by reference in its entirety.

In some embodiments, CRISPR systems including a Cas12g protein described herein exhibit increased activity (e.g., increased nuclease activity) when the effector protein targets a transcriptionally-active genomic region, and can be used to detect active expression (e.g., transcription) of a target sequence of interest. In some embodiments, the level of expression (e.g., transcription) of a sequence of interest (e.g., a gene of interest) may be quantitated by determining a level of activity (e.g., nuclease activity) of a CRISPR system described herein that targets the sequence of interest, and the level of activity of the CRISPR system may be used to determine the expression level of the sequence of interest.

In some embodiments, the CRISPR systems described herein can be used to detect a target RNA in a sample (e.g., a clinical sample, a cell, or a cell lysate). The collateral RNAse activity of the Type V-G CRISPR-Cas effector proteins (CLUST.019143) described herein is activated when the effector proteins bind to a target nucleic acid. Upon binding to the target RNA of interest, the effector protein cleaves a labeled detector RNA to generate or change a signal (e.g., an increased signal or a decreased signal) thereby allowing for the qualitative and quantitative detection of the target RNA in the sample. The specific detection and quantification of RNA in the sample allows for a multitude of applications including diagnostics.

In some embodiments, the methods include a) contacting a sample with: (i) an RNA guide (e.g., crRNA) and/or a nucleic acid encoding the RNA guide, wherein the RNA guide consists of a direct repeat sequence and a spacer sequence capable of hybridizing to the target RNA; (ii) a Type V-G CRISPR-Cas effector protein and/or a nucleic acid encoding the effector protein; and (iii) a labeled detector RNA; wherein the effector protein associates with the RNA guide to form a complex; wherein the RNA guide hybridizes to the target RNA; and wherein upon binding of the complex to the target RNA, the effector protein exhibits collateral RNAse activity and cleaves the labeled detector RNA; and b) measuring a detectable signal produced by cleavage of the labeled detector RNA, wherein said measuring provides for detection of the single-stranded target RNA in the sample.

In some embodiments, the methods further include comparing the detectable signal with a reference signal and determining the amount of target RNA in the sample. In some embodiments, the measuring is performed using gold nanoparticle detection, fluorescence polarization, colloid phase transition/dispersion, electrochemical detection, and semiconductor based-sensing. In some embodiments, the labeled detector RNA includes a fluorescence-emitting dye pair, a fluorescence resonance energy transfer (FRET) pair, or a quencher/fluor pair. In some embodiments, upon cleavage of the labeled detector RNA by the effector protein, an amount of detectable signal produced by the labeled detector RNA is decreased or increased. In some embodiments, the labeled detector RNA produces a first detectable signal prior to cleavage by the effector protein and a second detectable signal after cleavage by the effector protein.

In some embodiments, a detectable signal is produced when the labeled detector RNA is cleaved by the effector protein. In some embodiments, the labeled detector RNA includes a modified nucleobase, a modified sugar moiety, a modified nucleic acid linkage, or a combination thereof.

In some embodiments, the methods include the multi-channel detection of multiple independent target RNAs in a sample (e.g., two, three, four, five, six, seven, eight, nine, ten, fifteen, twenty, thirty, forty, or more target RNAs) by using multiple Type V-G CRISPR-Cas effector protein systems, each including a distinct orthologous effector protein and corresponding RNA guides, allowing for the differentiation of multiple target RNAs in the sample. In some embodiments, the methods include the multi-channel detection of multiple independent target RNAs in a sample, with the use of multiple instances of Type V-G CRISPR-Cas effector protein systems, each containing an orthologous effector protein with differentiable collateral RNAse substrates. Methods of detecting an RNA in a sample using CRISPR-associated proteins are described, for example, in U.S. Patent Publication No. 2017/0362644, the entire contents of which are incorporated herein by reference.

### Tracking and Labeling of Nucleic Acids

Cellular processes depend on a network of molecular interactions among proteins, RNAs, and DNAs. Accurate detection of protein-DNA and protein-RNA interactions is key to understanding such processes. In vitro proximity labeling techniques employ an affinity tag combined with, a reporter group, e.g., a photoactivatable group, to label polypeptides and RNAs in the vicinity of a protein or RNA of interest in vitro. After UV irradiation, the photoactivatable groups react with proteins and other molecules that are in close proximity to the tagged molecules, thereby labelling them. Labelled interacting molecules can subsequently be recovered and identified. The RNA targeting effector proteins can for instance be used to target probes to selected RNA sequences. These applications can also be applied in animal models for in vivo imaging of diseases or difficult-to culture cell types. The methods of tracking and labeling of nucleic acids are described, e.g., in U.S. Pat. No. 8,795,965; WO 2016205764; and WO 2017070605; each of which is incorporated herein by reference in its entirety.

RNA Isolation, Purification, Enrichment, and/or Depletion

The CRISPR systems (e.g., CRISPR-associated proteins) described herein can be used to isolate and/or purify RNA. For example, the CRISPR-associated proteins can be fused to an affinity tag that can be used to isolate and/or purify the RNA-CRISPR-associated protein complex. These applications are useful, e.g., for the analysis of gene expression profiles in cells.

In some embodiments, the CRISPR-associated proteins can be used to target a specific noncoding RNA (ncRNA) thereby blocking its activity. In some embodiments, the CRISPR-associated proteins can be used to specifically enrich a particular RNA (including but not limited to increasing stability, etc.), or alternatively, to specifically deplete a particular RNA (e.g., particular splice variants, isoforms, etc.).

These methods are generally described, e.g., in U.S. Pat. No. 8,795,965, WO 2016205764, and WO 2017070605, each of which is incorporated herein by reference herein in its entirety.

### High-Throughput Screening

The CRISPR systems described herein can be used for preparing next generation sequencing (NGS) libraries. For example, to create a cost-effective NGS library, the CRISPR systems can be used to disrupt the coding sequence of a target gene, and the CRISPR enzyme transfected clones can be screened simultaneously by next-generation sequencing (e.g., on the Ion Torrent PGM system). A detailed description regarding how to prepare NGS libraries can be found, e.g., in Bell et al., “A high-throughput screening strategy for detecting CRISPR-Cas9 induced mutations using next-generation sequencing,” BMC Genomics, 15.1 (2014): 1002, which is incorporated herein by reference in its entirety.

### Engineered Microorganisms

Microorganisms (e.g., E. coli, yeast, and microalgae) are widely used for synthetic biology. The development of synthetic biology has a wide utility, including various clinical applications. For example, the programmable CRISPR systems described herein can be used to split proteins of toxic domains for targeted cell death, e.g., using cancer-linked RNA as target transcript. Further, pathways involving protein-protein interactions can be influenced in synthetic biological systems with e.g. fusion complexes with the appropriate effectors such as kinases or enzymes.

In some embodiments, RNA guide sequences that target phage sequences can be introduced into the microorganism. Thus, the disclosure also provides methods of vaccinating a microorganism (e.g., a production strain) against phage infection.

In some embodiments, the CRISPR systems provided herein can be used to engineer microorganisms, e.g., to improve yield or improve fermentation efficiency. For example, the CRISPR systems described herein can be used to engineer microorganisms, such as yeast, to generate biofuel or biopolymers from fermentable sugars, or to degrade plant-derived lignocellulose derived from agricultural waste as a source of fermentable sugars. More particularly, the methods described herein can be used to modify the expression of endogenous genes required for biofuel production and/or to modify endogenous genes, which may interfere with the biofuel synthesis. These methods of engineering microorganisms are described e.g., in Verwaal et al., “CRISPR/Cpf1 enables fast and simple genome editing of Saccharomyces cerevisiae,” Yeast, 2017 Sep. 8. doi: 10.1002/yea.3278; and Hlavova et al., “Improving microalgae for biotechnology—from genetics to synthetic biology,” Biotechnol. Adv., 2015 Nov. 1; 33:1194-203, both of which are incorporated herein by reference in their entirety.

In some embodiments, the CRISPR systems provided herein can be used to induce death or dormancy of a cell (e.g., a microorganism such as an engineered microorganism). These methods can be used to induce dormancy or death of a multitude of cell types including prokaryotic and eukaryotic cells, including, but not limited to, mammalian cells (e.g., cancer cells, or tissue culture cells), protozoans, fungal cells, cells infected with a virus, cells infected with an intracellular bacteria, cells infected with an intracellular protozoan, cells infected with a prion, bacteria (e.g., pathogenic and non-pathogenic bacteria), protozoans, and unicellular and multicellular parasites. For instance, in the field of synthetic biology it is highly desirable to have mechanisms of controlling engineered microorganisms (e.g., bacteria) in order to prevent their propagation or dissemination. The systems described herein can be used as “kill-switches” to regulate and/or prevent the propagation or dissemination of an engineered microorganism. Further, there is a need in the art for alternatives to current antibiotic treatments.

The systems described herein can also be used in applications where it is desirable to kill or control a specific microbial population (e.g., a bacterial population). For example, the systems described herein may include an RNA guide (e.g., a crRNA) that targets a nucleic acid (e.g., an RNA) that is genus-, species-, or strain-specific, and can be delivered to the cell. Upon complexing and binding to the target nucleic acid, the collateral RNAse activity of the CLUST.019143 (Type V-G) CRISPR-Cas effector proteins is activated leading to the cleavage of collateral RNA within the microorganisms, ultimately resulting in dormancy or death. In some embodiments, the methods comprise contacting the cell with a system described herein including a CLUST.019143 (Type V-G) CRISPR-Cas effector proteins or a nucleic acid encoding the effector protein, and a RNA guide (e.g., a crRNA) or a nucleic acid encoding the RNA guide, wherein the spacer sequence is complementary to at least 15 nucleotides (e.g., 16, 17, 18, 19, 20, 21, 22, 23, 24, 25, 26, 27, 28, 29, 30, 35, 40, 45, 50 or more nucleotides) of a target nucleic acid (e.g., a genus-, strain-, or species-specific RNA guide).

Without wishing to be bound by any particular theory, the cleavage of collateral RNA by the CLUST.019143 (Type V-G) CRISPR-Cas effector proteins can induce programmed cell death, cell toxicity, apoptosis, necrosis, necroptosis, cell death, cell cycle arrest, cell anergy, a reduction of cell growth, or a reduction in cell proliferation. For example, in bacteria, the cleavage of collateral RNA by the CLUST.019143 (Type V-G) CRISPR-Cas effector proteins can be bacteriostatic or bacteriocidal.

### Application in Plants

The CRISPR systems described herein have a wide variety of utility in plants. In some embodiments, the CRISPR systems can be used to engineer genomes of plants (e.g., improving production, making products with desired post-translational modifications, or introducing genes for producing industrial products). In some embodiments, the CRISPR systems can be used to introduce a desired trait to a plant (e.g., with or without heritable modifications to the genome), or regulate expression of endogenous genes in plant cells or whole plants.

In some embodiments, the CRISPR systems can be used to identify, edit, and/or silence genes encoding specific proteins, e.g., allergenic proteins (e.g., allergenic proteins in peanuts, soybeans, lentils, peas, green beans, and mung beans). A detailed description regarding how to identify, edit, and/or silence genes encoding proteins is described, e.g., in Nicolaou et al., “Molecular diagnosis of peanut and legume allergy,” Curr. Opin. Allergy Clin. Immunol. 11(3):222-8 (2011), and WO 2016205764 A1; both of which are incorporated herein by reference in their entirety.

### Gene Drives

Gene drive is the phenomenon in which the inheritance of a particular gene or set of genes is favorably biased. The CRISPR systems described herein can be used to build gene drives. For example, the CRISPR systems can be designed to target and disrupt a particular allele of a gene, causing the cell to copy the second allele to fix the sequence. Because of the copying, the first allele will be converted to the second allele, increasing the chance of the second allele being transmitted to the offspring. A detailed method regarding how to use the CRISPR systems described herein to build gene drives is described, e.g., in Hammond et al., “A CRISPR-Cas9 gene drive system targeting female reproduction in the malaria mosquito vector Anopheles gambiae,” Nat. Biotechnol., 2016 January; 34(1):78-83, which is incorporated herein by reference in its entirety.

### Pooled-Screening

As described herein, pooled CRISPR screening is a powerful tool for identifying genes involved in biological mechanisms such as cell proliferation, drug resistance, and viral infection. Cells are transduced in bulk with a library of RNA guide-encoding vectors described herein, and the distribution of RNA guides is measured before and after applying a selective challenge. Pooled CRISPR screens work well for mechanisms that affect cell survival and proliferation, and they can be extended to measure the activity of individual genes (e.g., by using engineered reporter cell lines). Arrayed CRISPR screens, in which only one gene is targeted at a time, make it possible to use RNA-seq as the readout. In some embodiments, the CRISPR systems as described herein can be used in single-cell CRISPR screens. A detailed description regarding pooled CRISPR screenings can be found, e.g., in Datlinger et al., “Pooled CRISPR screening with single-cell transcriptome read-out,” Nat. Methods., 2017 March; 14(3):297-301, which is incorporated herein by reference in its entirety.

### Saturation Mutagenesis (“Bashing”)

The CRISPR systems described herein can be used for in situ saturating mutagenesis. In some embodiments, a pooled RNA guide library can be used to perform in situ saturating mutagenesis for particular genes or regulatory elements. Such methods can reveal critical minimal features and discrete vulnerabilities of these genes or regulatory elements (e.g., enhancers). These methods are described, e.g., in Canver et al., “BCL11A enhancer dissection by Cas9-mediated in situ saturating mutagenesis,” Nature, 2015 Nov. 12; 527(7577):192-7, which is incorporated herein by reference in its entirety.

### RNA-Related Applications

The CRISPR systems described herein have various RNA-related applications, e.g., modulating gene expression, degrading a RNA molecule, inhibiting RNA expression, screening RNA or RNA products, determining functions of lincRNA or non-coding RNA, inducing cell dormancy, inducing cell cycle arrest, reducing cell growth and/or cell proliferation, inducing cell anergy, inducing cell apoptosis, inducing cell necrosis, inducing cell death, and/or inducing programmed cell death. A general description of these types of applications can be found, e.g., in WO 2016/205764 A1, which is incorporated herein by reference in its entirety. In different embodiments, the methods described herein can be performed in vitro, in vivo, or ex vivo.

For example, the CRISPR systems described herein can be administered to a subject having a disease or disorder to target and induce cell death in a particular cell or a group of particular cells in a diseased state (e.g., cancer cells or cells infected with an infectious agent). For instance, in some embodiments, the CRISPR systems described herein can be used to target and induce cell death in a cancer cell, wherein the cancer cell is from a subject having a Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, or urinary bladder cancer.

### Modulating Gene Expression

The CRISPR systems described herein can be used to modulate gene expression. The CRISPR systems can be used, together with suitable RNA guides, to target gene expression, via control of RNA processing. The control of RNA processing can include, e.g., RNA processing reactions such as RNA splicing (e.g., alternative splicing), viral replication, and tRNA biosynthesis. The RNA targeting proteins in combination with suitable RNA guides can also be used to control RNA activation (RNAa). RNA activation is a small RNA-guided and Argonaute (Ago)-dependent gene regulation phenomenon in which promoter-targeted short double-stranded RNAs (dsRNAs) induce target gene expression at the transcriptional/epigenetic level. RNAa leads to the promotion of gene expression, so control of gene expression may be achieved that way through disruption or reduction of RNAa. In some embodiments, the methods include the use of the RNA targeting CRISPR as substitutes for e.g., interfering ribonucleic acids (such as siRNAs, shRNAs, or dsRNAs). The methods of modulating gene expression are described, e.g., in WO 2016205764, which is incorporated herein by reference in its entirety.

### Controlling RNA Interference

Control over interfering RNAs or microRNAs (miRNA) can help reduce off-target effects by reducing the longevity of the interfering RNAs or miRNAs in vivo or in vitro. In some embodiments, the target RNAs can include interfering RNAs, i.e., RNAs involved in the RNA interference pathway, such as small hairpin RNAs (shRNAs), small interfering (siRNAs), etc. In some embodiments, the target RNAs include, e.g., miRNAs or double stranded RNAs (dsRNA).

In some embodiments, if the RNA targeting protein and suitable RNA guides are selectively expressed (for example spatially or temporally under the control of a regulated promoter, for example a tissue- or cell cycle-specific promoter and/or enhancer), the new systems described herein can be used to protect the cells or systems (in vivo or in vitro) from RNA interference (RNAi) in those cells. These new methods are useful in neighboring tissues or cells where RNAi is not required or for the purposes of comparison of the cells or tissues where the CRISPR-associated proteins and suitable crRNAs are and are not expressed (i.e., where the RNAi is not controlled and where it is, respectively). The RNA targeting proteins can be used to control or bind to molecules comprising or consisting of RNAs, such as ribozymes, ribosomes, or riboswitches. In some embodiments, the RNA guides can recruit the RNA targeting proteins to these molecules so that the RNA targeting proteins are able to bind to them. These methods are described, e.g., in WO 2016205764 and WO 2017070605, both of which are incorporated herein by reference in the entirety.

### Modifying Riboswitches and Controlling Metabolic Regulations

Riboswitches are regulatory segments of messenger RNAs that bind to small molecules and in turn regulate gene expression. This mechanism allows the cell to sense the intracellular concentration of these small molecules. A specific riboswitch typically regulates its adjacent gene by altering the transcription, the translation or the splicing of this gene. Thus, in some embodiments, the riboswitch activity can be controlled by the use of the RNA targeting proteins described herein in combination with suitable RNA guides to target the riboswitches. This can be achieved through cleavage of, or binding to, the riboswitch. Methods of using CRISPR systems to control riboswitches are described, e.g., in WO 2016205764 and WO 2017070605, both of which are incorporated herein by reference in their entireties.

### RNA Modification

In some embodiments, the CRISPR-associated proteins described herein can be fused to a base-editing domain, such as ADAR1, ADAR2, APOBEC, or activation-induced cytidine deaminase (AID), and can be used to modify an RNA sequence (e.g., an mRNA). In some embodiments, the CRISPR-associated protein includes one or more mutations (e.g., in a catalytic domain), which renders the CRISPR-associated protein incapable of cleaving RNA.

In some embodiments, the CRISPR-associated proteins can be used with an RNA-binding fusion polypeptide comprising a base-editing domain (e.g., ADAR1, ADAR2, APOBEC, or AID) fused to an RNA-binding domain, such as MS2 (also known as MS2 coat protein), Qbeta (also known as Qbeta coat protein), or PP7 (also known as PP7 coat protein). The amino acid sequences of the RNA-binding domains MS2, Qbeta, and PP7 are provided below:

MS2 (MS2 coat protein)
(SEQ ID NO: 316)
MASNFTQFVLVDNGGTGDVTVAPSNFANGVAEWISSNSRSQAYKVTCSVR
QSSAQKRKYTIKVEVPKVATQTVGGVELPVAAWRSYLNMELTIPIFATNS
DCELIVKAMQGLLKDGNPIPSAIAANSGIY
Qbeta (Qbeta coat protein)
(SEQ ID NO: 317)
MAKLETVTLGNIGKDGKQTLVLNPRGVNPTNGVASLSQAGAVPALEKRVT
VSVSQPSRNRKNYKVQVKIQNPTACTANGSCDPSVTRQAYADVTFSFTQY
STDEERAFVRTELAALLASPLLIDAIDQLNPAY
PP7 (PP7 coat protein)
(SEQ ID NO: 318)
MSKTIVLSVGEATRTLTEIQSTADRQIFEEKVGPLVGRLRLTASLRQNGA
KTAYRVNLKLDQADVVDCSTSVCGELPKVRYTQVWSHDVTIVANSTEASR
KSLYDLTKSLVVQATSEDLVVNLVPLGR

In some embodiments, the RNA binding domain can bind to a specific sequence (e.g., an aptamer sequence) or secondary structure motifs on a crRNA of the system described herein (e.g., when the crRNA is in an effector-crRNA complex), thereby recruiting the RNA binding fusion polypeptide (which has a base-editing domain) to the effector complex. For example, in some embodiments, the CRISPR system includes a CRISPR associated protein, a crRNA having an aptamer sequence (e.g., an MS2 binding loop, a QBeta binding loop, or a PP7 binding loop), and a RNA-binding fusion polypeptide having a base-editing domain fused to an RNA-binding domain that specifically binds to the aptamer sequence. In this system, the CRISPR-associated protein forms a complex with the crRNA having the aptamer sequence. Further the RNA-binding fusion polypeptide binds to the crRNA (via the aptamer sequence) thereby forming a tripartite complex that can modify a target RNA.

Methods of using CRISPR systems for base editing are described, e.g., in International Publication No. WO 2017/219027, which is incorporated herein by reference in its entirety, and in particular with respect to its discussion of RNA modification.

### RNA Splicing

In some embodiments, an inactivated CRISPR-associated protein described herein (e.g., a CRISPR associated protein having one or more mutations in a catalytic domain) can be used to target and bind to specific splicing sites on RNA transcripts. Binding of the inactivated CRISPR-associated protein to the RNA may sterically inhibit interaction of the spliceosome with the transcript, enabling alteration in the frequency of generation of specific transcript isoforms. Methods of using CRISPR systems to alter splicing are described, e.g., in International Publication No. WO 2017/219027, which is incorporated herein by reference in its entirety, and in particular with respect to its discussion of RNA splicing.

Quantitative Trait Mapping (crisprQTL)

The CRISPR systems described herein can be used for mapping coding and non-coding regions of a genome that influence gene expression. For example, in some embodiments a population of cells may be transduced with multiple random, barcoded, CRISPR RNA guide-programmed perturbations. Single-cell RNA-sequencing may then be used to profile gene expression and the collection of RNA guides in each cell. The generated data can be used to identify associations between RNA guides and quantitative changes in gene expression, which facilitates the analysis of the cis-regulatory architecture of the cells. These methods are described, for example, in Gasperini et al., “crisprQTL mapping as a genome-wide association framework for cellular genetic screens,” bioRxiv, posted May 4, 2018, doi: doi.org/10.1101/314344, which is incorporated herein by reference.

### Lineage Tracing

The CRISPR systems described herein can be used for cell lineage tracing. CRISPR systems can be used to combinatorially edit a ‘barcode’ sequence in the genomes of the cells of an embryo as it develops. Based on knowledge of the on-target and off-target editing characteristics of the CRISPR system, this combinatorial editing activity can be tuned such that genomic editing events accumulate in informative ways as the cells grow and divide. The patterns of barcodes observed in the edited cells are used to reconstruct the developmental lineage relationships for cells in an organism. These methods, and variants thereof, are described, for example, in McKenna et al., “Whole organism lineage tracing by combinatorial and cumulative genome editing”, Science, 29 Jul. 2016; 353(6298): aaf7907, which is incorporated herein by reference. These methods can also be used in cancer cells for example, to track the evolution of a tumor over time, and space, in the case of metastasis. In some embodiments, the Cas12g proteins that exhibit increased activity when the effector protein targets a transcriptionally-active genomic region (e.g., Cas12g1) can be used to perform lineage tracing of cells in which marker genes of interest are expressed. For example, in some embodiments, these systems can be used to trace the evolution of cancer cells expressing genes relevant to the diagnosis or treatment of disease.

### Therapeutic Applications

The CRISPR systems described herein that have activity in a mammalian cellular context can have a diverse range of therapeutic applications. In some embodiments, the new CRISPR systems targeting RNA can be used to directly treat diseases and disorders. From the development of therapeutics based on RNA interference, it has been demonstrated that a number of diseases can be treatable by targeting endogenous RNA. The RNA targeting CRISPR systems described herein can thus be applied in a similar way.

In one aspect, the CRISPR systems described herein can be used for treating a disease caused by overexpression of RNAs, toxic RNAs, and/or mutated RNAs (e.g., splicing defects or truncations). For example, expression of the toxic RNAs may be associated with the formation of nuclear inclusions and late-onset degenerative changes in brain, heart, or skeletal muscle. In some embodiments, the disorder is myotonic dystrophy. In myotonic dystrophy, the main pathogenic effect of the toxic RNAs is to sequester binding proteins and compromise the regulation of alternative splicing (see, e.g., Osborne et al., “RNA-dominant diseases,” Hum. Mol. Genet., 2009 Apr. 15; 18(8):1471-81). Myotonic dystrophy (dystrophia myotonica (DM)) is of particular interest to geneticists because it produces an extremely wide range of clinical features. The classical form of DM, which is now called DM type 1 (DM1), is caused by an expansion of CTG repeats in the 3′-untranslated region (UTR) of DMPK, a gene encoding a cytosolic protein kinase. The CRISPR systems as described herein can target overexpressed RNA or toxic RNA, e.g., the DMPK gene or any of the mis-regulated alternative splicing in DM1 skeletal muscle, heart, or brain. Another example is Huntington's disease (HD), in which the trinucleotide expansion of CAG in the gene coding for the Huntingtin protein yields an aberrant protein. The specific knockdown of the mutated or allele-specific RNA encoding the abnormal Huntingtin protein may be beneficial. Mutations in APOB gene that cause familial hypobetalipoproteinemia (FHBL) cause mutated transcripts that interfere with the generation of the two natural forms of the transcript in unaffected individuals, and specific knockdown of the mutated APOB transcript may be beneficial to treat FHBL. Likewise, the recent FDA approval of Patisiran highlights the therapeutic potential of RNAi to knock down the toxic misfolded transthyretin protein (Kristen, et al. Neurodegener Dis Manag. 2019 February; 9(1):5-23.) that causes hereditary transthyretin-mediated amyloidosis, and utilizing the CRISPR systems described herein for targeting the toxic mutation can be expected to have similar benefits.

In another aspect, the CRISPR systems described herein can target genes identified from genetic studies that provide a protective effect when knocked down. These may confer protective mutations, such as PCSK9 knockdown for reduction of plasma LDL and lowering the risk of cardiovascular disease (Frank-Kamenetsky, et al. Proc Natl Acad Sci USA. 2008 Aug. 19; 105(33): 11915-11920), to CCR5 knockdown for conferring protection against HIV infection (Shimizu et al, Mol Ther Nucleic Acids. 2015 February; 4(2): e227.).

The CRISPR systems described herein can also target trans-acting mutations affecting RNA-dependent functions that cause various diseases such as, e.g., Prader Willi syndrome, Spinal muscular atrophy (SMA), and Dyskeratosis congenita. A list of diseases that can be treated using the CRISPR systems described herein is summarized in Cooper et al., “RNA and disease,” Cell, 136.4 (2009): 777-793, and WO 2016205764 A1, both of which are incorporated herein by reference in their entirety.

The CRISPR systems described herein can also be used in the treatment of various tauopathies, including, e.g., primary and secondary tauopathies, such as primary age-related tauopathy (PART)/Neurofibrillary tangle (NFT)-predominant senile dementia (with NFTs similar to those seen in Alzheimer Disease (AD), but without plaques), dementia pugilistica (chronic traumatic encephalopathy), and progressive supranuclear palsy. A useful list of tauopathies and methods of treating these diseases are described, e.g., in WO 2016205764, which is incorporated herein by reference in its entirety.

The CRISPR systems described herein can also be used to target mutations disrupting the cis-acting splicing codes that can cause splicing defects and diseases. These diseases include, e.g., motor neuron degenerative disease that results from deletion of the SMN1 gene (e.g., spinal muscular atrophy), Duchenne or Becker Muscular Dystrophy (DMD/BMD), frontotemporal dementia, and Parkinsonism linked to chromosome 17 (FTDP-17), and cystic fibrosis. In the example of Friedreich's ataxia, the disease is caused by a trinucleotide repeat expansion of GAA in the Frataxin (FXN) gene that interferes with normal splicing. The RNA-targeting effectors described herein can be targeted to affect the splice site and restore normal protein activity for therapeutic benefit.

The CRISPR systems described herein can also be used to treat a cancer in a subject (e.g., a human subject). For example, the CRISPR-associated proteins described herein can be programmed with a crRNA targeting an RNA molecule that is aberrant (e.g., comprises a point mutation or is alternatively spliced) and found in cancer cells, to induce cell death in the cancer cells (e.g., via apoptosis). One instance is the BCR-ABL fusion oncogene found in many patients with chronic myelogenous leukemia (CML), a mutation specific to the cancer and thus able to be targeted without affecting healthy somatic cells.

Furthermore, the CRISPR systems described herein can be used to treat an infectious disease in a subject. For example, the CRISPR-associated proteins described herein can be programmed with crRNA targeting RNA expressed by an infectious agent (e.g., a bacteria, a virus, a parasite or a protozoan) in order to target and induce cell death in the infectious agent cell. The CRISPR systems may also be used to treat diseases where an intracellular infectious agent infects the cells of a host subject. By programming the CRISPR-associated protein to target an RNA molecule encoded by an infectious agent gene, cells infected with the infectious agent can be targeted and cell death induced. The CRISPR systems can further be used for antiviral activity, in particular against RNA viruses such as HIV, respiratory syncytial virus (RSV) and Hepatitis C virus (HCV) although DNA viruses with RNA intermediates in their life cycle can also be targeted (such as herpes simplex virus types 1 and 2, and Hepatitis B virus). The effector proteins can target the viral RNAs using suitable RNA guides selected to target viral RNA sequences.

Furthermore, in vitro RNA sensing assays can be used to detect specific RNA substrates. The RNA targeting effector proteins can be used for RNA-based sensing in living cells. Examples of applications are diagnostics by sensing of, for examples, disease-specific RNAs.

A detailed description of therapeutic applications of the CRISPR systems described herein can be found, e.g., in U.S. Pat. No. 8,795,965, EP 3009511, WO 2016205764, and WO 2017070605; each of which is incorporated herein by reference in its entirety.

In another aspect, the CRISPR systems described herein can be engineered to enable additional functions that utilize enzymatically inactive Cas12g as a chassis on top of which protein domains can be attached to confer activities such as base editing (Cox et al., Science. 2017 Nov. 24; 358(6366):1019-1027), and RNA splice modification (Konermann, et al. Cell. 2018 Apr. 19; 173(3):665-676.e14).

Suitably, there is provided CRISPR-Cas systems and cells of the present invention for use in the treatment or prevention of any of the disease disclosed herein.

### Delivery of CRISPR Systems

The CRISPR systems described herein, or components thereof, nucleic acid molecules thereof, or nucleic acid molecules encoding or providing components thereof, can be delivered by various delivery systems such as vectors, e.g., plasmids, viral delivery vectors. The new CRISPR enzymes and/or any of the RNAs (e.g., RNA guides) can be delivered using suitable vectors, e.g., plasmids or viral vectors, such as adeno-associated viruses (AAV), lentiviruses, adenoviruses, and other viral vectors, or combinations thereof. The proteins and one or more RNA guides can be packaged into one or more vectors, e.g., plasmids or viral vectors. For bacterial applications, the nucleic acids encoding any of the components of the CRISPR systems described herein can be delivered to the bacteria using a phage. Exemplary phages, include, but are not limited to, T4 phage, Mu, λ phage, T5 phage, T7 phage, T3 phage, Φ29, M13, MS2, Qβ, and ΦX174.

In some embodiments, the vectors, e.g., plasmids or viral vectors, are delivered to the tissue of interest by, e.g., intramuscular injection, intravenous administration, transdermal administration, intranasal administration, oral administration, or mucosal administration. Such delivery may be either via a single dose or multiple doses. One skilled in the art understands that the actual dosage to be delivered herein may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

In certain embodiments, the delivery is via adenoviruses, which can be administered in a single dose containing at least 1×10⁵ particles (also referred to as particle units, pu) of adenoviruses. In some embodiments, the dose is at least about 1×10⁶ particles, at least about 1×10⁷ particles, at least about 1×10⁸ particles, or at least about 1×10⁹ particles of the adenoviruses. The delivery methods and the doses are described, e.g., in WO 2016205764 and U.S. Pat. No. 8,454,972, both of which are incorporated herein by reference in their entirety.

In some embodiments, the delivery is via a recombinant adeno-associated virus (rAAV) vector. For example, in some embodiments, a modified AAV vector may be used for delivery. Modified AAV vectors can be based on one or more of several capsid types, including AAV1, AV2, AAV5, AAV6, AAV8, AAV8.2. AAV9, AAV rhIO, modified AAV vectors (e.g., modified AAV2, modified AAV3, modified AAV6) and pseudotyped AAV (e.g., AAV2/8, AAV2/5 and AAV2/6). Exemplary AAV vectors and techniques that may be used to produce rAAV particles are known in the art (see, e.g., Aponte-Ubillus et al. (2018) Appl. Microbiol. Biotechnol. 102(3): 1045-54; Zhong et al. (2012) J. Genet. Syndr. Gene Ther. S1: 008; West et al. (1987) Virology 160:38-47 (1987); Tratschin et al. (1985) Mol. Cell. Biol. 5:3251-60); U.S. Pat. Nos. 4,797,368 and 5,173,414; and International Publication Nos. WO 2015/054653 and WO 93/24641, each of which is incorporated by reference).

In some embodiments, the delivery is via plasmids. The dosage can be a sufficient number of plasmids to elicit a response. In some cases, suitable quantities of plasmid DNA in plasmid compositions can be from about 0.1 to about 2 mg. Plasmids will generally include (i) a promoter; (ii) a sequence encoding a nucleic acid-targeting CRISPR enzymes, operably linked to the promoter; (iii) a selectable marker; (iv) an origin of replication; and (v) a transcription terminator downstream of and operably linked to (ii). The plasmids can also encode the RNA components of a CRISPR complex, but one or more of these may instead be encoded on different vectors. The frequency of administration is within the ambit of the medical or veterinary practitioner (e.g., physician, veterinarian), or a person skilled in the art.

In another embodiment, the delivery is via liposomes or lipofectin formulations and the like, and can be prepared by methods known to those skilled in the art. Such methods are described, for example, in WO 2016205764 and U.S. Pat. Nos. 5,593,972; 5,589,466; and 5,580,859; each of which is incorporated herein by reference in its entirety.

In some embodiments, the delivery is via nanoparticles or exosomes. For example, exosomes have been shown to be particularly useful in the delivery RNA.

Further means of introducing one or more components of the new CRISPR systems into cells is by using cell-penetrating peptides (CPP). In some embodiments, a cell-penetrating peptide is linked to the CRISPR enzymes. In some embodiments, the CRISPR enzymes and/or RNA guides are coupled to one or more CPPs to transport them inside cells effectively (e.g., plant protoplasts). In some embodiments, the CRISPR enzymes and/or RNA guide(s) are encoded by one or more circular or non-circular DNA molecules that are coupled to one or more CPPs for cell delivery.

CPPs are short peptides of fewer than 35 amino acids derived either from proteins or from chimeric sequences capable of transporting biomolecules across cell membrane in a receptor independent manner. CPPs can be cationic peptides, peptides having hydrophobic sequences, amphipathic peptides, peptides having proline-rich and anti-microbial sequences, and chimeric or bipartite peptides. Examples of CPPs include, e.g., Tat (which is a nuclear transcriptional activator protein required for viral replication by HIV type 1), penetratin, Kaposi fibroblast growth factor (FGF) signal peptide sequence, integrin β3 signal peptide sequence, polyarginine peptide Args sequence, Guanine rich-molecular transporters, and sweet arrow peptide. CPPs and methods of using them are described, e.g., in Hällbrink et al., “Prediction of cell-penetrating peptides,” Methods Mol. Biol., 2015; 1324:39-58; Ramakrishna et al., “Gene disruption by cell-penetrating peptide-mediated delivery of Cas9 protein and guide RNA,” Genome Res., 2014 June; 24(6):1020-7; and WO 2016205764 A1; each of which is incorporated herein by reference in its entirety.

Delivery of the Type V-G CRISPR system as a ribonucleoprotein complex by electroporation or nucleofection, in which purified Cas12g protein is pre-incubated with a RNA guide and electroporated (or nucleofected) into cells of interest, is another method of efficiently introducing the CRISPR system to cells for gene editing. This is particularly useful for ex vivo genome editing and the development of cellular therapies, and such methods are described in Roth et al. “Reprogramming human T cell function and specificity with non-viral genome targeting,” Nature. 2018 July; 559(7714): 405-409.

Various delivery methods for the CRISPR systems described herein are also described, e.g., in U.S. Pat. No. 8,795,965, EP 3009511, WO 2016205764, and WO 2017070605; each of which is incorporated herein by reference in its entirety.

### Kits

This disclosure also encompasses kits for carrying out the various methods of the disclosure utilizing the CRISPR systems described herein. One exemplary kit of the present disclosure comprises (a) one or more nucleic acids encoding a CRISPR-associated protein and a cognate crRNA, and/or (b) a ribonucleoprotein complex of a CRISPR-associated protein and a cognate crRNA. In some embodiments, the kit comprises a Cas12g protein and a Cas12g RNA guide. As described above, a complex of the protein and RNA guide has an editing activity such as SSB formation, RNA cleavage, CRISPR interference, nucleobase modification, DNA methylation or demethylation, chromatin modification, etc. In certain embodiments, the CRISPR-associated protein is a variant, such as a variant having reduced endonuclease activity.

Kits of this disclosure also optionally include additional reagents, including one or more of a reaction buffer, a wash buffer, one or more control materials (e.g., a substrate or a nucleic acid encoding a CRISPR system component), etc. A kit of the present disclosure also optionally includes instructions for performing a method of this disclosure using materials provided in the kit. The instructions are provided in physical form, e.g., as a printed document physically packaged with another item of the kit, and/or in digital form, e.g., a digitally published document downloadable from a website or provided on computer readable media.

### EXAMPLES

The invention is further described in the following examples, which do not limit the scope of the invention described in the claims.

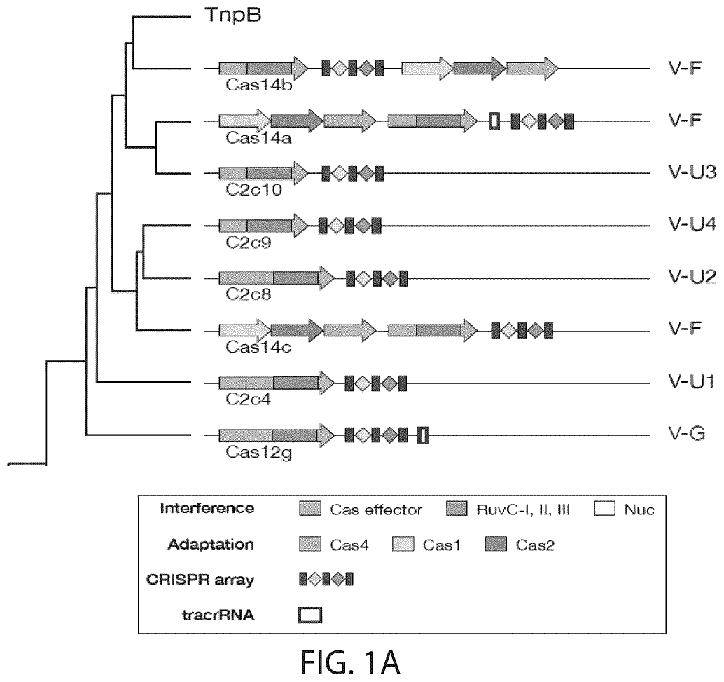

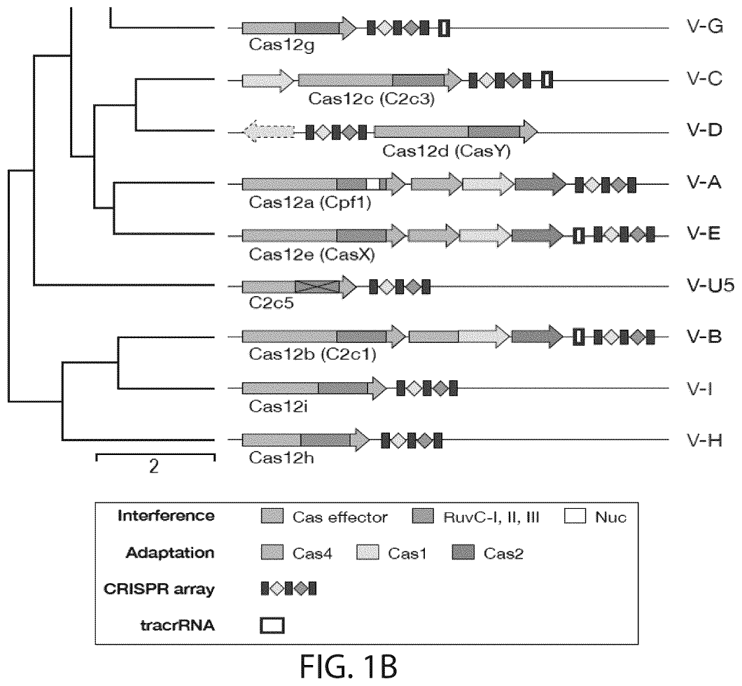

### Example 1: Identification of Minimal Components for CLUST.019143 (Type V-G CRISPR-Cas Effector Protein) System (FIGS. 1-3)

To achieve the broadest possible starting set of type V families, we explored additional members of the type V diversity within an expanded database of microbial genomic and metagenomic sequences. Genome and metagenome sequences were downloaded from NCBI (Benson et al., 2013; Pruitt et al., 2012), NCBI whole genome sequencing (WGS), and DOE JGI Integrated Microbial Genomes (Markowitz et al., 2012) and compiled to construct a database of 293,985 putative CRISPR-Cas systems, each including a predicted effector and a CRISPR array, and where present, adaptation modules, accessory proteins, and tracrRNA (Yan et al., 2018).

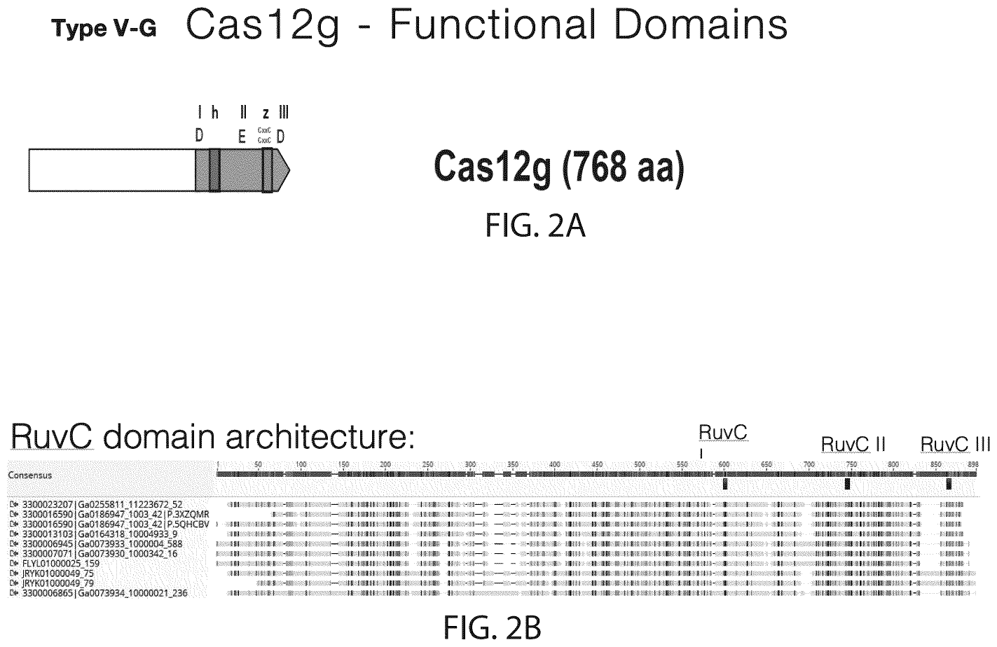

A search of this database for CRISPR-Cas systems in which the effector protein contained a domain homologous to RuvC (TnpB) revealed a diversity of novel type V CRISPR-Cas families beyond the previously described subtypes V-A-E and V-U. The loci encoding these effectors are highly enriched for CRISPR arrays of different lengths, but the adaptation module consisting of Cas1, Cas2 and Cas4 is associated only with Cas12a, Cas12b and Cas12e, whereas subtype V-C and V-D loci, in accord with the previous observations, contain only the Cas1 gene (FIG. 1) (Burstein et al., 2017; Shmakov et al., 2015). The predicted effectors in the newly identified families ranged in size from 720aa to 1093aa, each including a C-terminal TnpB-derived RuvC domain with conserved catalytic amino acids typical of the RuvC-like nucleases, and N-terminal extensions that were variable in length and sequence (FIG. 2A-B).

The classification tree depicted in FIG. 1 was constructed by comparing sequence profiles extracted from multiple alignments of groups of readily alignable Cas12 proteins. Profile-profile comparisons were performed using HHsearch (Söding et al., 2005); scores between two profiles were normalized by the minimum of the self-scores and converted to a distance matrix on the natural log scale. The UPGMA dendrogram was reconstructed from the distance matrix. The tree at the depth of 2 distance unites (corresponding to the pairwise HHsearch score of e 2D=0.02 relative to the self-score) typically reliably recovers profile similarity and can serve as a guide for subtype classification (Shmakov et al., 2017).

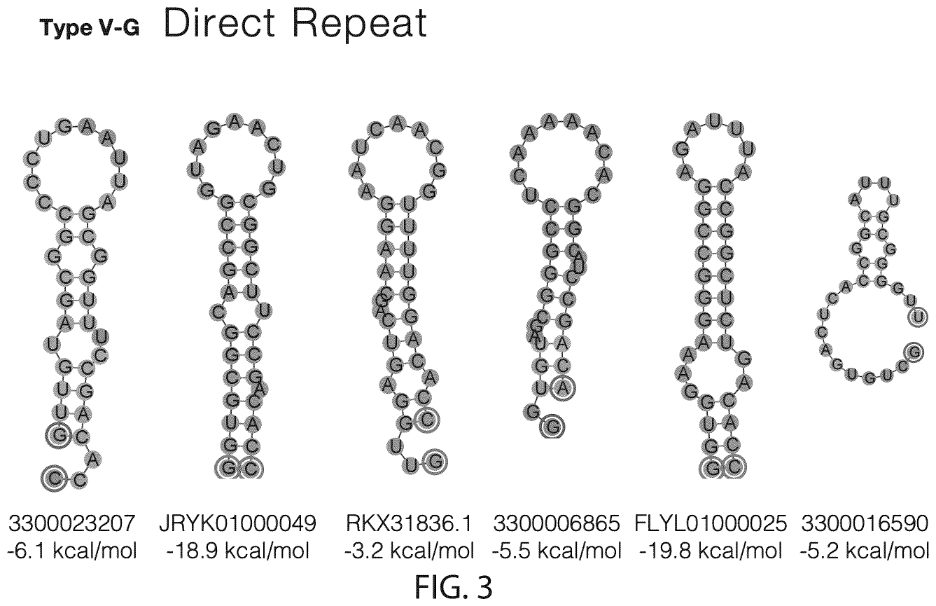

The domain architecture of Cas12g, depicted in FIGS. 2A-2B indicate that the effector contains the active catalytic residues of the RuvC nuclease domain. The direct repeat sequence for CLUST.019143 (Type V-G) shows a consensus 5′-GACACC-3′ nucleotide sequence proximal to the 3′ end of the direct repeat sequence. The predicted secondary structure of direct repeat sequences of example CLUST.019143 (Type V-G) CRISPR-Cas system is depicted in FIG. 3.

### Pipeline for Class 2 CRISPR-Cas Loci Identification

Genome and metagenome sequences were downloaded from NCBI (Benson et al., 2013; Pruitt et al., 2012), NCBI whole genome sequencing (WGS), and DOE JGI Integrated Microbial Genomes (Markowitz et al., 2012) and compiled to construct a database of 293,985 putative CRISPR-Cas loci within which we searched for novel RuvC containing type V effectors. As previously described in detail (Yan et al., 2018), this approach to pipeline engineering performs minimal filtering in the intermediate stages in order to expand the search space for novel CRISPR effector discovery and reduce biases.

### Construction of the Classification Tree of Class 2 Effectors

The classification tree was constructed by comparing sequence profiles extracted from multiple alignments of groups of readily alignable Cas12 proteins Profile-profile comparisons were performed using HHsearch (Söding, 2005); scores between two profiles were normalized by the minimum of the self-scores and converted to a distance matrix on the natural log scale. The UPGMA dendrogram was reconstructed from the distance matrix. The tree at the depth of 2 distance unites (corresponding to the pairwise HHsearch score of e^{-2D}=0.02 relative to the self-score) typically reliably recovers profile similarity and can serve as a guide for subtype classification (Shmakov et al., 2017).

TABLE 3
Representative CLUST.019143 (Type V-G) Effector Proteins

| | | # | cas | cas | effector |
|---|---|---|---|---|---|
| Source | Effector accession | spacers | 1 | 2 | size |
| Planctomycetes | RIK66295.1 | 5 | N | N | 761 |
| bacterium | | | | | |
| (QEVG01000016) | | | | | |
| activated sludge | JRYK01000049_75\|P | 13 | N | N | 821 |
| metagenome | | | | | |
| (JRYK01000049) | | | | | |
| activated sludge | JRYK01000049_79\|M | 13 | N | N | 786 |
| metagenome | | | | | |
| (JRYK01000049) | | | | | |
| anaerobic digester | UOQF01000587_2\|M | 10 | N | N | 772 |
| metagenome | | | | | |
| (UOQF01000587) | | | | | |
| anaerobic digester | UOQK01013695_2\|M | 3 | N | N | 772 |
| metagenome | | | | | |
| (UOQK01013695) | | | | | |
| aquatic-marine-marine | 3300028920\|Ga0272441_10052 | 4 | N | N | 696 |
| sediment | 198_2\|M | | | | |
| (3300028920\|Ga0272441_1 | | | | | |
| 0052198) | | | | | |
| aquatic-marine-marine | 3300028920\|Ga0272441_10052 | 4 | N | N | 768 |
| sediment | 198_4\|P | | | | |
| (3300028920\|Ga0272441_1 | | | | | |
| 0052198) | | | | | |
| aquatic-sediment-hot | 3300016590\|Ga0186947_1003_ | 2 | N | N | 774 |
| spring sediment | 42\|P | | | | |
| (3300016590\|Ga0186947_1 | | | | | |
| 003) | | | | | |
| aquatic-sediment-hot | 3300016590\|Ga0186947_1003_ | 2 | N | N | 720 |
| spring sediment | 42\|M | | | | |
| (3300016590\|Ga0186947_1 | | | | | |
| 003) | | | | | |
| aquatic-thermal | 3300010938\|Ga0137716_10003 | 14 | N | N | 855 |
| springs-hot spring | 017_35\|M | | | | |
| fesi sediment | | | | | |
| (3300010938\|Ga0137716_1 | | | | | |
| 0003017) | | | | | |
| aquatic-thermal | 3300010938\|Ga0137716_10003 | 14 | N | N | 803 |
| springs-hot spring | 017_32\|P | | | | |
| fesi sediment | | | | | |
| (3300010938\|Ga0137716_1 | | | | | |
| 0003017) | | | | | |
| aquatic-thermal | 3300006865\|Ga0073934_10000 | 2 | N | N | 830 |
| springs-hot spring | 021_236\|M | | | | |
| sediment | | | | | |
| (3300006865\|Ga0073934_1 | | | | | |
| 0000021) | | | | | |
| aquatic-thermal | 3300006945\|Ga0073933_10000 | 12 | N | N | 767 |
| springs-hot spring | 04_588\|P | | | | |
| sediment | | | | | |
| (3300006945\|Ga0073933_1 | | | | | |
| 000004) | | | | | |
| aquatic-thermal | 3300007071\|Ga0073930_10003 | 12 | N | N | 767 |
| springs-hot spring | 42_16\|M | | | | |
| sediment | | | | | |
| (3300007071\|Ga0073930_1 | | | | | |
| 000342) | | | | | |
| aquatic-thermal | 3300022554\|Ga0212093_10000 | 12 | N | N | 767 |
| springs-hot spring | 11_56\|M | | | | |
| sediment | | | | | |
| (3300022554\|Ga0212093_1 | | | | | |
| 000011) | | | | | |
| aquatic-thermal | 3300025094\|Ga0209478_10000 | 12 | N | N | 767 |
| springs-hot spring | 05_814\|P | | | | |
| sediment | | | | | |
| (3300025094\|Ga0209478_1 | | | | | |
| 000005) | | | | | |
| aquatic-thermal | 3300025105\|Ga0209479_10000 | 12 | N | N | 767 |
| springs-hot spring | 06_88\|P | | | | |
| sediment | | | | | |
| (3300025105\|Ga0209479_1 | | | | | |
| 000006) | | | | | |
| aquatic-thermal | 3300025310\|Ga0209172_10000 | 2 | N | N | 830 |
| springs-hot spring | 050_165\|P | | | | |
| sediment | | | | | |
| (3300025310\|Ga0209172_1 | | | | | |
| 0000050) | | | | | |
| aquatic-thermal | 3300019846\|Ga0197078_1002_ | 4 | N | N | 853 |
| springs-sediment | 15\|M | | | | |
| (3300019846\|Ga0197078_1 | | | | | |
| 002) | | | | | |
| bioreactor-anaerobic- | 3300023207\|Ga0255811_11223 | 4 | N | N | 772 |
| anaerobic digester | 672_52\|P | | | | |
| digestate | | | | | |
| (3300023207\|Ga0255811_1 | | | | | |
| 1223672) | | | | | |
| candidate division | RKX31836.1 | 11 | N | N | 769 |
| Zixibacteria bacterium | | | | | |
| Bacteria. | | | | | |
| (QNAH01000034) | | | | | |
| hot springs metagenome | FLYL01000025_159\|P | 13 | N | N | 767 |
| (FLYL01000025) | | | | | |
| sediment metagenome | ORFD01000444_4\|P | 12 | N | N | 767 |
| (ORFD01000444) | | | | | |
| sediment metagenome | ORXB01000005_848\|P | 12 | N | N | 767 |
| (ORXB01000005) | | | | | |
| terrestrial-deep | 3300027640\|Ga0209347_10037 | 10 | N | N | 774 |
| subsurface | 58_14\|P | | | | |
| (3300027640\|Ga0209347_1 | | | | | |
| 003758) | | | | | |
| terrestrial-deep | 3300027640\|Ga0209347_10037 | 10 | N | N | 763 |
| subsurface | 58_16\|M | | | | |
| (3300027640\|Ga0209347_1 | | | | | |
| 003758) | | | | | |
| terrestrial-soil | 3300027657\|Ga0256865_10030 | 3 | N | N | 752 |
| (3300027657\|Ga0256865_1 | 50_2\|M | | | | |
| 003050) | | | | | |

TABLE 4
Amino Acid Sequences of Representative
CLUST.019143 (Type V-G) Effector Proteins

>RIK66295.1
[Planctomycetes bacterium]
MGLRKRYVELCDNSGLRADLSSFGTLGNFLLEPSLDGSEADERERDRWRLAVFDIVAGFRADIQLDGRTLPNELRQ
AITDAADQMRNTIADGKKKTGLRRLFQRLGDLEEAHRLVLLKSAAEWIVARYLRSVENWVRQHTEWEKEKQQWESH
KDNKTDHTLLTPDIREKYTAVFKLLVWDKDKPPGVRNKRPRICPYDRLKQNIDNCCYAGQKGHGPLCWKYNEFVKA
RKARNPSFNDKKFAEDVAKYLPPRAKGEQRHLILKAIFPNKQQDQQRFSDNWTAYLSAMKLNEQTLVQHQQLPHCL
KIGETFEKSACVWNPHTDLCGQYHRALVNPNNGFDDAMLSLEPLYRAWRRNYLAGPRKPSFRYPASRDLPMPKIFG
DGFHEVDFENSIVRLRLDDMPRGQWLDLGFTPWPRGYKPSRAEISEPGRVTSVHVHFVGVRARIGFRFEVAHAPSR
FGCTQDEIDELRSRHYPRQAQDQEFLEAARKCLLDSMTEGSENDLRIMAVDLGEKGACAAVYRGRTREMDIPLAIV
KINKLYEKPPKTLEPDRHSRPEESKRKFEEADPRGVRKEHVGRHLERVAAMSQEIAKHRQPAVATTVTVSVNDLRG
LKRHVAWMIRDWARHNASQIVKAAEEHQCDVIVFESLRGFRPPGYDKLDEMSAKKKRWLAMFAYGRVRRKVIEKAV
ERGMRVVTVPYFKSSQVCSDCGREQVNVGLLRKNKLSKGQFVCENCKVTLTSDANAARVLARVFRGEIMLPKARPG
A (SEQ ID NO: 15)
>JRYK01000049_75|P
[activated sludge metagenome]
MLPTRYKPARTLVRPLGRLPHEPRKEFVEKCRRVRMHFEQFNIDVADLCQWLMSLRPNTRIGDAQSTVFWDFFLNP
SILTVEADEKERDRWRLAAFDELLQIRFGHDPNAPPWSEEFRSAIRHVAQRPKSATAQRLFDRLRSLTAPHRLVLL
KSAAEWIIARYQRGMENWQRQFAEWQREKEEWEAAHPNLTPEVRDAFTRVFKNLFENPDGDGKIGVRRKNPRICSW
ERLKLNKDNCVYAGQKGHGPLCWEFSKFVKAQKNAGTIKTFFVDVANKYLHVRRNLSKPGVKLKKSPRQEAFKRLY
NQKGMEKARNWFTDAWSGYLTALNLNEKTILDHGCLKHCGAIGAEFEKSLCQFNPHTHLCVQYRNALESLEPAIRE
LEGDYREWRRLFLAPPRKPSFRYPSSRRLPMPKIFGEHFHQIDFDQSILRLRLEDMAEGEWIEFGFKPWPKDYRPG
KDEVRVTSVHVNFHGNRMRAGFHFEAPAKPSRFACTQDELDDLRSKQFPRQSQDRQLLEVARRRLLESFDGMLESD
LRILAVDLGEKGAAAAVYQGHGHEADVAIPIVKIDRLYDHVPDVLDVESARVPPPKFDDSRDPRGVRKEHVGRHLG
QLQRGAQTLAQHRQQDESAPAALRRHDFRSLTRHIRWMIRDWTRHNAAQITAAAETHRCHLIVFESLRGFKPRGYD
QMDFAQKARLAFFAYGRVRRKVVEKAVERGLRVVTVPYGFTSQICSECGHRQRNKGRLRKNKYQRRFVCECGEPKK
SANKTAAPDRSATVSPCTCRLQLGSDVNAARVLARVFWDEIVLPTREEMREPAVDSAPPSK (SEQ ID NO: 2)
>JRYK01000049_79|M
[activated sludge metagenome]
MHFEQFNIDVADLCQWLMSLRPNTRIGDAQSTVFWDFFLNPSILTVEADEKERDRWRLAAFDELLQIRFGHDPNAP
PWSEEFRSAIRHVAQRPKSATAQRLFDRLRSLTAPHRLVLLKSAAEWIIARYQRGMENWQRQFAEWQREKEEWEAA
HPNLTPEVRDAFTRVFKNLFENPDGDGKIGVRRKNPRICSWERLKLNKDNCVYAGQKGHGPLCWEFSKFVKAQKNA
GTIKTFFVDVANKYLHVRRNLSKPGVKLKKSPRQEAFKRLYNQKGMEKARNWFTDAWSGYLTALNLNEKTILDHGC
LKHCGAIGAEFEKSLCQFNPHTHLCVQYRNALESLEPAIRELEGDYREWRRLFLAPPRKPSFRYPSSRRLPMPKIF
GEHFHQIDFDQSILRLRLEDMAEGEWIEFGFKPWPKDYRPGKDEVRVTSVHVNFHGNRMRAGFHFEAPAKPSRFAC
TQDELDDLRSKQFPRQSQDRQLLEVARRRLLESFDGMLESDLRILAVDLGEKGAAAAVYQGHGHEADVAIPIVKID
RLYDHVPDVLDVESARVPPPKFDDSRDPRGVRKEHVGRHLGQLQRGAQTLAQHRQQDESAPAALRRHDFRSLTRHI
RWMIRDWTRHNAAQITAAAETHRCHLIVFESLRGFKPRGYDQMDFAQKARLAFFAYGRVRRKVVEKAVERGLRVVT
VPYGFTSQICSECGHRQRNKGRLRKNKYQRRFVCECGEPKKSANKTAAPDRSATVSPCTCRLQLGSDVNAARVLAR
VFWDEIVLPTREEMREPAVDSAPPSK (SEQ ID NO: 3)
>UOQF01000587_2|M
[anaerobic digester metagenome]
MTRTKYREERTLVRGLQRLPNQDKTEFNKNVLRLRRHFEQFNLNVAELCQWLISFKPENPNSVCETKLFWEFMLEP
ENFCPDTYDRGSDWLRHEIFLLVAGWQTFEDMKAYNIRESLLTSAKMASSRPRSKTAEVLFIRLKSLEASHVMVLL
KSASEWLSTRYIRQSENWKRHVQEWQKEKAAWEDKHPKLTPAIRDKYNAIFVELGISEKRSRVCSWKKLSENKDNC
DWAGERKNIGDKWVSHSALCSKFNEFHRSLKGSPRKYFVTNANQYLELRRKNPQWTRDMAMQGLFRNVPGAQNWFP
NAWTNYLNALNILETTILKDYNGHLPHCNKLSDECLFNQHTFNCRQYKRLLVGKLSSQERDLEETYREWRREYLAP
PDKPFLRYPSAQKLPTPKLFGRGYYDLDFTRHVVKLRLDDMPAENFVSFGFKPWPRDYDKKPGEINITSVHVHFIG
TRARVGFRFAVPHSYSRFSVSQDKIDELRSRVFPRKSQDLEFLNEARLRLLDGMNENQKSALRIMAVDLGTHRAAA
AFFTGRIFDKPKLIKLKKIDLLTESITDTTQPEKHSANEKKIQREKGLTQHHVGKHLDTLETRTKEIISKRQNIKI
TPSDDTLGTLGDHDLRHLTSHIQRMIRDWVRLNAQQITELAEKEKVDLIVFESLRGFRAHAYDKLDLEQKRRLAFF
AYGRIRRKVAEKAVERGMRVLTVPYFESSQICAQCGRSQNDKNKLRDNKRKQSFQCEFSDCNYKTHSDENAARVLG
RVFWGEITLPTD (SEQ ID NO: 16)
>UOQK01013695_2|M
[anaerobic digester metagenome]
MTRTKYREERTLVRGLQRLPNQDKTEFNKNVLRLRRHFEQFNLNVAELCQWLISFKPENPNSVCETKLFWEFMLEP
ENFCPDTYDRGSDWLRHEIFLLVAGWQTFEDMKAYNIRESLLTSAKMASSRPRSKTAEVLFIRLKSLEASHVMVLL
KSASEWLSTRYIRQSENWKRHVQEWQKEKAAWEDKHPKLTPAIRDKYNAIFVELGISEKRSRVCSWKKLSENKDNC
DWAGERKNIGDKWVSHSALCSKFNEFHRSLKGSPRKYFVTNANQYLELRRKNPQWTRDMAMQGLFRNVPGAQNWFP
NAWTNYLNALNILETTILKDYNGHLPHCNKLSDECLFNQHTFNCRQYKRLLVGKLSSQERDLEETYREWRREYLAP
PDKPFLRYPSAQKLPTPKLFGRGYYDLDFTRHVVKLRLDDMPAENFVSFGFKPWPRDYDKKPGEINITSVHVHFIG
TRARVGFRFAVPHSYSRFSVSQDKIDELRSRVFPRKSQDLEFLNEARLRLLDGMNENQKSALRIMAVDLGTHRAAA
AFFTGRIFDKPKLIKLKKIDLLTESITDTTQPEKHSANEKKIQREKGLTQHHVGKHLDTLETRTKEIISKRQNIKI
TPSDDTLGTLGDHDLRHLTSHIQRMIRDWVRLNAQQITELAEKEKVDLIVFESLRGFRAHAYDKLDLEQKRRLAFF
AYGRIRRKVAEKAVERGMRVLTVPYFESSQICAQCGRSQNDKNKLRDNKRKQSFQCEFSDCNYKTHSDENAARVLG
RVFWGEITLPTD (SEQ ID NO: 16)
>3300028920|Ga0272441_10052198_2|M
[aquatic-marine-marine sediment]
MLEPERFCARNDPGDPRGDWIRHAVFEVVAGWKLSDNLDQYNLNEELTASIEAAMDKTRTATAEALFERLMRREAS
NVMVLLKVAAEWIAAKYVHQMENWKRQKEEWEKEKAEWENSHTELTEQVRDKYNRIFKELDIKNKRPRVCTWKRLS
ENKDNCDWAGKRKLIGKSWVNHAALCYKYHEYSEAPKVKHRDHFIANANKYIKIRREYPQWSRDQAMKTLFKNEPR
ASYWFPKEWKMYLGALGIEENTIIGNYTGCLPHCLKITHKCRFNKHTNECRRYKDLMHERLTNEERQLEELYREWR
RNYLIAPGKPALRYPSARTLPTPKIFGSGYYRLDFERNQVHLRLDDMSQGDFISFGIKAWPRKYDYQPDTIDITSV
QVHFVGTRARIGFRFKVPHRESIFTIRQDDIDELRSRKYPRESQDQKFLEEVRKRILNGFSEDQIAKLKIMAVDLG
SDEGGVAFFKGHVFEKGESLKIIKIDELFESKKNEEAEKAKGLNVHHVGRHLDVLQKKSQEIALLRQGMTNAPSND
MVQSLYPNDMRRLTSHIRRMIRDWVRLNSSQIIKLAEREQVELIVFESMRGFLAPGYDKIDPDKKRRLAFFAFGS1
RRKVAEKAVERGMRVVTVPYHCSSQVCAKCGKEQEDKKRFRKNKEKREFVCEDKKCNHKTNSDINAAHVCGRVFWG
EINLLGKKIKIK (SEQ ID NO: 17)
>3300028920|Ga0272441_10052198_4|P
[aquatic-marine-marine sediment]
MTRTKYREERTLVRSINRLPKQDKAEFKKNVLRLRRYFEQYNLDVSETCQWLISYRGLNLDEICKTQLFWEFMLEP
ERFCARNDPGDPRGDWIRHAVFEVVAGWKLSDNLDQYNLNEELTASIEAAMDKTRTATAEALFERLMRREASNVMV
LLKVAAEWIAAKYVHQMENWKRQKEEWEKEKAEWENSHTELTEQVRDKYNRIFKELDIKNKRPRVCTWKRLSENKD
NCDWAGKRKLIGKSWVNHAALCYKYHEYSEAPKVKHRDHFIANANKYIKIRREYPQWSRDQAMKTLFKNEPRASYW
FPKEWKMYLGALGIEENTIIGNYTGCLPHCLKITHKCRFNKHTNECRRYKDLMHERLTNEERQLEELYREWRRNYL
IAPGKPALRYPSARTLPTPKIFGSGYYRLDFERNQVHLRLDDMSQGDFISFGIKAWPRKYDYQPDTIDITSVQVHF
VGTRARIGFRFKVPHRESIFTIRQDDIDELRSRKYPRESQDQKFLEEVRKRILNGFSEDQIAKLKIMAVDLGSDEG
GVAFFKGHVFEKGESLKIIKIDELFESKKNEEAEKAKGLNVHHVGRHLDVLQKKSQEIALLRQGMTNAPSNDMVQS
LYPNDMRRLTSHIRRMIRDWVRLNSSQIIKLAEREQVELIVFESMRGFLAPGYDKIDPDKKRRLAFFAFGSIRRKV
AEKAVERGMRVVTVPYHCSSQVCAKCGKEQEDKKRFRKNKEKREFVCEDKKCNHKTNSDINAAHVCGRVFWGEINL
LGKKIKIK (SEQ ID NO: 18)
>3300016590|Ga0186947_1003_42|P
[aquatic-sediment-hot spring sediment]
MDNNRTRYREERTLVRSLRPLDGENRNAFKNRVNRLRIHFKNFNLDVSEICQWLMSIRPDAKKPDKETKSFWDFFL
NPESFFDPTINNVDIIRLNLFKVITGRESEANLIRYNLPLLLYESIILLKKQEPSDTARRLFARLKKMEPVHVMIL
LKAAAEWVYARYQRLMDNHEYQYKVWHDEKSAWENKHPELTPEIREKYNSIFKELGRKQGVTIRKNPRICNWEKLE
ENKDNCGYNGKRIQFGDKWKAHSMLCIEYRNFLRDNKITGKRIGFFATHAYNYLKLRAHQPRLTKDEAFKRIFKSA
PNGIYWFPKAWKNYLQFMNLNELNLIRKYNANLPHCLEFKGDKDCQYNKHTELCQEYKTLLLEKFTEDELKLEGLY
REWRKQYLSGPSKPAFRYPSCSKLPTPKIFGKRFHEIDFENSIVRLRLDDMPDGEYLTFKFKPWPNDYQPQPEEAE
ISSVHVHFVGTRARVGFRFKIAHKQSRFKTSQDEIDELRSRKYPRQAQDADFLKAAREKLLQSFKGENPTKEIKIM
AVDLGEYRGYISVYKGENIEISEPLSILKIDKLYDSLESAGVDKTDLAKYIKDHKGLIKEHVDSHLKVISEKANEI
TKHRPAGKKTGASNLKDYDLRSLTAHTGWMIRDWVRLNVSQIIRIAEKHEVDLIVLESLRGWKAPGYDEFDLRKKR
WLAFFSYGRIRHKLKEKAVERGMMVVTVPYYKSSQICSKCGKEQENKGLWKKNKNERLFICDYPGCGHRDNSDANA
AKVLAKIFWGEIVL (SEQ ID NO: 8)
>3300016590|Ga0186947_1003_42|M
[aquatic-sediment-hot spring sediment]
MSIRPDAKKPDKETKSFWDFFLNPESFFDPTINNVDIIRLNLFKVITGRESEANLIRYNLPLLLYESIILLKKQEP
SDTARRLFARLKKMEPVHVMILLKAAAEWVYARYQRLMDNHEYQYKVWHDEKSAWENKHPELTPEIREKYNSIFKE
LGRKQGVTIRKNPRICNWEKLEENKDNCGYNGKRIQFGDKWKAHSMLCIEYRNFLRDNKITGKRIGFFATHAYNYL
KLRAHQPRLTKDEAFKRIFKSAPNGIYWFPKAWKNYLQFMNLNELNLIRKYNANLPHCLEFKGDKDCQYNKHTELC
QEYKTLLLEKFTEDELKLEGLYREWRKQYLSGPSKPAFRYPSCSKLPTPKIFGKRFHEIDFENSIVRLRLDDMPDG
EYLTFKFKPWPNDYQPQPEEAEISSVHVHFVGTRARVGFRFKIAHKQSRFKTSQDEIDELRSRKYPRQAQDADFLK
AAREKLLQSFKGENPTKEIKIMAVDLGEYRGYISVYKGENIEISEPLSILKIDKLYDSLESAGVDKTDLAKYIKDH
KGLIKEHVDSHLKVISEKANEITKHRPAGKKTGASNLKDYDLRSLTAHTGWMIRDWVRLNVSQIIRIAEKHEVDLI
VLESLRGWKAPGYDEFDLRKKRWLAFFSYGRIRHKLKEKAVERGMMVVTVPYYKSSQICSKCGKEQENKGLWKKNK
NERLFICDYPGCGHRDNSDANAAKVLAKIFWGEIVL (SEQ ID NO: 7)
>3300010938|Ga0137716_10003017_35|M
[aquatic-thermal springs-hot spring fe si sediment]
MVASRYREARTLVRRLGRLPGEGEASFRAKLARLRKHFERFNVDVSELCQWLMGLRKQHCHKAGTASFGVLGDFLL
HPESSNVAPGEAEADRWRLLVFDAVAGICPAKQLTNAPMPRGLPEAMEREAKRLADLEWRRRNTTQSKLIDRLAIL
EPAHRLVLLKAAAEWVVSRYQRGLENWAARRGEWEKERHAWEKRHPALSEEVRQRFTEVFKSLNDPERTDKPGVRR
KNPRICPYERLRANIDNCIYAGEKGHGALCWKYAEFVKARKTRQPQFNDKRFAEDAEKVLPLLKQGMKRHQALQRL
FPRDRPHGQLAQQRFNENWTAYLQALGLKEERVVNRGRLPHCLKIGETHEKSKCAWNPHTELCKQYKRALDQFDEE
TLKLEPLYREWRRDYLAGPGKPQFRYPSSRELPMPKIFGAGFHEIDFDRSILRLRLEDMPEGEWIEFGFAPWPRGY
RPSKEEVKVKGAISSVHVNFVGVRARAGFRFDVRHRASRFQCTQDELDQLRSRAYPRRAQDREYLDAARKRLLESF
AEGEEAAKRELRLLAVDLGETGACAAVYHGHAHQKDVQLAILKINRLYTQLPEALEPDPHGRPEEGKRKYERDDPR
GVRKEHMGRHLKRMADGAASIAARRQGTMPATVTMAGHDFRGLKRHVTWMIRDWARHNAARIVAAAEEHGCDLIVF
ESLRGQKVPGYHELSSEKERDKRQLAMLSYGRIRHKVREKAVERGMRVVMVPDYRSSRLCSSCGHEQCAEKWQERR
WRENKKKRLFKCVCGEPAPTEKPHHGGSAPDRQRAARRDGPGPGKRPGMAADQAKQRCRCGAEMNSDANAARVLAR
VFWGEITPPASERSFAGSA (SEQ ID NO: 19)
>3300010938|Ga0137716_10003017_32|P
[aquatic-thermal springs-hot spring fe si sediment]
MGLRKQHCHKAGTASFGVLGDFLLHPESSNVAPGEAEADRWRLLVFDAVAGICPAKQLTNAPMPRGLPEAMEREAK
RLADLEWRRRNTTQSKLIDRLAILEPAHRLVLLKAAAEWVVSRYQRGLENWAARRGEWEKERHAWEKRHPALSEEV
RQRFTEVFKSLNDPERTDKPGVRRKNPRICPYERLRANIDNCIYAGEKGHGALCWKYAEFVKARKTRQPQFNDKRF
AEDAEKVLPLLKQGMKRHQALQRLFPRDRPHGQLAQQRFNENWTAYLQALGLKEERVVNRGRLPHCLKIGETHEKS
KCAWNPHTELCKQYKRALDQFDEETLKLEPLYREWRRDYLAGPGKPQFRYPSSRELPMPKIFGAGFHEIDFDRSIL
RLRLEDMPEGEWIEFGFAPWPRGYRPSKEEVKVKGAISSVHVNFVGVRARAGFRFDVRHRASRFQCTQDELDQLRS
RAYPRRAQDREYLDAARKRLLESFAEGEEAAKRELRLLAVDLGETGACAAVYHGHAHQKDVQLAILKINRLYTQLP
EALEPDPHGRPEEGKRKYERDDPRGVRKEHMGRHLKRMADGAASIAARRQGTMPATVTMAGHDFRGLKRHVTWMIR
DWARHNAARIVAAAEEHGCDLIVFESLRGQKVPGYHELSSEKERDKRQLAMLSYGRIRHKVREKAVERGMRVVMVP
DYRSSRLCSSCGHEQCAEKWQERRWRENKKKRLFKCVCGEPAPTEKPHHGGSAPDRQRAARRDGPGPGKRPGMAAD
QAKQRCRCGAEMNSDANAARVLARVFWGEITPPASERSFAGSA (SEQ ID NO: 20)
>3300006865|Ga0073934_10000021_236|M
[aquatic-thermal springs-hot spring sediment]
MHPSRYKTARTLVRRLCRLPGEDRSAFRSKVGLLRGHFEQFNVDVSELCQWLMSLRKRNKVPENPATFGALGDFLL
QPGLPGEETDEKEADRLRLAVFDAVAGFRMLEDRLAASIPASLSDAIRDEAVFLAGVRAAGKPSGLARVLARLEAC
APAQRLVLLKSAAEWIVARFLRGTENWMRQRAEWEKEKAAWEAAHPHLTPEVRAQFNKIFESLHDPENSGKPGVSR
KNPRICPWDRLKQNLDNCCYGEKGHSALCWRYQDFLKQRMGENRRDKKNFSATAMDLAQICREWKIQHSRNALNNP
RVLDRLFAEHERRKQDKTKKESRSPKPRQGGYKANPKADYLRSFKAHWKAYLEHMKLNDTTVLERGCLPHCLSIKK
NGKESTCKWNKHTELCLEYKRSLAPLPDSVLELEPEYREWRRLYLHGPGRPHFRYPSAGELPLPKVFGEGFHQVDL
DRSIVRLRLEGAAEGEWLEFGFIPWPRGYQPSRREVLITSVQVHFVGTRPRAGFRFDVSHRTSRFGCSQDELDELR
SRRYPRQAQDKEFLAAARAQLIQTFEGGEGAARQQMRVMSVDLGEGGACASIYEGRTHQKDESLKVIKIDRRYDQH
PEVLEKDVGAAKPQKFEKSDPRGVRKEHVARHLNRIAAGASAIAEHRRKERSDAECSVGELQEHDFRSLKRHIAWM
IRDWVRLNAAQIIDVAKQHCCDLIVFESQRGFRLPGYDELDRGKKQRFAILAFGRIRRKVVEKAVEHGMRVVTVPY
FASSQVCSACKRVQENRGSWRENKKKRVFACEFCKLKLNSDANASRVLARVFWGEIELPEPTRAHLPSKA (SEQ
ID NO: 5)
>3300006945|Ga0073933_1000004_588|P
[aquatic-thermal springs-hot spring sediment]
MAQASSTPAVSPRPRPRYREERTLVRKLLPRPGQSKQEFRENVKKLRKAFLQFNADVSGVCQWAIQFRPRYGKPAE
PTETFWKFFLEPETSLPPNDSRSPEFRRLQAFEAAAGINGAAALDDPAFTNELRDSILAVASRPKTKEAQRLFSRL
KDYQPAHRMILAKVAAEWIESRYRRAHQNWERNYEEWKKEKQEWEQNHPELTPEIREAFNQIFQQLEVKEKRVRIC
PAARLLQNKDNCQYAGKNKHSVLCNQFNEFKKNHLQGKAIKFFYKDAEKYLRCGLQSLKPNVQGPFREDWNKYLRY
MNLKEETLRGKNGGRLPHCKNLGQECEFNPHTALCKQYQQQLSSRPDLVQHDELYRKWRREYWREPRKPVFRYPSV
KRHSIAKIFGENYFQADFKNSVVGLRLDSMPAGQYLEFAFAPWPRNYRPQPGETEISSVHLHFVGTRPRIGFRFRV
PHKRSRFDCTQEELDELRSRTFPRKAQDQKFLEAARKRLLETFPGNAEQELRLLAVDLGTDSARAAFFIGKTFQQA
FPLKIVKIEKLYEQWPNQKQAGDRRDASSKQPRPGLSRDHVGRHLQKMRAQASEIAQKRQELTGTPAPETTTDQAA
KKATLQPFDLRGLTVHTARMIRDWARLNARQIIQLAEENQVDLIVLESLRGFRPPGYENLDQEKKRRVAFFAHGRI
RRKVTEKAVERGMRVVTVPYLASSKVCAECRKKQKDNKQWEKNKKRGLFKCEGCGSQAQVDENAARVLGRVFWGEI
ELPTAIP (SEQ ID NO: 6)
>3300007071|Ga0073930_1000342_16|M
[aquatic-thermal springs-hot spring sediment]
MAQASSTPAVSPRPRPRYREERTLVRKLLPRPGQSKQEFRENVKKLRKAFLQFNADVSGVCQWAIQFRPRYGKPAE
PTETFWKFFLEPETSLPPNDSRSPEFRRLQAFEAAAGINGAAALDDPAFTNELRDSILAVASRPKTKEAQRLFSRL
KDYQPAHRMILAKVAAEWIESRYRRAHQNWERNYEEWKKEKQEWEQNHPELTPEIREAFNQIFQQLEVKEKRVRIC
PAARLLQNKDNCQYAGKNKHSVLCNQFNEFKKNHLQGKAIKFFYKDAEKYLRCGLQSLKPNVQGPFREDWNKYLRY
MNLKEETLRGKNGGRLPHCKNLGQECEFNPHTALCKQYQQQLSSRPDLVQHDELYRKWRREYWREPRKPVFRYPSV
KRHSIAKIFGENYFQADFKNSVVGLRLDSMPAGQYLEFAFAPWPRNYRPQPGETEISSVHLHFVGTRPRIGFRFRV
PHKRSRFDCTQEELDELRSRTFPRKAQDQKFLEAARKRLLETFPGNAEQELRLLAVDLGTDSARAAFFIGKTFQQA
FPLKIVKIEKLYEQWPNQKQAGDRRDASSKQPRPGLSRDHVGRHLQKMRAQASEIAQKRQELTGTPAPETTTDQAA
KKATLQPFDLRGLTVHTARMIRDWARLNARQIIQLAEENQVDLIVLESLRGFRPPGYENLDQEKKRRVAFFAHGRI
RRKVTEKAVERGMRVVTVPYLASSKVCAECRKKQKDNKQWEKNKKRGLFKCEGCGSQAQVDENAARVLGRVFWGEI
ELPTAIP (SEQ ID NO: 6)
>3300022554|Ga0212093_1000011_56|M
[aquatic-thermal springs-hot spring sediment]
MAQASSTPAVSPRPRPRYREERTLVRKLLPRPGQSKQEFRENVKKLRKAFLQFNADVSGVCQWAIQFRPRYGKPAE
PTETFWKFFLEPETSLPPNDSRSPEFRRLQAFEAAAGINGAAALDDPAFTNELRDSILAVASRPKTKEAQRLFSRL
KDYQPAHRMILAKVAAEWIESRYRRAHQNWERNYEEWKKEKQEWEQNHPELTPEIREAFNQIFQQLEVKEKRVRIC
PAARLLQNKDNCQYAGKNKHSVLCNQFNEFKKNHLQGKAIKFFYKDAEKYLRCGLQSLKPNVQGPFREDWNKYLRY
MNLKEETLRGKNGGRLPHCKNLGQECEFNPHTALCKQYQQQLSSRPDLVQHDELYRKWRREYWREPRKPVFRYPSV
KRHSIAKIFGENYFQADFKNSVVGLRLDSMPAGQYLEFAFAPWPRNYRPQPGETEISSVHLHFVGTRPRIGFRFRV
PHKRSRFDCTQEELDELRSRTFPRKAQDQKFLEAARKRLLETFPGNAEQELRLLAVDLGTDSARAAFFIGKTFQQA
FPLKIVKIEKLYEQWPNQKQAGDRRDASSKQPRPGLSRDHVGRHLQKMRAQASEIAQKRQELTGTPAPETTTDQAA
KKATLQPFDLRGLTVHTARMIRDWARLNARQIIQLAEENQVDLIVLESLRGFRPPGYENLDQEKKRRVAFFAHGRI
RRKVTEKAVERGMRVVTVPYLASSKVCAECRKKQKDNKQWEKNKKRGLFKCEGCGSQAQVDENAARVLGRVFWGEI
ELPTAIP (SEQ ID NO: 6)
>3300025094|Ga0209478_1000005_814|P
[aquatic-thermal springs-hot spring sediment]
MAQASSTPAVSPRPRPRYREERTLVRKLLPRPGQSKQEFRENVKKLRKAFLQFNADVSGVCQWAIQFRPRYGKPAE
PTETFWKFFLEPETSLPPNDSRSPEFRRLQAFEAAAGINGAAALDDPAFTNELRDSILAVASRPKTKEAQRLFSRL
KDYQPAHRMILAKVAAEWIESRYRRAHQNWERNYEEWKKEKQEWEQNHPELTPEIREAFNQIFQQLEVKEKRVRIC
PAARLLQNKDNCQYAGKNKHSVLCNQFNEFKKNHLQGKAIKFFYKDAEKYLRCGLQSLKPNVQGPFREDWNKYLRY
MNLKEETLRGKNGGRLPHCKNLGQECEFNPHTALCKQYQQQLSSRPDLVQHDELYRKWRREYWREPRKPVFRYPSV
KRHSIAKIFGENYFQADFKNSVVGLRLDSMPAGQYLEFAFAPWPRNYRPQPGETEISSVHLHFVGTRPRIGFRFRV
PHKRSRFDCTQEELDELRSRTFPRKAQDQKFLEAARKRLLETFPGNAEQELRLLAVDLGTDSARAAFFIGKTFQQA
FPLKIVKIEKLYEQWPNQKQAGDRRDASSKQPRPGLSRDHVGRHLQKMRAQASEIAQKRQELTGTPAPETTTDQAA
KKATLQPFDLRGLTVHTARMIRDWARLNARQIIQLAEENQVDLIVLESLRGFRPPGYENLDQEKKRRVAFFAHGRI
RRKVTEKAVERGMRVVTVPYLASSKVCAECRKKQKDNKQWEKNKKRGLFKCEGCGSQAQVDENAARVLGRVFWGEI
ELPTAIP (SEQ ID NO: 6)
>3300025105|Ga0209479_1000006_88|P
[aquatic-thermal springs-hot spring sediment]
MAQASSTPAVSPRPRPRYREERTLVRKLLPRPGQSKQEFRENVKKLRKAFLQFNADVSGVCQWAIQFRPRYGKPAE
PTETFWKFFLEPETSLPPNDSRSPEFRRLQAFEAAAGINGAAALDDPAFTNELRDSILAVASRPKTKEAQRLFSRL
KDYQPAHRMILAKVAAEWIESRYRRAHQNWERNYEEWKKEKQEWEQNHPELTPEIREAFNQIFQQLEVKEKRVRIC
PAARLLQNKDNCQYAGKNKHSVLCNQFNEFKKNHLQGKAIKFFYKDAEKYLRCGLQSLKPNVQGPFREDWNKYLRY
MNLKEETLRGKNGGRLPHCKNLGQECEFNPHTALCKQYQQQLSSRPDLVQHDELYRKWRREYWREPRKPVFRYPSV
KRHSIAKIFGENYFQADFKNSVVGLRLDSMPAGQYLEFAFAPWPRNYRPQPGETEISSVHLHFVGTRPRIGFRFRV
PHKRSRFDCTQEELDELRSRTFPRKAQDQKFLEAARKRLLETFPGNAEQELRLLAVDLGTDSARAAFFIGKTFQQA
FPLKIVKIEKLYEQWPNQKQAGDRRDASSKQPRPGLSRDHVGRHLQKMRAQASEIAQKRQELTGTPAPETTTDQAA
KKATLQPFDLRGLTVHTARMIRDWARLNARQIIQLAEENQVDLIVLESLRGFRPPGYENLDQEKKRRVAFFAHGRI
RRKVTEKAVERGMRVVTVPYLASSKVCAECRKKQKDNKQWEKNKKRGLFKCEGCGSQAQVDENAARVLGRVFWGEI
ELPTAIP (SEQ ID NO: 6)
>3300025310|Ga0209172_10000050_165|P
[aquatic-thermal springs-hot spring sediment]
MHPSRYKTARTLVRRLCRLPGEDRSAFRSKVGLLRGHFEQFNVDVSELCQWLMSLRKRNKVPENPATFGALGDFLL
QPGLPGEETDEKEADRLRLAVFDAVAGFRMLEDRLAASIPASLSDAIRDEAVFLAGVRAAGKPSGLARVLARLEAC
APAQRLVLLKSAAEWIVARFLRGTENWMRQRAEWEKEKAAWEAAHPHLTPEVRAQFNKIFESLHDPENSGKPGVSR
KNPRICPWDRLKQNLDNCCYGEKGHSALCWRYQDFLKQRMGENRRDKKNFSATAMDLAQICREWKIQHSRNALNNP
RVLDRLFAEHERRKQDKTKKESRSPKPRQGGYKANPKADYLRSFKAHWKAYLEHMKLNDTTVLERGCLPHCLSIKK
NGKESTCKWNKHTELCLEYKRSLAPLPDSVLELEPEYREWRRLYLHGPGRPHFRYPSAGELPLPKVFGEGFHQVDL
DRSIVRLRLEGAAEGEWLEFGFIPWPRGYQPSRREVLITSVQVHFVGTRPRAGFRFDVSHRTSRFGCSQDELDELR
SRRYPRQAQDKEFLAAARAQLIQTFEGGEGAARQQMRVMSVDLGEGGACASIYEGRTHQKDESLKVIKIDRRYDOH
PEVLEKDVGAAKPQKFEKSDPRGVRKEHVARHLNRIAAGASAIAEHRRKERSDAECSVGELQEHDFRSLKRHIAWM
IRDWVRLNAAQIIDVAKQHCCDLIVFESQRGFRLPGYDELDRGKKQRFAILAFGRIRRKVVEKAVEHGMRVVTVPY
FASSQVCSACKRVQENRGSWRENKKKRVFACEFCKLKLNSDANASRVLARVFWGEIELPEPTRAHLPSKA (SEQ
ID NO: 5)
>3300019846|Ga0197078_1002_15|M
[aquatic-thermal springs-sediment]
MKNPKYREERTLTMGIRSFPLEDKSHFKEKVRKLRKFFEIFNKDVADLCQWLIVFRKGGKSENIPIWEFFINPLES
IKDISEDQADELKRKVLDVIIGNESINNIRNEKLPTEVLKYLENLDDNWNQSVQELFKRMKDKKPSHRQILLKAAS
NLIYSRYYKTLENREKQKEEWKKERDTWQRKNPDLKEDIRTKYTEVYKQLGIKRKAPRICEWEKLKNWKHNCTYSS
NKQHTERCFDFNKLFSSADNKKKRKWFIDNAHLYLQYLSKSNQKDALKQLYHKKKGSEKWFGRLWNDYLKLMKISE
QDILTKYHCILPHCISENLGSAKYREYFDNSEYGFARSEFGKCANTYLGSRKKGSNKADSLQMVYGKHKSNIDERF
EEIWNDYLKFIKKTEEEAIIEYNHSLPVYLKSDYCQFRPHGDKCKQYRDLLADFTEVEKSLEKKYREWRKSYLSGP
GKPQFRYPSAKTIAIPKLFGSGYFRIDFEESILNLKLENDEWLSLGFKPWPKRKDYDIHYSDIEIPSVSIHFVGTR
AKVGFRFKVKHKESRFKVAQEEIDKLRSQKYPRQYQDNDFLKEARELLLKDFDGDKNEMKILAFDWGETGAYAALF
TGEKYEKGFQLPVLKFEKLYCSDKFKEAWDQKKKEQKWSKDESKAKLKEYKLKGLTKGHVGKHLENISEKAVKIAE
IRGEKKDEKLLRPSDLRRLFSHSAWMIRDWVRLNTKQLIKIAEKNEVDLIVFESMRGSAPPSYDKLEEITEKIKWA
FFSLGRIRHKVTEKAVERGMRTITVPYVKSSQVCFDCGKEAEDKKKWQHHKTELTKFICEHCPADLNSDENAARVL
CKVFWGDITLPSTEWEK (SEQ ID NO: 21)
>3300023207|Ga0255811_11223672_52|P
[bioreactor-anaerobic-anaerobic digester digestate]
MTRIKYRQERTLVRGLQRLPNQDKAEFNKNVLGLRRHFEQFNLNVAELCQWLISFKPENPGSVCETKLFWEFMLEP
ENFCPDTYDRGPDWLRHEIFVLAAGWRTFEDIKAHNMPESLFESIKIASSKPRSKTAEALFIRLKSLEASHVMVLL
KSASEWLSTRYVRQSENWKQNEQEWQKKKAAWEDKHPELTPAIRDKYNAIFVELGISEKRPHVCSWKKLSENKDNC
DWAGERKNIGDKWISHSDLCIKYHEFARKLRSKQRQHFVDNANQYLELRRRYPQWTRDMAMNGLFKNVPLARNWFR
NAWTNYLNALNILETTILENYSGHLPHCEKLSDECVFKKHTDNCRRYKLLLGEKLSNQERELEETYREWRREFLAP
PNKPFLRYPSAQKLPTPKLFGRGYYDLDFTRHVVKLRLDDMPADNFVSFGFKPWPRGYDKKPGEINITSVHVHFIG
TRARVGFRFAVPHSDSRFSVSQDKIDELRSGGFPGKSQDQEFLNEARQRLLDGMNENQKSALRIMAVDLGTHRAAA
AFFTGCIFNKAKLLKLKKIDLLTEPKTDTTKPEKLSADEKKIQREKGLTQHHVGKHFETLEARTKEIISKRQNMKM
APSDDTPDIVGDHDLRHLTSHIRRMIRDWVRLNARQITELAEEENVDLIVFESMRGFRAPGYDKLDLKKKRRLAFF
AYGQIRRKVAEKAVERGMRVITVPYFKSSQICAQCGRSQNDKNKLRDNKWKQSFQCEFSDCNYKTHSDENAARVLG
RVFWGEITLPTD (SEQ ID NO: 1)
>RKX31836.1
[candidate division Zixibacteria bacterium Bacteria.]
MNRTRYREERTITRGMRRLPGEERKSFKAKVITLRRNFEQFNTDVSEICQWLMSIRPNGKHNIPNTEPFWDFILEP
HNFVVNQEETNIDSVRLVVFEMAVGWRQVTDVANFELERQLLMSLESIQSVPRTIAAKRMLQRIKNYEFQHKMVLL
RSAVEWINTRFIRTYKNWEMNIKEFLEKKKVWENDHPKLTEEIRNTFNKVFDELEISKKNPNICRWSHLKKNRDNC
NYAGVRIKVGGEYNNHSEKCKRYQDFLKKHSAHKKYFAANAMMYINIRKKRRDLTKREAIKVLLDKIPQARSWFPQ
AWDNYLEYLGLNEISLINKFDGQLPHCLRLDTECIYNVHTQSCRKYYVLLKDLPDKYLSLEETYREWRKYFLREPR
KPVFAYPSTRQRTVSKIFGRDYFEADYDNSIIKLRLDDMAEGQFLSFGFKPWPVDYDVQPIDTEITSVLVHFIGTR
ARVGFRFKMPHRPSRINIKQDELDELRSRSRLIQEKDQALLEKVRLRLRDGFIGIFDKELRVLAVDLGTSSCATAF
FVGRQFQESSRLQIVKYDRVYKSNYEIKKRRNNKGIDKQKQLLFKEKGLNQYHIKVHLDKLAEQNKQIIKKREASG
NPTPTEQDMRRLSLHIGWMHRDWVRINASQIIKSAKKLRADLIVFESLRDFRPMMFNEFDIDKKRRLAFFPFGLIR
HKVIEKAVESGMRVVTVPYMFSSQFCGACGRQQNDKKRLQKNKTDKRGACFICEYNDCAFEGDPDENAARVLGGVF
WGNIGLPLS (SEQ ID NO: 4)
>FLYL01000025_159|P
[hot springs metagenome]
MAQASSTPAVSPRPRPRYREERTLVRKLLPRPGQSKQEFRENVKKLRKAFLQFNADVSGVCQWAIQFRPRYGKPAE
PTETFWKFFLEPETSLPPNDSRSPEFRRLQAFEAAAGINGAAALDDPAFTNELRDSILAVASRPKTKEAQRLFSRL
KDYQPAHRMILAKVAAEWIESRYRRAHQNWERNYEEWKKEKQEWEQNHPELTPEIREAFNQIFQQLEVKEKRVRIC
PAARLLQNKDNCQYAGKNKHSVLCNQFNEFKKNHLQGKAIKFFYKDAEKYLRCGLQSLKPNVQGPFREDWNKYLRY
MNLKEETLRGKNGGRLPHCKNLGQECEFNPHTALCKQYQQQLSSRPDLVQHDELYRKWRREYWREPRKPVFRYPSV
KRHSIAKIFGENYFQADFKNSVVGLRLDSMPAGQYLEFAFAPWPRNYRPQPGETEISSVHLHFVGTRPRIGFRFRV
PHKRSRFDCTQEELDELRSRTFPRKAQDQKFLEAARKRLLETFPGNAEQELRLLAVDLGTDSARAAFFIGKTFQQA
FPLKIVKIEKLYEQWPNQKQAGDRRDASSKQPRPGLSRDHVGRHLQKMRAQASEIAQKRQELTGTPAPETTTDQAA
KKATLQPFDLRGLTVHTARMIRDWARLNARQIIQLAEENQVDLIVLESLRGFRPPGYENLDQEKKRRVAFFAHGRI
RRKVTEKAVERGMRVVTVPYLASSKVCAECRKKQKDNKQWEKNKKRGLFKCEGCGSQAQVDENAARVLGRVFWGEI
ELPTAIP (SEQ ID NO: 6)
>ORFD01000444_4|P
[sediment metagenome]
MAQASSTPAVSPRPRPRYREERTLVRKLLPRPGQSKQEFRENVKKLRKAFLQFNADVSGVCQWAIQFRPRYGKPAE
PTETFWKFFLEPETSLPPNDSRSPEFRRLQAFEAAAGINGAAALDDPAFTNELRDSILAVASRPKTKEAQRLFSRL
KDYQPAHRMILAKVAAEWIESRYRRAHQNWERNYEEWKKEKQEWEQNHPELTPEIREAFNQIFQQLEVKEKRVRIC
PAARLLQNKDNCQYAGKNKHSVLCNQFNEFKKNHLQGKAIKFFYKDAEKYLRCGLQSLKPNVQGPFREDWNKYLRY
MNLKEETLRGKNGGRLPHCKNLGQECEFNPHTALCKQYQQQLSSRPDLVQHDELYRKWRREYWREPRKPVFRYPSV
KRHSIAKIFGENYFQADFKNSVVGLRLDSMPAGQYLEFAFAPWPRNYRPQPGETEISSVHLHFVGTRPRIGFRFRV
PHKRSRFDCTQEELDELRSRTFPRKAQDQKFLEAARKRLLETFPGNAEQELRLLAVDLGTDSARAAFFIGKTFQQA
FPLKIVKIEKLYEQWPNQKQAGDRRDASSKQPRPGLSRDHVGRHLQKMRAQASEIAQKRQELTGTPAPETTTDQAA
KKATLQPFDLRGLTVHTARMIRDWARLNARQIIQLAEENQVDLIVLESLRGFRPPGYENLDQEKKRRVAFFAHGRI
RRKVTEKAVERGMRVVTVPYLASSKVCAECRKKQKDNKQWEKNKKRGLFKCEGCGSQAQVDENAARVLGRVFWGEI
ELPTAIP (SEQ ID NO: 6)
>ORXB01000005_848|P
[sediment metagenome]
MAQASSTPAVSPRPRPRYREERTLVRKLLPRPGQSKQEFRENVKKLRKAFLQFNADVSGVCQWAIQFRPRYGKPAE
PTETFWKFFLEPETSLPPNDSRSPEFRRLQAFEAAAGINGAAALDDPAFTNELRDSILAVASRPKTKEAQRLFSRL
KDYQPAHRMILAKVAAEWIESRYRRAHQNWERNYEEWKKEKQEWEQNHPELTPEIREAFNQIFQQLEVKEKRVRIC
PAARLLQNKDNCQYAGKNKHSVLCNQFNEFKKNHLQGKAIKFFYKDAEKYLRCGLQSLKPNVQGPFREDWNKYLRY
MNLKEETLRGKNGGRLPHCKNLGQECEFNPHTALCKQYQQQLSSRPDLVQHDELYRKWRREYWREPRKPVFRYPSV
KRHSIAKIFGENYFQADFKNSVVGLRLDSMPAGQYLEFAFAPWPRNYRPQPGETEISSVHLHFVGTRPRIGFRFRV
PHKRSRFDCTQEELDELRSRTFPRKAQDQKFLEAARKRLLETFPGNAEQELRLLAVDLGTDSARAAFFIGKTFQQA
FPLKIVKIEKLYEQWPNQKQAGDRRDASSKQPRPGLSRDHVGRHLQKMRAQASEIAQKRQELTGTPAPETTTDQAA
KKATLQPFDLRGLTVHTARMIRDWARLNARQIIQLAEENQVDLIVLESLRGFRPPGYENLDQEKKRRVAFFAHGRI
RRKVTEKAVERGMRVVTVPYLASSKVCAECRKKQKDNKQWEKNKKRGLFKCEGCGSQAQVDENAARVLGRVFWGEI
ELPTAIP (SEQ ID NO: 6)
>3300027640|Ga0209347_1003758_14|P
[terrestrial-deep subsurface]
MSNTRYSESRTLVRRLRRLPGESREEFRGKVRRLRKQFAQFNVNASELCQWLMSLRPGGKKASDRTKEFWEFFLEP
ERFLENQDDDRCDACRLAVFDVAAGLAPADRLGDYGVSQALAESVHVIGQISLTPTAAKLFTRLCGFEASHRQVLL
KAAAEWIVAHYLRGYENWVRRHEEWEKEKARWEASHPELTQAARDDFNRIFKDLGIERKRPRVCTGERLKANKDDC
DWAGERIPVGGTWRNHSSLCVKYWRFLKEYPRKARVPRQFREFFVTNAKTYMDLRRTSRGDRSVTMAAFLRKQRNA
QWFPQAWEAYLKALEVNEQTVLAAGYGLPHCTEIGPDADCQFNKHTADCEKYRRALDARPDLLPLEKLYRHWRREY
LSGPGKPCFQYPSQRKLPMPKIFGRGYFRVDLASSIIELRMEGGRDFERFRIAAWPSDYTPSAQEAQITSVHVSFV
GTRALAGFRFEVPHKASRFAAGQDQIDELRSRKYPRRAQDAEFLVAARKRLLESFAGGAEHDVRILAVDLGTSNGA
VAVFRGRSLEKAMPLDVIKLEKLHSSSPKENRGAGPEPSEEERKKARARGLRPSHVGRHLENWALAAREIANQRGN
EADGPATLGDHDLRRFSLHIRWMIRDWVRLNVSQIIEAAEGNHVDLIVFESMRGWRAPGYDTVDDEKKRRLAFFAH
GRIRHKIREKAVERGMRVVTVPYFMSSQFCGSCGTQQQDTRKLKTNKRERTSFTCENCGHRANSDENAAQVLAKVF
WGDVVLPEDPDDCS (SEQ ID NO: 22)
>3300027640|Ga0209347_1003758_16|M
[terrestrial-deep subsurface]
MVRRLRRLPGESREEFRGKVRRLRKQFAQFNVNASELCQWLMSLRPGGKKASDRTKEFWEFFLEPERFLENQDDDR
CDACRLAVFDVAAGLAPADRLGDYGVSQALAESVHVIGQISLTPTAAKLFTRLCGFEASHRQVLLKAAAEWIVAHY
LRGYENWVRRHEEWEKEKARWEASHPELTQAARDDFNRIFKDLGIERKRPRVCTGERLKANKDDCDWAGERIPVGG
TWRNHSSLCVKYWRFLKEYPRKARVPRQFREFFVTNAKTYMDLRRTSRGDRSVTMAAFLRKQRNAQWFPQAWEAYL
KALEVNEQTVLAAGYGLPHCTEIGPDADCQFNKHTADCEKYRRALDARPDLLPLEKLYRHWRREYLSGPGKPCFQY
PSQRKLPMPKIFGRGYFRVDLASSIIELRMEGGRDFERFRIAAWPSDYTPSAQEAQITSVHVSFVGTRALAGFRFE
VPHKASRFAAGQDQIDELRSRKYPRRAQDAEFLVAARKRLLESFAGGAEHDVRILAVDLGTSNGAVAVFRGRSLEK
AMPLDVIKLEKLHSSSPKENRGAGPEPSEEERKKARARGLRPSHVGRHLENWALAAREIANQRGNEADGPATLGDH
DLRRFSLHIRWMIRDWVRLNVSQIIEAAEGNHVDLIVFESMRGWRAPGYDTVDDEKKRRLAFFAHGRIRHKIREKA
VERGMRVVTVPYFMSSQFCGSCGTQQQDTRKLKTNKRERTSFTCENCGHRANSDENAAQVLAKVFWGDVVLPEDPD
DCS (SEQ ID NO: 23)
>3300027657|Ga0256865_1003050_2 M
[terrestrial-soil]
MAVTRYREERTLVRGLRRLPGQSREQFRKNVLLLRRHFERFNVDVSDICQWMMGLRPKDGEVTPATQPLWDFMLEP
SDGQGNAQGDPDRMRLLAFRVATGVEHSQSGVRLPLHVQESLRHVAALTSTESARRLILRFQQLEQSHQMILLKSA
SEWVRTRYSNANENWQRNRPLWEKEKAEWEKEHPALTPDACRKFSDIFKELGIKDKRPRICGWNRLKLPKDNCDYA
GERVGGGRHAPLCKFYREFQAGLRREYKKQFPDNALKYLALRKQKGHTQAVVLQQFCAKDRRKSGWFPKAWMTYLQ
KLNVTEETLIQRYQGQLPHCVKIDNKTGCSFNPHTNDCLEYKKRILKLPESDRELETQYREWRRDYLSGPRKPSFR
YPSSRNLPTPKIFGAGYYEADFTRSMLRLRLDDMPRGRFIEFGFKPWPSDYDIQPVSTQITSAHIHFIGTRARVGF
RFAVAARPSRLRISQDEIDALRRQYPRAAQDQQFLDHVRPLILDSFAGNPKQELRILTIDLGTSGGAAAAFCGVTL
VKSEVLKVIKLDKLYDLLDREDKRSPTSGLGEGHVGRHLEALSKEAAKIAQHRTTWKNPGLRPFDERQLTSHIRWM
IRDWVRLNAQQIIEIAERENADLILFESMRGYYPKARDKYDSAQKVRLGFFSYGAIRRKVAEKAVERGMRILTLPY
KFSSQICSKCGRKQENRGLKTKKAKRLFKCEHTGCGTELNSDENAARVLAGVFWGTIKLPEKAVVSHT (SEQ ID
NO: 24)

TABLE 5A
Representative CLUST.019143 (Type V-G) Effectors and Direct Repeats

| Effector Accession | Direct Repeat Nucleotide Sequence |
|---|---|
| RIK66295.1 (SEQ ID NO: 15) | GGTGCGACCGGCCGTCGATTGACCGGTTTCGACACC (SEQ |
| | ID NO: 25) |
| JRYK01000049_75\|P (SEQ ID NO: 2) | GGTGCGGCAGCCGGTAGAACTGCGGCTTCCGACACC (SEQ |
| | ID NO: 10) |
| JRYK01000049_79\|M (SEQ ID NO: 3) | GGTGCGGCAGCCGGTAGAACTGCGGCTTCCGACACC (SEQ |
| | ID NO: 10) |
| UOQF01000587_2\|M (SEQ ID NO: 16) | GTTGTAGCGCCCCCTGAATTAGCGGTTTCCGACACC (SEQ |
| | ID NO: 26) |
| UOQK01013695_2\|M (SEQ ID NO: 16) | GTTGTAGCGCCCCCTGAATTAGCGGTTTCCGACACC (SEQ |
| | ID NO: 26) |
| 3300028920\|Ga0272441_10052198_2\| | GGTGTAAGTCCGACTGAATGCGAGGTTTCCGACACC (SEQ |
| M (SEQ ID NO: 17) | ID NO: 27) |
| 3300028920\|Ga0272441_10052198_4\| | GGTGTAAGTCCGACTGAATGCGAGGTTTCCGACACC (SEQ |
| P (SEQ ID NO: 18) | ID NO: 27) |
| 3300016590\|Ga0186947_1003_42\|P | GCTGTGACTCACCGGCATTTGCGGGGTTCTTACACC (SEQ |
| (SEQ ID NO: 8) | ID NO: 28) |
| 3300016590\|Ga0186947_1003_42\|M | GCTGTGACTCACCGGCATTTGCGGGGTTCTTACACC (SEQ |
| (SEQ ID NO: 7) | ID NO: 28) |
| 3300010938\|Ga0137716_10003017_35\| | GTTGTGACCGCCCCCGGAATCACGGTTTGCGACACC (SEQ |
| M (SEQ ID NO: 19) | ID NO: 29) |
| 3300010938\|Ga0137716_10003017_32\| | GTTGTGACCGCCCCCGGAATCACGGTTTGCGACACC (SEQ |
| P (SEQ ID NO: 20) | ID NO: 29) |
| 3300006865\|Ga0073934_10000021_236\| | GGTGTAGCGGGCCTCAAAAACACGGCATCCGACACC (SEQ |
| M (SEQ ID NO: 5) | ID NO: 30) |
| 3300006945\|Ga0073933_1000004_588\| | GGTGGAAAGGGCCGGAGATTTACCGGCTCTGACACC (SEQ |
| P (SEQ ID NO: 6) | ID NO: 13) |
| 3300007071\|Ga0073930_1000342_16\| | GGTGGAAAGGGCCGGAGATTTACCGGCTCTGACACC (SEQ |
| M (SEQ ID NO: 6) | ID NO: 13) |
| 3300022554\|Ga0212093_1000011_56\| | GGTGGAAAGGGCCGGAGATTTACCGGCTCTGACACC (SEQ |
| M (SEQ ID NO: 6) | ID NO: 13) |
| 3300025094\|Ga0209478_1000005_814\| | GGTGGAAAGGGCCGGAGATTTACCGGCTCTGACACC (SEQ |
| P (SEQ ID NO: 6) | ID NO: 13) |
| 3300025105\|Ga0209479_1000006_88\| | GGTGGAAAGGGCCGGAGATTTACCGGCTCTGACACC (SEQ |
| P (SEQ ID NO: 6) | ID NO: 13) |
| 3300025310\|Ga0209172_10000050_165\| | GGTGTAGCGGGCCTCAAAAACACGGCATCCGACACC (SEQ |
| P (SEQ ID NO: 5) | ID NO: 30) |
| 3300019846\|Ga0197078_1002_15\|M | GGTTTGCAAACAGCGAGAACATCTGTGTTTTGGTGGTAGTTA |
| (SEQ ID NO: 21) | CAAC (SEQ ID NO: 31) |
| 3300019846\|Ga0197078_1002_15\|M | GTTTGCAAACAGCGAGAACATCTGTGTTTTGGTGGT (SEQ |
| (SEQ ID NO: 21) | ID NO: 32) |
| 3300023207\|Ga0255811_11223672_52\| | GTTGTAGCGGCCCCTGAATTAGCGGTTTCCGACACC (SEQ |
| P (SEQ ID NO: 1) | ID NO: 9) |
| RKX31836.1 (SEQ ID NO: 4) | GTTGGAGTCAGCAAGGAATCAACGGTTTTGGACACC (SEQ |
| | ID NO: 11) |
| FLYL01000025_159\|P (SEQ ID NO: | GGTGGAAAGGGCCGGAGATTTACCGGCTCTGACACC (SEQ |
| 6) | ID NO: 13) |
| ORFD01000444_4\|P (SEQ ID NO: 6) | GGTGGAAAGGGCCGGAGATTTACCGGCTCTGACACC (SEQ |
| | ID NO: 13) |
| ORXB01000005_848\|P (SEQ ID NO: | GGTGGAAAGGGCCGGAGATTTACCGGCTCTGACACC (SEQ |
| 6) | ID NO: 13) |
| 3300027640\|Ga0209347_1003758_14\| | GGTGGAGCCGGCCGTGAATTAGGGGGTTTCGACACC (SEQ |
| P (SEQ ID NO: 22) | ID NO: 33) |
| 3300027640\|Ga0209347_1003758_16\| | GGTGGAGCCGGCCGTGAATTAGGGGGTTTCGACACC (SEQ |
| M (SEQ ID NO: 23) | ID NO: 33) |
| 3300027657\|Ga0256865_1003050_2\|M | GGTGTTGTGAACCCTGAAAGAACGGATTCCGACACC (SEQ |
| (SEQ ID NO: 24) | ID NO: 34) |

TABLE 5B
Example CLUST.019143 (Type V-G) pre-crRNA sequences

| | | Spacer |
|---|---|---|
| Effector Accession | Example pre-crRNA sequence | Lens |
| UOQK01013695_2\|M | GUUGUAGCGCCCCCUGAAUUAGCGGUUUCCGACACCNNNNNNNNNNNNNN | 27- |
| (SEQ ID NO: 16) | NNNNNNNNNNNNNGUUGUAGCGCCCCCUGAAUUAGCGGUUUCCGACACC | 28 |
| | (SEQ ID NO: 150) | |
| JRYK01000049_75\|P | GGUGCGGCAGCCGGUAGAACUGCGGCUUCCGACACCNNNNNNNNNNNNNN | 29- |
| (SEQ ID NO: 2) | NNNNNNNNNNNNNNNGGUGCGGCAGCCGGUAGAACUGCGGCUUCCGACAC | 30 |
| | C (SEQ ID NO: 151) | |
| 3300028920\|Ga0272441_ | GGUGUAAGUCCGACUGAAUGCGAGGUUUCCGACACCNNNNNNNNNNNNNN | 29- |
| 10052198_2\|M | NNNNNNNNNNNNNNNGGUGUAAGUCCGACUGAAUGCGAGGUUUCCGACAC | 30 |
| (SEQ ID NO: 17) | C (SEQ ID NO: 152) | |
| 3300019846\|Ga0197078_ | GGUUUGCAAACAGCGAGAACAUCUGUGUUUUGGUGGUAGUUACAACNNNN | 29- |
| 1002_15\|M (SEQ | NNNNNNNNNNNNNNNNNNNNNNNNNGGUUUGCAAACAGCGAGAACAUCUG | 30 |
| ID NO: 21) | UGUUUUGGUGGUAGUUACAAC (SEQ ID NO: 153) | |
| 3300019846\|Ga0197078_ | GUUUGCAAACAGCGAGAACAUCUGUGUUUUGGUGGUNNNNNNNNNNNNNN | 29- |
| 1002_15\|M (SEQ | NNNNNNNNNNNNNNNGUUUGCAAACAGCGAGAACAUCUGUGUUUUGGUGG | 30 |
| ID NO: 21) | U (SEQ ID NO: 154) | |
| 3300025105\|Ga0209479_ | GGUGGAAAGGGCCGGAGAUUUACCGGCUCUGACACCNNNNNNNNNNNNNN | 29- |
| 1000006_88\|P | NNNNNNNNNNNNNNNNGGUGGAAAGGGCCGGAGAUUUACCGGCUCUGACA | 30 |
| (SEQ ID NO: 6) | CC (SEQ ID NO: 155) | |
| FLYL01000025_159\|P | GGUGGAAAGGGCCGGAGAUUUACCGGCUCUGACACCNNNNNNNNNNNNNN | 29- |
| (SEQ ID NO: 6) | NNNNNNNNNNNNNNNNGGUGGAAAGGGCCGGAGAUUUACCGGCUCUGACA | 30 |
| | CC (SEQ ID NO: 156) | |
| 3300027640\|Ga0209347_ | GGUGGAGCCGGCCGUGAAUUAGGGGGUUUCGACACCNNNNNNNNNNNNNN | 27- |
| 1003758_14\|P | NNNNNNNNNNNNNNNNGGUGGAGCCGGCCGUGAAUUAGGGGGUUUCGACA | 30 |
| (SEQ ID NO: 22) | CC (SEQ ID NO: 157) | |
| 3300027640\|Ga0209347_ | GGUGGAGCCGGCCGUGAAUUAGGGGGUUUCGACACCNNNNNNNNNNNNNN | 27- |
| 1003758_16\|M | NNNNNNNNNNNNNNNNGGUGGAGCCGGCCGUGAAUUAGGGGGUUUCGACA | 30 |
| (SEQ ID NO: 23) | CC (SEQ ID NO: 158) | |
| 3300027657\|Ga0256865_ | GGUGUUGUGAACCCUGAAAGAACGGAUUCCGACACCNNNNNNNNNNNNNN | 29- |
| 1003050_2 M | NNNNNNNNNNNNNNNNGGUGUUGUGAACCCUGAAAGAACGGAUUCCGACA | 30 |
| (SEQ ID NO: 24) | CC (SEQ ID NO: 159) | |
| UOQF01000587_2\|M | GUUGUAGCGCCCCCUGAAUUAGCGGUUUCCGACACCNNNNNNNNNNNNNN | 29- |
| (SEQ ID NO: 16) | NNNNNNNNNNNNNNNNGUUGUAGCGCCCCCUGAAUUAGCGGUUUCCGACA | 31 |
| | CC (SEQ ID NO: 160) | |
| 3300023207\|Ga0255811_ | GUUGUAGCGGCCCCUGAAUUAGCGGUUUCCGACACCNNNNNNNNNNNNNN | 29- |
| 11223672_52\|P | NNNNNNNNNNNNNNNNGUUGUAGCGGCCCCUGAAUUAGCGGUUUCCGACA | 31 |
| (SEQ ID NO: 1) | CC (SEQ ID NO: 161) | |
| 3300010938\|Ga0137716_ | GUUGUGACCGCCCCCGGAAUCACGGUUUGCGACACCNNNNNNNNNNNNNN | 29- |
| 10003017_35\|M | NNNNNNNNNNNNNNNNGUUGUGACCGCCCCCGGAAUCACGGUUUGCGACA | 33 |
| (SEQ ID NO: 19) | CC (SEQ ID NO: 162) | |
| 3300010938\|Ga0137716_ | GUUGUGACCGCCCCCGGAAUCACGGUUUGCGACACCNNNNNNNNNNNNNN | 29- |
| 10003017_32\|P | NNNNNNNNNNNNNNNNGUUGUGACCGCCCCCGGAAUCACGGUUUGCGACA | 33 |
| (SEQ ID NO: 20) | CC (SEQ ID NO: 163) | |
| 3300006865\|Ga0073934_ | GGUGUAGCGGGCCUCAAAAACACGGCAUCCGACACCNNNNNNNNNNNNNN | 31- |
| 10000021_236\|M | NNNNNNNNNNNNNNNNNNGGUGUAGCGGGCCUCAAAAACACGGCAUCCGA | 33 |
| (SEQ ID NO: 5) | CACC (SEQ ID NO: 164) | |
| 3300025310\|Ga0209172_ | GGUGUAGCGGGCCUCAAAAACACGGCAUCCGACACCNNNNNNNNNNNNNN | 31- |
| 10000050_165\|P | NNNNNNNNNNNNNNNNNNGGUGUAGCGGGCCUCAAAAACACGGCAUCCGA | 33 |
| (SEQ ID NO: 5) | CACC (SEQ ID NO: 165) | |
| 3300016590\|Ga0186947_ | GCUGUGACUCACCGGCAUUUGCGGGGUUCUUACACCNNNNNNNNNNNNNN | 37- |
| 1003_42\|P (SEQ | NNNNNNNNNNNNNNNNNNNNNNNNGCUGUGACUCACCGGCAUUUGCGGGG | 39 |
| ID NO: 8) | UUCUUACACC (SEQ ID NO: 166) | |
| 3300016590\|Ga0186947_ | GCUGUGACUCACCGGCAUUUGCGGGGUUCUUACACCNNNNNNNNNNNNNN | 37- |
| 1003_42\|M (SEQ | NNNNNNNNNNNNNNNNNNNNNNNNGCUGUGACUCACCGGCAUUUGCGGGG | 39 |
| ID NO: 7) | UUCUUACACC (SEQ ID NO: 167) | |

### Example 2. Identification of Transactivating RNA Elements

In addition to an effector protein and an RNA guide (e.g., a crRNA), some CRISPR systems also include an additional small RNA that activates robust enzymatic activity referred to as a transactivating RNA (tracrRNA). Such tracrRNAs typically include a complementary region that hybridizes to the crRNA. The crRNA-tracrRNA hybrid forms a complex with an effector resulting in the activation of programmable enzymatic activity.
tracrRNA sequences can be identified by searching genomic sequences flanking CRISPR arrays for short sequence motifs that are complementary to the direct repeat portion of the crRNA. Search methods include exact or degenerate sequence matching for the complete direct repeat (DR) or DR subsequences. For example, a DR of length n nucleotides can be decomposed into a set of overlapping 6-10 nt kmers. These kmers can be aligned to sequences flanking a CRISPR locus, and regions of homology with 1 or more kmer alignments can be identified as DR homology regions for experimental validation as tracrRNAs. Alternatively, RNA cofold free energy can be calculated for the complete DR or DR subsequences and short kmer sequences from the genomic sequence flanking the elements of a CRISPR system. Flanking sequence elements with low minimum free energy structures can be identified as DR homology regions for experimental validation as tracrRNAs.
tracrRNA elements frequently occur within close proximity to CRISPR associated genes or a CRISPR array. As an alternative to searching for DR homology regions to identify tracrRNA elements, non-coding sequences flanking CRISPR associated proteins or the CRISPR array can be isolated by cloning or gene synthesis for direct experimental validation of tracrRNAs.
Experimental validation of tracrRNA elements can be performed using small RNA sequencing of the host organism for a CRISPR system or synthetic sequences expressed heterologously in non-native species. Alignment of small RNA sequences from the originating genomic locus can be used to identify expressed RNA products containing DR homology regions and stereotyped processing typical of complete tracrRNA elements.
Complete tracrRNA candidates identified by RNA sequencing can be validated in vitro or in vivo by expressing the crRNA and effector in combination with or without the tracrRNA candidate, and monitoring the activation of effector enzymatic activity.
In engineered constructs, the expression of tracrRNAs can be driven by promoters including, but not limited to U6, U1, and H1 promoters for expression in mammalian cells or J23119 promoter for expression in bacteria.
In some instances, a tracrRNA can be fused with a crRNA and expressed as a single RNA guide.
In some instances, the CLUST.019143 (Type V-G CRISPR-Cas effector protein) system includes a tracrRNA that includes a direct repeat homology sequence listed in TABLE 6.
In some instances, the CLUST.019143 (Type V-G CRISPR-Cas effector protein) system includes a tracrRNA encoded by a non-coding sequence (or fragment thereof) listed in TABLE 7. Note that in (SEQ ID NO: 131), the Cas12g1 tracrRNA sequence is indicated in bold font.

TABLE 6
Direct Repeat Homology-containing Regions
of Representative CLUST.019143 (Type V-G) Systems

| Effector accession | Homologous Region |
|---|---|
| 3300027657Ga0256865_1003050_ | TCCGTTCTT |
| 2\|M | (SEQ ID NO: 100) |
| 3300027640\|Ga0209347_1003758_ | AAACCCC |
| 16\|M | (SEQ ID NO: 101) |
| 3300027640Ga0209347_1003758_ | CCTAATT |
| 16\|M | (SEQ ID NO: 102) |
| 3300028920\|Ga0272441_10052198_ | GGTGTCGGAA |
| 2\|M | (SEQ ID NO: 103) |
| JRYK01000049_79\|M | CGGCTGCCG |
| | (SEQ ID NO: 104) |
| RIK66295.1 | TCGACGG |
| | (SEQ ID NO: 105) |
| RIK66295.1 | GGTGTCG |
| | (SEQ ID NO: 106) |
| RIK66295.1 | TCGACGG |
| | (SEQ ID NO: 105) |
| RIK66295.1 | CGACGGC |
| | (SEQ ID NO: 107) |
| 3300006865\|Ga0073934_10000021_ | GTGTTTTTG |
| 236\|M | (SEQ ID NO: 108) |
| 3300010938\|Ga0137716_10003017_ | TTCCGGGG |
| 35\|M | (SEQ ID NO: 109) |
| 3300023207\|Ga0255811_11223672_ | GGAAACCG |
| 52\|P | (SEQ ID NO: 110) |
| 3300023207\|Ga0255811_11223672_ | GGGGCCGC |
| 52\|P | (SEQ ID NO: 111) |
| 3300023207\|Ga0255811_11223672_ | GAAACCGC |
| 52\|P | (SEQ ID NO: 112) |
| 3300006945\|Ga0073933_1000004_ | GAGCCGGTAAAT |
| 588\|P | (SEQ ID NO: 113) |
| 3300016590\|Ga0186947_1003_42\|M | AATGCCG |
| | (SEQ ID NO: 114) |
| 3300019846\|Ga0197078_1002_15\|M | TGTAACT |
| | (SEQ ID NO: 115) |
| UOQF01000587_2\|M | GCTAATT |
| | (SEQ ID NO: 116) |

TABLE 7
Direct Repeat Homology-containing Sequences
of Representative CLUST.019143 (Type V-G) CRISPR-Cas Systems

>3300027657|Ga0256865_1003050_2|M
TCCGTTCTTCTTTGTAGGGTGTTGTGAACCCTGAAAGAACGGCGGGTGGCCCGGACCTTGGGGCGTGTTGAAAAA
CCCGTTTGTCGTCAGTTGATCGGTGAAGGTTATCAAGCGATGGCGATCCTGTAGTTGAGATCGACGGGGTTGGTG
TCAGACCGA (SEQ ID NO: 117)
>3300027640|Ga0209347_1003758_16|M
AACAGCGACGAAAACGCCGCACAGGTGCTGGCGAAGGTGTTCTGGGGCGACGTCGTTCTACCCGAGGATCCGGAC
GATTGCTCTTGACGAAGATATCGAAGGTGGTATAGTGCCCTTGTGTCCTCGTCGTGAGTCGATAATGGCTCGGAC
AAACCCCGGCGGAGCCTTCGGTGCCAACGGGAGGAACCTGAGCCCGGAGGATACAAACTCTCTAATTCGTGGTTG
ATATGGCGATCAAGAATGCGGTTTCTTTCAGGCAATTGTGTCGCAACTCTTTGTCGATCCAGGGATTACGACGGG
ACA (SEQ ID NO: 118)
>3300027640|Ga0209347_1003758_16|M
CCAGACAGCGAAAATGCTGAATAATCCCGGGGACGGCATACCTAATTGGGTTCCAGATTGCCCAGCAGGCAGAAG
AGAGCGTTTATCGCATTTGGGGTGCCGGCGGAGAGGGGGCGGGCCAAGGGTGTCACTGGATGTCTCCGCTGGTAC
CCGGCAAAGCGTCAAACGCAATCAATCCCGGCCGAAGGCCTTCCGCT (SEQ ID NO: 119)
>3300028920|Ga0272441_10052198_2|M
AGTACGCGGGCACGCGGGGGCGTGTTTAGTACGCAGGCACGCGGGGGCGTGTTTAGTACGCGGGGGCGTGATTAG
TACAAAGGGGCAAGTCAGGTATACATCATTAAAGATGTCGAAACCGCAGGGGTTTCGACCTACTCGAAGAAAGAA
GGTGTCGGAACGGCCTGCATCATGGGGTTTGGGAGGGGGATTGGGGGCTATTTGAATCGAAACAAAGTTGATATT
GTCCCTATGCGGGGGATTGTTTGTAGTGTTGTTCGAGTAAGACCAGGTTTTCATCGGGGATGCGGCCTTTTTCAA
AAGAGTCGAC (SEQ ID NO: 120)
>JRYK01000049_79|M
CGAGCGCGCGCTTCAGCACGAAGCACGAGGTGCGGCTGCCGGTTGCATTGCGGCAGAATGATTGGCCCGCCTGTT
CACGCTTTCCCGACCGGCAGTGGAAAACAAACGTCCCGCACGTCGCAGTTCGTGGCTTAGTTGATGCGTGCGCGT
GGGCCGATTGACGGGCGCGCTCGGCTCCGCCTCGCGGCTAA (SEQ ID NO: 121)
>RIK66295.1
TTTCGACACCTGCGCACCGACTTCAAAGCACCATCGGTTGGGTGCGACCGGCCGTCGATTGACCGGTTTCGACAC
CCCGCTTTGCACAGCGGCAGCAGACAAGGATGGTGCGACCGGCCGTCGATTGACCGGTTTCGACACCACTCCGCC
TCGACGGTCTTAACCAGTTCTGGGTGCGACCGGCCGTCGATTGACCGGTTTCGACACCCTGGACGTGTCGGGCAA
TACTTCTGTCGGAGGTGCGACCGGCCGTCGATTGACCGGTTTCGACACCTACGCCACGGCAAAGCACCTGATCAG
CTCGGGT (SEQ ID NO: 122)
>RIK66295.1
TGCTCCACTGGGCGATGTATGCCTGCACCACTGGGGGTGCTGCACGCGGCGGCGCAGCGCCTACAATGGCGCCGC
GGAAGGGGAGCGCGGCGCGGTCATGGAACCCACGACGATTGAGATCAGGAACGCGCGGGAGCATAACCTGCGGTC
GGTGTCGCTTTCGTTGCCGCGGGGGAAGCTGATTGTCTTCACCGGGGTTTCGGGCAGCGGGAAGAGTTCGCTGGC
GTTTGACACGCTCTATGCCGAGGGGCAGCGGCGGTATATCGAGTCGTTGTCGTCGTATGCCCGACAGTTCATGGG
GCAGATG (SEQ ID NO: 123)
>RIK66295.1
TATGCCGAGGGGCAGCGGCGGTATATCGAGTCGTTGTCGTCGTATGCCCGACAGTTCATGGGGCAGATGGCCAAG
CCGGACTGCGATCAGATCACGGGGCTTTCGCCATCGATCGCGATTCAGCAGAAGACGACGGGATGGAATCCGCGG
TCGACGGTGGGGACGACGACGGCGATCTATGACTTTCTGCGCGTGCTGTACGCGCGGATCGGGACGCAGCATTGC
ACGCAGTGTGGGCGGGCGATCACGGCGCAGTCTCGCGAGCAGATCGCGGCGGGGATCTTGAGCGCGTTTAATCCG
CGCCGCG (SEQ ID NO: 124)
>RIK66295.1
GGCGGTATATCGAGTCGTTGTCGTCGTATGCCCGACAGTTCATGGGGCAGATGGCCAAGCCGGACTGCGATCAGA
TCACGGGGCTTTCGCCATCGATCGCGATTCAGCAGAAGACGACGGGATGGAATCCGCGGTCGACGGTGGGGACGA
CGACGGCGATCTATGACTTTCTGCGCGTGCTGTACGCGCGGATCGGGACGCAGCATTGCACGCAGTGTGGGCGGG
CGATCACGGCGCAGTCTCGCGAGCAGATCGCGGCGGGGATCTTGAGCGCGTTTAATCCGCGCCGCGATCCCACCT
ATCAGAG (SEQ ID NO: 125)
>3300006865|Ga0073934_10000021_236|M
AGCCGACGAGAGCGCATTTGCCCTCCAAGGCTTGACTCTTTTTCCAGCCGTGGCTACGCTGTTAATGCCCTTCGA
GTCGCATTGTCCTGCATACGCCCGGCGGAACTTCGGTACCAACGGGACGGACCCCGGACACGAAAGGCAAATGCT
GTGTTTTTGTGGCCTGCCGGTTTGGAACGTCACGGTTTTGTGGGCGGTTTTTTTTGACGCAAGTCCTTGTCAGAA
GCGAATTTGCAGACGAGAC (SEQ ID NO: 126)
>3300010938|Ga0137716_10003017_35|M
TTCGAGTCATTTGCCTGGAATACGCCCTGCGGACGTTTTCGACCAACGGGATGGACCCCTGGCGCTGAAGGCAGA
ATCGGTTTTTCCCGGGCGGTTTCGGGAGGCGAACGAGCGGTTTTTTCGAGGTGGAACTGGCGCAAGTCGTGTCCG
TTCCGGGGCTTGTTTGCCAAGAG (SEQ ID NO: 127)
>3300023207|Ga0255811_11223672_52|P
AATTACTCTCCCCACCGATTGATAGTTGAAAAAACTTGACAGACAGCTTAATTTAAGTTATGTATAATCAGTTGC
TTCGTGGTCCCTCTGGCCGGAAAAACCTCTGGCGGAGCGCAAGCACCAATAGAGAGGAACTCGGCCCAGAAGCGA
GGAAACCGACAATTAGGGGTTTAATTATTGAAGGAACTTTAAAGATTTTGTGACGGACGGAATAAATTAAGTTCA
AATCGCGTATGATTATATGAATGATGA (SEQ ID NO: 128)
>3300023207|Ga0255811_11223672_52|P
AAGGTCGACAAAGATTTCGGTTGGTTATAAATGAACGAGGAGGGGCAGATGTGGACATCTGCCGCCCACTTGAGT
CTGGGGCGATAAGCTGATTGTCGGGGGAGGTTTCACCACCGCTGGGGGGATCGATGTGAATCGTATTGCCGCCTG
GGGGCCGCAATAATAAATTAAACGGCGTCAGGAAGAGGTTCCAGATGCCGCTCAATACCCATAGCACTTAGCCCT
TGCCTTAACAATGAATTGTGTCTATTTTGAGGGCTGTCTTATAAATAGAAAAGATATCAAGATAAGTTGTCGAAA
CCGCAGGG (SEQ ID NO: 129)
>3300023207|Ga0255811_11223672_52|P
CCTGGGGGCCGCAATAATAAATTAAACGGCGTCAGGAAGAGGTTCCAGATGCCGCTCAATACCCATAGCACTTAG
CCCTTGCCTTAACAATGAATTGTGTCTATTTTGAGGGCTGTCTTATAAATAGAAAAGATATCAAGATAAGTTGTC
GAAACCGCAGGGGTTTCGACCTAAAAGAATTATGTCTGACGAAGAAAAAAAAGAGAAGACCAAATCCGAGATGCC
GTTTCTGGATCATATCGAGGAACTGCGCTGGCGGTTGATCAAGTCCATTTTATCGGTTGCCGTGATGGCCATCCT
GGCCTTTA (SEQ ID NO: 130)
>3300006945|Ga0073933_1000004_588|P
CGAAATCGAGCTGCCGACCGCAATCCCCTGACGACCCTTGACGGAACTGGCCGATTTGCTAAAATATTTTTGATG
CTTACTTAGTCATCTGGTTGGCAAACCTCCGCGGACCTTCGGGACCAATGGAGAGGAACCCAGCCGAGAAGCATC
GAGCCGGTAAATGCCGGAAATTTTTGAGTCAGGAGAAGCTAACTTCTTCAAACTCAGGCCCTTGGAGCCGGTCA
(SEQ ID NO: 131)
>3300016590|Ga0186947_1003_42|M
CTGTCCTACTGTTTTGACTTACCGATGTGTTACTCCAATACGGGCGGACTGAAGTCCGCCCTCCAGGTGATAATG
ATGGAGTTTACGTCTGCGTCACATAAGGACTTGTGACGCCAAATAGCTGTGATTCTACATCACGACGGCAGCCGG
AATGCCGCCGCTCCCATGTTGTAATTCACTGGTGTTTGTGATTGGAGCTCACGAATAAGAGGTGGGAGAATAAAA
AAAACTGCCACTTGCGGACAGGTGGCAGAACAAAATTCTCCGAAACGCATGGGTTAAGGCTACCTGCCTGAGGGA
ACAGAGA (SEQ ID NO: 132)
>3300019846|Ga0197078_1002_15|M
AGTTTTGACTGGAAAATCTCCCTTTGGACGTTTGACCTAGGGGAGGGAACCCCGTCTGAAGATGGATAATTACCG
TCAAAATTTGGATAAACTTGGAGAAAATTAGACAAAAGAGTCGGTTTTTTTGATGTTACTGATCGGCGTAACTGA
TGTAACTGTCGTTAGATACAGCGAGTCG (SEQ ID NO: 133)
>UOQF01000587_2|M
AATAAGAGGAAACAATCCTTTCAATGTGAGTTTAGTGACTGTAATTATAAAACCCATAGCGATGAGAATGCGGCC
CGTGTCCTCGGACGGGTATTTTGGGGTGAAATTACCCTCCCCACCGATTGAGTGTTGAAAATTCTTGACATATAG
GCTAATTTAAGATATGTATAATTAGTCGCTTCGTGGTCCATCTGGCCGGAAAAACCTCTGGCGGGCGCAAGCACC
AATAGAGAGGAACTCGGCCCAGAAGCGAAGAAACCAATAATTAGGGGTTTAATTATTGAAGGAACTTTAAAGATT
TTGCGAC (SEQ ID NO: 134)

### Example 3: Comprehensive and Scalable In Vivo Screening of Synthetic CRISPR-Cas Systems

To comprehensively evaluate the functionality of the computationally identified type V systems, we added several key attributes to the E. coli-based in vivo negative selection screen of synthetic CRISPR-Cas systems previously described for the discovery of the type VI-D subtype (Yan et al., 2018). In particular, these key features were the inclusion of non-coding sequences into the synthetic CRISPR-Cas screening system, targeting of spacers sequences derived from both pACYC184 plasmid and E. coli essential genes in the screening library of minimal CRISPR arrays, and finally, the inclusion of unique molecular identifiers (UMIs) on screening constructs. Together, this enabled a greater ability to systematically and quantitatively interrogate CRISPR-Cas systems.

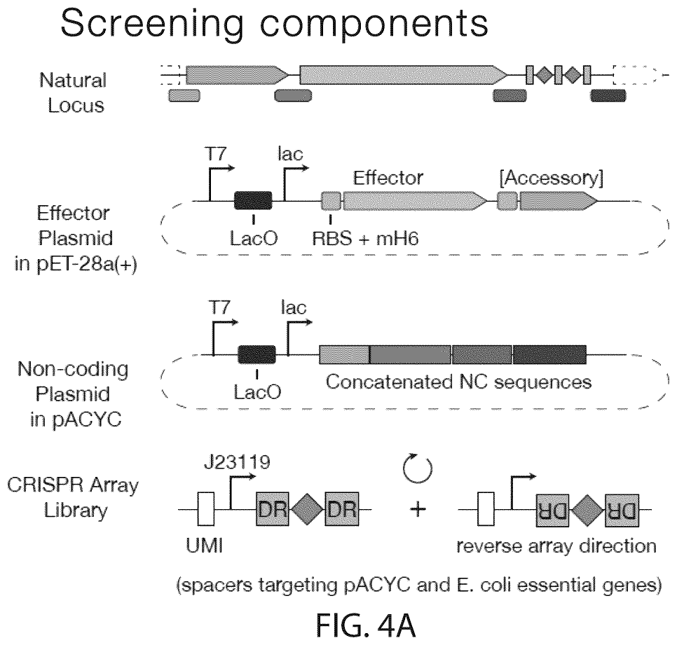

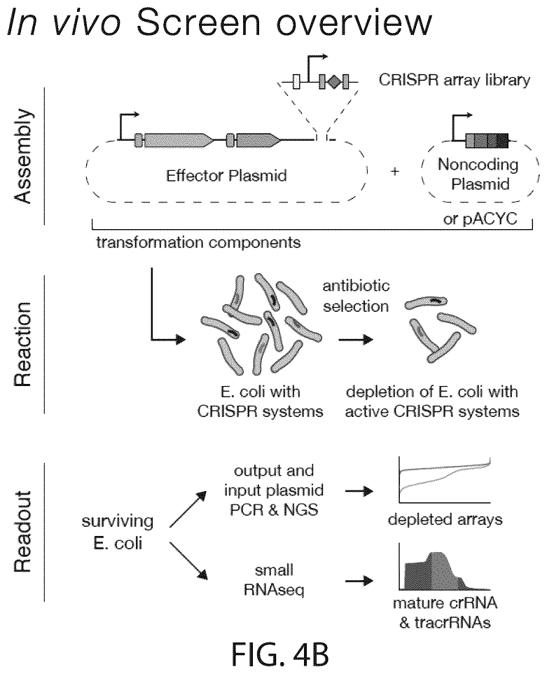

As an overview of the in vivo negative selection screen, we first synthesized a plasmid containing E. coli codon-optimized sequences encoding putative effectors and accessory proteins under the control of lac and IPTG-inducible T7 promoters (Effector Plasmid, FIG. 4A). To capture the required non-coding sequences for each system, such as the tracrRNA, we concatenated non-coding sequences flanking the CRISPR array, putative effector or accessory open reading frames, and predicted anti-repeats indicative of tracrRNA elements. Non-coding sequences were cloned into pACYC184 and expressed by lac and IPTG-inducible T7 promoters (Non-coding Plasmid, FIG. 4A). For the minimal CRISPR array, we designed oligonucleotide library synthesis (OLS) pools comprising two direct repeats flanking natural-length spacer sequences targeting the pACYC184 plasmid, select E. coli essential genes, and non-targeting negative control spacers for a total of 8900 elements in the array library. These sequences were placed under the control of a J23119 promoter and cloned into the Effector Plasmid in both the forward and reverse orientations for a total library of ˜18,000 plasmid elements (FIG. 4A).

The CRISPR Effector Plasmid library and/or the separate Non-coding Plasmid were then co-transformed into electro-competent E. coli, followed by 11 hours of outgrowth. During outgrowth, RNA-programmable CRISPR-Cas interference would result in loss of plasmid-based antibiotic resistance or self-targeting of essential genes, leading to depletion of bacteria containing functioning CRISPR-Cas systems. Next generation sequencing of CRISPR array elements in the Effector Plasmid library and comparison of the frequency of individual elements in the pre-transformation and post-outgrowth samples were used to identify strongly depleted CRISPR arrays conferring interference activity. Simultaneous small RNA sequencing of the surviving cells enabled the identification of required non-coding elements for activity, such as the mature crRNA and tracrRNA (FIG. 4B). The pipeline for systematic discovery, reconstruction, and evaluation of the different components of putative CRISPR-Cas systems is a generalizable method for the efficient and comprehensive search for novel CRISPR-Cas systems with in vivo interference activity. Using this approach, we identified robust activity for the subtype V-G discovered in this work.

### Gene Synthesis and Oligonucleotide Library Cloning

The E. coli codon-optimized protein sequences for CRISPR effector[?] accessory proteins were cloned into pET-28a (+) (EMD-Millipore) to create the Effector Plasmid. Noncoding sequences flanking Cas genes (including 150 nt of terminal CDS coding sequence) or the CRISPR array were synthesized (Genscript) into pACYC184 (New England Biolabs) to create the Non-coding Plasmid (FIG. 4A). Effector mutants (e.g., D513A or A513D) plasmids were cloned by site directed mutagenesis using the indicated primers in the sequence table: sequence changes were first introduced into PCR fragments, which were then re-assembled into a plasmid using NEBuilder HiFi DNA Assembly Master Mix or NEB Gibson Assembly Master Mix (New England Biolabs) following the manufacturer's instructions.

For the pooled spacer library, we first computationally designed an oligonucleotide library synthesis (OLS) pool (Agilent) to express a minimal CRISPR array of “repeat-spacer-repeat” sequences. The “repeat” elements were derived from the consensus direct repeat sequence found in the CRISPR array associated with the effector, and “spacer” represents ˜8,900 sequences targeting the pACYC184 plasmid and E. coli essential genes, or negative control non-targeting sequences. The spacer length was determined by the mode of the spacer lengths found in the endogenous CRISPR array. Flanking the minimal CRISPR array were unique PCR priming sites that enabled amplification of a specific library from a larger pool of oligo synthesis.

We next cloned the minimal CRISPR array library into the Effector Plasmid to create an Effector Plasmid library. We appended flanking restriction sites, a unique molecular identifier, and a J23119 promoter for array expression onto the oligo library using PCR (NEBNext High-Fidelity 2×PCR Master Mix), and then used NEB Golden Gate Assembly Master Mix (New England Biolabs) to assemble the full plasmid library of effectors with their targeting arrays. This represented the “input library” for the screen.

In Vivo E. coli Screen

The in vivo screen was performed as previously described (Yan et al., Mol Cell. 2018 Apr. 19; 70(2):327-339) with the following modifications: we performed the screen using electrocompetent E. cloni EXPRESS® BL21(DE3) E. coli cells (Lucigen) unless otherwise indicated. Competent cells were co-transformed with the Effector Plasmid and/or Non-coding (FIG. 4B). The cells were electroporated with the “input library” according to the manufacturer's protocols using a Gene Pulser Xcell (Bio-rad) with a 1.0 mm cuvette. The cells were plated onto bioassay plates containing both Chloramphenicol (Fisher) and Kanamycin (Alfa Aesar), and grown for 11 hours, after which we estimated the approximate colony count to ensure sufficient library representation and harvested the cells.

Plasmid DNA fractions were extracted from the harvested cells to create the ‘output library’ using a QIAprep Spin Miniprep Kit (Qiagen), while total RNA≥17 nt was harvested by lysing the harvested cells in Direct-zol (Zymo Research), followed by extraction using the Direct-zol RNA miniprep kit (Zymo Research).

To identify specific parameters resulting in enzymatic activity and bacterial cell death, we used next generation sequencing (NGS) to quantify and compare the representation of individual CRISPR arrays (i.e., repeat-spacer-repeat) in the PCR product of the input and output plasmid libraries. We defined the fold depletion for each CRISPR array as the normalized input read count divided by the normalized output read count (with 1 added to avoid division by zero). An array was considered to be “strongly depleted” if the fold depletion was greater than 3. When calculating the array fold depletion across biological replicates, we took the maximum fold depletion value for a given CRISPR array across all experiments (i.e. a strongly depleted array must be strongly depleted in all biological replicates). We generated a matrix including array fold depletion and the following features for each spacer target: target strand, transcript targeting, ORI targeting, target sequence motifs, flanking sequence motifs, and target secondary structure. We investigated the degree to which different features in this matrix explained target depletion for Type V-G systems, thereby yielding a broad survey of functional parameters within a single screen. Furthermore, the RNA fractions were prepared for small RNA library preparation and sequencing as described previously (Yan et al., 2018).

### Example 4. Transcription-Dependent Interference Activity of Cas12g

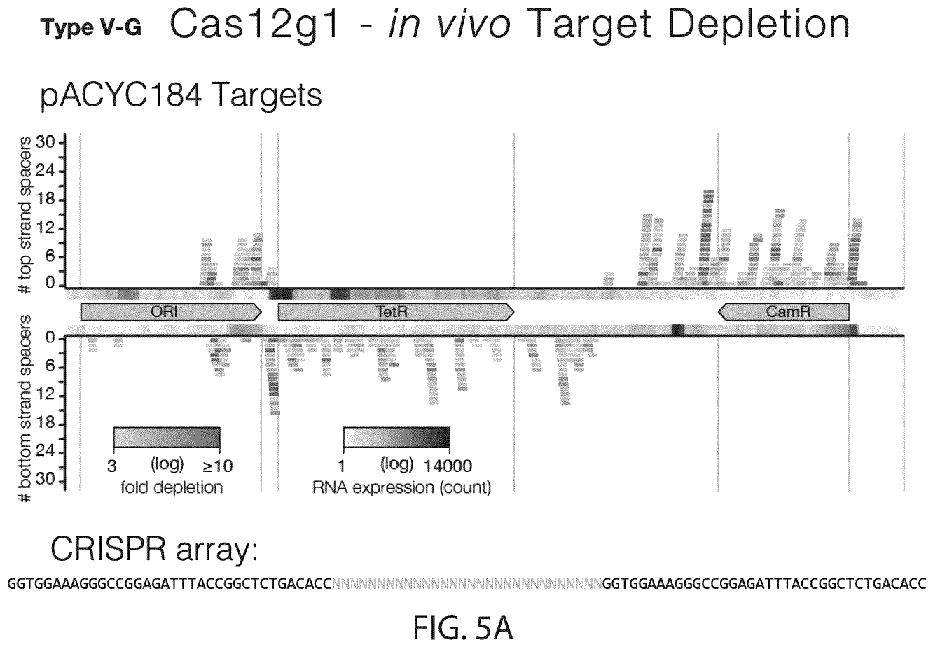

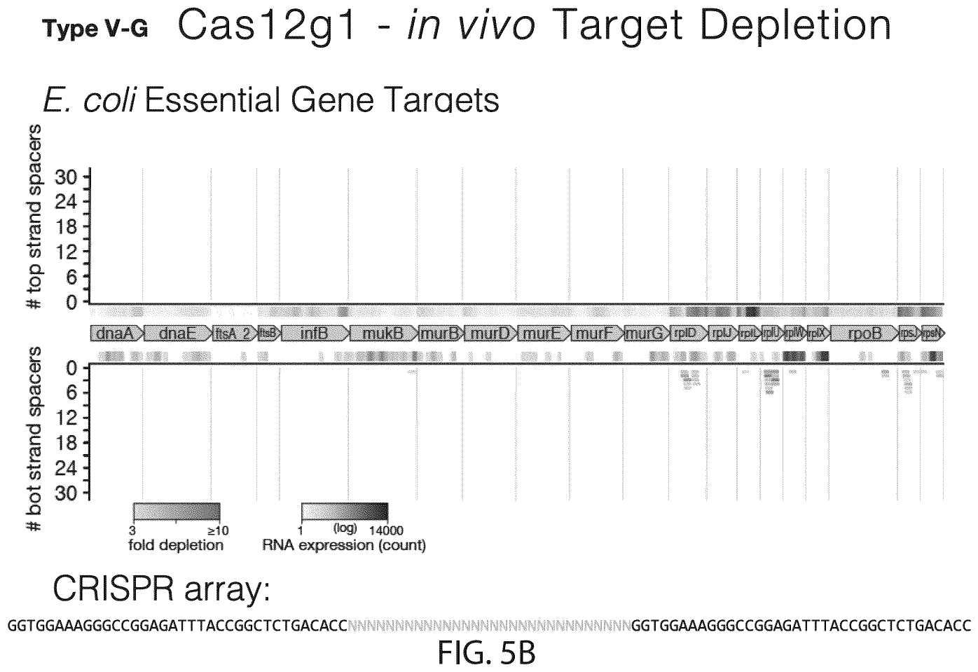

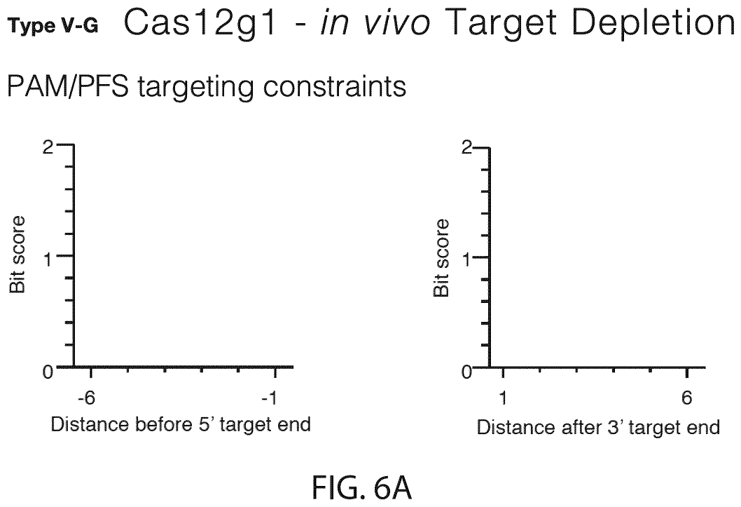

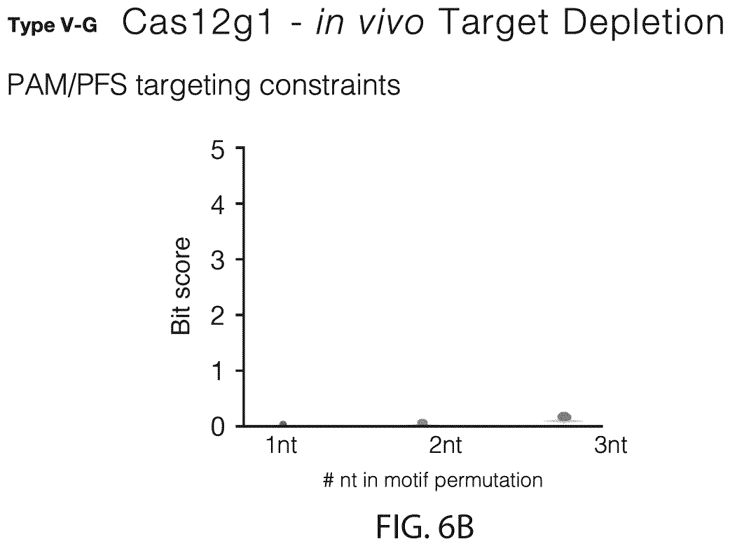

We first investigated the function of the CRISPR-Cas subtype V-G effector, Cas12g, which ranges in size from 696 aa-853 aa. Our initial subtype V-G in vivo screening system, including an Cas12g1 Effector Plasmid library and Non-coding Plasmid, displayed interference activity that specifically targeted the non-template, sense DNA strand of actively transcribed regions of pACYC184 (FIG. 5A) or E. coli essential gene substrates (FIG. 5B). This pattern of interference activity is strikingly similar to that observed for the RNA-targeting subtype VI-D CRISPR-Cas system when evaluated using a comparable screening strategy (Yan et al., 2018). Additional analysis of target flanking sequences of strongly depleted CRISPR arrays revealed no protospacer adjacent motif (PAM) or protospacer flanking sequence (PFS) requirements for Cas12g1 interference (FIG. 6A-B).

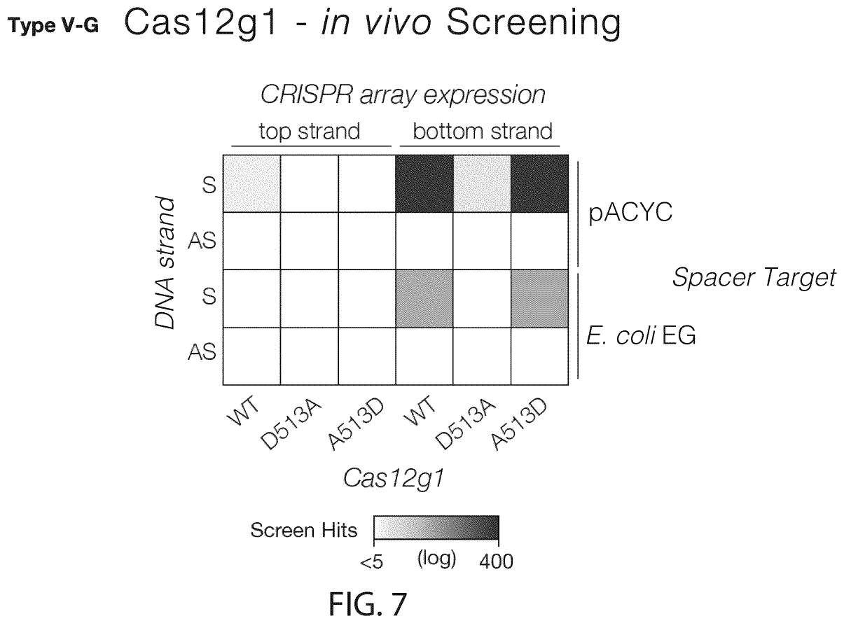

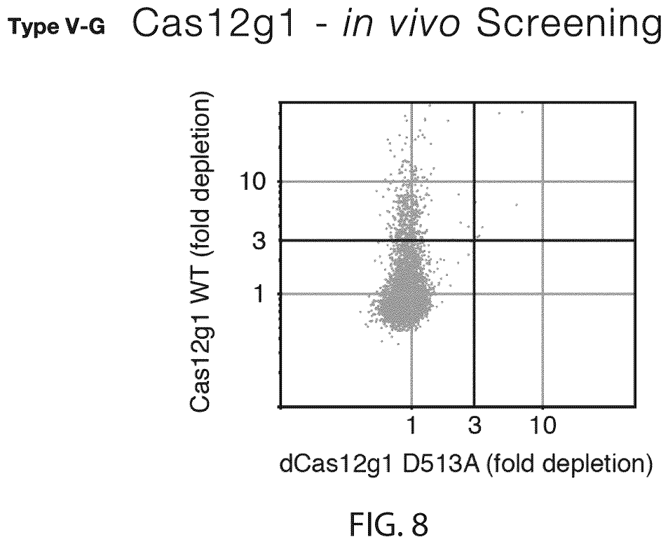

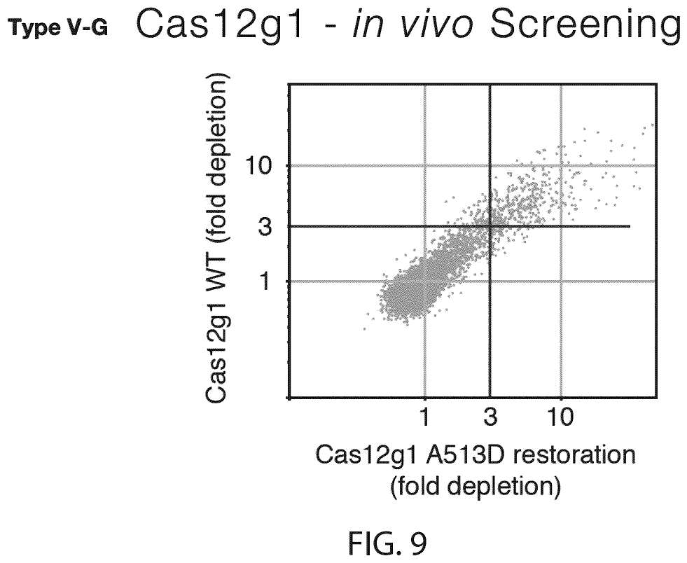

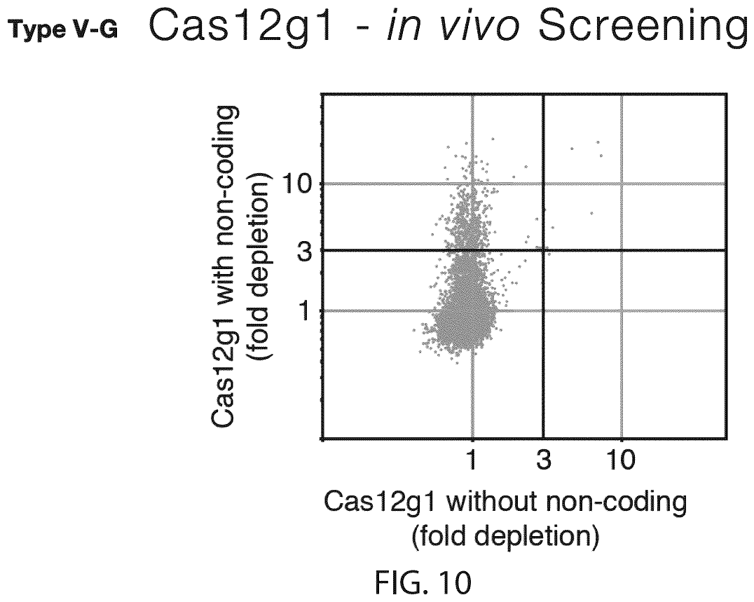

This pattern of interference implies either transcription-dependent DNA targeting or RNA targeting, which would be surprising given that Cas12g contains a highly conserved C-terminal RuvC nuclease domain (FIG. 7) previously known to cleave only DNA. To test the requirement of the RuvC domain for this unexpected pattern of effector targeting by Cas12g, we mutated the conserved aspartate residue in the RuvC-I motif of Cas12g1 to alanine (D513A) and found that interference was abolished (FIG. 7, FIG. 8). Subsequent reversion of the mutated dCas12g1 (D513A) Effector Plasmid to the Cas12g1 wild-type sequence restored the original interference pattern, demonstrating the requirement of an intact RuvC domain for Cas12g1 in vivo interference (FIG. 7, FIG. 9). In an effort to identify the minimal in vivo screening system producing the observed interference pattern, we removed the Non-coding Plasmid and observed a substantial decrease in activity, indicative of a tracrRNA requirement for the RuvC-dependent Cas12g1 interference (FIG. 7, FIG. 10).

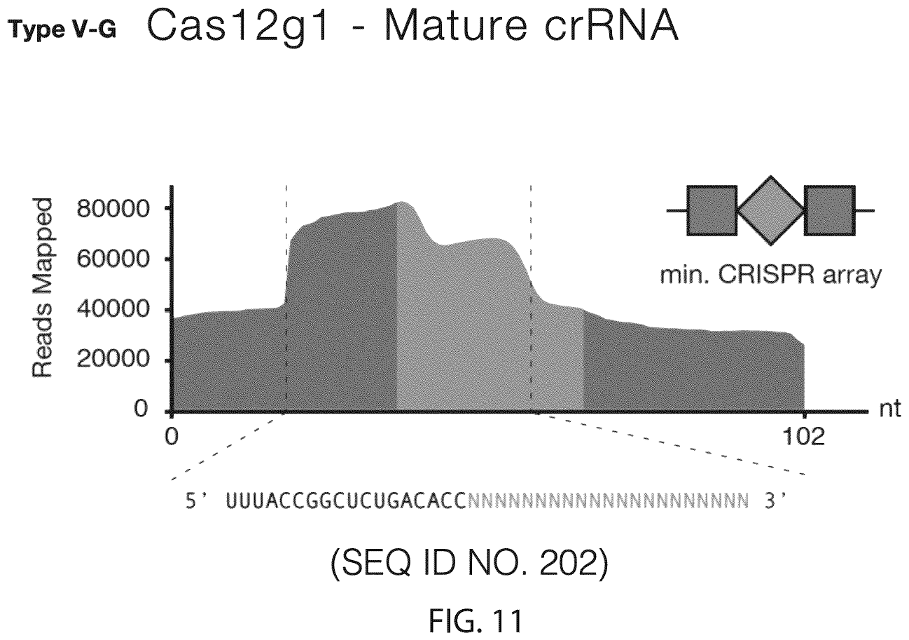

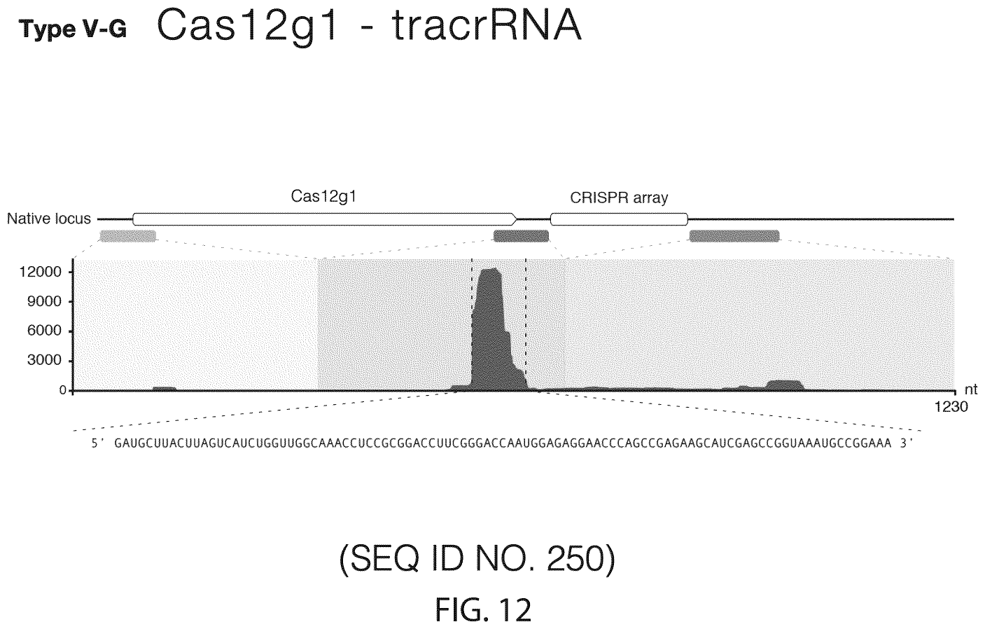

RNA sequencing of samples from in vivo screens containing the Non-coding Plasmid revealed a highly expressed tracrRNA element in the non-coding sequence of the Cas12g1 locus containing an anti-repeat with 11 nt of complementarity to the direct repeat (FIG. 11-13). Alignment of the RNA sequencing reads to the CRISPR array library showed that the type V-G pre-crRNA is processed in vivo, yielding a distribution of spacer lengths ranging from 18-23 nt and a 5′ direct repeat that is truncated 18-nt from the terminus (FIG. 11).

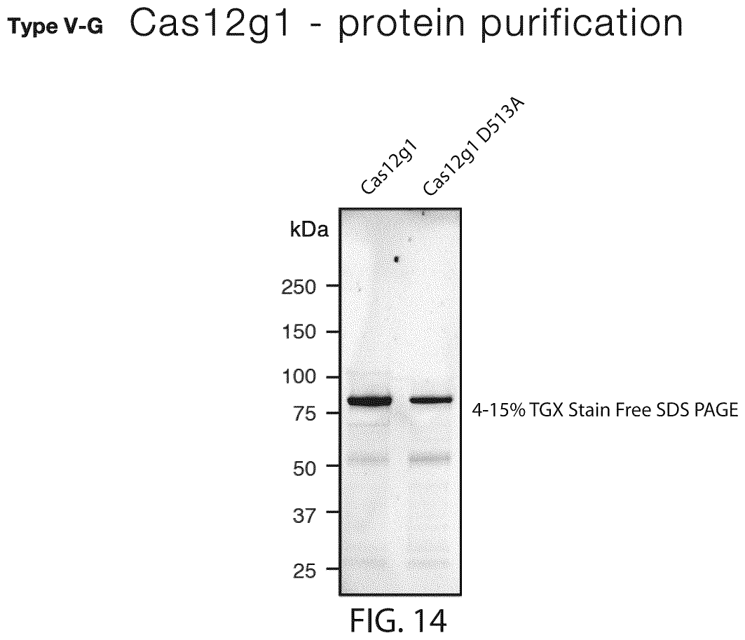

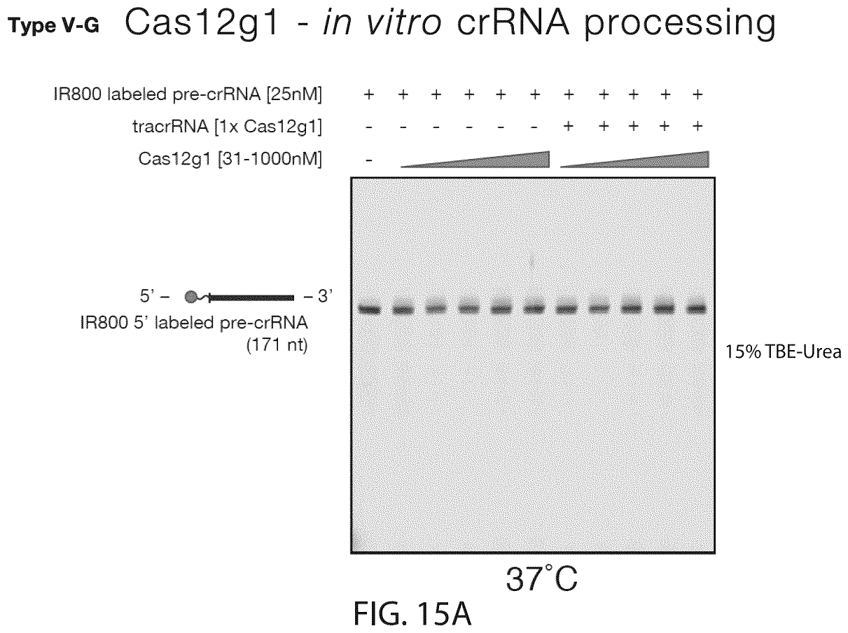

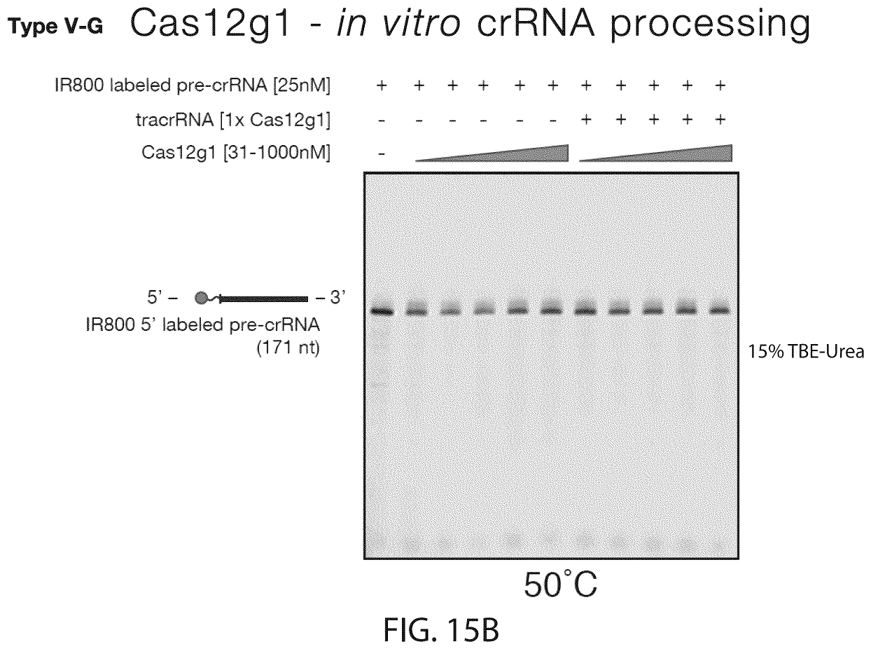

Having identified a putative minimal type V-G CRISPR-Cas system, we purified a recombinant stock of the Cas12g1 effector (FIG. 14) and in vitro transcribed the mature crRNA and tracrRNA to reconstitute the complete system for in vitro analysis of the interference mechanism. We first explored the biogenesis of the activated CRISPR complex. Incubation of Cas12g1 with a pre-crRNA containing a DR-spacer-DR-spacer-DR structure resulted in no observable pre-crRNA processing by Cas12g1, in the presence or absence of the tracrRNA (FIG. 15A-B, TABLE 8-9). Given that mature crRNA and tracrRNA were identified by RNA sequencing of the Cas12g1 system expressed in vivo, these findings suggest that biogenesis of the subtype V-G crRNA requires an endogenous nuclease(s) absent from our in vitro reaction.

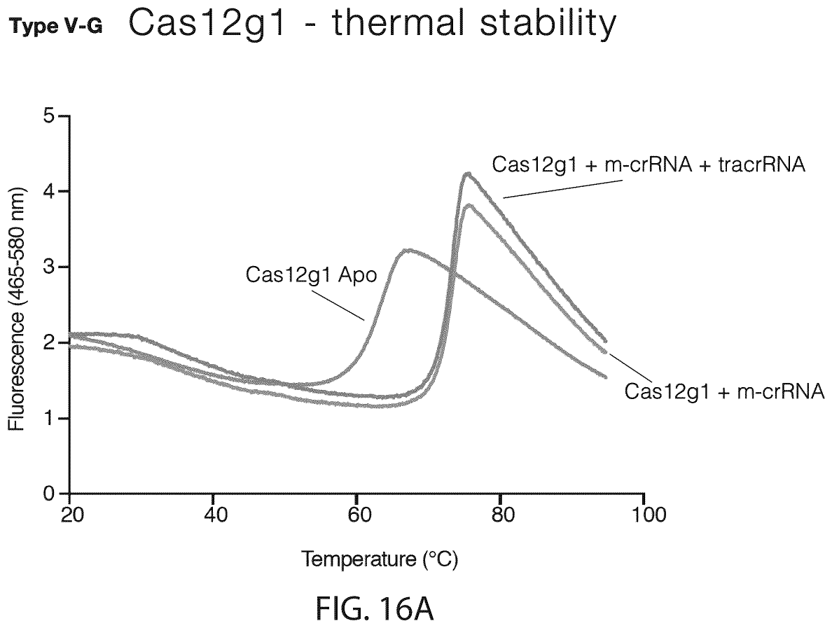

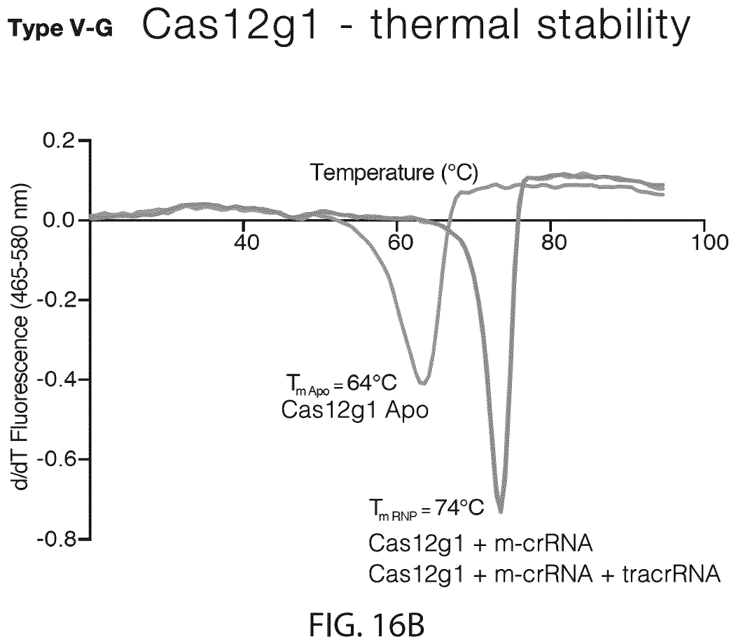

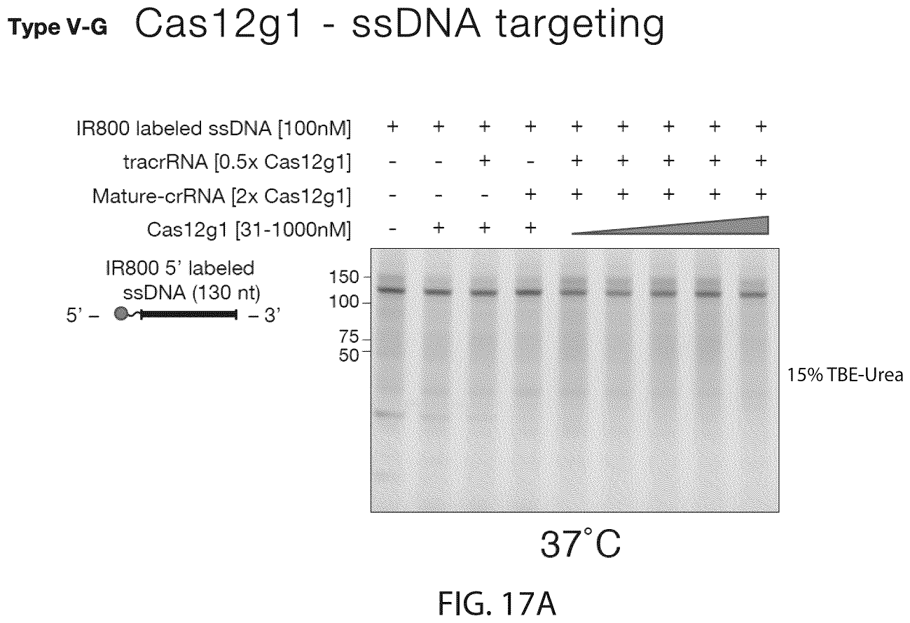

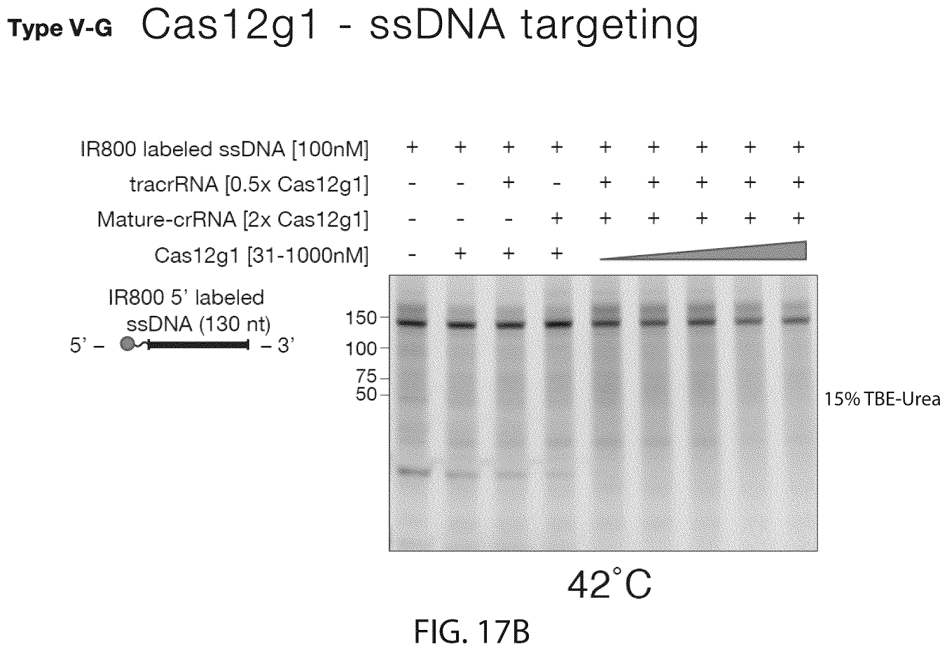

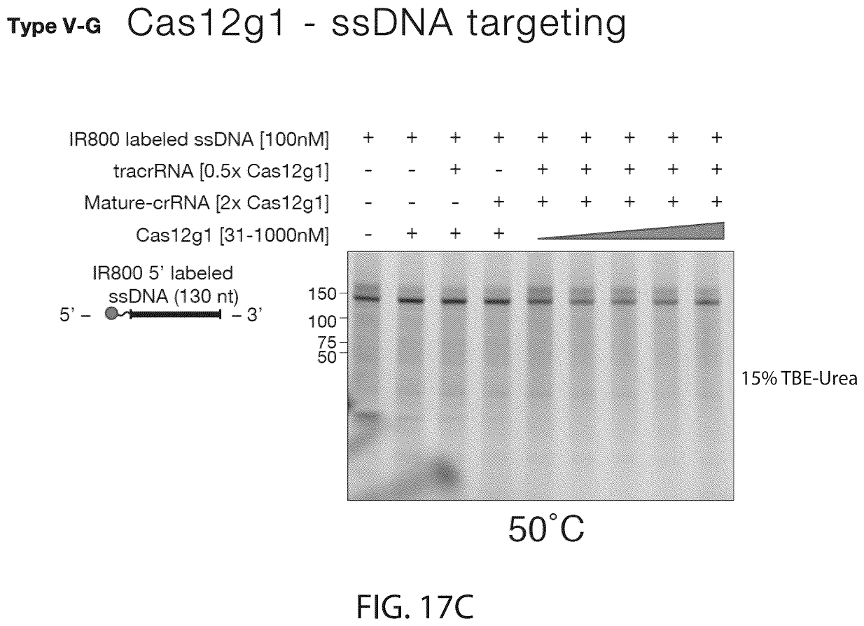

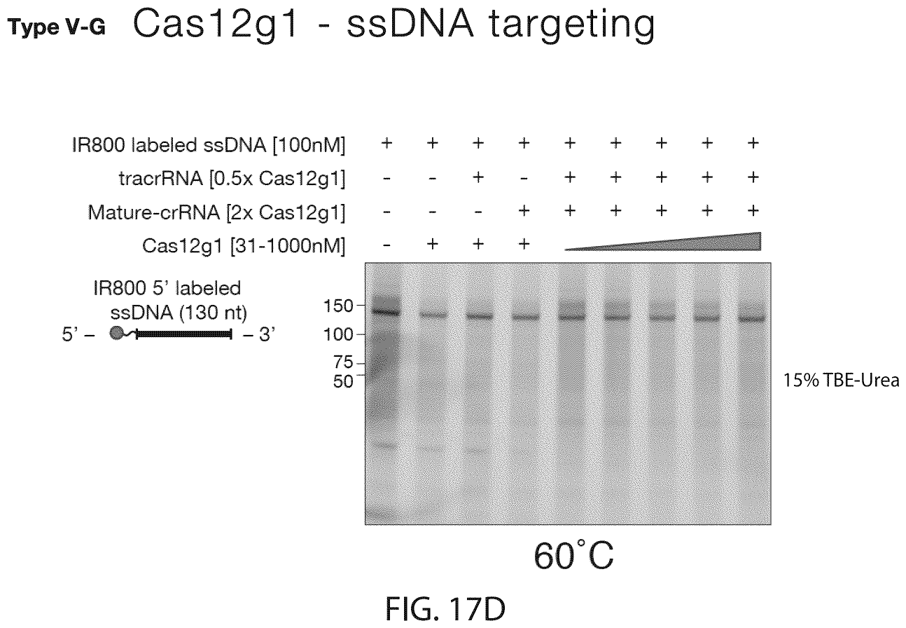

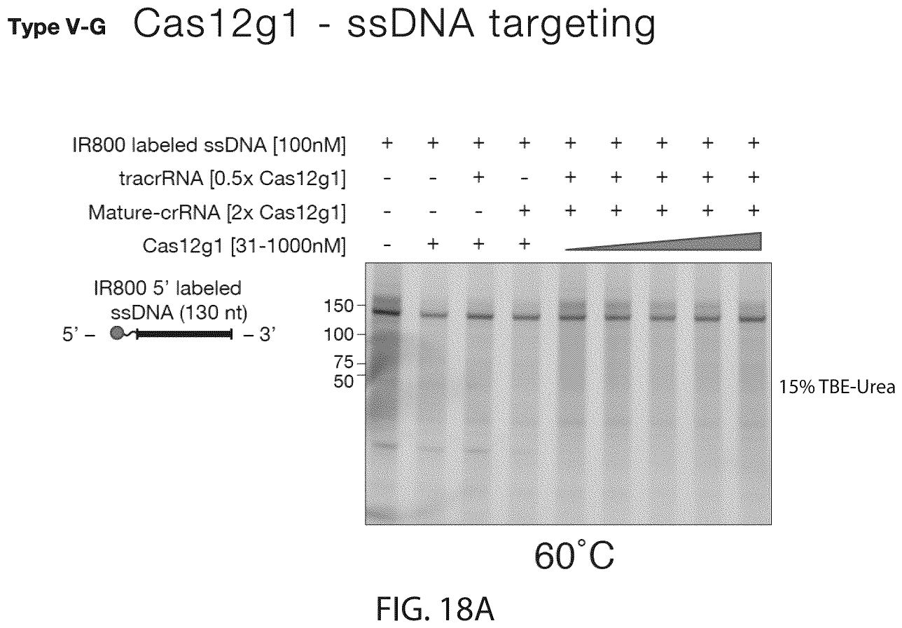

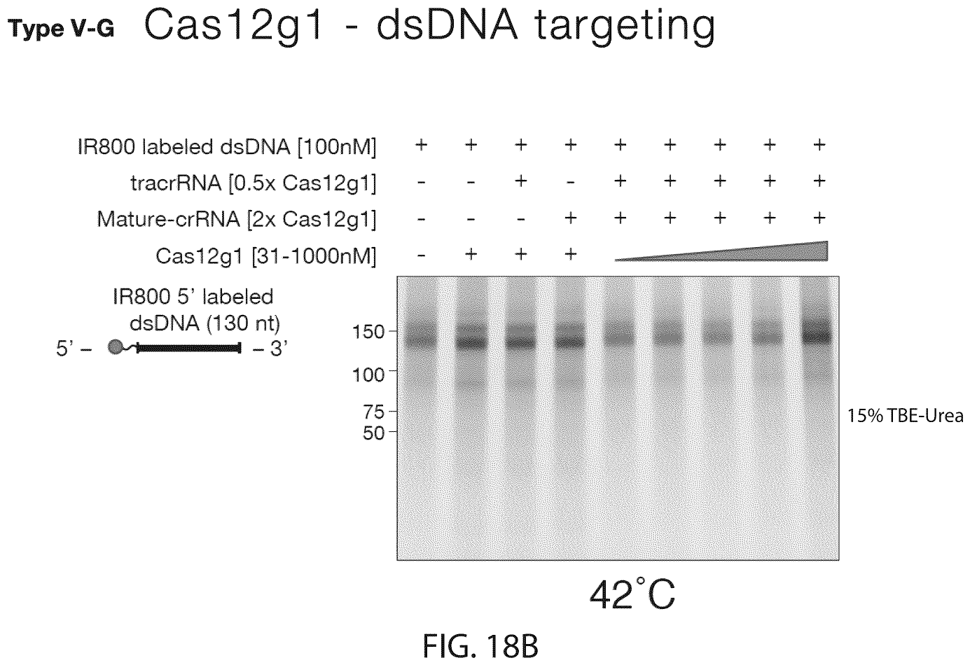

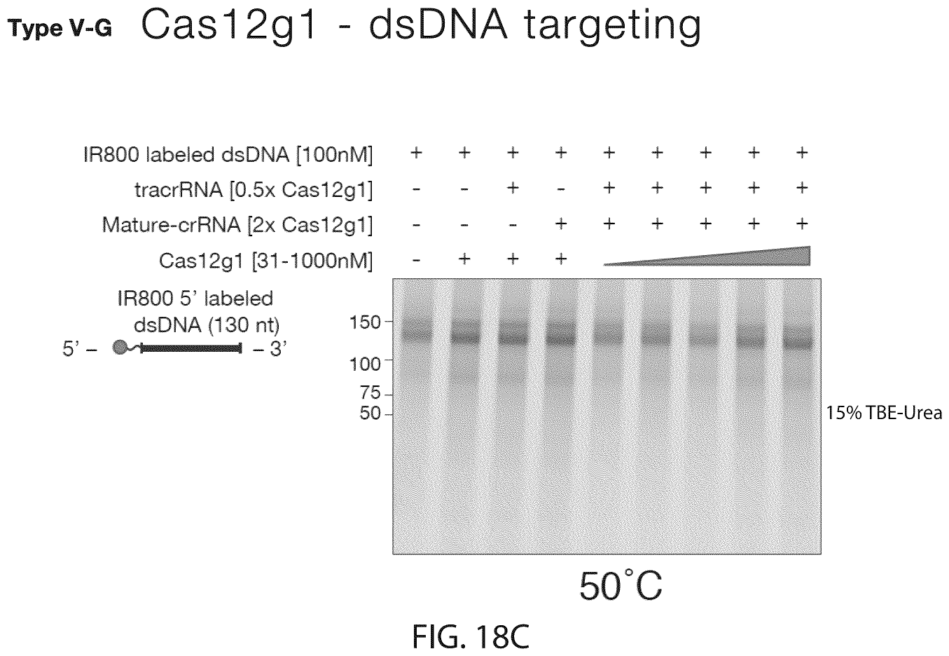

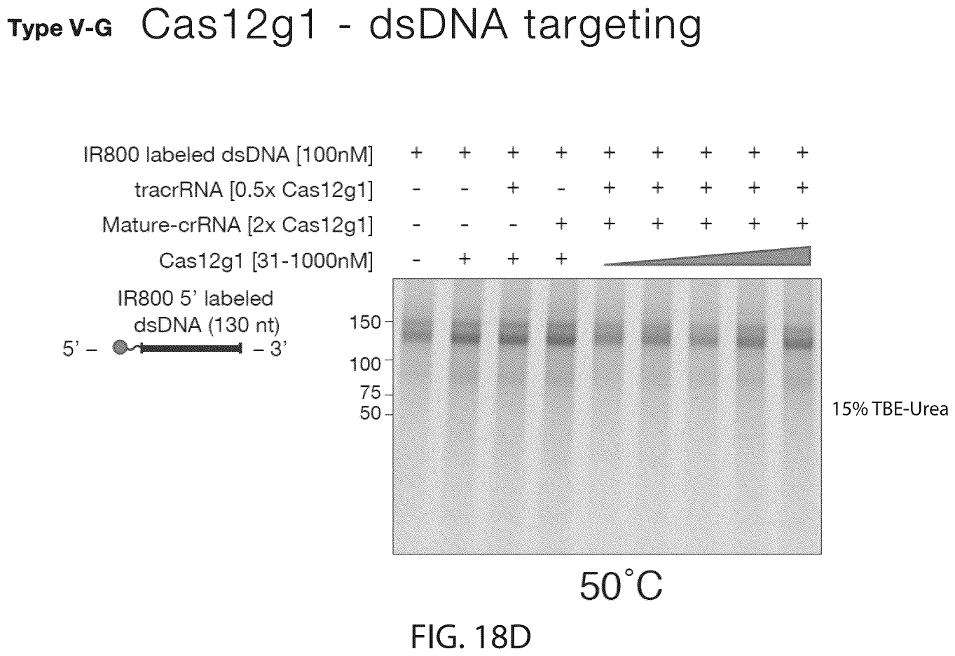

To explore the mechanism of Cas12g interference further, we first assessed the cleavage of ssDNA and dsDNA in the presence of the mature crRNA and tracrRNA. Cas12g1 surveillance complexes were constructed containing the Cas12g1 effector, tracrRNA, and mature crRNAs derived from highly depleted CRISPR arrays from the in vivo screen. Cas12g1 surveillance complexes showed no cleavage of the cognate ssDNA or dsDNA substrates at 37° C. (FIG. 17A, FIG. 18A, and TABLES 9-11). Given that the Cas12g1 locus originates from a hot spring metagenome, we used a thermal melt assay to assess the stability of this protein and its complexes with RNA at temperatures above 37° C. The thermal melt results show that, in the absence of crRNA or tracrRNA, Cas12g1 is thermostable with a melting temperature (Tₘ) of 64° C. (FIGS. 16A-B). Furthermore, when complexed with the crRNA and tracrRNA, the Cas12g1 surveillance complex showed increased thermostability with a Tₘ of 74° C. (FIGS. 16A-B). However, despite this, we observed no ssDNA or dsDNA cleavage by the Cas12g1 surveillance complex at 42° C., 50° C., or 60° C. (FIGS. 17B-D, FIGS. 18B-D).

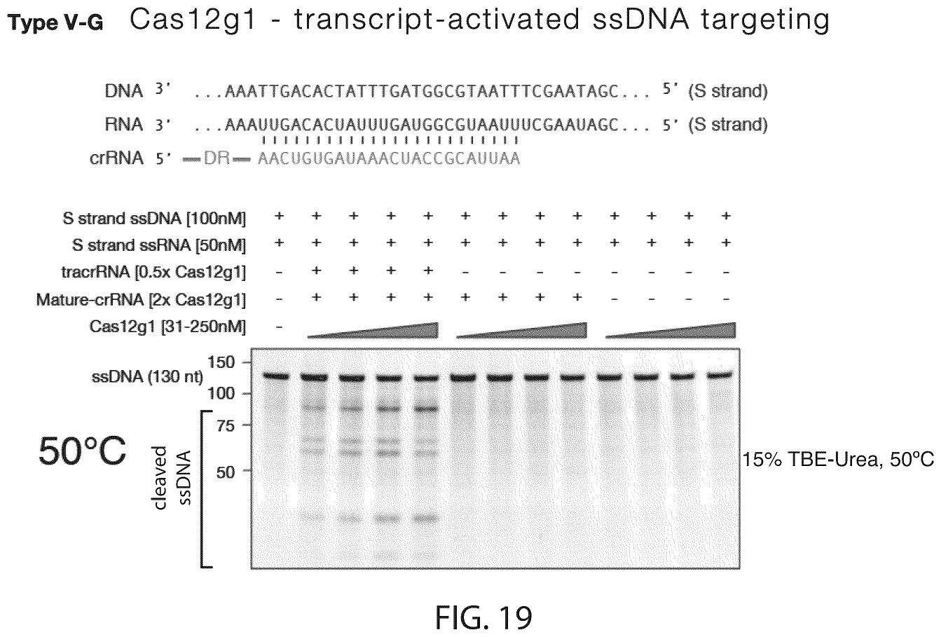

Given the observed strong bias of in vivo interference by Cas12g towards targeting the non-template DNA strand, we studied the effect of RNA transcripts on Cas12g1 effector activity. To this end, ssDNA and ssRNA substrates were generated with either sense (containing crRNA spacer-commentary target) or antisense sequences. We tested the cleavage of ssDNA for each of the four substrate combinations (sense ssDNA: sense ssRNA; sense ssDNA: antisense ssRNA; antisense ssDNA: sense ssRNA; antisense ssDNA: antisense ssRNA). For the sense-antisense pairings of ssDNA and ssRNA, the substrates were pre-annealed, creating a DNA-RNA hybrid. We observed that, at 50° C., the Cas12g1 surveillance complex cleaves sense ssDNA in the presence of a sense RNA transcript in an efficient and dose-dependent manner (FIG. 19).

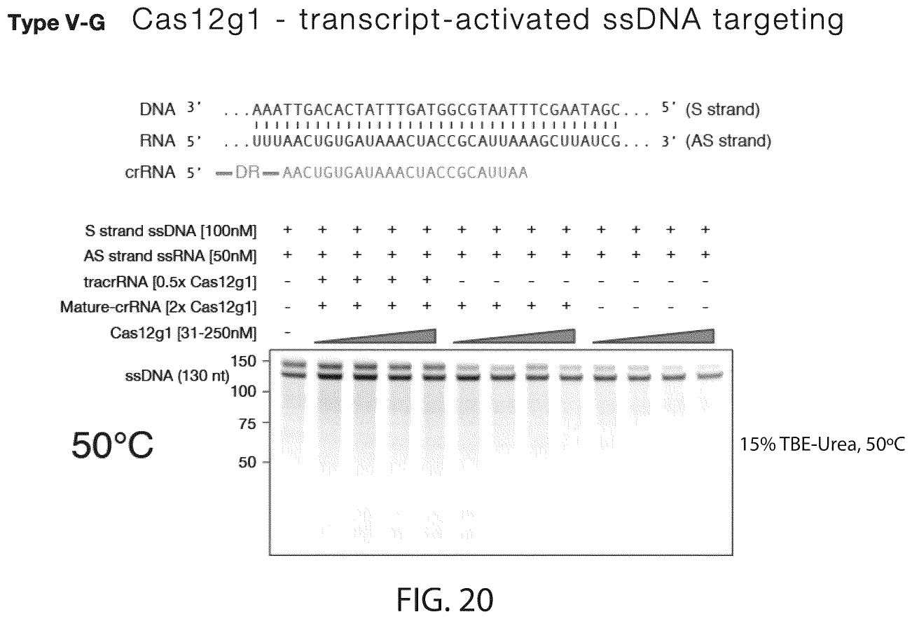

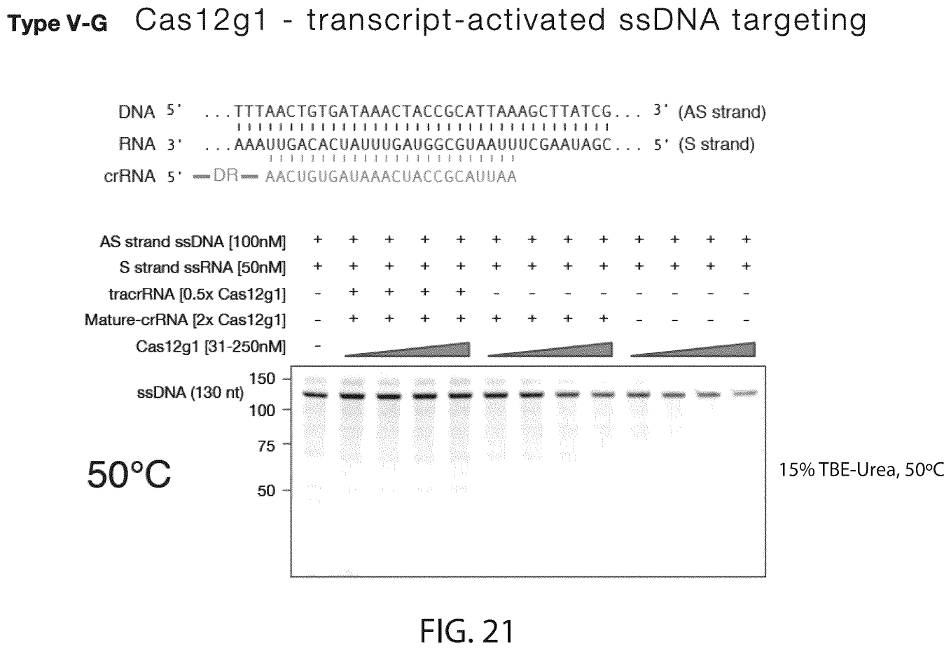

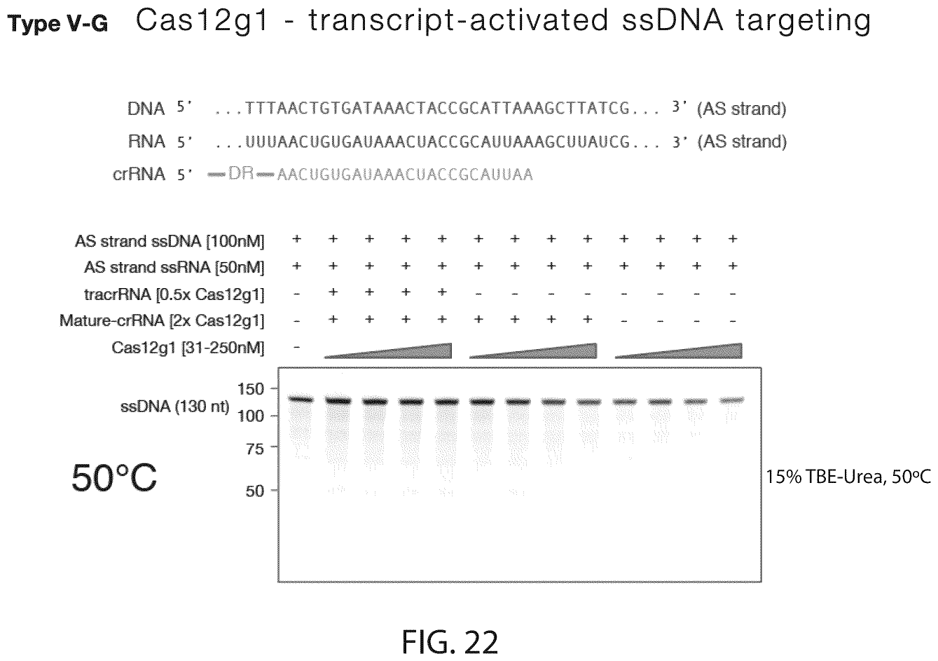

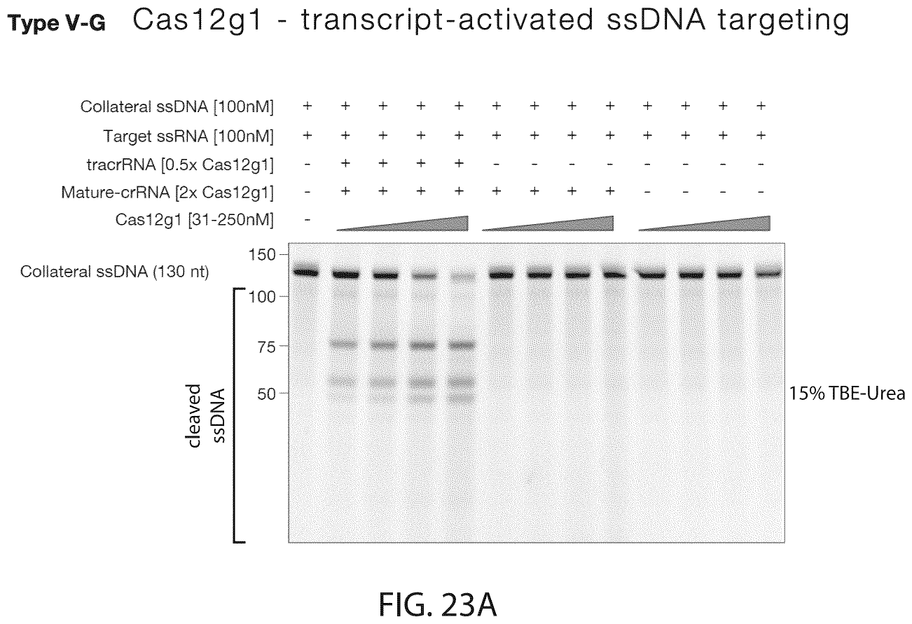

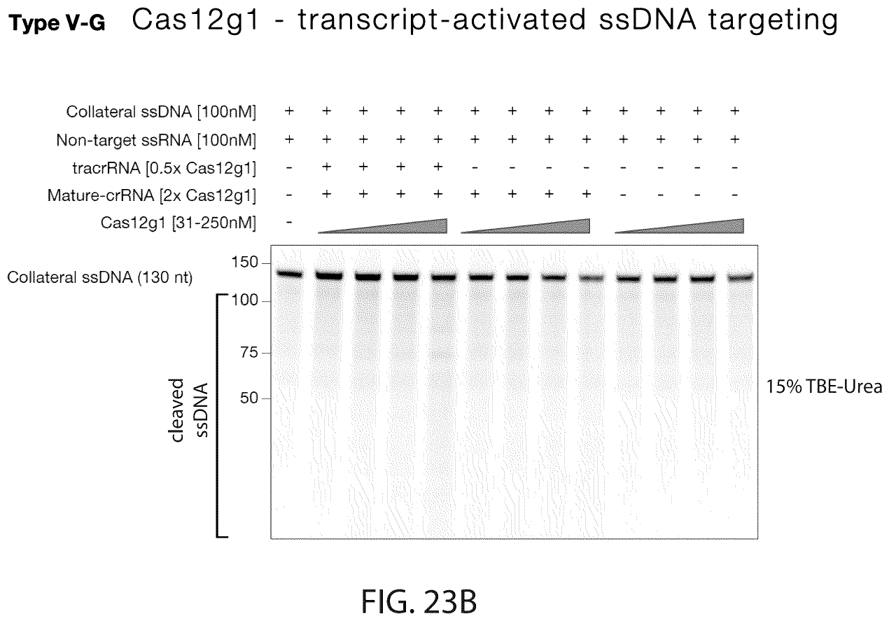

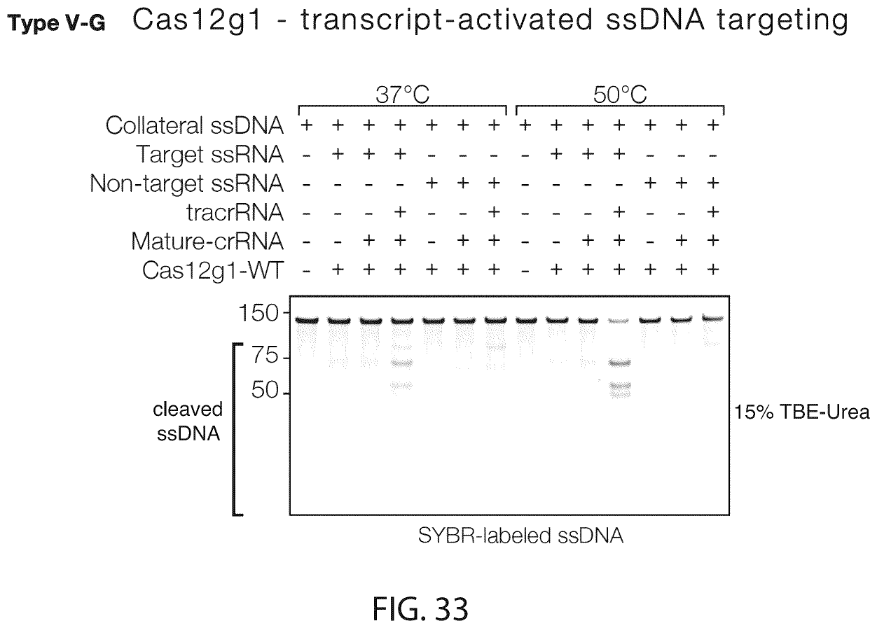

We then investigated whether this interference activity depended on the target sequence present in the ssDNA substrate and found that ssDNA containing no target sequence or complementarity to the crRNA spacer (“collateral ssDNA”) was efficiently cleaved into shorter fragments at 50° C. in the presence of a sense ssRNA with crRNA spacer complementarity (FIGS. 23A-B, FIG. 33). No ssDNA cleavage was observed for any other DNA-RNA substrate combination (FIGS. 20-22). These results demonstrate that ssDNA cleavage by Cas12g1 surveillance complex is activated by an RNA transcript containing a target complementary to the crRNA spacer, hereafter referred to as the target RNA. Cleavage of ssDNA into multiple products of decreasing size by the target RNA-activated Cas12g1 complex is indicative of non-specific collateral (trans) ssDNA cleavage activity.

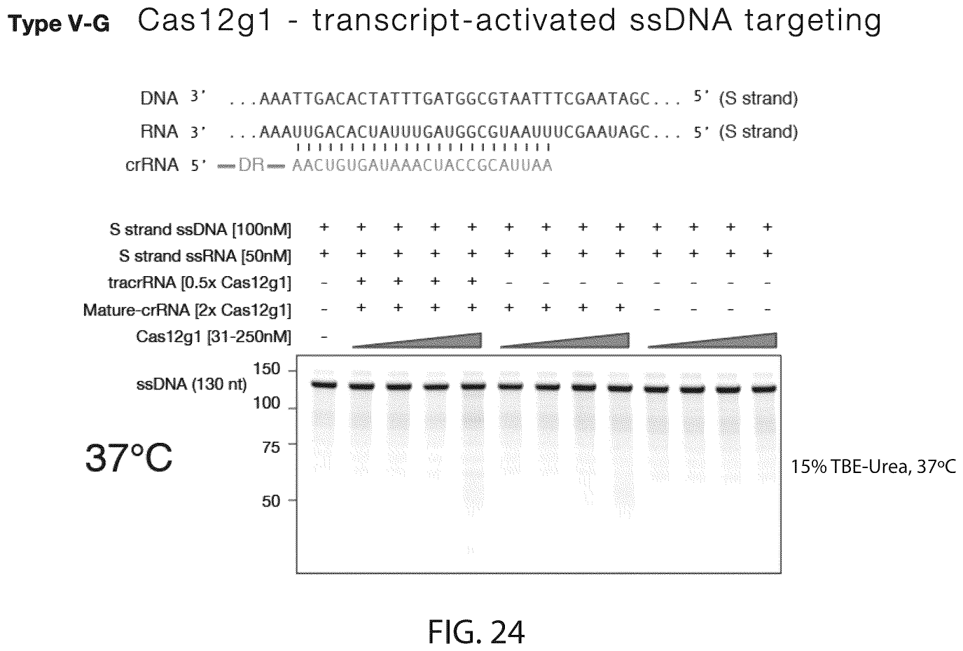

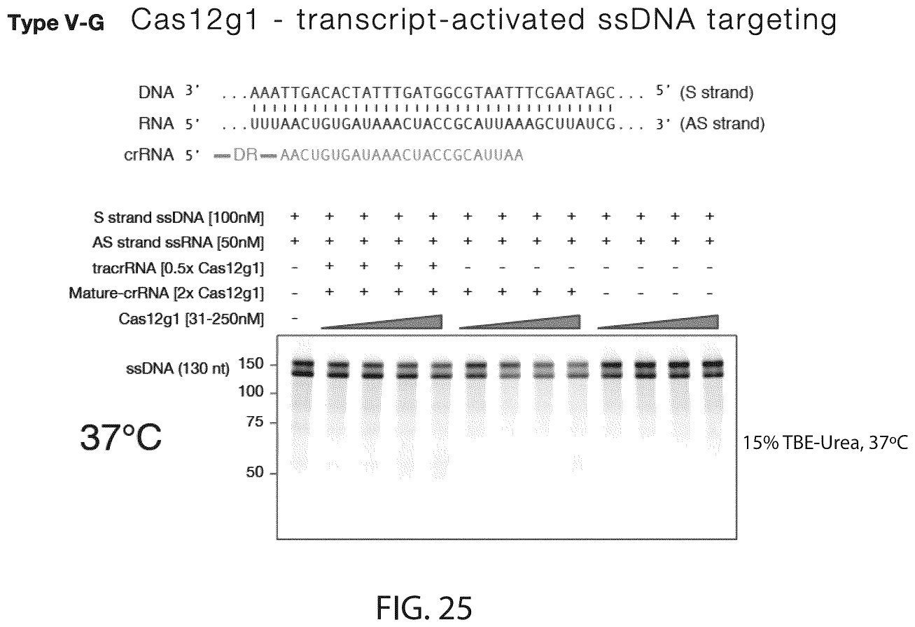

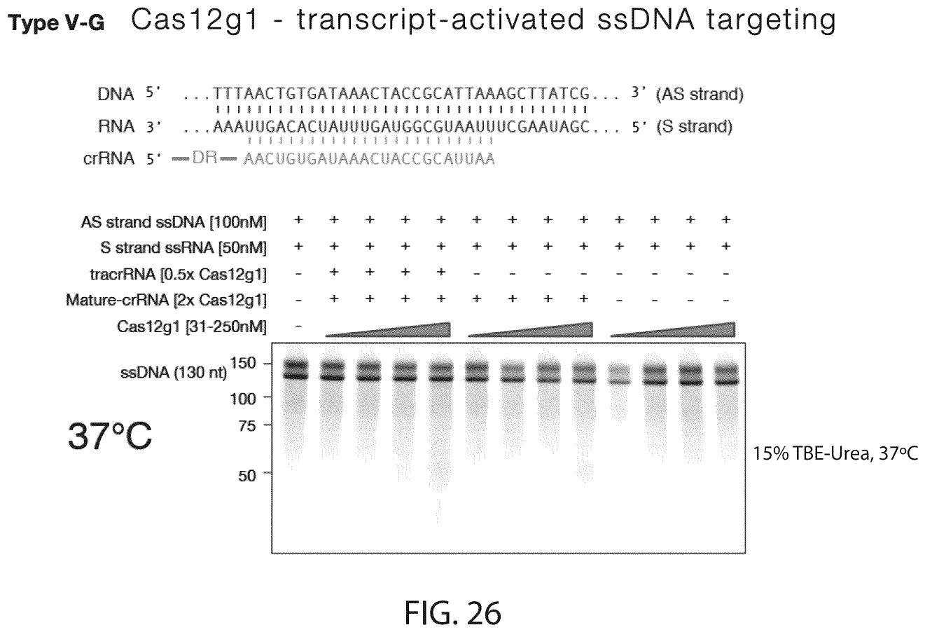

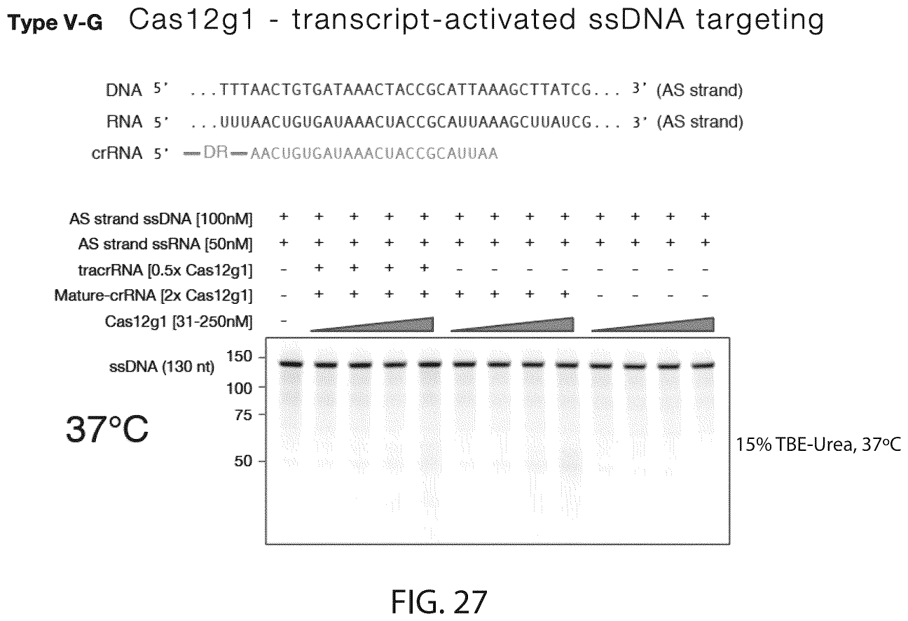

Cas12g1 target RNA activated collateral ssDNA cleavage presents a potential mechanism underlying in vivo interference observed for the subtype V-G CRISPR system, whereby binding of a target RNA transcript would result in cleavage of unprotected ssDNA strands at the transcriptional fork. However, target RNA-dependent ssDNA cleavage by the Cas12g1 surveillance complex was not evident at 37° C. (FIGS. 24-27). The lack of robust in vitro ssDNA cleavage by the Cas12g1 surveillance complex in the presence of a target RNA transcript at 37° C. is at odds with the strong interference observed in the in vivo screens performed at the same temperature, suggesting that the collateral ssDNA cleavage is likely not the only mode of interference for the subtype V-G system.

### Purification of Effector Proteins

Effector vectors were transformed into E. coli NiCo21 (DE3) (New England BioLabs) and expressed under a T7 promoter. Transformed cells were initially grown overnight in 3 mL Luria Broth (Sigma)+50 μg/mL kanamycin, followed by inoculation of 1 L of Terrific Broth media (Sigma)+50 μg/mL kanamycin with 1 mL of overnight culture. Cells were grown at 37° C. until an OD600 of 1-1.5, then protein expression was induced with 0.2 mM IPTG. Cultures were then grown at 20° C. for an additional 14-18 hours. Cultures were harvested and pelleted via centrifugation, then resuspended in 80 mL of lysis buffer (50 mM HEPES pH 7.6, 0.5M NaCl, 10 mM imidazole, 14 mM 2-mercaptoethanol, and 5% glycerol)+protease inhibitors (Sigma). Cells were lysed via cell disruptor (Constant System Limited), then centrifuged twice at 28,000×g for 20 minutes at 4 C in order to clarify the lysate. The lysate was loaded onto a 5 mL HisTrap® FF column (GE Life Sciences), then purified via FPLC (AKTA Pure, GE Life Sciences) over an imidazole gradient from 10 mM to 250 mM. Cas12g1 was purified in low salt buffer (50 mM HEPES-KOH PH 7.8, 500 mM KCl, 10 mM MgCl₂, 14 mM 2-mercaptoethanol, and 5% glycerol) and high salt buffer (50 mM HEPES-KOH PH 7.8, 1M NH₄Cl, 10 mM MgCl₂, 14 mM 2-mercaptoethanol, and 5% glycerol), respectively. After purification, fractions were run on SDS-PAGE gels and fractions containing protein of the appropriate size were pooled and concentrated using 10 kD Amicon Ultra-15 Centrifugal Units. Cas12g1 was further dialyzed into a buffer without imidazole (25 mM HEPES-KOH PH 7.8, 1 M NH₄Cl, 10 mM MgCl₂, 1 mM DTT, 7 mM 2-mercaptoethanol, and 30% glycerol). Protein concentration was determined by Qubit protein assay (Thermo Fisher).

crRNA, tracrRNA and Substrate RNA Preparation

Single-stranded DNA oligo templates for crRNA, tracrRNA, and substrate RNA were ordered from Integrated DNA Technologies, Inc. (Coralville, Iowa) (IDT). Substrate RNA and pre-crRNA templates were PCR amplified to generate a double stranded in vitro transcription (IVT) template DNA using NEBNEXT Hifi 2× master mix (New England Biolabs). Double stranded DNA templates for mature cr-RNA and tracrRNA were generated by annealing T7 primer with templates followed by extension using DNA Polymerase I, Large (Klenow) Fragment (New England Biolabs). Annealing was performed by incubating for 5 min at 95° C. followed by a −5° C./min ramp down to 4° C. In vitro transcription was performed by incubating the dsDNA templates with T7 RNA polymerase at 37° C. for 3 hours using HiScribe® T7 Quick High Yield RNA kit (New England Biolabs). After incubation, IVT samples were treated with Turbo DNase (Thermo Scientific) and then purified using RNA Clean & Concentrator kit (Zymo Research). For Cas12g1, equimolar amounts of mature cr-RNA containing 5′ hydroxyl and 5′ monophosphate end group were used. Mature cr-RNA generated from IVT was treated with Calf Intestinal Alkaline Phosphatase (Thermo Fisher) or RNA 5′-polyphoshpatase (Lucigen) for 2 hours at 37° C. to generate 5′-hydroxyl or 5′-monophosphate, respectively, followed by clean up with RNA Clean & Concentrator kit (Zymo Research). Concentrations were measured via Nanodrop 2000 (Thermo Fisher). Pre-crRNA, tracrRNA, and mature-crRNA sequences used in biochemical characterization of Cas12g is included in TABLE 8, TABLE 9, and TABLE 10, respectively. Oligonucleotide templates and primers for preparation of crRNAs are included in TABLE 14.

### Preparation of IR-800 Labeled Substrate RNA and DNA

RNA substrates from IVT were treated with Calf Intestinal Alkaline Phosphatase (Thermo Fisher) for 30 minutes at 37° C. to convert the 5′-triphosphate to 5′ terminal hydroxyl group and purified using RNA Clean & Concentrator kit (Zymo Research). A thiol end group was added to the 5′ terminal hydroxyl group of the DNA and RNA substrates via 5′ EndTag Labeling Kit (Vector Labs), then substrates were labeled with IRDye 800CW Maleimide (LI-COR Biosciences). Substrates were purified using DNA Clean & Concentrator kit or RNA Clean & Concentrator kit (Zymo Research). Labeled dsDNA substrates were generated by labeling the non-spacer complementary ssDNA strand, annealing with a primer, then extending with DNA Polymerase I, Large (Klenow) Fragment (New England Biolabs) for 15 minutes at 25° C. These substrates were purified with DNA Clean & Concentrator kit (Zymo Research). Concentrations were measured via Nanodrop 2000 (Thermo Fisher).

RNA and DNA substrate sequences used in biochemical characterization of Cas12g are included in TABLE 11-13. Oligonucleotide templates and primers for preparation of RNA substrates are included in TABLE 15.

Cas12g1 pre-cr-RNA Processing Assay

Pre-cr-RNA processing assays for Cas12g1 were performed at 37° C. for 1 hour in cleavage buffer at a final pre-cr-RNA concentration of 25 nM. For Cas12g, pre-crRNA processing was performed with 5′ IR800-labeled pre-crRNA to distinguish from the tracr-RNA. The tracrRNA was added at equimolar concentrations to Cas12g1. Reactions were quenched with the addition of 1 ug/uL of proteinase K (Ambion) and incubated at 37° C. for 15 minutes. 50 mM EDTA was added to the reactions before mixing with equal volume of 2×TBE-Urea sample buffer (Invitrogen) and denaturing at 65° C. for 3 minutes. Samples were analyzed on 15% TBE-Urea gels (Invitrogen). Gels were stained for 5 minutes with SYBR Gold nucleic acid stain (Invitrogen) and imaged on Gel Doc EZ (Biorad). Gels containing labeled pre-crRNA were first imaged on Odyssey CLx scanner (LI-COR Biosciences) prior to SYBR staining.

Target Cleavage Assays with Cas12g

ssRNA, ssDNA and dsDNA substrates: Cas12g target cleavage assays were performed for 1 hour at the specified temperature in cleavage buffer. Cas12g surveillance complex was formed by incubating a 1:2:0.5 molar ratio of Cas12g:mature crRNA:tracrRNA for 10 minutes at 37° C. followed by transfer to ice. Since mature cr-RNA generated from processing of pre-crRNA is expected to contain a mix of 5′ hydroxyl and monophosphate end groups, mature cr-RNA for the assay was prepared by mixing equimolar amounts of hydroxyl end group and monophosphate end group mature cr-RNAs. Cas12g surveillance complex was further diluted on ice keeping the molar ratio of protein to RNA fixed. The complex was then added to IR800 labeled substrates and incubated for 1 hour at the specified temperature. The final concentration of all labeled substrates was 100 nM. For collateral activity assays, the final concentration of both labeled and unlabeled substrates was 100 nM each unless otherwise specified. Reactions were treated with RNAse cocktail (Thermo Scientific) or Turbo DNase Thermo Scientific) to visualize DNA and RNA substrates respectively. Reactions were quenched by adding 1 ug/uL of proteinase K (Ambion) and incubating for 15 minutes at 37° C., followed by adding 50 mM EDTA. Samples were then mixed with equal volume of 2×TBE-Urea sample buffer and denatured at 65° C. for 3 minutes for RNA samples and 95° C. for 3 minutes for DNA samples.

Samples were analyzed on denaturing gel electrophoresis on 15% TBE-Urea gel (Invitrogen). Gels were first imaged on Odyssey CLx scanner (LI-COR Biosciences) followed by a 5 minute stain with SYBR Gold nucleic acid stain (Invitrogen) and imaged on Gel Doc EZ (Biorad). RNA:DNA hybrid substrates: Cleavage assays for RNA:DNA hybrid substrates were performed as described above using unlabeled RNA and 5′-IR800 labeled DNA. All RNA:DNA hybrid substrates were mixed at a 1:2 ratio of RNA:DNA and annealed in hybridization buffer (50 mM Tris pH 8.0 and 100 mM NaCl) by incubating at 70° C. for 2 minutes followed by a 5° C./min ramp down to 4° C. Final concentration of the labeled DNA was 100 nM. Reactions were treated with RNAse cocktail prior to the Proteinase K addition.

### Thermal Melt Assays for Cas12g

Melting curves of effector proteins were determined through differential scanning fluorimetry (DSF). All reactions took place at effector concentration of 0.62 uM in DSF buffer (50 mM Tris-HCl pH 7.9, 100 mM NaCl, 1 mM DTT) with the addition of 5×SYPRO orange dye (Sigma). Binary (effector+m-crRNA) and ternary (effector+m-crRNA+tracrRNA) complexes were formed at a 2.5:1 ratio of RNA to protein. Apo and effector complexes were incubated at 37° C. for 10 minutes prior to melting to assure complex formation. The reactions were then split into four 5 uL technical replicates. Protein melting was performed in the Lightcycler 480 (Roche) over a temperature range from 20° C. to 80° C., at a rate of 0.06° C./sec, with 10 acquisitions/sec. The first derivative of the raw fluorescence data was taken in order to determine the Tₘ of the effector.

### Example 5. Cas12g1 has a RuvC-Dependent ssRNase Activity

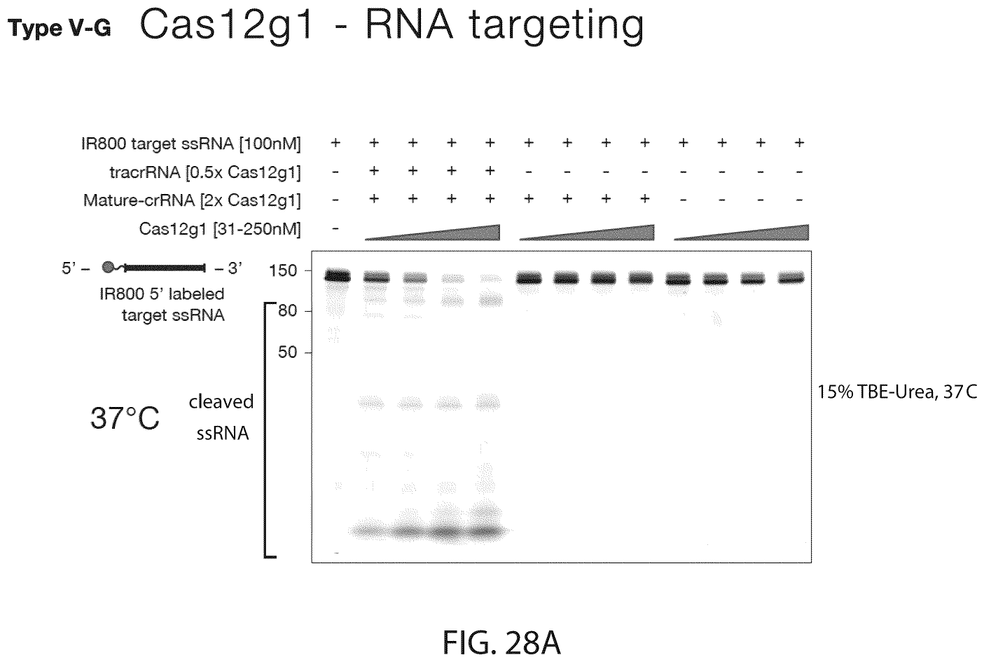

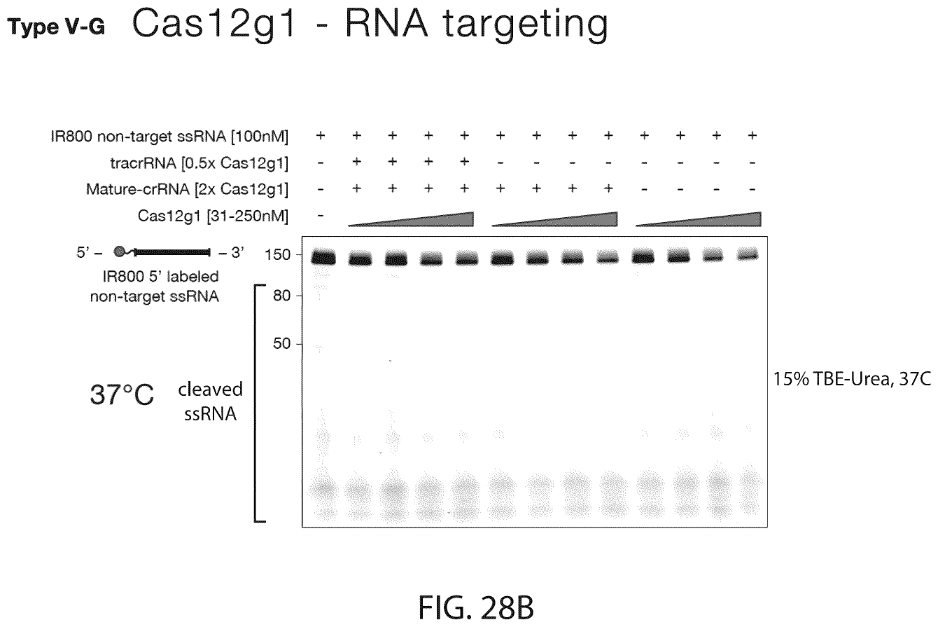

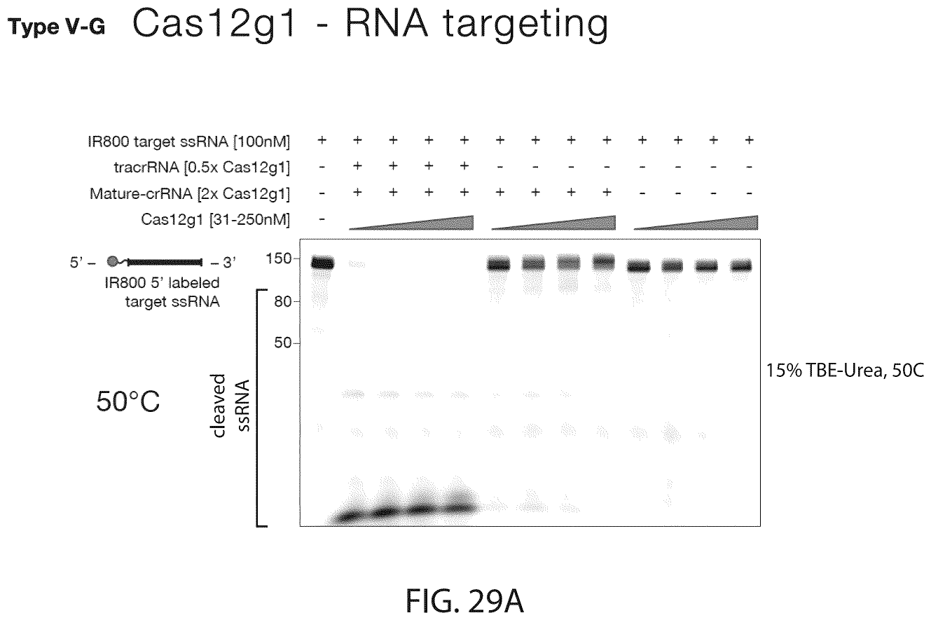

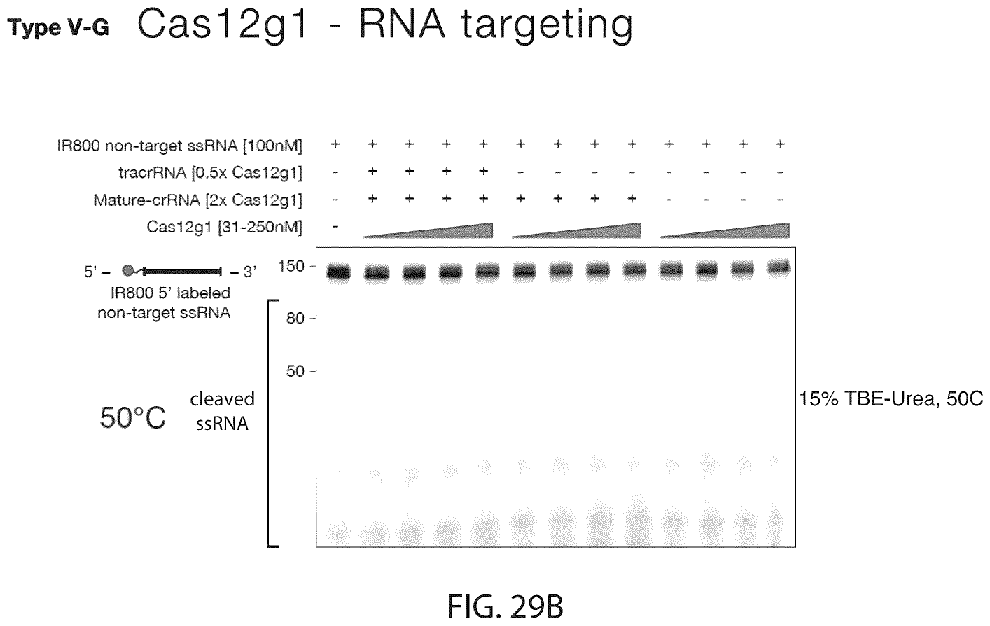

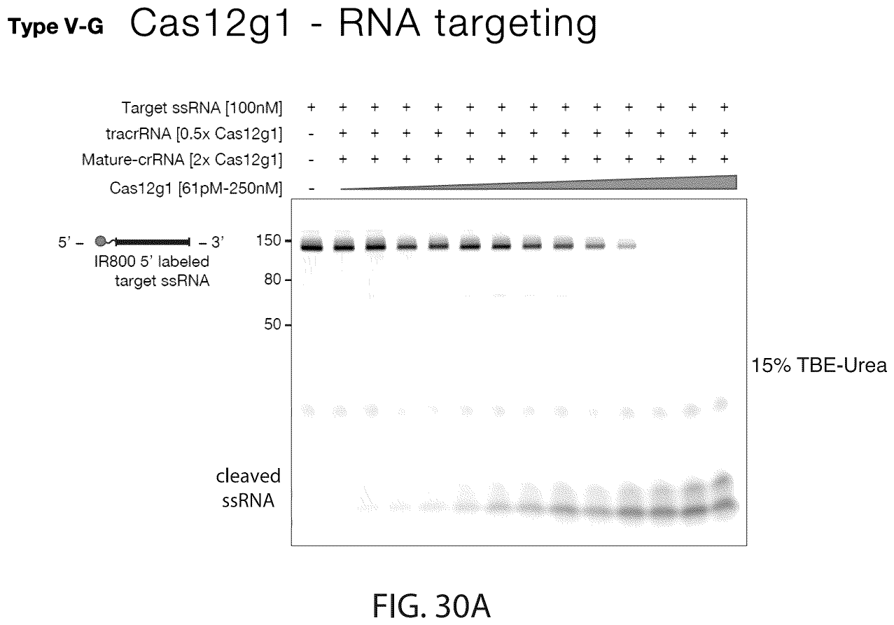

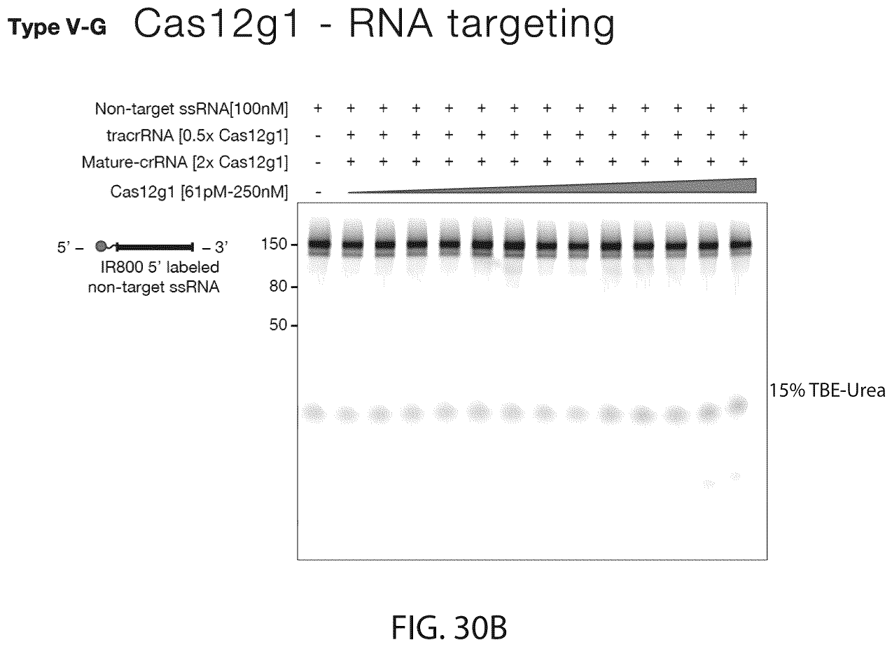

To assess additional Cas12g1 enzymatic activities that might enable the interference activity at 37° C., we tested whether, in addition to the ssDNA cleavage activation by the target RNA binding, the Cas12g1 surveillance complex cleaves RNA directly. We IR800 dye-labeled target RNA transcripts shown to activate Cas12g1 ssDNA cleavage and incubated these substrates with the Cas12g1 surveillance complex at 37° C. and 50° C. (FIG. 28A-B, FIG. 29A-B, TABLES 7-9). Under these conditions, Cas12g1 showed robust, target-specific and tracrRNA-dependent RNA cleavage at 37° C. (FIG. 28A-B). RNA cleavage by the Cas12g1 surveillance complex was further enhanced at 50° C., and we observed complete degradation of the target RNA substrate to single nucleotides at complex concentrations as low as 31 nM (FIG. 29A-B) and detectable cleavage at complex concentrations as low as 125 pM (FIG. 30A). Cas12g1 appears to be highly specific, and no cleavage of non-target RNA was observed with the highest dose of surveillance complex tested (250 nM), over 1000-fold higher than the concentrations sufficient to observe detectable cleavage of target-containing RNAs (FIG. 30B). The high efficiency of the target RNA cleavage at 37° C. suggests that transcript targeting is the primary source of the observed in vivo interference activity by the subtype V-G CRISPR Cas system.

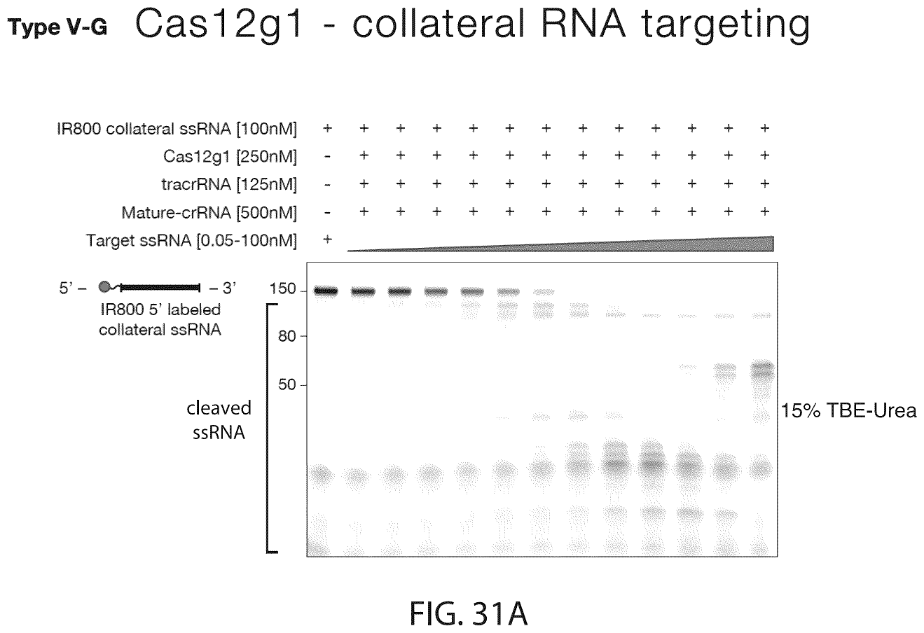

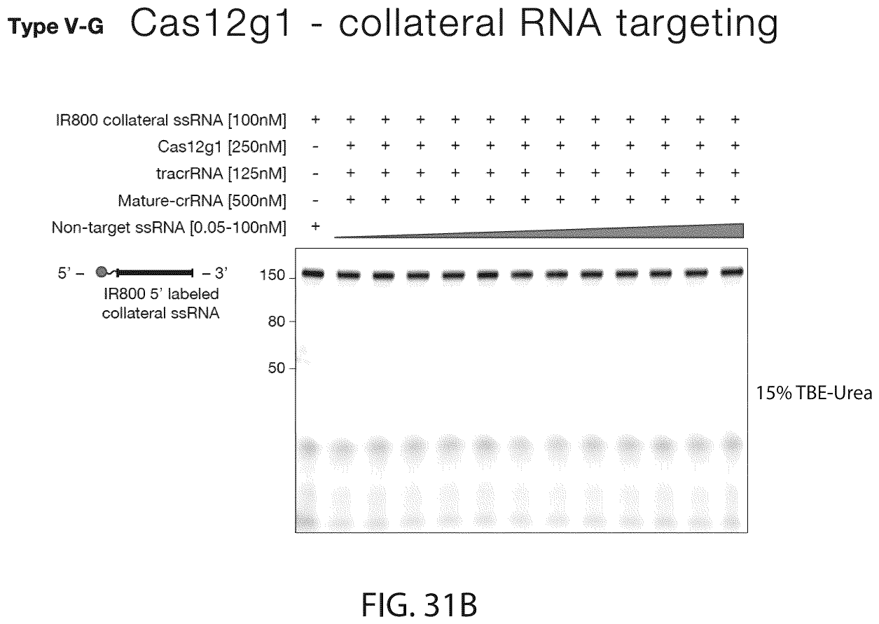

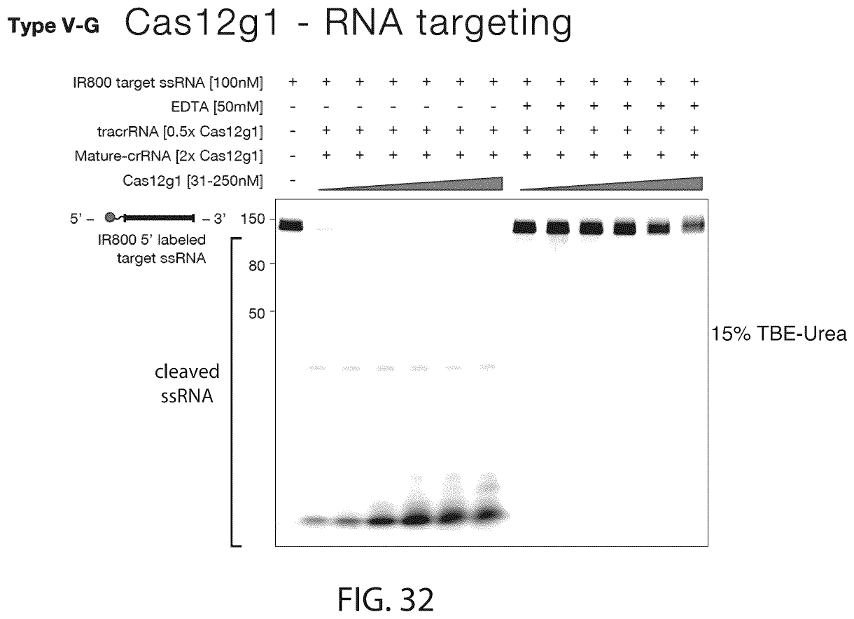

Having observed efficient cleavage of target transcripts to small fragments or single nucleotides, we next investigated whether the Cas12g RNA cleavage of target ssRNA substrates is accompanied by trans cleavage of collateral RNA transcripts containing no sequence similarity to the crRNA spacer. To distinguish the target and collateral RNA species, ssRNA cleavage reactions were prepared with IR800 dye-labeled collateral substrates mixed with unlabeled target transcripts. We observed robust collateral RNA cleavage by the Cas12g1 complex at 50° C. at concentrations of target RNA as low as 125 pM (FIG. 31A), demonstrating that the standalone RNA detection sensitivity of Cas12g1 is comparable to the highest performing Cas13 variants (Gootenberg et al., 2018). Finally, we observed that both target and collateral cleavage by the Cas12g1 is inhibited by an excess of EDTA, establishing the divalent cation dependency of RNA cleavage by Cas12g1 (FIG. 32).

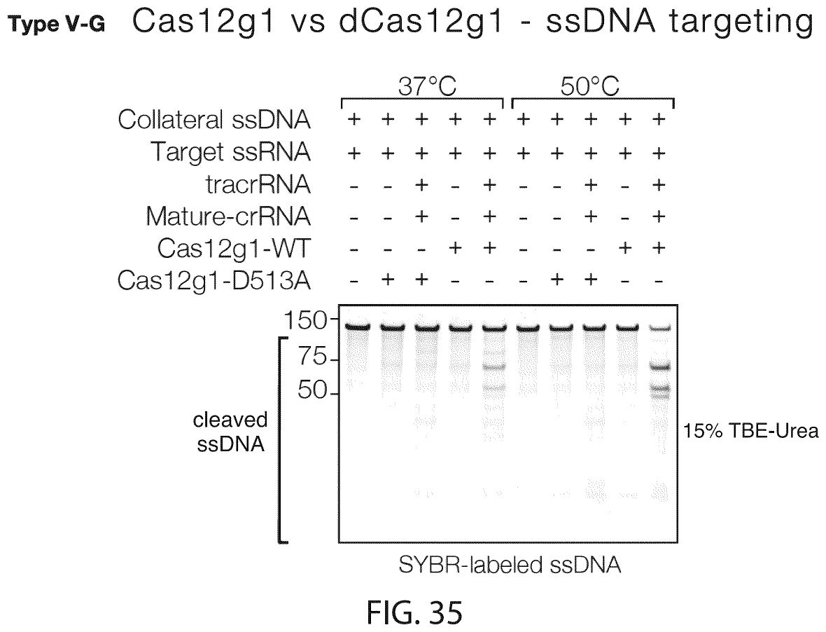

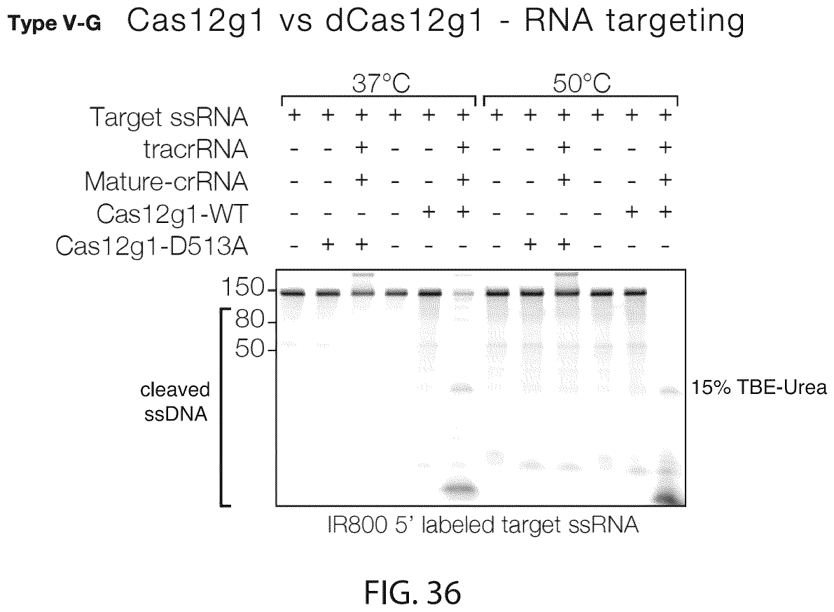

We additionally purified the dCas12g1 (D513A) protein with the RuvC domain point mutation that lacked interference activity on the in vivo screen (FIG. 14). The dCas12g1 (D513A) mutant showed neither target RNA cleavage nor collateral cleavage of ssDNA, suggesting that an intact RuvC domain is necessary for the cleavage of both RNA and ssDNA (FIGS. 35-36). Thus, the compact Cas12g1 effector demonstrates target and collateral RNA cleavage as well as temperature-dependent RNA-activated collateral ssDNA cleavage activity, all dependent on a single, intact RuvC domain.

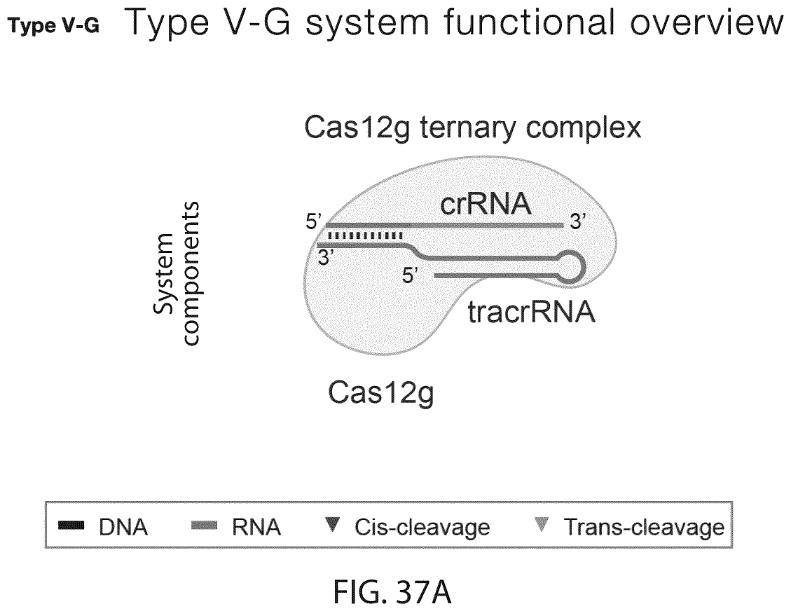

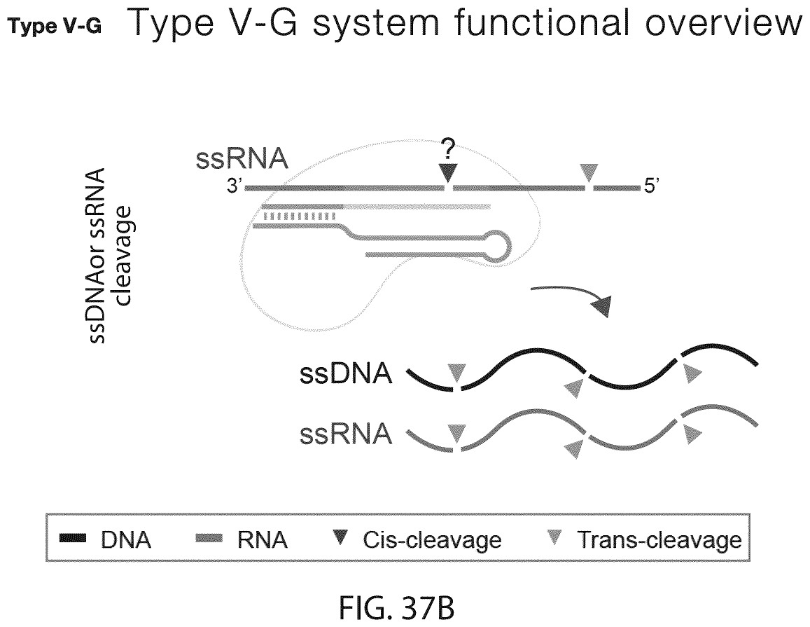

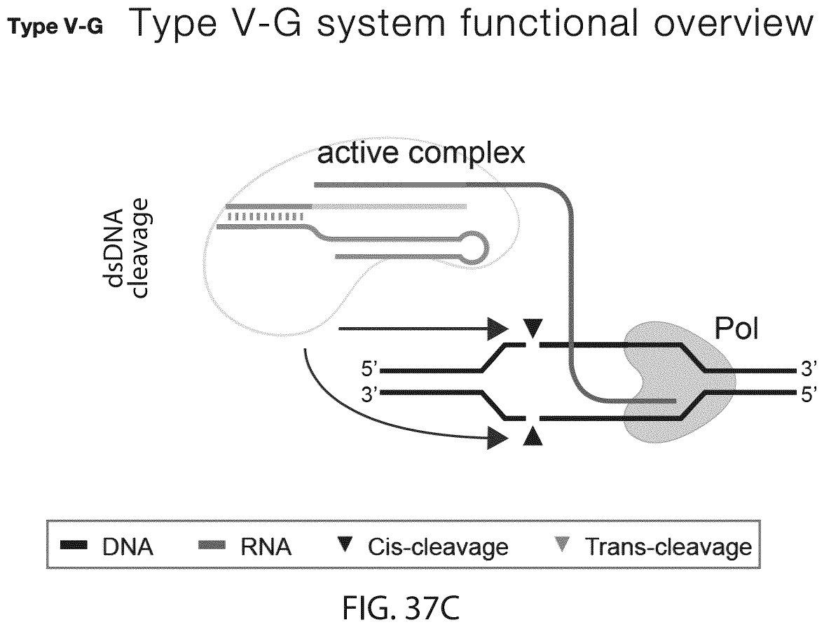

The Cas12g effector is a particularly remarkable case of convergent functional evolution that could help in understanding the origin and evolution of Class 2 CRISPR-Cas effectors. Cas12g contains a C-terminal RuvC-like domain characteristic of Cas12 nucleases and clearly is a derivative of TnpB (FIG. 1, FIG. 2A-B). However, the activity of Cas12g1 dramatically differs from those of all known type V systems in its ability to target RNA, in both the RNA guide-dependent and the guide-independent, collateral modes (FIG. 37A-B). In fact, the RNA targeting and the ensuing collateral RNA cleavage by Cas12g are closely analogous to the properties of the unrelated, HEPN-domain containing Cas13 effectors of the type VI CRISPR-Cas systems (Abudayyeh et al., 2016; East-Seletsky et al., 2016; Shmakov et al., 2015).

Although PAM-independent RNA targeting has recently been demonstrated for the Type II Cas9 nucleases from Campylobacter jejuni (CjCas9) and Neisseria meningitidis (NmeCas9), the cleavage in these cases is guide-dependent and site-specific (Dugar et al., 2018; Rousseau et al., 2018) as opposed to the collateral RNA cleavage observed with Cas12g1. While mutational analysis implicates the HNH domain as the nuclease responsible for the RNA cleavage by NmeCas9 and CjCas9 (Dugar et al., 2018; Rousseau et al., 2018), the results presented here demonstrate the requirement of an intact RuvC domain for the RNA cleavage by Cas12g1. To our knowledge, this is the first reported instance of RNA cleavage by the RuvC domain, one heretofore associated only with DNA cleavage.

In addition to the targeted and collateral RNA cleavage, we found that Cas12g1 catalyzes collateral cleavage of ssDNA (FIG. 35), resembling Cas12a in that respect (FIG. 37A-B). In the native environment, CLUST.019143 (Type V-G) interference activities are likely to include cleavage of genomic RNA, RNA transcripts, or ssDNA of invading species. Additionally, Cas12g might be capable of PAM-independent dsDNA cleavage via transcript-activated ssDNA cleavage at the transcriptional fork, somewhat analogously to the interference by type III CRISPR-Cas systems (FIG. 37C) (Samai et al., 2015). These multiple modes of efficient interference suggest that Cas12g may not be a nascent intermediate in the convergent evolution from TnpB to large CRISPR effectors, but a stable state along one particular path from TnpB to functional CRISPR systems. Comparative structural and biochemical studies of Cas12g as well as further investigation of additional subtype V-U systems is of great interest to further elucidate the evolution of TnpB into functional type V effectors.

The efficiency and specificity, combined with the differential of temperature control of RNA and ssDNA cleavage and stability of the thermophilic Cas12g1 surveillance complex, present potential opportunities for a range of applications. Perhaps, most importantly, Cas12g1 is the smallest active RNA editing CRISPR effector identified to date and, at 768aa, is 160aa smaller than the 928aa median size of the recently discovered Cas13d family proteins (Konermann et al., 2018; Yan et al., 2018). This compactness of Cas12g1 has the advantage of being readily packaged into various delivery vehicles. Additional applications of Cas12g could involve both efficient inactivation of target RNAs, and enhanced RNA and DNA sensing (Chen et al., 2018; East-Seletsky et al., 2016; Gootenberg et al., 2017, 2018; Li et al., 2018).

TABLE 8
Pre-crRNAs used for CLUST.019143 (Type V-G) in vitro biochemistry

| Name | Sequence | DR | Spacer1 | Spacer2 | Target | Fig |
|---|---|---|---|---|---|---|
| Cas12g1 | gggGGUGGAAAGGGCCGGAGA | GGUGGAAAGGG | CCAUUCAU | GCUUGGAG | Cas12g1 | FIGS. |
| pre- | UUUACCGGCUCUGACACCCCA | CCGGAGAUUUA | CCGCUUAU | GAGCGCAG | Target 1 | 15A- |
| crRNA 1 | UUCAUCCGCUUAUUAUCACUU | CCGGCUCUGAC | UAUCACUU | UCACCAAA | | 15B |
| | AUUCAGGGUGGAAAGGGCCGG | ACC (SEQ ID | AUUCAG | ACUUGU | | |
| | AGAUUUACCGGCUCUGACACC | NO: 401) | (SEQ ID | (SEQ ID | | |
| | GCUUGGAGGAGCGCAGUCACC | | NO: 402) | NO: 403) | | |
| | AAAACUUGUGGUGGAAAGGGC | | | | | |
| | CGGAGAUUUACCGGCUCUGAC | | | | | |
| | ACC (SEQ ID NO: 400) | | | | | |

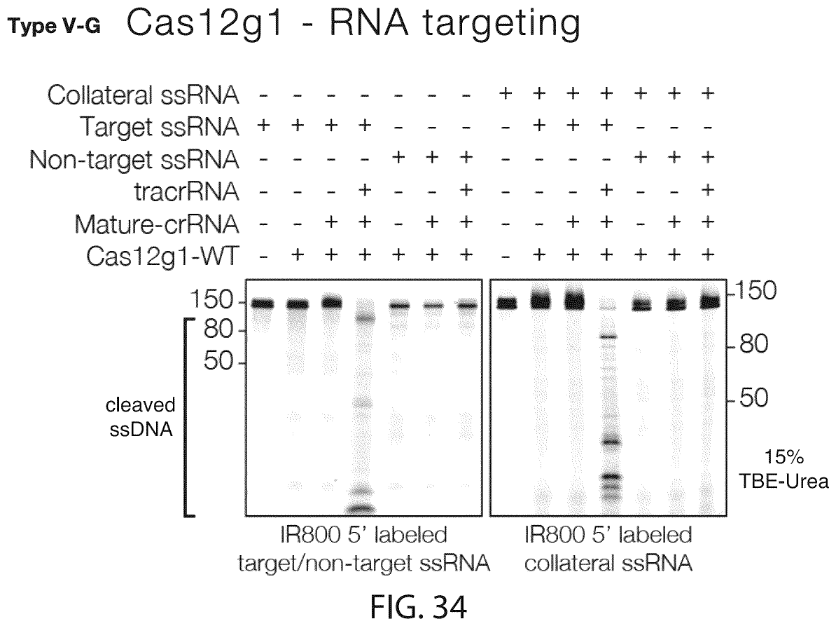

TABLE 9
tracrRNAs used for CLUST.019143 (Type V-G) in vitro biochemistry

| Name | Sequence | Fig |
|---|---|---|
| Cas12g1 | gggGAUGCUUACUUAGUCAUCUGGUUGGCAAACCUCCGCGGACCUUCGGGACCAA | FIGS. 15A- |
| tracr 1 | UGGAGAGGAACCCAGCCGAGAAGCAUCGAGCCGGUAAAUGCCGGAAAUUUUU | 15B |
| | (SEQ ID NO: 404) | |
| Cas12g1 | gggGAUGCUUACUUAGUCAUCUGGUUGGCAAACCUCCGCGGACCUUCGGGACCAA | FIG. 7, |
| tracr 2 | UGGAGAGGAACCCAGCCGAGAAGCAUCGAGCCGGUAAAUGCCGGAAA (SEQ ID | FIGS. 17A-36 |
| | NO: 405) | |
| Cas12g1 | gggGAUGCUUACUUAGUCAUCUGGUUGGCAAACCUCCGCGGACCUUCGGGACCAA | FIGS. 16A- |
| tracr 3 | UGGAGAGGAACCCAGCCGAGAAGCAUCGAGCCGGUAAAU (SEQ ID NO: | 16B |
| | 406) | |

TABLE 10
Mature-crRNAs used for CLUST.019143 (Type V-G) in vitro biochemistry

| | | | Processed | | |
|---|---|---|---|---|---|
| Name | Sequence | Processed DR | Spacer | Target | Fig |
| Cas12g1 | gggUUUACCGGCUCUGAC | UUUACCGGCUCU | GCUUGGAGGAGCG | Cas12g1 | FIGS. 16A- |
| mcrRNA | ACCGCUUGGAGGAGCGCA | GACACC (SEQ | CAGUCACCAAA | ssRNA 1 | 16B |
| 1 | GUCACCAAA (SEQ ID | ID NO: 408) | (SEQ ID NO: | | |
| | NO: 407) | | 409) | | |
| Cas12g1 | gggUUUACCGGCUCUGAC | UUUACCGGCUCU | AACUGUGAUAAAC | Cas12g1 | FIGS. 19-36 |
| mcrRNA | ACCAACUGUGAUAAACUA | GACACC (SEQ | UACCGCAUUAA | ssRNA 2 | |
| 2 | CCGCAUUAA (SEQ ID | ID NO: 411) | (SEQ ID NO: | | |
| | NO: 410) | | 412) | | |
| Cas12g1 | gggUUUACCGGCUCUGAC | UUUACCGGCUCU | CUUCCUUAGCUCC | Cas12g1 | FIGS. 30A- |
| mcrRNA | ACCCUUCCUUAGCUCCUG | GACACC (SEQ | UGAAAAUCUCG | ssRNA 3 | 31B |
| 3 | AAAAUCUCG (SEQ ID | ID NO: 414) | (SEQ ID NO: | | |
| | NO: 413) | | 415) | | |
| Cas12g1 | gggUUUACCGGCUCUGAC | UUUACCGGCUCU | CAUCUUGCGAAUA | Cas12g1 | FIGS. 17A- |
| mcrRNA | ACCCAUCUUGCGAAUAUA | GACACC (SEQ | UAUGUGUAGAA | ssDNA 4 | 18D |
| 4 | UGUGUAGAA (SEQ ID | ID NO: 417) | (SEQ ID NO: | | |
| | NO: 416) | | 418) | | |

TABLE 11
Target substrates used for CLUST.019143 (Type V-G) in vitro biochemistry

| | | Nucleic | |
|---|---|---|---|
| Name | Sequence | acid | Fig |
| Cas12g1 | CAACGAGGUAUUUAUCCGAUUAAUUCUCAUGUUUGACAGCUUAU | RNA | FIGS. 19-36 |
| ssRNA 2 | CAUCGAUAAGCUUUAAUGCGGUAGUUUAUCACAGUUAAAUUGCU | | |
| | AACGCAGUCAGGCACCGUGUAUUUGUAGCCCGGGGACUGUUU | | |
| | (SEQ ID NO: 419) | | |
| Cas12g1 | CAACGAGGUAUUUAUCCGAUAAAUAAGAUCACUACCGGGCGUAU | RNA | FIGS. 30A- |
| SSRNA 3 | UUUUUGAGUUAUCGAGAUUUUCAGGAGCUAAGGAAGCUAAAAUG | | 31B |
| | GAGAAAAAAAUCACUGGAUAUAUUGUAGCCCGGGGACUGUUU | | |
| | (SEQ ID NO: 420) | | |
| Cas12g1 | CAACGAGGUAUUUAUCCGAUUAAUUCUCAUGUUUGACAGCUUAU | RNA | FIGS. 19- |
| sense | CAUCGAUAAGCUUUAAUGCGGUAGUUUAUCACAGUUAAAUUGCU | | 22, FIGS. |
| RNA 2 | AACGCAGUCAGGCACCGUGUAUUUGUAGCCCGGGGACUGUUU | | 24-27 |
| | (SEQ ID NO: 421) | | |
| Cas12g1 | CAACGAGGTATTTATCCGATTAATTCTCATGTTTGACAGCTTAT | DNA | FIGS. 19- |
| sense | CATCGATAAGCTTTAATGCGGTAGTTTATCACAGTTAAATTGCT | | 22, FIGS. |
| DNA 2 | AACGCAGTCAGGCACCGTGTATTTGTAGCCCGGGGACTGTTT | | 24-27 |
| | (SEQ ID NO: 422) | | |
| Cas12g1 | AAACAGUCCCCGGGCUACAAAUACACGGUGCCUGACUGCGUUAG | RNA | FIGS. 19- |
| antisense | CAAUUUAACUGUGAUAAACUACCGCAUUAAAGCUUAUCGAUGAU | | 22, FIGS. |
| RNA 2 | AAGCUGUCAAACAUGAGAAUUAAUCGGAUAAAUACCUCGUUG | | 24-27 |
| | (SEQ ID NO: 423) | | |
| Cas12g1 | AAACAGTCCCCGGGCTACAAATACACGGTGCCTGACTGCGTTAG | DNA | FIGS. 19- |
| antisense | CAATTTAACTGTGATAAACTACCGCATTAAAGCTTATCGATGAT | | 22, FIGS. |
| DNA 2 | AAGCTGTCAAACATGAGAATTAATCGGATAAATACCTCGTTG | | 24-27 |
| | (SEQ ID NO: 424) | | |
| Cas12g1 | CAACGAGGTATTTATCCGATCGCTCTGGAGTGAATACCACGACG | DNA | FIGS. 17A- |
| ssDNA 4, | ATTTCCGGCAGTTTCTACACATATATTCGCAAGATGTGGCGTGT | | 18D |
| dsDNA 4 | TACGGTGAAAACCTGGCCTATTTTGTAGCCCGGGGACTGTTT | | |
| | (SEQ ID NO: 425) | | |

TABLE 12
Non-target substrates used for CLUST.019143 (Type
V-G) in vitro biochemistry

| | | Nucleic | |
|---|---|---|---|
| Name | Sequence | acid | Fig |
| Cas12g1 | AAACAGUCCCCGGGCUACAAA | RNA | FIGS. 23A-B, |
| SSRNA | UACACGGUGCCUGACUGCGUU | | 28A-B, 29A-B, |
| 2_RC | AGCAAUUUAACUGUGAUAAAC | | 31B, 33-34 |
| | UACCGCAUUAAAGCUUAUCGA | | |
| | UGAUAAGCUGUCAAACAUGAG | | |
| | AAUUAAUCGGAUAAAUACCUC | | |
| | GUUG (SEQ ID NO: 426) | | |
| Cas12g1 | AAACAGUCCCCGGGCUACAAU | RNA | FIG 30B |
| SSRNA | AUAUCCAGUGAUUUUUUUCUC | | |
| 3_RC | CAUUUUAGCUUCCUUAGCUCC | | |
| | UGAAAAUCUCGAUAACUCAAA | | |
| | AAAUACGCCCGGUAGUGAUCU | | |
| | UAUUUAUCGGAUAAAUACCUC | | |
| | GUUG (SEQ ID NO: 427) | | |

TABLE 13
Collateral substrates used in this study for in
vitro biochemistry

| | | Nucleic | |
|---|---|---|---|
| Name | Sequence | acid | Fig |
| Cas12g1 | AAACAGUCCCCGGGCUACAAU | RNA | FIGS. 30A- |
| SSRNA | CUCGCCGAAACGUUUGGUGGC | | B, 31A-B, |
| 5_RC | GGGACCAGUGACGAAGGCUUG | | 34 |
| | AGCGAGGGCGUGCAAGAUUCC | | |
| | GAAUACCGCAAGCGACAGGCC | | |
| | GAUCAAUCGGAUAAAUACCUC | | |
| | GUUG (SEQ ID NO: 428) | | |
| Cas12g1 | AAACAGTCCCCGGGCTACAAA | DNA | FIGS. 23A- |
| ssDNA | ATAGGCCAGGTTTTCACCGTA | | B, 33, 35 |
| 4_RC | ACACGCCACATCTTGCGAATA | | |
| | TATGTGTAGAAACTGCCGGAA | | |
| | ATCGTCGTGGTATTCACTCCA | | |
| | GAGCGATCGGATAAATACCTC | | |
| | GTTG (SEQ ID NO: 429) | | |

TABLE 14
IDT Template oligos and primers for crRNAs used

| | | T7 fwd | |
|---|---|---|---|
| Name | Template Sequence | primer | Rev primer |
| Cas12g1 | GGTGTCAGAGCCGGTAAATCTCCGGCCCTTTCCA | TAATACGACTC | GGTGTCAGAGCCGGTAAATC |
| pre- | CCACAAGTTTTGGTGACTGCGCTCCTCCAAGCGG | ACTATAG | TCCGGCCCTTTCCACCACAA |
| crRNA 1 | TGTCAGAGCCGGTAAATCTCCGGCCCTTTCCACC | (SEQ ID NO: | GTTTTGGTGACTGCGCTCCT |
| | CTGAATAAGTGATAATAAGCGGATGAATGGGGTG | 431) | CCAAGC (SEQ ID NO: |
| | TCAGAGCCGGTAAATCTCCGGCCCTTTCCACCcc | | 432) |
| | ctatagtgagtcgtatta (SEQ ID NO: | | |
| | 430) | | |
| Cas12g1 | TTAATGCGGTAGTTTATCACAGTTGGTGTCAGAG | TAATACGACTC | |
| mcrRNA 2 | CCGGTAAAccctatagtgagtcgtattaTTAATG | ACTATAGTAAT | |
| | CGGTAGTTTATCACAGTTGGTGTCAGAGCCGGTA | ACGACTCACTA | |
| | AAccctatagtgagtcgtatta (SEQ ID NO: | TAG (SEQ ID | |
| | 433) | NO: 434) | |
| Cas12g1 | CGAGATTTTCAGGAGCTAAGGAAGGGTGTCAGAG | TAATACGACTC | |
| mcrRNA 3 | CCGGTAAAccctatagtgagtcgtattaCGAGAT | ACTATAGTAAT | |
| | TTTCAGGAGCTAAGGAAGGGTGTCAGAGCCGGTA | ACGACTCACTA | |
| | AAccctatagtgagtcgtatta (SEQ ID NO: | TAG (SEQ ID | |
| | 435) | NO: 436) | |
| Cas12g1 | TTCTACACATATATTCGCAAGATGGGTGTCAGAG | TAATACGACTC | |
| mcrRNA 4 | CCGGTAAAccctatagtgagtcgtattaTTCTAC | ACTATAGTAAT | |
| | ACATATATTCGCAAGATGGGTGTCAGAGCCGGTA | AGGACTCACTA | |
| | AAccctatagtgagtcgtatta (SEQ ID NO: | TAG (SEQ ID | |
| | 437) | NO: 438) | |
| Cas12g1 | AAAAATTTCCGGCATTTACCGGCTCGATGCTTCT | TAATACGACTC | |
| tracr 1 | CGGCTGGGTTCCTCTCCATTGGTCCCGAAGGTCC | ACTATAGTAAT | |
| | GCGGAGGTTTGCCAACCAGATGACTAAGTAAGCA | AGGACTCACTA | |
| | TCccctatagtgagtcgtattaAAAAATTTCCGG | TAG (SEQ ID | |
| | CATTTACCGGCTCGATGCTTCTCGGCTGGGTTCC | NO: 440) | |
| | TCTCCATTGGTCCCGAAGGTCCGCGGAGGTTTGC | | |
| | CAACCAGATGACTAAGTAAGCATCccctatagtg | | |
| | agtcgtatta (SEQ ID NO: 439) | | |
| Cas12g1 | TTTCCGGCATTTACCGGCTCGATGCTTCTCGGCT | TAATACGACTC | |
| tracr 2 | GGGTTCCTCTCCATTGGTCCCGAAGGTCCGCGGA | ACTATAGTAAT | |
| | GGTTTGCCAACCAGATGACTAAGTAAGCATCccc | AGGACTCACTA | |
| | tatagtgagtcgtattaTTTCCGGCATTTACCGG | TAG (SEQ ID | |
| | CTCGATGCTTCTCGGCTGGGTTCCTCTCCATTGG | NO: 442) | |
| | TCCCGAAGGTCCGCGGAGGTTTGCCAACCAGATG | | |
| | ACTAAGTAAGCATCccctatagtgagtcgtatta | | |
| | (SEQ ID NO: 441) | | |
| Cas12g1 | ATTTACCGGCTCGATGCTTCTCGGCTGGGTTCCT | TAATACGACTC | |
| tracr 3 | CTCCATTGGTCCCGAAGGTCCGCGGAGGTTTGCC | ACTATAGTAAT | |
| | AACCAGATGACTAAGTAAGCATCccctatagtga | AGGACTCACTA | |
| | gtcgtattaATTTACCGGCTCGATGCTTCTCGGC | TAG (SEQ ID | |
| | TGGGTTCCTCTCCATTGGTCCCGAAGGTCCGCGG | NO: 444) | |
| | AGGTTTGCCAACCAGATGACTAAGTAAGCATCcc | | |
| | ctatagtgagtcgtatta (SEQ ID NO: | | |
| | 443) | | |

TABLE 15
IDT template oligos and primers for ssRNA
substrates used in this study for in vitro biochemistry

| | | −sense | | | |
|---|---|---|---|---|---|
| | | RNA fwd | −sense RNA | + sense RNA | + sense RNA |
| Name | Template Sequence | primer | rev primer | fwd primer | rev primer |
| Cas12g1 | Cas12g1 | | | | |
| SSRNA 1 | | | | | |
| Cas12g1 | ssRNA 1 | AAACAGTC | AAACAGTCCCC | AAATGTAATAA | AAATGTAATAA |
| ssRNA 2 | | CCCGGGCT | GGGCTACAA | ggatccctcga | ggatccctcga |
| | | ACAAAGCT | (SEQ ID NO: | gtaatacgact | gtaatacgact |
| | | ACCAACTC | 446) | cactatagggC | cactatagggA |
| | | TTTGAACC | | AACGAGGTATT | AACAGTCCCCG |
| | | GAGGTAAC | | TATCCGAT | GGCTACAA |
| | | TGGCTTGG | | (SEQ ID NO: | (SEQ ID NO: |
| | | AGGAGCGC | | 447) | 448) |
| | | AGTCACCA | | | |
| | | AAACTTGT | | | |
| | | CCTTTCAG | | | |
| | | TTTAGCCT | | | |
| | | TAACCGGC | | | |
| | | GCATGAAT | | | |
| | | CGGATAAA | | | |
| | | TACCTCGT | | | |
| | | TG (SEQ | | | |
| | | ID NO: | | | |
| | | 445) | | | |
| Cas12g1 | Cas12g1 | | | | |
| ssRNA 3 | | | | | |
| Cas12g1 | ssRNA 2 | AAACAGTC | AAACAGTCCCC | AAATGTAATAA | AAATGTAATAA |
| SSRNA 4 | | CCCGGGCT | GGGCTACAA | ggatccctcga | ggatccctcga |
| | | ACAAATAC | (SEQ ID NO: | gtaatacgact | gtaatacgact |
| | | ACGGTGCC | 451) | cactatagggC | cactatagggA |
| | | TGACTGGG | | AACGAGGTATT | AACAGTCCCCG |
| | | TTAGCAAT | | TATCCGAT | GGCTACAA |
| | | TTAACTGT | | (SEQ ID NO: | (SEQ ID NO: |
| | | GATAAACT | | 452) | 453) |
| | | ACCGGATT | | | |
| | | AAAGCTTA | | | |
| | | TCGATGAT | | | |
| | | AAGCTGTC | | | |
| | | AAACATGA | | | |
| | | GAATTAAT | | | |
| | | CGGATAAA | | | |
| | | TACCTCGT | | | |
| | | TG (SEQ | | | |
| | | ID NO: | | | |
| | | 450) | | | |
| Cas12g1 | Cas12g1 | | | | |
| ssRNA 5 | | | | | |

### Example 6. CLUST.019143 (Type V-G) CRISPR-Cas Systems can be Used with a Fluorescent Reporter for the Specific Detection of Nucleic Acid Species

The nuclease activities of Cas12g proteins (i.e., non-specific collateral RNase and DNase activities activated by a target RNA substrate complementary to the crRNA spacer) make these effectors promising candidates for use in the detection of nucleic acid species. Some of these methods have been previously described (see, e.g., East-Seletsky et al. (2016), Gootenberg et al. (2017), and Gootenberg et al. (2018) “Multiplexed and portable nucleic acid detection platform with Cas13, Cas12a, and Csm6” Science 15 Feb. 2018: eaaq0179), describing the general principle of RNA detection using Cas13a (East-Seletsky et al. (2016)), supplemented by amplification to increase the detection sensitivity and optimization of additional Cas13a enzymes (Gootenberg et al. (2017)), and most recently, the inclusion of additional RNA targets, orthologous and paralogous enzymes, and Csm6 activator to enable multiplexed detection of nucleic acids along with an increase in detection sensitivity (Gootenberg et al. (2018)). The addition of Cas12g to this toolkit provides an additional channel of orthogonal activity for nucleic acid detection.

The in vitro biochemical activity of Cas12g1 suggests that it may have promise in applications for sensitive nucleic acid detection, given that a dye-labeled, collateral RNA was efficiently cleaved at low target RNA concentrations (FIG. 31A) and background nuclease activity was limited with a non-targeting substrate over a wide range of Cas12g1 complex (FIG. 30B) and substrate concentrations (FIG. 31B). Adapting Cas12g1 towards sensitive nucleic acid detection application requires several steps, including, but not limited to, engineering a single RNA guide from the crRNA and tracrRNA for enhanced enzyme activity, optimizing the substrate for sensitive readout of the collateral activity and identifying per-base mismatch tolerance between the spacer and the target substrate.

Identification of the optimal substrate for nucleic acid detection can be informed by performing next generation sequencing (NGS) on the cleavage products of Cas12g collateral activity on both ssDNA and ssRNA substrates. The enzyme concentration may have to be titrated or incubation time adjusted in order to yield cleavage fragments that are still of a sufficient size to be prepared into a next generation sequencing library. The NGS data reveal the enzyme cleavage sites and the adjacent base preferences. It has been demonstrated that the individual effectors within the Cas13a and Cas13b families have different dinucleotide base preferences for RNA cleavage, yielding markedly different cleavage magnitudes and signal to noise ratios (Gootenberg et al. (2018)). The collateral NGS data thus enable better insight into the preferences for Cas12g. A separate experimental approach to identifying the dinucleotide preference of Cas12g collateral cleavage is to create a collateral RNA or DNA substrate with degenerate N's in sequential positions so as to have a broader sequence space than a defined collateral nucleic sequence. The library prep and analysis of the NGS data would proceed similarly to identify base preferences for cleavage. To verify the preference, collateral substrates containing synthesized short DNA/RNAs with a fluorophore/quencher pair on the 5′ and 3′ ends can be introduced into a cleavage reaction to assess the signal to noise ratio. Further optimization can be done on the length of the collateral DNA/RNA substrate to determine whether Cas12g1 has a length preference.

Having identified the collateral nuclease substrate preferences, another important parameter to determine is the mismatch tolerance of the Cas12g system, as it has implications for guide design that affects the ability of the enzyme to distinguish single base pair mismatches. The mismatch tolerance can be determined by designing a panel of targets bearing different positions and types of mismatches (for example, insertion/deletions, single base pair mismatches, adjacent double mismatches, separated double mismatches, triple mismatches, and more). Mismatch tolerance can be measured by assessing the amount of cleavage of collateral DNA for targets containing varying amounts of mismatches. As an example, the collateral RNA substrate could be a short ssRNA probe containing a fluorophore and quencher on opposite sides. For reactions containing the Cas12g effector, an RNA guide, and a target substrate containing different numbers of mismatches, insertions and deletions in the target sequence, successful activation of the Cas12g system by targeting of altered target RNA sequence will result in collateral cleavage of the fluorescent probe. Resulting fluorescent measurements denoting cleaved collateral substrate can be background subtracted using negative control samples and normalized to the signal from perfectly matching targets to estimate the impact of target alterations on the efficiency of collateral cleavage by Cas12g. Resulting maps of mismatch, insertion, and deletion tolerance by the Cas12g enzyme over the target length relative to the PAM can be used to design optimal RNA guides to distinguish between different RNA sequences or genotypes for specific detection or distinction between different Nucleic Acid Species. The lack of PAM or PFS of Cas12g1 makes it position the a target sequence over a site of genotypic variation to optimize the specificity of discrimination between the two genotypes given the Cas12g mismatch tolerance map.

The optimization process can be furthermore applied to other Cas12g orthologs to yield other systems that may have different properties. For example, orthogonal dinucleotide preferences of collateral cleavage would be helpful in generating separate channels of detection.

For nucleic acid detection applications, the unique properties of Cas12g1 may provide advantages. In some embodiments, the dual ssDNA and ssRNA collateral cleavage may serve to distinguish target-activated cleavage from background nuclease contamination. In such an application, ssDNA and ssRNA collateral substrates are labeled with fluorophores on separate channels and are both introduced into a detection reaction. Fluorescence in both ssDNA and ssRNA channels are required to be considered as a positive signal, thus potentially reducing the number of false positives especially since RNase contamination is widespread. The thermostability of Cas12g1, as evidenced by its high melting temperature and ssDNA/ssRNA cleavage activity at 50 C (FIGS. 16B, 19, 29A) may provide additional robustness in the assay. Thermostable Cas proteins have been demonstrated to be more resistant to inactivation in plasma (Harrington et al. (2017)), and the thermostability of Cas12g1 may enable it to be used directly in collected samples without the need for extensive processing. Furthermore, increased thermostability often confers robustness to environmental degradation or denaturation. Particularly since such nucleic acid detection assays such as SHERLOCK have been engineered into more portable formats for point of care applications (such as paper detection strips) (Gootenberg, et al. (2018), Myhrvold et al. (2018)), a prolonged shelf life enabled by the increased protein stability from Cas12g1 may provide greater versatility and utility.

### Example 7. CLUST.019143 (Type V-G) CRISPR-Cas Systems can be Used to Provide Genotype-Gated Control of Cell Death or Dormancy

Hybridization of the CLUST.019143 (Type V-G) CRISPR-Cas effector protein and crRNA with an RNA target complementary to the crRNA spacer forms an active complex that may exhibit nonspecific, “collateral” RNase activity. Such collateral RNAse activity can be used to provide genotype-gated control of cell death or dormancy. The dependence of such activity on the presence of a specific RNA target in a cell is valuable since it enables targeting of specific cell populations based on specific underlying transcriptional states or genotypes. Numerous applications exist in both eukaryotic and prokaryotic settings for such control of cell death or dormancy.

For prokaryotic applications, a CLUST.019143 (Type V-G) CRISPR-Cas system (e.g., including a Cas12g and RNA guide) is delivered (e.g., in vitro or in vivo) in order to induce cell death or dormancy of specific prokaryote populations (e.g., bacterial populations) in a genotype and transcriptome-specific way. For instance, the CLUST.019143 (Type V-G) CRISPR-Cas system includes one or more crRNAs that specifically target a particular prokaryotic genus, species, or strain. This specific targeting has many therapeutic benefits as it may be used to induce death or dormancy of undesirable bacteria (e.g., pathogenic bacteria such as Clostridium difficile) while protecting an individual's microbiome. In addition, the CLUST.019143 (Type V-G) systems provided herein are used to target prokaryotic cells having specific genotypes or transcriptional states. Within the microbial diversity that colonizes humans, only a small number of bacterial strains can induce pathogenesis. Further, even within pathogenic strains such as Clostridium difficile, not all members of the bacterial population exist continuously in active, disease-causing states. Thus, using RNA-targeting to control the activity of a Cas12g based on the genotype and transcriptional state of a prokaryotic cell allows for specific control of which cells are targeted without disrupting the entire microbiome.

Additionally, bacterial strains are readily engineered with genetic circuits or environmentally-controlled expression elements to generate genetic kill switches that limit the growth, colonization, and/or shedding of the engineered bacterial strains. For example, the expression of a Cas12g proteins, specific crRNA, or specific target RNA, is controlled using promoters derived from the regulatory regions of genes encoding proteins expressed in response to external stimuli, such as cold sensitive proteins (PcspA), heat shock proteins (Hsp), chemically inducible systems (Tet, Lac, AraC). The controlled expression of one or more elements of the CLUST.019143 (Type V-G) system allows for the full functional system to be expressed only upon exposure to an environmental stimulus, which in turn activates the nonspecific RNase activity of the system and thereby induces cell death or dormancy. Kill switches including Cas12g proteins as those described herein may be advantageous over traditional kill switch designs such as toxin/antitoxin systems (e.g., CcdB/CcdA Type II toxin/antitoxin systems), since they are not dependent on relative protein expression ratios which may be affected by leaky expression from a promoter (e.g., an environmental-stimulus dependent promoter), and thus allow for more precise control of the kill-switch.

To assess the ability of Cas12g to induce the dormancy or death of bacteria cells directly upon recognition of a target RNA, a variation of the in vivo functional screening was performed, in which the antibiotic tetracycline was removed from the culture plate. Removing tetracycline selection meant that the survival of the host E. coli was no longer dependent on the successful natural expression of the tetracycline resistance protein by pACYC184. However, the targeting library still contained crRNAs with spacers to the tetracycline resistance gene, Tc^{R}. When the dependence of E. coli survival on successful Tc^{R} expression is removed, one would expect that there would be no impact on E. coli survival if the Cas12g directly cleaved Tc^{R} mRNA, and thus no Tc^{R} targeting spacers should register as a strong depletion event on the in vivo screen. Nevertheless, the screening data without tetracycline selection still showed strongly depleted spacers on the Tc^{R} gene (FIGS. 6A-B), suggesting that the effect of Cas12g targeting RNA alone can mediate a growth disadvantage or cell death, even without antibiotic selection.

For eukaryotic applications, many diseases result from specific genotypes or transcriptional states in the diseased cells that distinguish them from healthy cells. Disease related genotypes are often contained in regions of the genome that are expressed, generating transcripts that can be targeted by a Cas12g using a crRNA that specifically targets the genotype. Such targeting provides cell dormancy or cell death in a population of cells with a specific disease related mutations. An example of an application is the targeted depletion of cancer cells containing specific mutations, such as driver mutations that occur spontaneously in the tumor microenvironment. In addition, the CLUST.019143 (Type V-G) CRISPR-Cas systems described herein are as kill-switch mechanisms to induce the death or dormancy of recombinant eukaryotic cells, such as chimeric antigen receptor-expressing T-cells, to limit their activity in inappropriate environments or when no longer desired.

Additionally, in a therapeutic context, numerous disease processes often involve dysregulation of cellular pathways that result in transcriptional states that are different from the normal baseline. A CLUST.019143 (Type V-G) CRISPR-Cas system can be used to specifically induce the death or dormancy of cells that have an altered transcriptome. For example, the system can be used to induce the death or dormancy of cells having a temporally altered transcriptome, such as cells involved in an anti-inflammatory response during an autoimmune disease flare that are differentiated from normal cells.

In some embodiments, the expression of the CLUST.019143 (Type V-G) CRISPR-Cas systems described herein can be controlled using synthetic biology to induce or trigger cell death or dormancy. For example, the expression of genes encoding each of the components of the CLUST.019143 (Type V-G) CRISPR-Cas systems can be controlled using genetic elements including, but are not limited to, promoters that are regulated by environmental stimuli, such as hypoxia (hif), neuronal activity (fos, arc), heat-shock (HSF-1), or exogenous controls such as light (FixJ), steroids (LexA), alcohol (AlcA), tetracycline (Tet). These promoters are used to control the expression of components of the CLUST.019143 (Type V-G) CRISPR-Cas system and/or of a specific RNA target to activate the system, thereby inducing the death or dormancy of targeted cells in response to the particular environmental stimuli to which the promoters respond.

### Example 8. Adaptation of Type V-G CRISPR Cas Effector Protein Systems for Eukaryotic and Mammalian Activity

Beyond the biochemical and diagnostic applications described herein, programmable RNA-modifying CRISPR-Cas systems such as CLUST.019143 (Type V-G), e.g., Cas12g, systems described herein have important applications in eukaryotic cells. Therapeutic applications include but are not limited to: genotype correction, insertion of a protective genotype, transcriptome engineering (for example, splicing modification), RNA knockdown and gene expression modulation, base editing, cellular engineering, to research and development advances, such as for RNA visualization, nucleic acid detection, and RNA knockdown screening.

To develop CLUST.019143 (Type V-G) CRISPR Cas systems for eukaryotic applications, the constructs encoding the Type V-G CRISPR-Cas effector proteins are first codon-optimized for expression in mammalian cells, and specific localization tags are optionally appended to either or both the N-terminus or C-terminus of the effector protein. These localization tags include sequences such as nuclear localization signal (NLS) sequences, which localize the effector to the nucleus for modification of nascent RNAs, as well as nuclear export signal (NES) sequences, which target the effector to the cytoplasm in order to modify mature RNAs. These sequences are described above in the “Functional Mutations” section. Other accessory proteins, such as fluorescent proteins, may be further appended. It has been demonstrated that the addition of robust, “superfolding” proteins such as superfolding green fluorescent protein (GFP) can increase the activity of CLUST.019143 (Type V-G) enzymes in mammalian cells when appended to the effector (Abudayyeh et al. (2017) Nature 550(7675): 280-4, and Cox et al. (2017) Science 358(6366): 1019-27).

Further engineering to modify the crRNA and/or tracrRNA is used to optimize activity specifically in eukaryotic and/or mammalian cells. To simplify the expression and complexing of the dual crRNA and tracrRNA required for Cas12g activity, a single RNA guide sequence is engineered. In some embodiments, the crRNA and tracrRNA are engineered by using a short linker sequence connecting the 3′ end of the tracrRNA with the 5′ end of the crRNA. A key feature to preserve is the overall secondary structure of the co-folded RNAs. Furthermore, the length and composition of the linker as well as the sequence of the direct repeat adjacent to the linker are optimized to design a most effective single RNA guide. Additionally, chemical modifications (Hendel et. al. Nat Biotechnol. 2015 September; 33(9):985-989, Yin, et. al. Nat Biotechnol. 2017 December; 35(12):1179-1187) can be used to enhance the in vivo editing efficiency by conferring properties such as greater resistance to nuclease degradation and enhanced secondary structure formation. In one instance, the effect of these modifications are compared in their RNA knockdown ability against the composition in which the pre-crRNA and tracrRNA are expressed as separate RNAs. Together these engineered modifications provide greater nuclease activity in eukaryotic cells and increase the flexibility of usage in genome editing applications.

The codon-optimized sequence coding for the Cas12g and appended accessory proteins and localization signals is then cloned into a eukaryotic expression vector with the appropriate 5′ Kozak eukaryotic translation initiation sequence, eukaryotic promoters, and polyadenylation signals. In one embodiment, the expression of the effector is driven by a constitutive promoter, the elongation factor 1alpha short (EFS) promoter, and terminated by a bGH poly(A) signal. Expression of the RNA guide is driven by U6, a Polymerase III promoter; or if the crRNA and tracrRNA are expressed separately, by the Polymerase III promoters U6 and H1, respectively.

Depending on the application and mode of packaging, the eukaryotic expression vector can be a lentiviral plasmid backbone, adeno-associated viral (AAV) plasmid backbone. Notably, the small size of CLUST.019143 (Type V-G) CRISPR Cas effector proteins, e.g., Cas12g proteins, make them ideally suited for packaging along with its crRNA and appropriate control sequences into a single adeno-associated virus particle; the packaging size limit of 4.7 kb for AAV may preclude the use of larger effectors.

Additionally, multiplexing of Cas12g with multiple RNA guides targeting different sequences enables the manipulation of multiple RNA species for therapeutic applications requiring manipulation of multiple transcripts simultaneously.

After adapting the sequences, delivery vectors, and methods for mammalian use, the Cas12g constructs described herein are characterized for performance. For efficient testing of the mammalian activity levels of various constructs, we use a dual-luciferase reporter expressing both Gaussia luciferase (Gluc) and Cypridinia luciferase (Cluc) (Abudayyeh et al. 2017). The dual-luciferase reporter construct along with plasmids expressing the CLUST.019143 (Type V-G) CRISPR-Cas system and cognate RNA guides are delivered using transient transfection (e.g., Lipofectamine® 2000) into model cell lines such as HEK 293T cells. Targeting the Gluc transcript and comparing the relative activity versus the internal control of the Cluc activity enables an estimation of Cas12g effectiveness in a mammalian context. This activity is then corroborated on the reporter through knockdown of endogenous transcripts, such as from the well-characterized KRAS genetic locus.

In addition to testing various construct configurations and accessory sequences on individual targets, pooled library-based approaches are used to determine 1) any targeting dependency of specific Cas12g proteins in mammalian cells, as well as 2) the effect of mismatch locations and combinations along the length of the targeting crRNA. Briefly, the pooled library includes a plasmid that expresses a target RNA containing different flanking sequences as well as mismatches to the guide or guides used in the screening experiment, such that the successful target recognition and cleavage results in depletion of the sequence from the library. Furthermore, mRNA sequencing is used to determine the off-target RNA cleavage effects of the CLUST.019143 (Type V-G) CRISPR-Cas system.

Complementary possibilities to transcriptome modification using the RNA cleavage activity of Cas12g are engineered applications using catalytically-inactive Cas12g proteins in which the conserved residues of the RuvC domain are mutated to alanine (such as the inactivating D513A mutation for Cas12g1). Catalytically inactive Cas12g retains its programmable RNA binding activity, though it will no longer be able to cleave target or collateral RNA.

In addition to direct uses of dCas12g such as in RNA immunoprecipitation, transcript labeling (when dCas12g is fused with fluorescent protein), and translation modification through site-specific targeted disruption of native translational machinery, other domains can be appended onto the dCas12g protein to provide further functionality. Activities of these domains include RNA base modification (ADAR1, ADAR2, APOBEC), RNA methylation (m6A methyltransferases and demethylases), splicing modifiers (hnRNPA1), localization factors (KDEL retention sequence, mitochondrial targeting signal, peroxisomal targeting signal), translation modification factors (EIF4G translation initiation factor, GLD2 poly(A) polymerase, transcriptional repressors). Additionally, additional control is provided by further appending domains, such as light-gated control (cryptochromes) and chemically inducible components (FKBP-FRB chemically inducible dimerization).

Optimizing the activity of such fusion proteins requires a systematic way of comparing linkers that connect the dCas12g with the appended domain. These linkers include flexible glycine-serine (GS) linkers in various combinations and lengths, rigid linkers such as the alpha-helix forming EAAAK sequence, XTEN linker (Schellenberger V, et al. Nat. Biotechnol. 2009; 27:1186-1190), as well as different combinations thereof (see TABLE 16). The various designs are then assayed in parallel over the same RNA guide/target complex and functional readout to determine which one yields the desired properties.

For adapting Cas12g for use in targeted RNA base modification (see, e.g., Cox D B T et al., Science 2017 10.1126/science.aaq0180), we begin with the Cas12g ortholog and NES combination that yielded the highest endogenous mammalian RNA knockdown activity and mutate the conserved residues of the RuvC domain to create a catalytically inactive enzyme. Next, a linker is used to create the fusion protein between Cas12g-NES and the base editing domain. Initially, this domain will consist of the ADAR2_{DD}(E488Q/T375G) mutant engineered previously for hyperactivity and greater specificity when used with Cas13b in REPAIRv2, but alternate deaminases such as ADAR1 and APOBEC1, among others, can be engineered and assayed in parallel (TABLE 16). Given the likely structural differences between the smaller Cas12g versus the previously characterized Cas13 effectors, alternate linker designs and lengths may yield the optimal design of the base editing fusion protein.

To evaluate the activity of the dCas12g-derived base editors, the HEK 293T cells are transiently transfected with the dCas12g-ADAR construct, a plasmid expressing the RNA guide, and optionally, a reporter plasmid if the base editor is targeting the reporter and not an endogenous locus. The cells are harvested 48 hours after transient transfection, the RNA is extracted and reverse-transcribed to yield a cDNA library that is prepared for next generation sequencing. Analysis of the base composition of loci of samples containing the targeting vs. negative control non-targeting crRNAs provide information about the editing efficiency, and analysis of broader changes to the transcriptome will yield information about the off-target activity.

One particular advantage of developing an RNA base editing system using Cas12g is that the small size, smaller than the existing Cas13a-d effectors, enables more ready packaging in AAV of dCas12g-ADAR along with its RNA guide and control elements without the need for protein truncations. This all-in-one AAV vector enables greater efficacy of in vivo base editing in tissues, which is particularly relevant as a path towards therapeutic applications of Cas12g. In base editing and other applications, the small size, the lack of a biochemical PFS, and robust activity of Cas12g proteins make it a valuable addition to the toolbox of programmable RNA modifying enzymes.

TABLE 16
Amino Acid Sequences of Motifs and Functional Domains in Engineered
Variants of CLUST.019143 (Type V-G) CRISPR-Cas Effector Proteins

>LINKER_1
GS
>LINKER_2
GSGGGGS
>LINKER_3
GGGGSGGGGSGGGGS
>LINKER_4
GGSGGSGGSGGSGGSGGS
[ADAR1, ADAR2: C-term fusion (or optionally N-term)]
>ADAR1DD-WT
SLGTGNRCVKGDSLSLKGETVNDCHAEIISRRGFIRFLYSELMKYNSQTAKDSIFEPAKGGEKLQIKKTVSFHLYI
STAPCGDGALFDKSCSDRAMESTESRHYPVFENPKQGKLRTKVENGEGTIPVESSDIVPTWDGIRLGERLRTMSCS
DKILRWNVLGLQGALLTHFLQPIYLKSVTLGYLFSQGHLTRAICCRVTRDGSAFEDGLRHPFIVNHPKVGRVSIYD
SKRQSGKTKETSVNWCLADGYDLEILDGTRGTVDGPRNELSRVSKKNIFLLFKKLCSFRYRRDLLRLSYGEAKKAA
RDYETAKNYFKKGLKDMGYGNWISKPQEEKNF
>ADAR1DD-E1008Q (Cox et al., 2017)
SLGTGNRCVKGDSLSLKGETVNDCHAEIISRRGFIRFLYSELMKYNSQTAKDSIFEPAKGGEKLQIKKTVSFHLYI
STAPCGDGALFDKSCSDRAMESTESRHYPVFENPKQGKLRTKVENGQGTIPVESSDIVPTWDGIRLGERLRTMSCS
DKILRWNVLGLQGALLTHFLQPIYLKSVTLGYLFSQGHLTRAICCRVTRDGSAFEDGLRHPFIVNHPKVGRVSIYD
SKRQSGKTKETSVNWCLADGYDLEILDGTRGTVDGPRNELSRVSKKNIFLLFKKLCSFRYRRDLLRLSYGEAKKAA
RDYETAKNYFKKGLKDMGYGNWISKPQEEKNF
>ADAR2DD-WT
QLHLPQVLADAVSRLVLGKFGDLTDNFSSPHARRKVLAGVVMTTGTDVKDAKVISVSTGTKCINGEYMSDRGLALN
DCHAEIISRRSLLRFLYTQLELYLNNKDDQKRSIFQKSERGGFRLKENVQFHLYISTSPCGDARIFSPHEPILEEP
ADRHPNRKARGQLRTKIESGEGTIPVRSNASIQTWDGVLQGERLLTMSCSDKIARWNVVGIQGSLLSIFVEPIYFS
SIILGSLYHGDHLSRAMYQRISNIEDLPPLYTLNKPLLSGISNAEARQPGKAPNFSVNWTVGDSAIEVINATTGKD
ELGRASRLCKHALYCRWMRVHGKVPSHLLRSKITKPNVYHESKLAAKEYQAAKARLFTAFIKAGLGAWVEKPTEQD
QFSLT
ADAR2DD-E4880 (Cox et al., 2017)
QLHLPQVLADAVSRLVLGKFGDLTDNFSSPHARRKVLAGVVMTTGTDVKDAKVISVSTGTKCINGEYMSDRGLALN
DCHAEIISRRSLLRFLYTQLELYLNNKDDQKRSIFQKSERGGFRLKENVQFHLYISTSPCGDARIFSPHEPILEEP
ADRHPNRKARGQLRTKIESGQGTIPVRSNASIQTWDGVLQGERLLTMSCSDKIARWNVVGIQGSLLSIFVEPIYFS
SIILGSLYHGDHLSRAMYQRISNIEDLPPLYTLNKPLLSGISNAEARQPGKAPNFSVNWTVGDSAIEVINATTGKD
ELGRASRLCKHALYCRWMRVHGKVPSHLLRSKITKPNVYHESKLAAKEYQAAKARLFTAFIKAGLGAWVEKPTEQD
QFSLT
[Cytidine deaminase, AID, APOBEC1: N-term fusion (or optionally C-term)]
>AID-APOBEC1 (Dickerson et al., 2003, Komor et al., 2017)
MDSLLMNRRKFLYQFKNVRWAKGRRETYLCYVVKRRDSATSFSLDFGYLRNKNGCHVELLFLRYISDWDLDPGRCY
RVTWFTSWSPCYDCARHVADFLRGNPNLSLRIFTARLYFCEDRKAEPEGLRRLHRAGVQIAIMTFKDYFYCWNTFV
ENHERTFKAWEGLHENSVRLSRQLRRILLPLYEVDDLRDAFRTLGL
>Lamprey_AID-APOBEC1 (Rogozin et al., 2007, Komor et al., 2017)
MTDAEYVRIHEKLDIYTFKKQFFNNKKSVSHRCYVLFELKRRGERRACFWGYAVNKPQSGTERGIHAEIFSIRKVE
EYLRDNPGQFTINWYSSWSPCADCAEKILEWYNQELRGNGHTLKIWACKLYYEKNARNQIGLWNLRDNGVGLNVMV
SEHYQCCRKIFIQSSHNQLNENRWLEKTLKRAEKRRSELSIMIQVKILHTTKSPAV
>APOBEC1_BE1 (Komor et al., 2016)
MSSETGPVAVDPTLRRRIEPHEFEVFFDPRELRKETCLLYEINWGGRHSIWRHTSQNTNKHVEVNFIEKFTTERYF
CPNTRCSITWFLSWSPCGECSRAITEFLSRYPHVTLFIYIARLYHHADPRNRQGLRDLISSGVTIQIMTEQESGYC
WRNFVNYSPSNEAHWPRYPHLWVRLYVLELYCIILGLPPCLNILRRKQPQLTFFTIALQSCHYQRLPPHILWATGL
K

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An engineered, non-naturally occurring Clustered Regularly Interspaced Short Palindromic Repeat (CRISPR)-associated (Cas) system comprising:
an RNA guide or a nucleic acid encoding the RNA guide, wherein the RNA guide comprises a direct repeat sequence, a spacer sequence capable of hybridizing to a target nucleic acid in a eukaryotic cell, and a tracrRNA; and
a RuvC nuclease domain-containing CRISPR-Cas effector protein or a nucleic acid encoding the RuvC nuclease domain-containing CRISPR-Cas effector protein, wherein the RuvC nuclease domain-containing CRISPR-Cas effector protein is capable of binding to the RNA guide and of targeting the target nucleic acid complementary to the spacer sequence;
wherein the RuvC nuclease domain-containing CRISPR-Cas effector protein comprises an amino acid sequence that is at least 80% identical to an amino acid sequence of any of SEQ ID NOs: 1-5, 7, 8, or 15-24.

2. The system of claim 1, wherein:
(i) the RuvC nuclease domain-containing CRISPR-Cas effector protein comprises the amino acid sequence of any of SEQ ID NOs: 1-5, 7, 8, or 15-24;
(ii) the spacer sequence comprises 15 to 30 nucleotides;
(iii) the direct repeat sequence comprises a nucleotide sequence of:
(a) X₁X₂X₃GNX₆TX₈X₉GACACC (SEQ ID NO: 200), wherein X₁ is A or G, X₂ is A or C or G, X₃ is C or G, X₆ is A or C or T or U, N is any nucleic acid, X₈ is C or G or Tor U, and X₉ is C or G or T or U; or
(b) X₁GX₃GGTX₇X₈TTACAX₁₄C (SEQ ID NO: 201), wherein X₁ is C or G, X₃ is G or Tor U, X₇ is A or T or U, X₈ is C or G, and X₁₄ is A or C; or
(iv) any one of SEQ ID NOs: 9-11 or 25-34.

3. The system of claim 1, wherein:
(i) the target nucleic acid is a single-stranded RNA;
(ii) the target nucleic acid is an RNA selected from the group consisting of an mRNA, a tRNA, a ribosomal RNA, a non-coding RNA, a lncRNA, or a nuclear RNA; or
(iii) the target nucleic acid is a DNA selected from the group consisting of chromosomal DNA, mitochondrial DNA, single-stranded DNA, or plasmid DNA.

4. The system of claim 1, wherein the targeting of the target nucleic acid by the RuvC nuclease domain-containing CRISPR-Cas effector protein and the RNA guide results in a modification of the target nucleic acid, wherein:
(i) the modification in the target nucleic acid is a cleavage event;
(ii) the modification in the target nucleic acid is a nicking event; or
(iii) the target nucleic acid is comprised in a eukaryotic cell and the modification results in cell toxicity.

5. The system of claim 1, wherein the RuvC nuclease domain-containing CRISPR-Cas effector protein includes one or more amino acid substitutions within the RuvC nuclease domain relative to any one of SEQ ID NOs: 1-5, 7, 8, or 15-24.

6. The system of claim 1, wherein the RuvC nuclease domain is catalytically inactivated.

7. The system of claim 1, wherein the RuvC nuclease domain-containing CRISPR-Cas effector protein is fused to a base-editing domain, an RNA methyltransferase, an RNA demethylase, a splicing modifier, a localization factor, or a translation modification factor.

8. The system of claim 7, wherein the base-editing domain comprises Adenosine Deaminase Acting on RNA 1 (ADAR1), ADAR2, apolipoprotein B mRNA editing enzyme, catalytic polypeptide-like (APOBEC), or activation-induced cytidine deaminase (AID).

9. The system of claim 7, wherein the RuvC nuclease domain-containing CRISPR-Cas effector protein comprises:
(i) at least one nuclear localization signal (NLS); or
(ii) at least one nuclear export signal (NES).

10. The system of claim 1, wherein the tracrRNA:
(a) comprises a nucleotide sequence provided in Table 6; or
(b) is encoded by a sequence or fragment thereof, listed in Table 7.

11. An activated CRISPR complex comprising:
a CRISPR-Cas system of claim 1; and
a target nucleic acid bound to the RNA guide of the CRISPR-Cas system.

12. A method of modifying a collateral nucleic acid, the method comprising contacting the collateral nucleic acid with an activated CRISPR complex of claim 11, wherein the collateral nucleic acid comprises a nucleic acid sequence that does not have sequence similarity to the target nucleic acid or a fragment thereof.

13. A eukaryotic cell comprising the CRISPR-Cas system of claim 1.

14. A method of targeting and editing a target nucleic acid, the method comprising contacting the target nucleic acid with a CRISPR-Cas system of claim 1.

15. A method of cleaving a single-stranded DNA substrate, a single-stranded RNA substrate, or a double-stranded RNA substrate with the CRISPR-Cas system of claim 1, the method comprising contacting the single-stranded DNA substrate, single-stranded RNA substrate, or double-stranded RNA substrate with the CRISPR-Cas system, optionally wherein the single-stranded DNA substrate, single-stranded RNA substrate, or double-stranded RNA substrate lacks a protospacer adjacent motif (PAM) sequence protospacer flanking sequence (PFS) with a target nucleic acid.

16. A method of non-specifically cleaving a collateral nucleic acid, the method comprising contacting the collateral nucleic acid with an activated CRISPR complex of claim 11, wherein the collateral nucleic acid is a single-stranded DNA, single-stranded RNA, or double-stranded RNA, and wherein the collateral nucleic acid comprises a nucleic acid sequence that does not have sequence similarity to the target nucleic acid.

17. A method of detecting a target RNA in a sample, the method comprising:
(a) contacting the sample with the CRISPR-Cas system of claim 1 and a labeled detector RNA, wherein hybridization of the RNA guide to the target RNA causes a cleavage of the labeled detector RNA; and
(b) measuring a detectable signal produced by cleavage of the labeled detector RNA, thereby detecting the target RNA in the sample.

18. A method of modifying a RNA molecule, the method comprising contacting the RNA molecule with a system of claim 1.


SEQUENCE LISTING (493 sequences)

SEQ ID NO: 1 - protein, 772 aa, unidentified
  REGION 1..772: note = source = /note="Description of Unknown: bioreactor-anaerobic-anaerobic digester digestate sequence"
MTRIKYRQERTLVRGLQRLPNQDKAEFNKNVLGLRRHFEQFNLNVAELCQWLISFKPENPGSVCETKLFWEFMLEPENFCPDTYDRGPDWLRHEIFVLAAGWRTFEDIKAHNMPESLFESIKIASSKPRSKTAEALFIRLKSLEASHVMVLLKSASEWLSTRYVRQSENWKQNEQEWQKKKAAWEDKHPELTPAIRDKYNAIFVELGISEKRPHVCSWKKLSENKDNCDWAGERKNIGDKWISHSDLCIKYHEFARKLRSKQRQHFVDNANQYLELRRRYPQWTRDMAMNGLFKNVPLARNWFRNAWTNYLNALNILETTILENYSGHLPHCEKLSDECVFKKHTDNCRRYKLLLGEKLSNQERELEETYREWRREFLAPPNKPFLRYPSAQKLPTPKLFGRGYYDLDFTRHVVKLRLDDMPADNFVSFGFKPWPRGYDKKPGEINITSVHVHFIGTRARVGFRFAVPHSDSRFSVSQDKIDELRSGGFPGKSQDQEFLNEARQRLLDGMNENQKSALRIMAVDLGTHRAAAAFFTGCIFNKAKLLKLKKIDLLTEPKTDTTKPEKLSADEKKIQREKGLTQHHVGKHFETLEARTKEIISKRQNMKMAPSDDTPDIVGDHDLRHLTSHIRRMIRDWVRLNARQITELAEEENVDLIVFESMRGFRAPGYDKLDLKKKRRLAFFAYGQIRRKVAEKAVERGMRVITVPYFKSSQICAQCGRSQNDKNKLRDNKWKQSFQCEFSDCNYKTHSDENAARVLGRVFWGEITLPTD

SEQ ID NO: 2 - protein, 821 aa, unidentified
  REGION 1..821: note = source = /note="Description of Unknown: activated sludge metagenome sequence"
MLPTRYKPARTLVRPLGRLPHEPRKEFVEKCRRVRMHFEQFNIDVADLCQWLMSLRPNTRIGDAQSTVFWDFFLNPSILTVEADEKERDRWRLAAFDELLQIRFGHDPNAPPWSEEFRSAIRHVAQRPKSATAQRLFDRLRSLTAPHRLVLLKSAAEWIIARYQRGMENWQRQFAEWQREKEEWEAAHPNLTPEVRDAFTRVFKNLFENPDGDGKIGVRRKNPRICSWERLKLNKDNCVYAGQKGHGPLCWEFSKFVKAQKNAGTIKTFFVDVANKYLHVRRNLSKPGVKLKKSPRQEAFKRLYNQKGMEKARNWFTDAWSGYLTALNLNEKTILDHGCLKHCGAIGAEFEKSLCQFNPHTHLCVQYRNALESLEPAIRELEGDYREWRRLFLAPPRKPSFRYPSSRRLPMPKIFGEHFHQIDFDQSILRLRLEDMAEGEWIEFGFKPWPKDYRPGKDEVRVTSVHVNFHGNRMRAGFHFEAPAKPSRFACTQDELDDLRSKQFPRQSQDRQLLEVARRRLLESFDGMLESDLRILAVDLGEKGAAAAVYQGHGHEADVAIPIVKIDRLYDHVPDVLDVESARVPPPKFDDSRDPRGVRKEHVGRHLGQLQRGAQTLAQHRQQDESAPAALRRHDFRSLTRHIRWMIRDWTRHNAAQITAAAETHRCHLIVFESLRGFKPRGYDQMDFAQKARLAFFAYGRVRRKVVEKAVERGLRVVTVPYGFTSQICSECGHRQRNKGRLRKNKYQRRFVCECGEPKKSANKTAAPDRSATVSPCTCRLQLGSDVNAARVLARVFWDEIVLPTREEMREPAVDSAPPSK

SEQ ID NO: 3 - protein, 786 aa, unidentified
  REGION 1..786: note = source = /note="Description of Unknown: activated sludge metagenome sequence"
MHFEQFNIDVADLCQWLMSLRPNTRIGDAQSTVFWDFFLNPSILTVEADEKERDRWRLAAFDELLQIRFGHDPNAPPWSEEFRSAIRHVAQRPKSATAQRLFDRLRSLTAPHRLVLLKSAAEWIIARYQRGMENWQRQFAEWQREKEEWEAAHPNLTPEVRDAFTRVFKNLFENPDGDGKIGVRRKNPRICSWERLKLNKDNCVYAGQKGHGPLCWEFSKFVKAQKNAGTIKTFFVDVANKYLHVRRNLSKPGVKLKKSPRQEAFKRLYNQKGMEKARNWFTDAWSGYLTALNLNEKTILDHGCLKHCGAIGAEFEKSLCQFNPHTHLCVQYRNALESLEPAIRELEGDYREWRRLFLAPPRKPSFRYPSSRRLPMPKIFGEHFHQIDFDQSILRLRLEDMAEGEWIEFGFKPWPKDYRPGKDEVRVTSVHVNFHGNRMRAGFHFEAPAKPSRFACTQDELDDLRSKQFPRQSQDRQLLEVARRRLLESFDGMLESDLRILAVDLGEKGAAAAVYQGHGHEADVAIPIVKIDRLYDHVPDVLDVESARVPPPKFDDSRDPRGVRKEHVGRHLGQLQRGAQTLAQHRQQDESAPAALRRHDFRSLTRHIRWMIRDWTRHNAAQITAAAETHRCHLIVFESLRGFKPRGYDQMDFAQKARLAFFAYGRVRRKVVEKAVERGLRVVTVPYGFTSQICSECGHRQRNKGRLRKNKYQRRFVCECGEPKKSANKTAAPDRSATVSPCTCRLQLGSDVNAARVLARVFWDEIVLPTREEMREPAVDSAPPSK

SEQ ID NO: 4 - protein, 769 aa, unidentified
  REGION 1..769: note = source = /note="Description of Unknown: candidate division Zixibacteria bacterium Bacteria. sequence"
MNRTRYREERTITRGMRRLPGEERKSFKAKVITLRRNFEQFNTDVSEICQWLMSIRPNGKHNIPNTEPFWDFILEPHNFVVNQEETNIDSVRLVVFEMAVGWRQVTDVANFELERQLLMSLESIQSVPRTIAAKRMLQRIKNYEFQHKMVLLRSAVEWINTRFIRTYKNWEMNIKEFLEKKKVWENDHPKLTEEIRNTFNKVFDELEISKKNPNICRWSHLKKNRDNCNYAGVRIKVGGEYNNHSEKCKRYQDFLKKHSAHKKYFAANAMMYINIRKKRRDLTKREAIKVLLDKIPQARSWFPQAWDNYLEYLGLNEISLINKFDGQLPHCLRLDTECIYNVHTQSCRKYYVLLKDLPDKYLSLEETYREWRKYFLREPRKPVFAYPSTRQRTVSKIFGRDYFEADYDNSIIKLRLDDMAEGQFLSFGFKPWPVDYDVQPIDTEITSVLVHFIGTRARVGFRFKMPHRPSRINIKQDELDELRSRSRLIQEKDQALLEKVRLRLRDGFIGIFDKELRVLAVDLGTSSCATAFFVGRQFQESSRLQIVKYDRVYKSNYEIKKRRNNKGIDKQKQLLFKEKGLNQYHIKVHLDKLAEQNKQIIKKREASGNPTPTEQDMRRLSLHIGWMHRDWVRINASQIIKSAKKLRADLIVFESLRDFRPMMFNEFDIDKKRRLAFFPFGLIRHKVIEKAVESGMRVVTVPYMFSSQFCGACGRQQNDKKRLQKNKTDKRGACFICEYNDCAFEGDPDENAARVLGGVFWGNIGLPLS

SEQ ID NO: 5 - protein, 830 aa, unidentified
  REGION 1..830: note = source = /note="Description of Unknown: aquatic-thermal springs-hot spring sediment sequence"
MHPSRYKTARTLVRRLCRLPGEDRSAFRSKVGLLRGHFEQFNVDVSELCQWLMSLRKRNKVPENPATFGALGDFLLQPGLPGEETDEKEADRLRLAVFDAVAGFRMLEDRLAASIPASLSDAIRDEAVFLAGVRAAGKPSGLARVLARLEACAPAQRLVLLKSAAEWIVARFLRGTENWMRQRAEWEKEKAAWEAAHPHLTPEVRAQFNKIFESLHDPENSGKPGVSRKNPRICPWDRLKQNLDNCCYGEKGHSALCWRYQDFLKQRMGENRRDKKNFSATAMDLAQICREWKIQHSRNALNNPRVLDRLFAEHERRKQDKTKKESRSPKPRQGGYKANPKADYLRSFKAHWKAYLEHMKLNDTTVLERGCLPHCLSIKKNGKESTCKWNKHTELCLEYKRSLAPLPDSVLELEPEYREWRRLYLHGPGRPHFRYPSAGELPLPKVFGEGFHQVDLDRSIVRLRLEGAAEGEWLEFGFIPWPRGYQPSRREVLITSVQVHFVGTRPRAGFRFDVSHRTSRFGCSQDELDELRSRRYPRQAQDKEFLAAARAQLIQTFEGGEGAARQQMRVMSVDLGEGGACASIYEGRTHQKDESLKVIKIDRRYDQHPEVLEKDVGAAKPQKFEKSDPRGVRKEHVARHLNRIAAGASAIAEHRRKERSDAECSVGELQEHDFRSLKRHIAWMIRDWVRLNAAQIIDVAKQHCCDLIVFESQRGFRLPGYDELDRGKKQRFAILAFGRIRRKVVEKAVEHGMRVVTVPYFASSQVCSACKRVQENRGSWRENKKKRVFACEFCKLKLNSDANASRVLARVFWGEIELPEPTRAHLPSKA

SEQ ID NO: 6 - protein, 767 aa, unidentified
  REGION 1..767: note = source = /note="Description of Unknown: aquatic-thermal springs-hot spring sediment sequence"
MAQASSTPAVSPRPRPRYREERTLVRKLLPRPGQSKQEFRENVKKLRKAFLQFNADVSGVCQWAIQFRPRYGKPAEPTETFWKFFLEPETSLPPNDSRSPEFRRLQAFEAAAGINGAAALDDPAFTNELRDSILAVASRPKTKEAQRLFSRLKDYQPAHRMILAKVAAEWIESRYRRAHQNWERNYEEWKKEKQEWEQNHPELTPEIREAFNQIFQQLEVKEKRVRICPAARLLQNKDNCQYAGKNKHSVLCNQFNEFKKNHLQGKAIKFFYKDAEKYLRCGLQSLKPNVQGPFREDWNKYLRYMNLKEETLRGKNGGRLPHCKNLGQECEFNPHTALCKQYQQQLSSRPDLVQHDELYRKWRREYWREPRKPVFRYPSVKRHSIAKIFGENYFQADFKNSVVGLRLDSMPAGQYLEFAFAPWPRNYRPQPGETEISSVHLHFVGTRPRIGFRFRVPHKRSRFDCTQEELDELRSRTFPRKAQDQKFLEAARKRLLETFPGNAEQELRLLAVDLGTDSARAAFFIGKTFQQAFPLKIVKIEKLYEQWPNQKQAGDRRDASSKQPRPGLSRDHVGRHLQKMRAQASEIAQKRQELTGTPAPETTTDQAAKKATLQPFDLRGLTVHTARMIRDWARLNARQIIQLAEENQVDLIVLESLRGFRPPGYENLDQEKKRRVAFFAHGRIRRKVTEKAVERGMRVVTVPYLASSKVCAECRKKQKDNKQWEKNKKRGLFKCEGCGSQAQVDENAARVLGRVFWGEIELPTAIP

SEQ ID NO: 7 - protein, 720 aa, unidentified
  REGION 1..720: note = source = /note="Description of Unknown: aquatic-sediment-hot spring sediment sequence"
MSIRPDAKKPDKETKSFWDFFLNPESFFDPTINNVDIIRLNLFKVITGRESEANLIRYNLPLLLYESIILLKKQEPSDTARRLFARLKKMEPVHVMILLKAAAEWVYARYQRLMDNHEYQYKVWHDEKSAWENKHPELTPEIREKYNSIFKELGRKQGVTIRKNPRICNWEKLEENKDNCGYNGKRIQFGDKWKAHSMLCIEYRNFLRDNKITGKRIGFFATHAYNYLKLRAHQPRLTKDEAFKRIFKSAPNGIYWFPKAWKNYLQFMNLNELNLIRKYNANLPHCLEFKGDKDCQYNKHTELCQEYKTLLLEKFTEDELKLEGLYREWRKQYLSGPSKPAFRYPSCSKLPTPKIFGKRFHEIDFENSIVRLRLDDMPDGEYLTFKFKPWPNDYQPQPEEAEISSVHVHFVGTRARVGFRFKIAHKQSRFKTSQDEIDELRSRKYPRQAQDADFLKAAREKLLQSFKGENPTKEIKIMAVDLGEYRGYISVYKGENIEISEPLSILKIDKLYDSLESAGVDKTDLAKYIKDHKGLIKEHVDSHLKVISEKANEITKHRPAGKKTGASNLKDYDLRSLTAHTGWMIRDWVRLNVSQIIRIAEKHEVDLIVLESLRGWKAPGYDEFDLRKKRWLAFFSYGRIRHKLKEKAVERGMMVVTVPYYKSSQICSKCGKEQENKGLWKKNKNERLFICDYPGCGHRDNSDANAAKVLAKIFWGEIVL

SEQ ID NO: 8 - protein, 774 aa, unidentified
  REGION 1..774: note = source = /note="Description of Unknown: aquatic-sediment-hot spring sediment sequence"
MDNNRTRYREERTLVRSLRPLDGENRNAFKNRVNRLRIHFKNFNLDVSEICQWLMSIRPDAKKPDKETKSFWDFFLNPESFFDPTINNVDIIRLNLFKVITGRESEANLIRYNLPLLLYESIILLKKQEPSDTARRLFARLKKMEPVHVMILLKAAAEWVYARYQRLMDNHEYQYKVWHDEKSAWENKHPELTPEIREKYNSIFKELGRKQGVTIRKNPRICNWEKLEENKDNCGYNGKRIQFGDKWKAHSMLCIEYRNFLRDNKITGKRIGFFATHAYNYLKLRAHQPRLTKDEAFKRIFKSAPNGIYWFPKAWKNYLQFMNLNELNLIRKYNANLPHCLEFKGDKDCQYNKHTELCQEYKTLLLEKFTEDELKLEGLYREWRKQYLSGPSKPAFRYPSCSKLPTPKIFGKRFHEIDFENSIVRLRLDDMPDGEYLTFKFKPWPNDYQPQPEEAEISSVHVHFVGTRARVGFRFKIAHKQSRFKTSQDEIDELRSRKYPRQAQDADFLKAAREKLLQSFKGENPTKEIKIMAVDLGEYRGYISVYKGENIEISEPLSILKIDKLYDSLESAGVDKTDLAKYIKDHKGLIKEHVDSHLKVISEKANEITKHRPAGKKTGASNLKDYDLRSLTAHTGWMIRDWVRLNVSQIIRIAEKHEVDLIVLESLRGWKAPGYDEFDLRKKRWLAFFSYGRIRHKLKEKAVERGMMVVTVPYYKSSQICSKCGKEQENKGLWKKNKNERLFICDYPGCGHRDNSDANAAKVLAKIFWGEIVL

SEQ ID NO: 9 - DNA, 36 bp, synthetic construct
  misc_feature 1..36: note = source = /note="Description of Artificial Sequence: Synthetic oligonucleotide"
  source 1..36: mol_type = other DNA
gttgtagcggcccctgaattagcggtttccgacacc

SEQ ID NO: 10 - DNA, 36 bp, synthetic construct
  misc_feature 1..36: note = source = /note="Description of Artificial Sequence: Synthetic oligonucleotide"
  source 1..36: mol_type = other DNA
ggtgcggcagccggtagaactgcggcttccgacacc

SEQ ID NO: 11 - DNA, 36 bp, synthetic construct
  misc_feature 1..36: note = source = /note="Description of Artificial Sequence: Synthetic oligonucleotide"
  source 1..36: mol_type = other DNA
gttggagtcagcaaggaatcaacggttttggacacc

SEQ ID NO: 12 - skipped

SEQ ID NO: 13 - DNA, 36 bp, synthetic construct
  misc_feature 1..36: note = source = /note="Description of Artificial Sequence: Synthetic oligonucleotide"
  source 1..36: mol_type = other DNA
ggtggaaagggccggagatttaccggctctgacacc

SEQ ID NO: 14 - skipped

SEQ ID NO: 15 - protein, 761 aa, unidentified
  REGION 1..761: note = source = /note="Description of Unknown: Planctomycetes bacterium sequence"
MGLRKRYVELCDNSGLRADLSSFGTLGNFLLEPSLDGSEADERERDRWRLAVFDIVAGFRADIQLDGRTLPNELRQAITDAADQMRNTIADGKKKTGLRRLFQRLGDLEEAHRLVLLKSAAEWIVARYLRSVENWVRQHTEWEKEKQQWESHKDNKTDHTLLTPDIREKYTAVFKLLVWDKDKPPGVRNKRPRICPYDRLKQNIDNCCYAGQKGHGPLCWKYNEFVKARKARNPSFNDKKFAEDVAKYLPPRAKGEQRHLILKAIFPNKQQDQQRFSDNWTAYLSAMKLNEQTLVQHQQLPHCLKIGETFEKSACVWNPHTDLCGQYHRALVNPNNGFDDAMLSLEPLYRAWRRNYLAGPRKPSFRYPASRDLPMPKIFGDGFHEVDFENSIVRLRLDDMPRGQWLDLGFTPWPRGYKPSRAEISEPGRVTSVHVHFVGVRARIGFRFEVAHAPSRFGCTQDEIDELRSRHYPRQAQDQEFLEAARKCLLDSMTEGSENDLRIMAVDLGEKGACAAVYRGRTREMDIPLAIVKINKLYEKPPKTLEPDRHSRPEESKRKFEEADPRGVRKEHVGRHLERVAAMSQEIAKHRQPAVATTVTVSVNDLRGLKRHVAWMIRDWARHNASQIVKAAEEHQCDVIVFESLRGFRPPGYDKLDEMSAKKKRWLAMFAYGRVRRKVIEKAVERGMRVVTVPYFKSSQVCSDCGREQVNVGLLRKNKLSKGQFVCENCKVTLTSDANAARVLARVFRGEIMLPKARPGA

SEQ ID NO: 16 - protein, 772 aa, unidentified
  REGION 1..772: note = source = /note="Description of Unknown: anaerobic digester metagenome sequence"
MTRTKYREERTLVRGLQRLPNQDKTEFNKNVLRLRRHFEQFNLNVAELCQWLISFKPENPNSVCETKLFWEFMLEPENFCPDTYDRGSDWLRHEIFLLVAGWQTFEDMKAYNIRESLLTSAKMASSRPRSKTAEVLFIRLKSLEASHVMVLLKSASEWLSTRYIRQSENWKRHVQEWQKEKAAWEDKHPKLTPAIRDKYNAIFVELGISEKRSRVCSWKKLSENKDNCDWAGERKNIGDKWVSHSALCSKFNEFHRSLKGSPRKYFVTNANQYLELRRKNPQWTRDMAMQGLFRNVPGAQNWFPNAWTNYLNALNILETTILKDYNGHLPHCNKLSDECLFNQHTFNCRQYKRLLVGKLSSQERDLEETYREWRREYLAPPDKPFLRYPSAQKLPTPKLFGRGYYDLDFTRHVVKLRLDDMPAENFVSFGFKPWPRDYDKKPGEINITSVHVHFIGTRARVGFRFAVPHSYSRFSVSQDKIDELRSRVFPRKSQDLEFLNEARLRLLDGMNENQKSALRIMAVDLGTHRAAAAFFTGRIFDKPKLIKLKKIDLLTESITDTTQPEKHSANEKKIQREKGLTQHHVGKHLDTLETRTKEIISKRQNIKITPSDDTLGTLGDHDLRHLTSHIQRMIRDWVRLNAQQITELAEKEKVDLIVFESLRGFRAHAYDKLDLEQKRRLAFFAYGRIRRKVAEKAVERGMRVLTVPYFESSQICAQCGRSQNDKNKLRDNKRKQSFQCEFSDCNYKTHSDENAARVLGRVFWGEITLPTD

SEQ ID NO: 17 - protein, 696 aa, unidentified
  REGION 1..696: note = source = /note="Description of Unknown: aquatic-marine-marine sediment sequence"
MLEPERFCARNDPGDPRGDWIRHAVFEVVAGWKLSDNLDQYNLNEELTASIEAAMDKTRTATAEALFERLMRREASNVMVLLKVAAEWIAAKYVHQMENWKRQKEEWEKEKAEWENSHTELTEQVRDKYNRIFKELDIKNKRPRVCTWKRLSENKDNCDWAGKRKLIGKSWVNHAALCYKYHEYSEAPKVKHRDHFIANANKYIKIRREYPQWSRDQAMKTLFKNEPRASYWFPKEWKMYLGALGIEENTIIGNYTGCLPHCLKITHKCRFNKHTNECRRYKDLMHERLTNEERQLEELYREWRRNYLIAPGKPALRYPSARTLPTPKIFGSGYYRLDFERNQVHLRLDDMSQGDFISFGIKAWPRKYDYQPDTIDITSVQVHFVGTRARIGFRFKVPHRESIFTIRQDDIDELRSRKYPRESQDQKFLEEVRKRILNGFSEDQIAKLKIMAVDLGSDEGGVAFFKGHVFEKGESLKIIKIDELFESKKNEEAEKAKGLNVHHVGRHLDVLQKKSQEIALLRQGMTNAPSNDMVQSLYPNDMRRLTSHIRRMIRDWVRLNSSQIIKLAEREQVELIVFESMRGFLAPGYDKIDPDKKRRLAFFAFGSIRRKVAEKAVERGMRVVTVPYHCSSQVCAKCGKEQEDKKRFRKNKEKREFVCEDKKCNHKTNSDINAAHVCGRVFWGEINLLGKKIKIK

SEQ ID NO: 18 - protein, 768 aa, unidentified
  REGION 1..768: note = source = /note="Description of Unknown: aquatic-marine-marine sediment sequence"
MTRTKYREERTLVRSINRLPKQDKAEFKKNVLRLRRYFEQYNLDVSETCQWLISYRGLNLDEICKTQLFWEFMLEPERFCARNDPGDPRGDWIRHAVFEVVAGWKLSDNLDQYNLNEELTASIEAAMDKTRTATAEALFERLMRREASNVMVLLKVAAEWIAAKYVHQMENWKRQKEEWEKEKAEWENSHTELTEQVRDKYNRIFKELDIKNKRPRVCTWKRLSENKDNCDWAGKRKLIGKSWVNHAALCYKYHEYSEAPKVKHRDHFIANANKYIKIRREYPQWSRDQAMKTLFKNEPRASYWFPKEWKMYLGALGIEENTIIGNYTGCLPHCLKITHKCRFNKHTNECRRYKDLMHERLTNEERQLEELYREWRRNYLIAPGKPALRYPSARTLPTPKIFGSGYYRLDFERNQVHLRLDDMSQGDFISFGIKAWPRKYDYQPDTIDITSVQVHFVGTRARIGFRFKVPHRESIFTIRQDDIDELRSRKYPRESQDQKFLEEVRKRILNGFSEDQIAKLKIMAVDLGSDEGGVAFFKGHVFEKGESLKIIKIDELFESKKNEEAEKAKGLNVHHVGRHLDVLQKKSQEIALLRQGMTNAPSNDMVQSLYPNDMRRLTSHIRRMIRDWVRLNSSQIIKLAEREQVELIVFESMRGFLAPGYDKIDPDKKRRLAFFAFGSIRRKVAEKAVERGMRVVTVPYHCSSQVCAKCGKEQEDKKRFRKNKEKREFVCEDKKCNHKTNSDINAAHVCGRVFWGEINLLGKKIKIK

SEQ ID NO: 19 - protein, 855 aa, unidentified
  REGION 1..855: note = source = /note="Description of Unknown: aquatic-thermal springs-hot spring fe si sediment sequence"
MVASRYREARTLVRRLGRLPGEGEASFRAKLARLRKHFERFNVDVSELCQWLMGLRKQHCHKAGTASFGVLGDFLLHPESSNVAPGEAEADRWRLLVFDAVAGICPAKQLTNAPMPRGLPEAMEREAKRLADLEWRRRNTTQSKLIDRLAILEPAHRLVLLKAAAEWVVSRYQRGLENWAARRGEWEKERHAWEKRHPALSEEVRQRFTEVFKSLNDPERTDKPGVRRKNPRICPYERLRANIDNCIYAGEKGHGALCWKYAEFVKARKTRQPQFNDKRFAEDAEKVLPLLKQGMKRHQALQRLFPRDRPHGQLAQQRFNENWTAYLQALGLKEERVVNRGRLPHCLKIGETHEKSKCAWNPHTELCKQYKRALDQFDEETLKLEPLYREWRRDYLAGPGKPQFRYPSSRELPMPKIFGAGFHEIDFDRSILRLRLEDMPEGEWIEFGFAPWPRGYRPSKEEVKVKGAISSVHVNFVGVRARAGFRFDVRHRASRFQCTQDELDQLRSRAYPRRAQDREYLDAARKRLLESFAEGEEAAKRELRLLAVDLGETGACAAVYHGHAHQKDVQLAILKINRLYTQLPEALEPDPHGRPEEGKRKYERDDPRGVRKEHMGRHLKRMADGAASIAARRQGTMPATVTMAGHDFRGLKRHVTWMIRDWARHNAARIVAAAEEHGCDLIVFESLRGQKVPGYHELSSEKERDKRQLAMLSYGRIRHKVREKAVERGMRVVMVPDYRSSRLCSSCGHEQCAEKWQERRWRENKKKRLFKCVCGEPAPTEKPHHGGSAPDRQRAARRDGPGPGKRPGMAADQAKQRCRCGAEMNSDANAARVLARVFWGEITPPASERSFAGSA

SEQ ID NO: 20 - protein, 803 aa, unidentified
  REGION 1..803: note = source = /note="Description of Unknown: aquatic-thermal springs-hot spring fe si sediment sequence"
MGLRKQHCHKAGTASFGVLGDFLLHPESSNVAPGEAEADRWRLLVFDAVAGICPAKQLTNAPMPRGLPEAMEREAKRLADLEWRRRNTTQSKLIDRLAILEPAHRLVLLKAAAEWVVSRYQRGLENWAARRGEWEKERHAWEKRHPALSEEVRQRFTEVFKSLNDPERTDKPGVRRKNPRICPYERLRANIDNCIYAGEKGHGALCWKYAEFVKARKTRQPQFNDKRFAEDAEKVLPLLKQGMKRHQALQRLFPRDRPHGQLAQQRFNENWTAYLQALGLKEERVVNRGRLPHCLKIGETHEKSKCAWNPHTELCKQYKRALDQFDEETLKLEPLYREWRRDYLAGPGKPQFRYPSSRELPMPKIFGAGFHEIDFDRSILRLRLEDMPEGEWIEFGFAPWPRGYRPSKEEVKVKGAISSVHVNFVGVRARAGFRFDVRHRASRFQCTQDELDQLRSRAYPRRAQDREYLDAARKRLLESFAEGEEAAKRELRLLAVDLGETGACAAVYHGHAHQKDVQLAILKINRLYTQLPEALEPDPHGRPEEGKRKYERDDPRGVRKEHMGRHLKRMADGAASIAARRQGTMPATVTMAGHDFRGLKRHVTWMIRDWARHNAARIVAAAEEHGCDLIVFESLRGQKVPGYHELSSEKERDKRQLAMLSYGRIRHKVREKAVERGMRVVMVPDYRSSRLCSSCGHEQCAEKWQERRWRENKKKRLFKCVCGEPAPTEKPHHGGSAPDRQRAARRDGPGPGKRPGMAADQAKQRCRCGAEMNSDANAARVLARVFWGEITPPASERSFAGSA

SEQ ID NO: 21 - protein, 853 aa, unidentified
  REGION 1..853: note = source = /note="Description of Unknown: aquatic-thermal springs-sediment sequence"
MKNPKYREERTLTMGIRSFPLEDKSHFKEKVRKLRKFFEIFNKDVADLCQWLIVFRKGGKSENIPIWEFFINPLESIKDISEDQADELKRKVLDVIIGNESINNIRNEKLPTEVLKYLENLDDNWNQSVQELFKRMKDKKPSHRQILLKAASNLIYSRYYKTLENREKQKEEWKKERDTWQRKNPDLKEDIRTKYTEVYKQLGIKRKAPRICEWEKLKNWKHNCTYSSNKQHTERCFDFNKLFSSADNKKKRKWFIDNAHLYLQYLSKSNQKDALKQLYHKKKGSEKWFGRLWNDYLKLMKISEQDILTKYHCILPHCISENLGSAKYREYFDNSEYGFARSEFGKCANTYLGSRKKGSNKADSLQMVYGKHKSNIDERFEEIWNDYLKFIKKTEEEAIIEYNHSLPVYLKSDYCQFRPHGDKCKQYRDLLADFTEVEKSLEKKYREWRKSYLSGPGKPQFRYPSAKTIAIPKLFGSGYFRIDFEESILNLKLENDEWLSLGFKPWPKRKDYDIHYSDIEIPSVSIHFVGTRAKVGFRFKVKHKESRFKVAQEEIDKLRSQKYPRQYQDNDFLKEARELLLKDFDGDKNEMKILAFDWGETGAYAALFTGEKYEKGFQLPVLKFEKLYCSDKFKEAWDQKKKEQKWSKDESKAKLKEYKLKGLTKGHVGKHLENISEKAVKIAEIRGEKKDEKLLRPSDLRRLFSHSAWMIRDWVRLNTKQLIKIAEKNEVDLIVFESMRGSAPPSYDKLEEITEKIKWAFFSLGRIRHKVTEKAVERGMRTITVPYVKSSQVCFDCGKEAEDKKKWQHHKTELTKFICEHCPADLNSDENAARVLCKVFWGDITLPSTEWEK

SEQ ID NO: 22 - protein, 774 aa, unidentified
  REGION 1..774: note = source = /note="Description of Unknown: terrestrial-deep subsurface sequence"
MSNTRYSESRTLVRRLRRLPGESREEFRGKVRRLRKQFAQFNVNASELCQWLMSLRPGGKKASDRTKEFWEFFLEPERFLENQDDDRCDACRLAVFDVAAGLAPADRLGDYGVSQALAESVHVIGQISLTPTAAKLFTRLCGFEASHRQVLLKAAAEWIVAHYLRGYENWVRRHEEWEKEKARWEASHPELTQAARDDFNRIFKDLGIERKRPRVCTGERLKANKDDCDWAGERIPVGGTWRNHSSLCVKYWRFLKEYPRKARVPRQFREFFVTNAKTYMDLRRTSRGDRSVTMAAFLRKQRNAQWFPQAWEAYLKALEVNEQTVLAAGYGLPHCTEIGPDADCQFNKHTADCEKYRRALDARPDLLPLEKLYRHWRREYLSGPGKPCFQYPSQRKLPMPKIFGRGYFRVDLASSIIELRMEGGRDFERFRIAAWPSDYTPSAQEAQITSVHVSFVGTRALAGFRFEVPHKASRFAAGQDQIDELRSRKYPRRAQDAEFLVAARKRLLESFAGGAEHDVRILAVDLGTSNGAVAVFRGRSLEKAMPLDVIKLEKLHSSSPKENRGAGPEPSEEERKKARARGLRPSHVGRHLENWALAAREIANQRGNEADGPATLGDHDLRRFSLHIRWMIRDWVRLNVSQIIEAAEGNHVDLIVFESMRGWRAPGYDTVDDEKKRRLAFFAHGRIRHKIREKAVERGMRVVTVPYFMSSQFCGSCGTQQQDTRKLKTNKRERTSFTCENCGHRANSDENAAQVLAKVFWGDVVLPEDPDDCS

SEQ ID NO: 23 - protein, 763 aa, unidentified
  REGION 1..763: note = source = /note="Description of Unknown: terrestrial-deep subsurface sequence"
MVRRLRRLPGESREEFRGKVRRLRKQFAQFNVNASELCQWLMSLRPGGKKASDRTKEFWEFFLEPERFLENQDDDRCDACRLAVFDVAAGLAPADRLGDYGVSQALAESVHVIGQISLTPTAAKLFTRLCGFEASHRQVLLKAAAEWIVAHYLRGYENWVRRHEEWEKEKARWEASHPELTQAARDDFNRIFKDLGIERKRPRVCTGERLKANKDDCDWAGERIPVGGTWRNHSSLCVKYWRFLKEYPRKARVPRQFREFFVTNAKTYMDLRRTSRGDRSVTMAAFLRKQRNAQWFPQAWEAYLKALEVNEQTVLAAGYGLPHCTEIGPDADCQFNKHTADCEKYRRALDARPDLLPLEKLYRHWRREYLSGPGKPCFQYPSQRKLPMPKIFGRGYFRVDLASSIIELRMEGGRDFERFRIAAWPSDYTPSAQEAQITSVHVSFVGTRALAGFRFEVPHKASRFAAGQDQIDELRSRKYPRRAQDAEFLVAARKRLLESFAGGAEHDVRILAVDLGTSNGAVAVFRGRSLEKAMPLDVIKLEKLHSSSPKENRGAGPEPSEEERKKARARGLRPSHVGRHLENWALAAREIANQRGNEADGPATLGDHDLRRFSLHIRWMIRDWVRLNVSQIIEAAEGNHVDLIVFESMRGWRAPGYDTVDDEKKRRLAFFAHGRIRHKIREKAVERGMRVVTVPYFMSSQFCGSCGTQQQDTRKLKTNKRERTSFTCENCGHRANSDENAAQVLAKVFWGDVVLPEDPDDCS

SEQ ID NO: 24 - protein, 752 aa, unidentified
  REGION 1..752: note = source = /note="Description of Unknown: terrestrial-soil sequence"
MAVTRYREERTLVRGLRRLPGQSREQFRKNVLLLRRHFERFNVDVSDICQWMMGLRPKDGEVTPATQPLWDFMLEPSDGQGNAQGDPDRMRLLAFRVATGVEHSQSGVRLPLHVQESLRHVAALTSTESARRLILRFQQLEQSHQMILLKSASEWVRTRYSNANENWQRNRPLWEKEKAEWEKEHPALTPDACRKFSDIFKELGIKDKRPRICGWNRLKLPKDNCDYAGERVGGGRHAPLCKFYREFQAGLRREYKKQFPDNALKYLALRKQKGHTQAVVLQQFCAKDRRKSGWFPKAWMTYLQKLNVTEETLIQRYQGQLPHCVKIDNKTGCSFNPHTNDCLEYKKRILKLPESDRELETQYREWRRDYLSGPRKPSFRYPSSRNLPTPKIFGAGYYEADFTRSMLRLRLDDMPRGRFIEFGFKPWPSDYDIQPVSTQITSAHIHFIGTRARVGFRFAVAARPSRLRISQDEIDALRRQYPRAAQDQQFLDHVRPLILDSFAGNPKQELRILTIDLGTSGGAAAAFCGVTLVKSEVLKVIKLDKLYDLLDREDKRSPTSGLGEGHVGRHLEALSKEAAKIAQHRTTWKNPGLRPFDERQLTSHIRWMIRDWVRLNAQQIIEIAERENADLILFESMRGYYPKARDKYDSAQKVRLGFFSYGAIRRKVAEKAVERGMRILTLPYKFSSQICSKCGRKQENRGLKTKKAKRLFKCEHTGCGTELNSDENAARVLAGVFWGTIKLPEKAVVSHT

SEQ ID NO: 25 - DNA, 36 bp, synthetic construct
  misc_feature 1..36: note = source = /note="Description of Artificial Sequence: Synthetic oligonucleotide"
  source 1..36: mol_type = other DNA
ggtgcgaccggccgtcgattgaccggtttcgacacc

SEQ ID NO: 26 - DNA, 36 bp, synthetic construct
  misc_feature 1..36: note = source = /note="Description of Artificial Sequence: Synthetic oligonucleotide"
  source 1..36: mol_type = other DNA
gttgtagcgccccctgaattagcggtttccgacacc

SEQ ID NO: 27 - DNA, 36 bp, synthetic construct
  misc_feature 1..36: note = source = /note="Description of Artificial Sequence: Synthetic oligonucleotide"
  source 1..36: mol_type = other DNA
ggtgtaagtccgactgaatgcgaggtttccgacacc

SEQ ID NO: 28 - DNA, 36 bp, synthetic construct
  misc_feature 1..36: note = source = /note="Description of Artificial Sequence: Synthetic oligonucleotide"
  source 1..36: mol_type = other DNA
gctgtgactcaccggcatttgcggggttcttacacc

SEQ ID NO: 29 - DNA, 36 bp, synthetic construct
  misc_feature 1..36: note = source = /note="Description of Artificial Sequence: Synthetic oligonucleotide"
  source 1..36: mol_type = other DNA
gttgtgaccgcccccggaatcacggtttgcgacacc

SEQ ID NO: 30 - DNA, 36 bp, synthetic construct
  misc_feature 1..36: note = source = /note="Description of Artificial Sequence: Synthetic oligonucleotide"
  source 1..36: mol_type = other DNA
ggtgtagcgggcctcaaaaacacggcatccgacacc

SEQ ID NO: 31 - DNA, 46 bp, synthetic construct
  misc_feature 1..46: note = source = /note="Description of Artificial Sequence: Synthetic oligonucleotide"
  source 1..46: mol_type = other DNA
ggtttgcaaacagcgagaacatctgtgttttggtggtagttacaac

SEQ ID NO: 32 - DNA, 36 bp, synthetic construct
  misc_feature 1..36: note = source = /note="Description of Artificial Sequence: Synthetic oligonucleotide"
  source 1..36: mol_type = other DNA
gtttgcaaacagcgagaacatctgtgttttggtggt

SEQ ID NO: 33 - DNA, 36 bp, synthetic construct
  misc_feature 1..36: note = source = /note="Description of Artificial Sequence: Synthetic oligonucleotide"
  source 1..36: mol_type = other DNA
ggtggagccggccgtgaattagggggtttcgacacc

SEQ ID NO: 34 - DNA, 36 bp, synthetic construct
  misc_feature 1..36: note = source = /note="Description of Artificial Sequence: Synthetic oligonucleotide"
  source 1..36: mol_type = other DNA
ggtgttgtgaaccctgaaagaacggattccgacacc

SEQ ID NO: 35-102 - skipped (68 entries)

SEQ ID NO: 103 - DNA, 10 bp, synthetic construct
  misc_feature 1..10: note = source = /note="Description of Artificial Sequence: Synthetic oligonucleotide"
  source 1..10: mol_type = other DNA
ggtgtcggaa

SEQ ID NO: 104-112 - skipped (9 entries)

SEQ ID NO: 113 - DNA, 12 bp, synthetic construct
  misc_feature 1..12: note = source = /note="Description of Artificial Sequence: Synthetic oligonucleotide"
  source 1..12: mol_type = other DNA
gagccggtaaat

SEQ ID NO: 114-116 - skipped (3 entries)

SEQ ID NO: 117 - DNA, 159 bp, synthetic construct
  misc_feature 1..159: note = source = /note="Description of Artificial Sequence: Synthetic polynucleotide"
  source 1..159: mol_type = other DNA
tccgttcttctttgtagggtgttgtgaaccctgaaagaacggcgggtggcccggaccttggggcgtgttgaaaaacccgtttgtcgtcagttgatcggtgaaggttatcaagcgatggcgatcctgtagttgagatcgacggggttggtgtcagaccga

SEQ ID NO: 118 - DNA, 303 bp, synthetic construct
  misc_feature 1..303: note = source = /note="Description of Artificial Sequence: Synthetic polynucleotide"
  source 1..303: mol_type = other DNA
aacagcgacgaaaacgccgcacaggtgctggcgaaggtgttctggggcgacgtcgttctacccgaggatccggacgattgctcttgacgaagatatcgaaggtggtatagtgcccttgtgtcctcgtcgtgagtcgataatggctcggacaaaccccggcggagccttcggtgccaacgggaggaacctgagcccggaggatacaaactctctaattcgtggttgatatggcgatcaagaatgcggtttctttcaggcaattgtgtcgcaactctttgtcgatccagggattacgacgggaca

SEQ ID NO: 119 - DNA, 197 bp, synthetic construct
  misc_feature 1..197: note = source = /note="Description of Artificial Sequence: Synthetic polynucleotide"
  source 1..197: mol_type = other DNA
ccagacagcgaaaatgctgaataatcccggggacggcatacctaattgggttccagattgcccagcaggcagaagagagcgtttatcgcatttggggtgccggcggagagggggcgggccaagggtgtcactggatgtctccgctggtacccggcaaagcgtcaaacgcaatcaatcccggccgaaggccttccgct

SEQ ID NO: 120 - DNA, 310 bp, synthetic construct
  misc_feature 1..310: note = source = /note="Description of Artificial Sequence: Synthetic polynucleotide"
  source 1..310: mol_type = other DNA
agtacgcgggcacgcgggggcgtgtttagtacgcaggcacgcgggggcgtgtttagtacgcgggggcgtgattagtacaaaggggcaagtcaggtatacatcattaaagatgtcgaaaccgcaggggtttcgacctactcgaagaaagaaggtgtcggaacggcctgcatcatggggtttgggagggggattgggggctatttgaatcgaaacaaagttgatattgtccctatgcgggggattgtttgtagtgttgttcgagtaagaccaggttttcatcggggatgcggcctttttcaaaagagtcgac

SEQ ID NO: 121 - DNA, 191 bp, synthetic construct
  misc_feature 1..191: note = source = /note="Description of Artificial Sequence: Synthetic polynucleotide"
  source 1..191: mol_type = other DNA
cgagcgcgcgcttcagcacgaagcacgaggtgcggctgccggttgcattgcggcagaatgattggcccgcctgttcacgctttcccgaccggcagtggaaaacaaacgtcccgcacgtcgcagttcgtggcttagttgatgcgtgcgcgtgggccgattgacgggcgcgctcggctccgcctcgcggctaa

SEQ ID NO: 122 - DNA, 307 bp, synthetic construct
  misc_feature 1..307: note = source = /note="Description of Artificial Sequence: Synthetic polynucleotide"
  source 1..307: mol_type = other DNA
tttcgacacctgcgcaccgacttcaaagcaccatcggttgggtgcgaccggccgtcgattgaccggtttcgacaccccgctttgcacagcggcagcagacaaggatggtgcgaccggccgtcgattgaccggtttcgacaccactccgcctcgacggtcttaaccagttctgggtgcgaccggccgtcgattgaccggtttcgacaccctggacgtgtcgggcaatacttctgtcggaggtgcgaccggccgtcgattgaccggtttcgacacctacgccacggcaaagcacctgatcagctcgggt

SEQ ID NO: 123 - DNA, 307 bp, synthetic construct
  misc_feature 1..307: note = source = /note="Description of Artificial Sequence: Synthetic polynucleotide"
  source 1..307: mol_type = other DNA
tgctccactgggcgatgtatgcctgcaccactgggggtgctgcacgcggcggcgcagcgcctacaatggcgccgcggaaggggagcgcggcgcggtcatggaacccacgacgattgagatcaggaacgcgcgggagcataacctgcggtcggtgtcgctttcgttgccgcgggggaagctgattgtcttcaccggggtttcgggcagcgggaagagttcgctggcgtttgacacgctctatgccgaggggcagcggcggtatatcgagtcgttgtcgtcgtatgcccgacagttcatggggcagatg

SEQ ID NO: 124 - DNA, 307 bp, synthetic construct
  misc_feature 1..307: note = source = /note="Description of Artificial Sequence: Synthetic polynucleotide"
  source 1..307: mol_type = other DNA
tatgccgaggggcagcggcggtatatcgagtcgttgtcgtcgtatgcccgacagttcatggggcagatggccaagccggactgcgatcagatcacggggctttcgccatcgatcgcgattcagcagaagacgacgggatggaatccgcggtcgacggtggggacgacgacggcgatctatgactttctgcgcgtgctgtacgcgcggatcgggacgcagcattgcacgcagtgtgggcgggcgatcacggcgcagtctcgcgagcagatcgcggcggggatcttgagcgcgtttaatccgcgccgcg

SEQ ID NO: 125 - DNA, 307 bp, synthetic construct
  misc_feature 1..307: note = source = /note="Description of Artificial Sequence: Synthetic polynucleotide"
  source 1..307: mol_type = other DNA
ggcggtatatcgagtcgttgtcgtcgtatgcccgacagttcatggggcagatggccaagccggactgcgatcagatcacggggctttcgccatcgatcgcgattcagcagaagacgacgggatggaatccgcggtcgacggtggggacgacgacggcgatctatgactttctgcgcgtgctgtacgcgcggatcgggacgcagcattgcacgcagtgtgggcgggcgatcacggcgcagtctcgcgagcagatcgcggcggggatcttgagcgcgtttaatccgcgccgcgatcccacctatcagag

SEQ ID NO: 126 - DNA, 244 bp, synthetic construct
  misc_feature 1..244: note = source = /note="Description of Artificial Sequence: Synthetic polynucleotide"
  source 1..244: mol_type = other DNA
agccgacgagagcgcatttgccctccaaggcttgactctttttccagccgtggctacgctgttaatgcccttcgagtcgcattgtcctgcatacgcccggcggaacttcggtaccaacgggacggaccccggacacgaaaggcaaatgctgtgtttttgtggcctgccggtttggaacgtcacggttttgtgggcggttttttttgacgcaagtccttgtcagaagcgaatttgcagacgagac

SEQ ID NO: 127 - DNA, 173 bp, synthetic construct
  misc_feature 1..173: note = source = /note="Description of Artificial Sequence: Synthetic polynucleotide"
  source 1..173: mol_type = other DNA
ttcgagtcatttgcctggaatacgccctgcggacgttttcgaccaacgggatggacccctggcgctgaaggcagaatcggtttttcccgggcggtttcgggaggcgaacgagcggttttttcgaggtggaactggcgcaagtcgtgtccgttccggggcttgtttgccaagag

SEQ ID NO: 128 - DNA, 252 bp, synthetic construct
  misc_feature 1..252: note = source = /note="Description of Artificial Sequence: Synthetic polynucleotide"
  source 1..252: mol_type = other DNA
aattactctccccaccgattgatagttgaaaaaacttgacagacagcttaatttaagttatgtataatcagttgcttcgtggtccctctggccggaaaaacctctggcggagcgcaagcaccaatagagaggaactcggcccagaagcgaggaaaccgacaattaggggtttaattattgaaggaactttaaagattttgtgacggacggaataaattaagttcaaatcgcgtatgattatatgaatgatga

SEQ ID NO: 129 - DNA, 308 bp, synthetic construct
  misc_feature 1..308: note = source = /note="Description of Artificial Sequence: Synthetic polynucleotide"
  source 1..308: mol_type = other DNA
aaggtcgacaaagatttcggttggttataaatgaacgaggaggggcagatgtggacatctgccgcccacttgagtctggggcgataagctgattgtcgggggaggtttcaccaccgctggggggatcgatgtgaatcgtattgccgcctgggggccgcaataataaattaaacggcgtcaggaagaggttccagatgccgctcaatacccatagcacttagcccttgccttaacaatgaattgtgtctattttgagggctgtcttataaatagaaaagatatcaagataagttgtcgaaaccgcaggg

SEQ ID NO: 130 - DNA, 308 bp, synthetic construct
  misc_feature 1..308: note = source = /note="Description of Artificial Sequence: Synthetic polynucleotide"
  source 1..308: mol_type = other DNA
cctgggggccgcaataataaattaaacggcgtcaggaagaggttccagatgccgctcaatacccatagcacttagcccttgccttaacaatgaattgtgtctattttgagggctgtcttataaatagaaaagatatcaagataagttgtcgaaaccgcaggggtttcgacctaaaagaattatgtctgacgaagaaaaaaaagagaagaccaaatccgagatgccgtttctggatcatatcgaggaactgcgctggcggttgatcaagtccattttatcggttgccgtgatggccatcctggccttta

SEQ ID NO: 131 - DNA, 224 bp, synthetic construct
  misc_feature 1..224: note = source = /note="Description of Artificial Sequence: Synthetic polynucleotide"
  source 1..224: mol_type = other DNA
cgaaatcgagctgccgaccgcaatcccctgacgacccttgacggaactggccgatttgctaaaatatttttgatgcttacttagtcatctggttggcaaacctccgcggaccttcgggaccaatggagaggaacccagccgagaagcatcgagccggtaaatgccggaaatttttgagtcaggagaagctaacttcttcaaactcaggcccttggagccggtca

SEQ ID NO: 132 - DNA, 307 bp, synthetic construct
  misc_feature 1..307: note = source = /note="Description of Artificial Sequence: Synthetic polynucleotide"
  source 1..307: mol_type = other DNA
ctgtcctactgttttgacttaccgatgtgttactccaatacgggcggactgaagtccgccctccaggtgataatgatggagtttacgtctgcgtcacataaggacttgtgacgccaaatagctgtgattctacatcacgacggcagccggaatgccgccgctcccatgttgtaattcactggtgtttgtgattggagctcacgaataagaggtgggagaataaaaaaaactgccacttgcggacaggtggcagaacaaaattctccgaaacgcatgggttaaggctacctgcctgagggaacagaga

SEQ ID NO: 133 - DNA, 178 bp, synthetic construct
  misc_feature 1..178: note = source = /note="Description of Artificial Sequence: Synthetic polynucleotide"
  source 1..178: mol_type = other DNA
agttttgactggaaaatctccctttggacgtttgacctaggggagggaaccccgtctgaagatggataattaccgtcaaaatttggataaacttggagaaaattagacaaaagagtcggtttttttgatgttactgatcggcgtaactgatgtaactgtcgttagatacagcgagtcg

SEQ ID NO: 134 - DNA, 307 bp, synthetic construct
  misc_feature 1..307: note = source = /note="Description of Artificial Sequence: Synthetic polynucleotide"
  source 1..307: mol_type = other DNA
aataagaggaaacaatcctttcaatgtgagtttagtgactgtaattataaaacccatagcgatgagaatgcggcccgtgtcctcggacgggtattttggggtgaaattaccctccccaccgattgagtgttgaaaattcttgacatataggctaatttaagatatgtataattagtcgcttcgtggtccatctggccggaaaaacctctggcgggcgcaagcaccaatagagaggaactcggcccagaagcgaagaaaccaataattaggggtttaattattgaaggaactttaaagattttgcgac

SEQ ID NO: 135-149 - skipped (15 entries)

SEQ ID NO: 150 - RNA, 99 nt, synthetic construct
  variation 37..63: note = a, c, u, g, unknown or other
  misc_feature 1..99: note = source = /note="Description of Artificial Sequence: Synthetic oligonucleotide"
  source 1..99: mol_type = other RNA
gttgtagcgccccctgaattagcggtttccgacaccnnnnnnnnnnnnnnnnnnnnnnnnnnngttgtagcgccccctgaattagcggtttccgacacc

SEQ ID NO: 151 - RNA, 101 nt, synthetic construct
  variation 37..65: note = a, c, u, g, unknown or other
  misc_feature 1..101: note = source = /note="Description of Artificial Sequence: Synthetic polynucleotide"
  source 1..101: mol_type = other RNA
ggtgcggcagccggtagaactgcggcttccgacaccnnnnnnnnnnnnnnnnnnnnnnnnnnnnnggtgcggcagccggtagaactgcggcttccgacacc

SEQ ID NO: 152 - RNA, 101 nt, synthetic construct
  variation 37..65: note = a, c, u, g, unknown or other
  misc_feature 1..101: note = source = /note="Description of Artificial Sequence: Synthetic polynucleotide"
  source 1..101: mol_type = other RNA
ggtgtaagtccgactgaatgcgaggtttccgacaccnnnnnnnnnnnnnnnnnnnnnnnnnnnnnggtgtaagtccgactgaatgcgaggtttccgacacc

SEQ ID NO: 153 - RNA, 121 nt, synthetic construct
  variation 47..75: note = a, c, u, g, unknown or other
  misc_feature 1..121: note = source = /note="Description of Artificial Sequence: Synthetic polynucleotide"
  source 1..121: mol_type = other RNA
ggtttgcaaacagcgagaacatctgtgttttggtggtagttacaacnnnnnnnnnnnnnnnnnnnnnnnnnnnnnggtttgcaaacagcgagaacatctgtgttttggtggtagttacaac

SEQ ID NO: 154 - RNA, 101 nt, synthetic construct
  variation 37..65: note = a, c, u, g, unknown or other
  misc_feature 1..101: note = source = /note="Description of Artificial Sequence: Synthetic polynucleotide"
  source 1..101: mol_type = other RNA
gtttgcaaacagcgagaacatctgtgttttggtggtnnnnnnnnnnnnnnnnnnnnnnnnnnnnngtttgcaaacagcgagaacatctgtgttttggtggt

SEQ ID NO: 155 - RNA, 102 nt, synthetic construct
  variation 37..66: note = a, c, u, g, unknown or other
  misc_feature 1..102: note = source = /note="Description of Artificial Sequence: Synthetic polynucleotide"
  source 1..102: mol_type = other RNA
ggtggaaagggccggagatttaccggctctgacaccnnnnnnnnnnnnnnnnnnnnnnnnnnnnnnggtggaaagggccggagatttaccggctctgacacc

SEQ ID NO: 156 - RNA, 102 nt, synthetic construct; identical to SEQ ID NO: 155
  variation 37..66: note = a, c, u, g, unknown or other
  misc_feature 1..102: note = source = /note="Description of Artificial Sequence: Synthetic polynucleotide"
  source 1..102: mol_type = other RNA

SEQ ID NO: 157 - RNA, 102 nt, synthetic construct
  variation 37..66: note = a, c, u, g, unknown or other
  misc_feature 1..102: note = source = /note="Description of Artificial Sequence: Synthetic polynucleotide"
  source 1..102: mol_type = other RNA
ggtggagccggccgtgaattagggggtttcgacaccnnnnnnnnnnnnnnnnnnnnnnnnnnnnnnggtggagccggccgtgaattagggggtttcgacacc

SEQ ID NO: 158 - RNA, 102 nt, synthetic construct; identical to SEQ ID NO: 157
  variation 37..66: note = a, c, u, g, unknown or other
  misc_feature 1..102: note = source = /note="Description of Artificial Sequence: Synthetic polynucleotide"
  source 1..102: mol_type = other RNA

SEQ ID NO: 159 - RNA, 102 nt, synthetic construct
  variation 37..66: note = a, c, u, g, unknown or other
  misc_feature 1..102: note = source = /note="Description of Artificial Sequence: Synthetic polynucleotide"
  source 1..102: mol_type = other RNA
ggtgttgtgaaccctgaaagaacggattccgacaccnnnnnnnnnnnnnnnnnnnnnnnnnnnnnnggtgttgtgaaccctgaaagaacggattccgacacc

SEQ ID NO: 160 - RNA, 102 nt, synthetic construct
  variation 37..66: note = a, c, u, g, unknown or other
  misc_feature 1..102: note = source = /note="Description of Artificial Sequence: Synthetic polynucleotide"
  source 1..102: mol_type = other RNA
gttgtagcgccccctgaattagcggtttccgacaccnnnnnnnnnnnnnnnnnnnnnnnnnnnnnngttgtagcgccccctgaattagcggtttccgacacc

SEQ ID NO: 161 - RNA, 102 nt, synthetic construct
  variation 37..66: note = a, c, u, g, unknown or other
  misc_feature 1..102: note = source = /note="Description of Artificial Sequence: Synthetic polynucleotide"
  source 1..102: mol_type = other RNA
gttgtagcggcccctgaattagcggtttccgacaccnnnnnnnnnnnnnnnnnnnnnnnnnnnnnngttgtagcggcccctgaattagcggtttccgacacc

SEQ ID NO: 162 - RNA, 102 nt, synthetic construct
  variation 37..66: note = a, c, u, g, unknown or other
  misc_feature 1..102: note = source = /note="Description of Artificial Sequence: Synthetic polynucleotide"
  source 1..102: mol_type = other RNA
gttgtgaccgcccccggaatcacggtttgcgacaccnnnnnnnnnnnnnnnnnnnnnnnnnnnnnngttgtgaccgcccccggaatcacggtttgcgacacc

SEQ ID NO: 163 - RNA, 102 nt, synthetic construct; identical to SEQ ID NO: 162
  variation 37..66: note = a, c, u, g, unknown or other
  misc_feature 1..102: note = source = /note="Description of Artificial Sequence: Synthetic polynucleotide"
  source 1..102: mol_type = other RNA

SEQ ID NO: 164 - RNA, 104 nt, synthetic construct
  variation 37..68: note = a, c, u, g, unknown or other
  misc_feature 1..104: note = source = /note="Description of Artificial Sequence: Synthetic polynucleotide"
  source 1..104: mol_type = other RNA
ggtgtagcgggcctcaaaaacacggcatccgacaccnnnnnnnnnnnnnnnnnnnnnnnnnnnnnnnnggtgtagcgggcctcaaaaacacggcatccgacacc

SEQ ID NO: 165 - RNA, 104 nt, synthetic construct; identical to SEQ ID NO: 164
  variation 37..68: note = a, c, u, g, unknown or other
  misc_feature 1..104: note = source = /note="Description of Artificial Sequence: Synthetic polynucleotide"
  source 1..104: mol_type = other RNA

SEQ ID NO: 166 - RNA, 110 nt, synthetic construct
  variation 37..74: note = a, c, u, g, unknown or other
  misc_feature 1..110: note = source = /note="Description of Artificial Sequence: Synthetic polynucleotide"
  source 1..110: mol_type = other RNA
gctgtgactcaccggcatttgcggggttcttacaccnnnnnnnnnnnnnnnnnnnnnnnnnnnnnnnnnnnnnngctgtgactcaccggcatttgcggggttcttacacc

SEQ ID NO: 167 - RNA, 110 nt, synthetic construct; identical to SEQ ID NO: 166
  variation 37..74: note = a, c, u, g, unknown or other
  misc_feature 1..110: note = source = /note="Description of Artificial Sequence: Synthetic polynucleotide"
  source 1..110: mol_type = other RNA

SEQ ID NO: 168-199 - skipped (32 entries)

SEQ ID NO: 200 - DNA, 15 bp, synthetic construct
  variation 5: note = a, c, u, g, unknown or other
  misc_feature 1..15: note = source = /note="Description of Artificial Sequence: Synthetic oligonucleotide"
  misc_feature 1..15: note = source = /note="Description of Combined DNA/RNA Molecule: Synthetic oligonucleotide"
  source 1..15: mol_type = other DNA
rvsgnhtbbgacacc

SEQ ID NO: 201 - DNA, 15 bp, synthetic construct
  misc_feature 1..15: note = source = /note="Description of Artificial Sequence: Synthetic oligonucleotide"
  misc_feature 1..15: note = source = /note="Description of Combined DNA/RNA Molecule: Synthetic oligonucleotide"
  source 1..15: mol_type = other DNA
sgkggtwsttacamc

SEQ ID NO: 202 - RNA, 18 nt, synthetic construct
  misc_feature 1..18: note = source = /note="Description of Artificial Sequence: Synthetic oligonucleotide"
  source 1..18: mol_type = other RNA
tttaccggctctgacacc

SEQ ID NO: 203-209 - skipped (7 entries)

SEQ ID NO: 210 - protein, 5 aa, synthetic construct
  VARIANT 1: note = /replace="Gly" or "Ser" or Thr"
  VARIANT 2: note = /replace="Phe" or "Ile" or "Leu" or "Met" or "Pro" or "Val" "Trp" or "Tyr"
  VARIANT 4: note = /replace="Phe" or "Ile" or "Leu" or "Met" or "Pro" or "Val" "Trp" or "Tyr"
  VARIANT 5: note = /replace="Gly" or "Ser" or Thr"
  SITE 1..5: note = /note="Variant residues given in the sequence have no preference with respect to those in the annotations for variant positions"
  REGION 1..5: note = source = /note="Description of Artificial Sequence: Synthetic peptide"
ACDCA

SEQ ID NO: 211 - protein, 5 aa, synthetic construct
  VARIANT 1..4: note = /replace="Phe" or "Ile" or "Leu" or "Met" or "Pro" or "Val" "Trp" or "Tyr"
  SITE 1..5: note = /note="Variant residues given in the sequence have no preference with respect to those in the annotations for variant positions"
  REGION 1..5: note = source = /note="Description of Artificial Sequence: Synthetic peptide"
CCCCE

SEQ ID NO: 212 - protein, 5 aa, synthetic construct
  MOD_RES 2: note = Any amino acid
  REGION 1..5: note = source = /note="Description of Artificial Sequence: Synthetic peptide"
DXNAA

SEQ ID NO: 213-249 - skipped (37 entries)

SEQ ID NO: 250 - RNA, 99 nt, synthetic construct
  misc_feature 1..99: note = source = /note="Description of Artificial Sequence: Synthetic oligonucleotide"
  source 1..99: mol_type = other RNA
gatgcttacttagtcatctggttggcaaacctccgcggaccttcgggaccaatggagaggaacccagccgagaagcatcgagccggtaaatgccggaaa

SEQ ID NO: 251-299 - skipped (49 entries)

SEQ ID NO: 300 - protein, 7 aa, Simian virus 40
PKKKRKV

SEQ ID NO: 301 - protein, 16 aa, unidentified
  REGION 1..16: note = source = /note="Description of Unknown: Nucleoplasmin bipartite NLS sequence"
KRPAATKKAGQAKKKK

SEQ ID NO: 302 - protein, 9 aa, unidentified
  REGION 1..9: note = source = /note="Description of Unknown: c-Myc NLS sequence"
PAAKRVKLD

SEQ ID NO: 303 - protein, 11 aa, unidentified
  REGION 1..11: note = source = /note="Description of Unknown: c-Myc NLS sequence"
RQRRNELKRSP

SEQ ID NO: 304 - protein, 38 aa, Homo sapiens
NQSSNFGPMKGGNFGGRSSGPYGGGGQYFAKPRNQGGY

SEQ ID NO: 305 - protein, 42 aa, unidentified
  REGION 1..42: note = source = /note="Description of Unknown: IBB domain from importin-alpha sequence"
RMRIZFKNKGKDTAELRRRRVEVSVELRKAKKDEQILKRRNV

SEQ ID NO: 306 - protein, 8 aa, unidentified
  REGION 1..8: note = source = /note="Description of Unknown: Myoma T protein sequence"
VSRKRPRP

SEQ ID NO: 307 - protein, 8 aa, unidentified
  REGION 1..8: note = source = /note="Description of Unknown: Myoma T protein sequence"
PPKKARED

SEQ ID NO: 308 - protein, 8 aa, Homo sapiens
PQPKKKPL

SEQ ID NO: 309 - protein, 12 aa, Mus sp.
SALIKKKKKMAP

SEQ ID NO: 310 - protein, 5 aa, Influenza virus
DRLRR

SEQ ID NO: 311 - protein, 7 aa, Influenza virus
PKQKKRK

SEQ ID NO: 312 - protein, 10 aa, Mus sp.
RKLKKKIKKL

SEQ ID NO: 313 - protein, 10 aa, Mus sp.
REKKKFLKRR

SEQ ID NO: 314 - protein, 20 aa, Homo sapiens
KRKGDEVDGVDEVAKKKSKK

SEQ ID NO: 315 - protein, 17 aa, Homo sapiens
RKCLQAGMNLEARKTKK

SEQ ID NO: 316 - protein, 130 aa, synthetic construct
  REGION 1..130: note = source = /note="Description of Artificial Sequence: Synthetic polypeptide"
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 317 - protein, 133 aa, synthetic construct
  REGION 1..133: note = source = /note="Description of Artificial Sequence: Synthetic polypeptide"
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 318 - protein, 128 aa, synthetic construct
  REGION 1..128: note = source = /note="Description of Artificial Sequence: Synthetic polypeptide"
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVVQATSEDLVVNLVPLGR

SEQ ID NO: 319-399 - skipped (81 entries)

SEQ ID NO: 400 - RNA, 171 nt, synthetic construct
  misc_feature 1..171: note = source = /note="Description of Artificial Sequence: Synthetic polynucleotide"
  source 1..171: mol_type = other RNA
gggggtggaaagggccggagatttaccggctctgacaccccattcatccgcttattatcacttattcagggtggaaagggccggagatttaccggctctgacaccgcttggaggagcgcagtcaccaaaacttgtggtggaaagggccggagatttaccggctctgacacc

SEQ ID NO: 401 - RNA, 36 nt, synthetic construct
  misc_feature 1..36: note = source = /note="Description of Artificial Sequence: Synthetic oligonucleotide"
  source 1..36: mol_type = other RNA
ggtggaaagggccggagatttaccggctctgacacc

SEQ ID NO: 402 - RNA, 30 nt, synthetic construct
  misc_feature 1..30: note = source = /note="Description of Artificial Sequence: Synthetic oligonucleotide"
  source 1..30: mol_type = other RNA
ccattcatccgcttattatcacttattcag

SEQ ID NO: 403 - RNA, 30 nt, synthetic construct
  misc_feature 1..30: note = source = /note="Description of Artificial Sequence: Synthetic oligonucleotide"
  source 1..30: mol_type = other RNA
gcttggaggagcgcagtcaccaaaacttgt

SEQ ID NO: 404 - RNA, 107 nt, synthetic construct
  misc_feature 1..107: note = source = /note="Description of Artificial Sequence: Synthetic polynucleotide"
  source 1..107: mol_type = other RNA
ggggatgcttacttagtcatctggttggcaaacctccgcggaccttcgggaccaatggagaggaacccagccgagaagcatcgagccggtaaatgccggaaattttt

SEQ ID NO: 405 - RNA, 102 nt, synthetic construct
  misc_feature 1..102: note = source = /note="Description of Artificial Sequence: Synthetic polynucleotide"
  source 1..102: mol_type = other RNA
ggggatgcttacttagtcatctggttggcaaacctccgcggaccttcgggaccaatggagaggaacccagccgagaagcatcgagccggtaaatgccggaaa

SEQ ID NO: 406 - RNA, 94 nt, synthetic construct
  misc_feature 1..94: note = source = /note="Description of Artificial Sequence: Synthetic oligonucleotide"
  source 1..94: mol_type = other RNA
ggggatgcttacttagtcatctggttggcaaacctccgcggaccttcgggaccaatggagaggaacccagccgagaagcatcgagccggtaaat

SEQ ID NO: 407 - RNA, 45 nt, synthetic construct
  misc_feature 1..45: note = source = /note="Description of Artificial Sequence: Synthetic oligonucleotide"
  source 1..45: mol_type = other RNA
gggtttaccggctctgacaccgcttggaggagcgcagtcaccaaa

SEQ ID NO: 408 - RNA, 18 nt, synthetic construct
  misc_feature 1..18: note = source = /note="Description of Artificial Sequence: Synthetic oligonucleotide"
  source 1..18: mol_type = other RNA
tttaccggctctgacacc

SEQ ID NO: 409 - RNA, 24 nt, synthetic construct
  misc_feature 1..24: note = source = /note="Description of Artificial Sequence: Synthetic oligonucleotide"
  source 1..24: mol_type = other RNA
gcttggaggagcgcagtcaccaaa

SEQ ID NO: 410 - RNA, 45 nt, synthetic construct
  misc_feature 1..45: note = source = /note="Description of Artificial Sequence: Synthetic oligonucleotide"
  source 1..45: mol_type = other RNA
gggtttaccggctctgacaccaactgtgataaactaccgcattaa

SEQ ID NO: 411 - RNA, 18 nt, synthetic construct
  misc_feature 1..18: note = source = /note="Description of Artificial Sequence: Synthetic oligonucleotide"
  source 1..18: mol_type = other RNA
tttaccggctctgacacc

SEQ ID NO: 412 - RNA, 24 nt, synthetic construct
  misc_feature 1..24: note = source = /note="Description of Artificial Sequence: Synthetic oligonucleotide"
  source 1..24: mol_type = other RNA
aactgtgataaactaccgcattaa

SEQ ID NO: 413 - RNA, 45 nt, synthetic construct
  misc_feature 1..45: note = source = /note="Description of Artificial Sequence: Synthetic oligonucleotide"
  source 1..45: mol_type = other RNA
gggtttaccggctctgacacccttccttagctcctgaaaatctcg

SEQ ID NO: 414 - RNA, 18 nt, synthetic construct
  misc_feature 1..18: note = source = /note="Description of Artificial Sequence: Synthetic oligonucleotide"
  source 1..18: mol_type = other RNA
tttaccggctctgacacc

SEQ ID NO: 415 - RNA, 24 nt, synthetic construct
  misc_feature 1..24: note = source = /note="Description of Artificial Sequence: Synthetic oligonucleotide"
  source 1..24: mol_type = other RNA
cttccttagctcctgaaaatctcg

SEQ ID NO: 416 - RNA, 45 nt, synthetic construct
  misc_feature 1..45: note = source = /note="Description of Artificial Sequence: Synthetic oligonucleotide"
  source 1..45: mol_type = other RNA
gggtttaccggctctgacacccatcttgcgaatatatgtgtagaa

SEQ ID NO: 417 - RNA, 18 nt, synthetic construct
  misc_feature 1..18: note = source = /note="Description of Artificial Sequence: Synthetic oligonucleotide"
  source 1..18: mol_type = other RNA
tttaccggctctgacacc

SEQ ID NO: 418 - RNA, 24 nt, synthetic construct
  misc_feature 1..24: note = source = /note="Description of Artificial Sequence: Synthetic oligonucleotide"
  source 1..24: mol_type = other RNA
catcttgcgaatatatgtgtagaa

SEQ ID NO: 419 - RNA, 130 nt, synthetic construct
  misc_feature 1..130: note = source = /note="Description of Artificial Sequence: Synthetic polynucleotide"
  source 1..130: mol_type = other RNA
caacgaggtatttatccgattaattctcatgtttgacagcttatcatcgataagctttaatgcggtagtttatcacagttaaattgctaacgcagtcaggcaccgtgtatttgtagcccggggactgttt

SEQ ID NO: 420 - RNA, 130 nt, synthetic construct
  misc_feature 1..130: note = source = /note="Description of Artificial Sequence: Synthetic polynucleotide"
  source 1..130: mol_type = other RNA
caacgaggtatttatccgataaataagatcactaccgggcgtattttttgagttatcgagattttcaggagctaaggaagctaaaatggagaaaaaaatcactggatatattgtagcccggggactgttt

SEQ ID NO: 421 - RNA, 130 nt, synthetic construct; identical to SEQ ID NO: 419
  misc_feature 1..130: note = source = /note="Description of Artificial Sequence: Synthetic polynucleotide"
  source 1..130: mol_type = other RNA

SEQ ID NO: 422 - DNA, 130 bp, synthetic construct
  misc_feature 1..130: note = source = /note="Description of Artificial Sequence: Synthetic polynucleotide"
  source 1..130: mol_type = other DNA
caacgaggtatttatccgattaattctcatgtttgacagcttatcatcgataagctttaatgcggtagtttatcacagttaaattgctaacgcagtcaggcaccgtgtatttgtagcccggggactgttt

SEQ ID NO: 423 - RNA, 130 nt, synthetic construct
  misc_feature 1..130: note = source = /note="Description of Artificial Sequence: Synthetic polynucleotide"
  source 1..130: mol_type = other RNA
aaacagtccccgggctacaaatacacggtgcctgactgcgttagcaatttaactgtgataaactaccgcattaaagcttatcgatgataagctgtcaaacatgagaattaatcggataaatacctcgttg

SEQ ID NO: 424 - DNA, 130 bp, synthetic construct
  misc_feature 1..130: note = source = /note="Description of Artificial Sequence: Synthetic polynucleotide"
  source 1..130: mol_type = other DNA
aaacagtccccgggctacaaatacacggtgcctgactgcgttagcaatttaactgtgataaactaccgcattaaagcttatcgatgataagctgtcaaacatgagaattaatcggataaatacctcgttg

SEQ ID NO: 425 - DNA, 130 bp, synthetic construct
  misc_feature 1..130: note = source = /note="Description of Artificial Sequence: Synthetic polynucleotide"
  source 1..130: mol_type = other DNA
caacgaggtatttatccgatcgctctggagtgaataccacgacgatttccggcagtttctacacatatattcgcaagatgtggcgtgttacggtgaaaacctggcctattttgtagcccggggactgttt

SEQ ID NO: 426 - RNA, 130 nt, synthetic construct; identical to SEQ ID NO: 423
  misc_feature 1..130: note = source = /note="Description of Artificial Sequence: Synthetic polynucleotide"
  source 1..130: mol_type = other RNA

SEQ ID NO: 427 - RNA, 130 nt, synthetic construct
  misc_feature 1..130: note = source = /note="Description of Artificial Sequence: Synthetic polynucleotide"
  source 1..130: mol_type = other RNA
aaacagtccccgggctacaatatatccagtgatttttttctccattttagcttccttagctcctgaaaatctcgataactcaaaaaatacgcccggtagtgatcttatttatcggataaatacctcgttg

SEQ ID NO: 428 - RNA, 130 nt, synthetic construct
  misc_feature 1..130: note = source = /note="Description of Artificial Sequence: Synthetic polynucleotide"
  source 1..130: mol_type = other RNA
aaacagtccccgggctacaatctcgccgaaacgtttggtggcgggaccagtgacgaaggcttgagcgagggcgtgcaagattccgaataccgcaagcgacaggccgatcaatcggataaatacctcgttg

SEQ ID NO: 429 - DNA, 130 bp, synthetic construct
  misc_feature 1..130: note = source = /note="Description of Artificial Sequence: Synthetic polynucleotide"
  source 1..130: mol_type = other DNA
aaacagtccccgggctacaaaataggccaggttttcaccgtaacacgccacatcttgcgaatatatgtgtagaaactgccggaaatcgtcgtggtattcactccagagcgatcggataaatacctcgttg

SEQ ID NO: 430 - DNA, 188 bp, synthetic construct
  misc_feature 1..188: note = source = /note="Description of Artificial Sequence: Synthetic polynucleotide"
  source 1..188: mol_type = other DNA
ggtgtcagagccggtaaatctccggccctttccaccacaagttttggtgactgcgctcctccaagcggtgtcagagccggtaaatctccggccctttccaccctgaataagtgataataagcggatgaatggggtgtcagagccggtaaatctccggccctttccaccccctatagtgagtcgtatta

SEQ ID NO: 431 - DNA, 18 bp, synthetic construct
  misc_feature 1..18: note = source = /note="Description of Artificial Sequence: Synthetic oligonucleotide"
  source 1..18: mol_type = other DNA
taatacgactcactatag

SEQ ID NO: 432 - DNA, 66 bp, synthetic construct
  misc_feature 1..66: note = source = /note="Description of Artificial Sequence: Synthetic oligonucleotide"
  source 1..66: mol_type = other DNA
ggtgtcagagccggtaaatctccggccctttccaccacaagttttggtgactgcgctcctccaagc

SEQ ID NO: 433 - DNA, 124 bp, synthetic construct
  misc_feature 1..124: note = source = /note="Description of Artificial Sequence: Synthetic polynucleotide"
  source 1..124: mol_type = other DNA
ttaatgcggtagtttatcacagttggtgtcagagccggtaaaccctatagtgagtcgtattattaatgcggtagtttatcacagttggtgtcagagccggtaaaccctatagtgagtcgtatta

SEQ ID NO: 434 - DNA, 36 bp, synthetic construct
  misc_feature 1..36: note = source = /note="Description of Artificial Sequence: Synthetic oligonucleotide"
  source 1..36: mol_type = other DNA
taatacgactcactatagtaatacgactcactatag

SEQ ID NO: 435 - DNA, 124 bp, synthetic construct
  misc_feature 1..124: note = source = /note="Description of Artificial Sequence: Synthetic polynucleotide"
  source 1..124: mol_type = other DNA
cgagattttcaggagctaaggaagggtgtcagagccggtaaaccctatagtgagtcgtattacgagattttcaggagctaaggaagggtgtcagagccggtaaaccctatagtgagtcgtatta

SEQ ID NO: 436 - DNA, 36 bp, synthetic construct; identical to SEQ ID NO: 434
  misc_feature 1..36: note = source = /note="Description of Artificial Sequence: Synthetic oligonucleotide"
  source 1..36: mol_type = other DNA

SEQ ID NO: 437 - DNA, 124 bp, synthetic construct
  misc_feature 1..124: note = source = /note="Description of Artificial Sequence: Synthetic polynucleotide"
  source 1..124: mol_type = other DNA
ttctacacatatattcgcaagatgggtgtcagagccggtaaaccctatagtgagtcgtattattctacacatatattcgcaagatgggtgtcagagccggtaaaccctatagtgagtcgtatta

SEQ ID NO: 438 - DNA, 36 bp, synthetic construct; identical to SEQ ID NO: 434
  misc_feature 1..36: note = source = /note="Description of Artificial Sequence: Synthetic oligonucleotide"
  source 1..36: mol_type = other DNA

SEQ ID NO: 439 - DNA, 248 bp, synthetic construct
  misc_feature 1..248: note = source = /note="Description of Artificial Sequence: Synthetic polynucleotide"
  source 1..248: mol_type = other DNA
aaaaatttccggcatttaccggctcgatgcttctcggctgggttcctctccattggtcccgaaggtccgcggaggtttgccaaccagatgactaagtaagcatcccctatagtgagtcgtattaaaaaatttccggcatttaccggctcgatgcttctcggctgggttcctctccattggtcccgaaggtccgcggaggtttgccaaccagatgactaagtaagcatcccctatagtgagtcgtatta

SEQ ID NO: 440 - DNA, 36 bp, synthetic construct; identical to SEQ ID NO: 434
  misc_feature 1..36: note = source = /note="Description of Artificial Sequence: Synthetic oligonucleotide"
  source 1..36: mol_type = other DNA

SEQ ID NO: 441 - DNA, 238 bp, synthetic construct
  misc_feature 1..238: note = source = /note="Description of Artificial Sequence: Synthetic polynucleotide"
  source 1..238: mol_type = other DNA
tttccggcatttaccggctcgatgcttctcggctgggttcctctccattggtcccgaaggtccgcggaggtttgccaaccagatgactaagtaagcatcccctatagtgagtcgtattatttccggcatttaccggctcgatgcttctcggctgggttcctctccattggtcccgaaggtccgcggaggtttgccaaccagatgactaagtaagcatcccctatagtgagtcgtatta

SEQ ID NO: 442 - DNA, 36 bp, synthetic construct; identical to SEQ ID NO: 434
  misc_feature 1..36: note = source = /note="Description of Artificial Sequence: Synthetic oligonucleotide"
  source 1..36: mol_type = other DNA

SEQ ID NO: 443 - DNA, 222 bp, synthetic construct
  misc_feature 1..222: note = source = /note="Description of Artificial Sequence: Synthetic polynucleotide"
  source 1..222: mol_type = other DNA
atttaccggctcgatgcttctcggctgggttcctctccattggtcccgaaggtccgcggaggtttgccaaccagatgactaagtaagcatcccctatagtgagtcgtattaatttaccggctcgatgcttctcggctgggttcctctccattggtcccgaaggtccgcggaggtttgccaaccagatgactaagtaagcatcccctatagtgagtcgtatta

SEQ ID NO: 444 - DNA, 36 bp, synthetic construct; identical to SEQ ID NO: 434
  misc_feature 1..36: note = source = /note="Description of Artificial Sequence: Synthetic oligonucleotide"
  source 1..36: mol_type = other DNA

SEQ ID NO: 445 - DNA, 130 bp, synthetic construct
  misc_feature 1..130: note = source = /note="Description of Artificial Sequence: Synthetic polynucleotide"
  source 1..130: mol_type = other DNA
aaacagtccccgggctacaaagctaccaactctttgaaccgaggtaactggcttggaggagcgcagtcaccaaaacttgtcctttcagtttagccttaaccggcgcatgaatcggataaatacctcgttg

SEQ ID NO: 446 - DNA, 20 bp, synthetic construct
  misc_feature 1..20: note = source = /note="Description of Artificial Sequence: Synthetic oligonucleotide"
  source 1..20: mol_type = other DNA
aaacagtccccgggctacaa

SEQ ID NO: 447 - DNA, 63 bp, synthetic construct
  misc_feature 1..63: note = source = /note="Description of Artificial Sequence: Synthetic oligonucleotide"
  source 1..63: mol_type = other DNA
aaatgtaataaggatccctcgagtaatacgactcactatagggcaacgaggtatttatccgat

SEQ ID NO: 448 - DNA, 63 bp, synthetic construct
  misc_feature 1..63: note = source = /note="Description of Artificial Sequence: Synthetic oligonucleotide"
  source 1..63: mol_type = other DNA
aaatgtaataaggatccctcgagtaatacgactcactatagggaaacagtccccgggctacaa

SEQ ID NO: 449 - DNA, 20 bp, synthetic construct
  misc_feature 1..20: note = source = /note="Description of Artificial Sequence: Synthetic oligonucleotide"
  source 1..20: mol_type = other DNA
caacgaggtatttatccgat

SEQ ID NO: 450 - DNA, 130 bp, synthetic construct; identical to SEQ ID NO: 424
  misc_feature 1..130: note = source = /note="Description of Artificial Sequence: Synthetic polynucleotide"
  source 1..130: mol_type = other DNA

SEQ ID NO: 451 - DNA, 20 bp, synthetic construct
  misc_feature 1..20: note = source = /note="Description of Artificial Sequence: Synthetic oligonucleotide"
  source 1..20: mol_type = other DNA
aaacagtccccgggctacaa

SEQ ID NO: 452 - DNA, 63 bp, synthetic construct; identical to SEQ ID NO: 447
  misc_feature 1..63: note = source = /note="Description of Artificial Sequence: Synthetic oligonucleotide"
  source 1..63: mol_type = other DNA

SEQ ID NO: 453 - DNA, 63 bp, synthetic construct; identical to SEQ ID NO: 448
  misc_feature 1..63: note = source = /note="Description of Artificial Sequence: Synthetic oligonucleotide"
  source 1..63: mol_type = other DNA

SEQ ID NO: 454 - DNA, 20 bp, synthetic construct
  misc_feature 1..20: note = source = /note="Description of Artificial Sequence: Synthetic oligonucleotide"
  source 1..20: mol_type = other DNA
caacgaggtatttatccgat

SEQ ID NO: 455 - DNA, 130 bp, synthetic construct
  misc_feature 1..130: note = source = /note="Description of Artificial Sequence: Synthetic polynucleotide"
  source 1..130: mol_type = other DNA
aaacagtccccgggctacaatatatccagtgatttttttctccattttagcttccttagctcctgaaaatctcgataactcaaaaaatacgcccggtagtgatcttatttatcggataaatacctcgttg

SEQ ID NO: 456 - DNA, 20 bp, synthetic construct
  misc_feature 1..20: note = source = /note="Description of Artificial Sequence: Synthetic oligonucleotide"
  source 1..20: mol_type = other DNA
aaacagtccccgggctacaa

SEQ ID NO: 457 - DNA, 63 bp, synthetic construct; identical to SEQ ID NO: 447
  misc_feature 1..63: note = source = /note="Description of Artificial Sequence: Synthetic oligonucleotide"
  source 1..63: mol_type = other DNA

SEQ ID NO: 458 - DNA, 63 bp, synthetic construct; identical to SEQ ID NO: 448
  misc_feature 1..63: note = source = /note="Description of Artificial Sequence: Synthetic oligonucleotide"
  source 1..63: mol_type = other DNA

SEQ ID NO: 459 - DNA, 20 bp, synthetic construct
  misc_feature 1..20: note = source = /note="Description of Artificial Sequence: Synthetic oligonucleotide"
  source 1..20: mol_type = other DNA
caacgaggtatttatccgat

SEQ ID NO: 460 - DNA, 130 bp, synthetic construct; identical to SEQ ID NO: 429
  misc_feature 1..130: note = source = /note="Description of Artificial Sequence: Synthetic polynucleotide"
  source 1..130: mol_type = other DNA

SEQ ID NO: 461 - DNA, 20 bp, synthetic construct
  misc_feature 1..20: note = source = /note="Description of Artificial Sequence: Synthetic oligonucleotide"
  source 1..20: mol_type = other DNA
aaacagtccccgggctacaa

SEQ ID NO: 462 - DNA, 63 bp, synthetic construct; identical to SEQ ID NO: 447
  misc_feature 1..63: note = source = /note="Description of Artificial Sequence: Synthetic oligonucleotide"
  source 1..63: mol_type = other DNA

SEQ ID NO: 463 - DNA, 63 bp, synthetic construct; identical to SEQ ID NO: 448
  misc_feature 1..63: note = source = /note="Description of Artificial Sequence: Synthetic oligonucleotide"
  source 1..63: mol_type = other DNA

SEQ ID NO: 464 - DNA, 20 bp, synthetic construct
  misc_feature 1..20: note = source = /note="Description of Artificial Sequence: Synthetic oligonucleotide"
  source 1..20: mol_type = other DNA
caacgaggtatttatccgat

SEQ ID NO: 465 - DNA, 130 bp, synthetic construct
  misc_feature 1..130: note = source = /note="Description of Artificial Sequence: Synthetic polynucleotide"
  source 1..130: mol_type = other DNA
aaacagtccccgggctacaatctcgccgaaacgtttggtggcgggaccagtgacgaaggcttgagcgagggcgtgcaagattccgaataccgcaagcgacaggccgatcaatcggataaatacctcgttg

SEQ ID NO: 466 - DNA, 20 bp, synthetic construct
  misc_feature 1..20: note = source = /note="Description of Artificial Sequence: Synthetic oligonucleotide"
  source 1..20: mol_type = other DNA
aaacagtccccgggctacaa

SEQ ID NO: 467 - DNA, 63 bp, synthetic construct; identical to SEQ ID NO: 447
  misc_feature 1..63: note = source = /note="Description of Artificial Sequence: Synthetic oligonucleotide"
  source 1..63: mol_type = other DNA

SEQ ID NO: 468 - DNA, 63 bp, synthetic construct; identical to SEQ ID NO: 448
  misc_feature 1..63: note = source = /note="Description of Artificial Sequence: Synthetic oligonucleotide"
  source 1..63: mol_type = other DNA

SEQ ID NO: 469 - DNA, 20 bp, synthetic construct
  misc_feature 1..20: note = source = /note="Description of Artificial Sequence: Synthetic oligonucleotide"
  source 1..20: mol_type = other DNA
caacgaggtatttatccgat

SEQ ID NO: 470 - RNA, 36 nt, synthetic construct
  misc_feature 1..36: note = source = /note="Description of Artificial Sequence: Synthetic oligonucleotide"
  source 1..36: mol_type = other RNA
gttgtagcggcccctgaattagcggtttccgacacc

SEQ ID NO: 471 - RNA, 36 nt, synthetic construct
  misc_feature 1..36: note = source = /note="Description of Artificial Sequence: Synthetic oligonucleotide"
  source 1..36: mol_type = other RNA
ggtgcggcagccggtagaactgcggcttccgacacc

SEQ ID NO: 472 - RNA, 36 nt, synthetic construct
  misc_feature 1..36: note = source = /note="Description of Artificial Sequence: Synthetic oligonucleotide"
  source 1..36: mol_type = other RNA
gttggagtcagcaaggaatcaacggttttggacacc

SEQ ID NO: 473 - RNA, 34 nt, synthetic construct
  misc_feature 1..34: note = source = /note="Description of Artificial Sequence: Synthetic oligonucleotide"
  source 1..34: mol_type = other RNA
ggtgtagcgggcctcaaaaacacggcatccgaca

SEQ ID NO: 474 - RNA, 28 nt, synthetic construct
  misc_feature 1..28: note = source = /note="Description of Artificial Sequence: Synthetic oligonucleotide"
  source 1..28: mol_type = other RNA
gctgtgactcaccggcatttgcggggtt

SEQ ID NO: 475 - DNA, 102 bp, synthetic construct
  variation 37..66: note = a, c, u, g, unknown or other
  misc_feature 1..102: note = source = /note="Description of Artificial Sequence: Synthetic polynucleotide"
  source 1..102: mol_type = other DNA
ggtggaaagggccggagatttaccggctctgacaccnnnnnnnnnnnnnnnnnnnnnnnnnnnnnnggtggaaagggccggagatttaccggctctgacacc

SEQ ID NO: 476 - RNA, 39 nt, synthetic construct
  variation 19..39: note = a, c, u, g, unknown or other
  misc_feature 1..39: note = source = /note="Description of Artificial Sequence: Synthetic oligonucleotide"
  source 1..39: mol_type = other RNA
tttaccggctctgacaccnnnnnnnnnnnnnnnnnnnnn

SEQ ID NO: 477 - DNA, 36 bp, synthetic construct
  misc_feature 1..36: note = source = /note="Description of Artificial Sequence: Synthetic oligonucleotide"
  source 1..36: mol_type = other DNA
cgataagctttaatgcggtagtttatcacagttaaa

SEQ ID NO: 478 - RNA, 36 nt, synthetic construct
  misc_feature 1..36: note = source = /note="Description of Artificial Sequence: Synthetic oligonucleotide"
  source 1..36: mol_type = other RNA
cgataagctttaatgcggtagtttatcacagttaaa

SEQ ID NO: 479 - RNA, 36 nt, synthetic construct
  misc_feature 1..36: note = source = /note="Description of Artificial Sequence: Synthetic oligonucleotide"
  source 1..36: mol_type = other RNA
tttaactgtgataaactaccgcattaaagcttatcg

SEQ ID NO: 480 - DNA, 36 bp, synthetic construct
  misc_feature 1..36: note = source = /note="Description of Artificial Sequence: Synthetic oligonucleotide"
  source 1..36: mol_type = other DNA
tttaactgtgataaactaccgcattaaagcttatcg

SEQ ID NO: 481 - protein, 5 aa, synthetic construct
  REGION 1..5: note = source = /note="Description of Artificial Sequence: Synthetic peptide"
GGGGS

SEQ ID NO: 482 - protein, 4 aa, unidentified
  REGION 1..4: note = source = /note="Description of Unknown: KDEL retention sequence"
KDEL

SEQ ID NO: 483 - protein, 5 aa, synthetic construct
  REGION 1..5: note = source = /note="Description of Artificial Sequence: Synthetic peptide"
EAAAK

SEQ ID NO: 484 - protein, 7 aa, synthetic construct
  REGION 1..7: note = source = /note="Description of Artificial Sequence: Synthetic peptide"
GSGGGGS

SEQ ID NO: 485 - protein, 15 aa, synthetic construct
  REGION 1..15: note = source = /note="Description of Artificial Sequence: Synthetic peptide"
GGGGSGGGGSGGGGS

SEQ ID NO: 486 - protein, 18 aa, synthetic construct
  REGION 1..18: note = source = /note="Description of Artificial Sequence: Synthetic peptide"
GGSGGSGGSGGSGGSGGS

SEQ ID NO: 487 - protein, 336 aa, unidentified
  REGION 1..336: note = source = /note="Description of Unknown: ADAR1DD-WT sequence"
SLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNF

SEQ ID NO: 488 - protein, 336 aa, synthetic construct
  REGION 1..336: note = source = /note="Description of Artificial Sequence: Synthetic polypeptide"
SLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGQGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNF

SEQ ID NO: 489 - protein, 385 aa, unidentified
  REGION 1..385: note = source = /note="Description of Unknown: ADAR2DD-WT sequence"
QLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 490 - protein, 385 aa, synthetic construct
  REGION 1..385: note = source = /note="Description of Artificial Sequence: Synthetic polypeptide"
QLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 491 - protein, 198 aa, synthetic construct [sequence omitted]

SEQ ID NO: 492 - protein, 208 aa, synthetic construct
  REGION 1..208: note = source = /note="Description of Artificial Sequence: Synthetic polypeptide"
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 493 - protein, 229 aa, synthetic construct [sequence omitted]